(19) United States
(12) Patent Application Publication
(10) Pub. No.: US 2025/0205169 A1
(43) Pub. Date: Jun. 26, 2025

(54) IONIZABLE CATIONIC LIPIDS AND LIPID NANOPARTICLES, AND METHODS OF SYNTHESIS AND USE THEREOF

(71) Applicant: Tidal Therapeutics, Inc., Cambridge, MA (US)

(72) Inventor: Mir ALI, Woburn, MA (US)

(21) Appl. No.: 18/979,339

(22) Filed: Dec. 12, 2024

Related U.S. Application Data
(60) Provisional application No. 63/609,852, filed on Dec. 13, 2023.

(51) Int. Cl. A61K 9/51; A61K 47/68; A61K 48/00; A61P 35/00; C07C 235/74

(52) U.S. Cl. CPC A61K 9/5123; A61K 47/6849; A61K 48/0033; A61P 35/00; C07C 235/74

(57) ABSTRACT

Provided are ionizable cationic lipids and lipid nanoparticles for the delivery of nucleic acids to cells (e.g., immune cells), and methods of making and using such lipids and targeted lipid nanoparticles.

### CROSS-REFERENCE TO RELATED APPLICATION

This application claims priority to and benefit of U.S. Provisional Application No. 63/609,852, filed Dec. 13, 2023, the disclosure of which is hereby incorporated herein by reference in its entirety.

### REFERENCE TO AN ELECTRONIC SEQUENCE LISTING

The content of the electronic sequence listing (183952035500seqlist.xml; Size: 166,103 bytes; and Date of Creation: Dec. 6, 2024) is herein incorporated by reference in its entirety.

### FIELD OF THE INVENTION

The invention provides ionizable cationic lipids and lipid nanoparticles for the delivery of nucleic acids to immune cells (e.g., macrophages, monocytes, or dendritic cells), and methods of making and using, such lipids and targeted lipid nanoparticles.

### BACKGROUND

In recent years, a number of therapeutic modalities have been developed that involve the delivery of one or more nucleic acids to a subject. Treatment modalities include, for example, gene therapies where a gene of interest in the form of deoxyribose nucleic acid (DNA) is introduced into a cell, which is then expressed to produce a gene product, for example, protein, for treating a disorder caused by or associated with a deficiency or absence of the gene product. In this approach, the gene is transcribed into a messenger ribonucleic acid (mRNA), whereupon the mRNA is translated to produce the gene product. In another approach, mRNA rather than a gene of interest can be delivered to the cell. The resulting expression product can ameliorate the deficiency or absence of a particular protein in a subject (for example, a protein deficiency occurring in certain forms of cystic fibrosis or lysosomal storage disorders), or can be used to modulate a cellular function, for example, reprogramming immune cells to initiate or otherwise modulate an immune response in the subject (for example, as a therapeutic agent for treating cancer or as a prophylactic vaccine for preventing or minimizing the risk or severity of a microbial or viral infection).

However, the delivery of mRNA to a cell for translation within the cell has been challenging for a variety of factors, such as nuclease degradation of the mRNA prior to entry into the cell and then after introduction into the cell but prior to translation.

RNA may be delivered to a subject using different delivery vehicles, for example, based on cationic polymers or lipids which, together with the RNA, form nanoparticles. The nanoparticles are intended to protect the RNA from degradation, enable delivery of the RNA to the target site and facilitate cellular uptake and processing by the target cells. For delivery efficacy, in addition to the molecular composition, parameters like particle size, charge, or grafting with molecular moieties, such as polyethylene glycol (PEG) or ligands, play a role. Grafting with PEG is believed to reduce serum interactions, increase serum stability and increase time in circulation, which can be helpful for certain targeting approaches.

Compared with DNA delivery technologies used in certain gene therapies, mRNA-based gene treatment has a number of superior features, for example, ease in manipulation, rapid and transient expression, and adaptive convertibility without mutagenesis.

However, the delivery of therapeutic RNAs to cells is difficult in view of the relative instability and low cell permeability of RNAs. Thus, there exists a need to develop methods and compositions to facilitate the delivery of RNAs such as mRNA to cells.

### SUMMARY

The invention provides ionizable cationic lipids, lipid-immune cell targeting group conjugates, and lipid nanoparticle compositions comprising such ionizable cationic lipids and/or lipid-immune cell (e.g., macrophage, monocytes, or dendritic cells) targeting group conjugates, medical kits containing such lipids and/or conjugates, and methods of making and using, such lipids and conjugates.

The lipid nanoparticle compositions provided herein may further comprise a nucleic acid, such as an RNA, e.g., a messenger RNA or mRNA. The lipid nanoparticle compositions may be used for mRNA delivery to a cell (e.g., an immune cell, such as macrophage, monocytes, or dendritic cells) in a subject. Messenger RNA based gene therapy requires efficient delivery of mRNA to circulating cells (e.g., immune cells, such as macrophage, monocytes, or dendritic cells) in plasma or to cells in a given tissue. The main challenges associated with efficient mRNA delivery to attain robust levels of protein expression include: (a) ability to protect the mRNA payload against prevalent serum nucleases upon administration to a subject; (b) the ability to specifically target mRNA delivery to, and thereby maximize protein expression in the target cell (e.g., macrophage, monocytes, or dendritic cells) population; and (c) the ability to maximally deliver the mRNA payload to the cytosolic compartment of cells (e.g., macrophage, monocytes, or dendritic cells) for translation into proteins within the cytoplasm.

The invention provides ionizable cationic lipids for producing lipid nanoparticle compositions that facilitate the delivery of a payload (e.g., a nucleic acid, such as a DNA or RNA, such as an mRNA) disposed therein to cells, for example, mammalian cells, for example human cells, for example, immune cells. The lipids are designed to enable intracellular delivery of a nucleic acid, e.g., mRNA, to the cytosolic compartment of a target cell type and rapidly degrade into non-toxic components. These complex functionalities are achieved by the interplay between chemistry and geometry of the ionizable lipid head group, the hydrophobic “acyl-tail” groups and the linker connecting the head group and the acyl tail groups in the ionizable cationic lipids.

In one aspect, the present invention provides a compound represented by Formula (I): A compound of Formula (I):

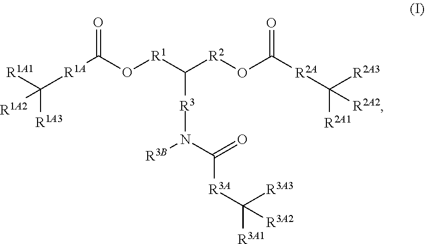

or a salt thereof. In some embodiments, R¹ and R² are each C₁₋₃ alkylene. In some embodiments. R³ is C₁₋₃ alkylene or a bond. In some embodiments, R^{1A} and R^{2A} are each a bond or C₁₋₁₀ alkylene. In some embodiments, R^{3A} is a bond or C₁₋₃ alkylene. In some embodiments, R^{1A1}, R^{2A1}, R^{3A1}, and R^{3A2} are each H. In some embodiments, R^{1A2} and R^{2A2} are each H, —(CH₂)₀₋₅C(O)OR^{a1}, or —(CH₂)₀₋₅OC(O)R². In some embodiments, R^{1A3} and R^{2A3} are each H, —(CH₂)₀₋₅C(O)OR^{a1}, or —(CH₂)₀₋₅OC(O)R². In some embodiments, R^{3A3} is —C(O)OR^{a1}. In some embodiments, R^{a1} and R^{a2} are each independently C₁₋₂₀ alkyl. In some embodiments, R^{3B} is

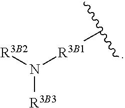

In some embodiments, R^{3B1} is C₄₋₆ alkylene. In some embodiments, R^{3B2} and R^{3B3} are each C₁₋₃ alkyl.

In some embodiments, R¹ and R² are each methylene. In some embodiments, R^{1A} and R^{2A} are each a bond, —CH₂—, —(CH₂)₂—, —(CH₂)₃—, —(CH₂)₄—, —(CH₂)₅—, —(CH₂)₆—, —(CH₂)₇—, —(CH₂)₅—, —(CH₂)₉—, or —(CH₂)₁₀—. In some embodiments, R^{1A} and R^{2A} are each a bond, —(CH₂)₂—, —(CH₂)₅—, —(CH₂)₇—, or —(CH₂)₉—. In some embodiments, R^{3A} is a bond, —CH₂—, or —(CH₂)₂—. In some embodiments, R^{3A} is —CH₂—.

In some embodiments, R^{1A2} and R^{2A2} are each —OC(O)(C₁₋₁₅ alkyl), —C(O)O(C₁₋₁₅ alkyl), —OC(O)CH(C₁₋₁₀ alkyl)(C₁₋₁₀ alkyl), —C(O)OCH(C₁₋₁₀ alkyl)(C₁₋₁₀ alkyl), —(CH₂)C(O)O(C₁₋₁₀ alkyl), or —(CH₂)OC(O)(C₁₋₁₀ alkyl). In some embodiments, R^{1A2} and R^{2A2} are each —OC(O)(C₁₋₁₀ alkyl), —C(O)O(C₁₋₁₀ alkyl), —OC(O)CH(C₆ alkyl)(C₈ alkyl), —C(O)OCH(C₂₋₃ alkyl)(C₅₋₆ alkyl), or —(CH₂)C(O)O(C₁₋₁₀ alkyl). In some embodiments, R^{1A2} and R^{2A2} are each

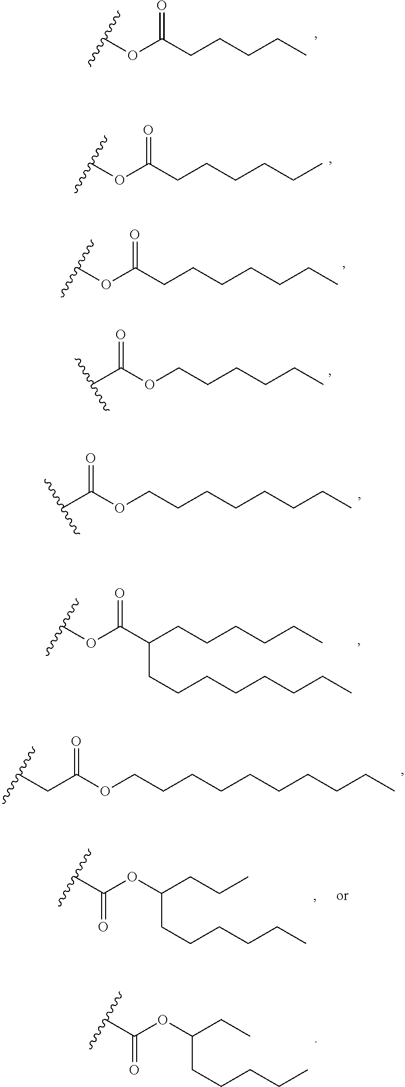

In some embodiments, R^{1A3} and R^{2A3} are each H, —OC(O)(C₁₋₁₅ alkyl), or —C(O)O(C₁₋₁₅ alkyl). In some embodiments, R^{1A3} and R^{2A3} are each H, —OC(O)(C₅₋₁₀ alkyl), —C(O)O(C₆₋₁₀ alkyl), or —(CH₂)C(O)O(C₁₀ alkyl). In some embodiments, R^{1A3} and R^{2A3} are each H,

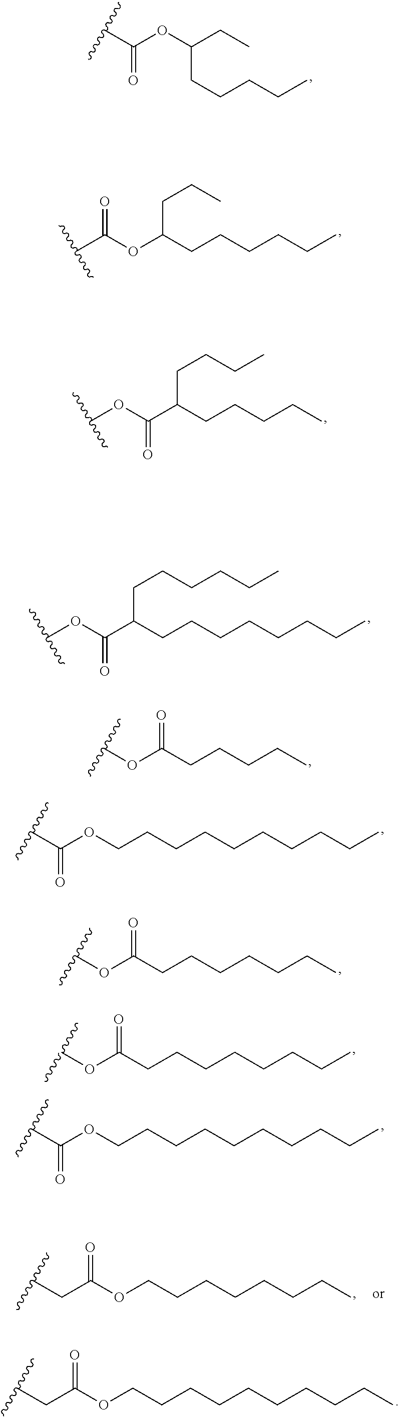

In some embodiments, R^{3A3} is —C(O)OCH(C₁₋₅ alkyl)(C₁₋₁₀ alkyl). In some embodiments R^{3A3} is —C(O)OCH(C₃ alkyl)(C₆ alkyl). In some embodiments, R^{3A3} is

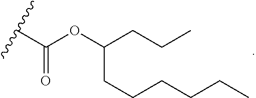 [SMILES: *C(=O)OC(CCC)CCCCCC]

In some embodiments, R^{3B1} is —(CH₂)₄—. In some embodiments, R^{3B2} and R^{3B3} are each methyl. In some embodiments,

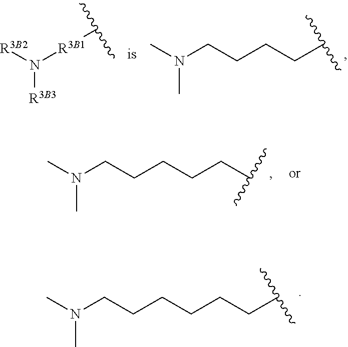

In some embodiments, the compound is selected from Table 1. In some embodiments, the compound is lipid 40, lipid 41, lipid 42, lipid 43, lipid 46, or lipid 52.

Also provided herein is a lipid nanoparticle (LNP). In some embodiments, the LNP comprises a lipid blend for targeted delivery of a nucleic acid into an immune cell. In some embodiments, the lipid blend comprises a lipid-immune cell targeting group conjugate comprising the compound of Formula (II): [Lipid]-[optional linker]-[immune cell targeting group]. In some embodiments, the lipid blend comprises an ionizable cationic lipid such as any of the lipids described herein. In some embodiments, the lipid blend comprises a nucleic acid, wherein the nucleic acid is encapsulated in the LNP. In some embodiments, the ionizable cationic lipid is selected from the lipids described herein.

In some embodiments, the immune cell targeting group comprises an antibody that binds a macrophage antigen, a monocyte antigen, and/or a dendritic antigen. In some embodiments, the macrophage comprises an M1 macrophage, an M2 macrophage, or both. In some embodiments, the macrophage comprises an M2a macrophage, an M2b macrophage, an M2c macrophage, or any combination thereof. In some embodiments, the macrophage antigen comprises CDIIB, CD68, CD80, CD86, TRL-2, TRL-4, iNOS, MHC-IL, CD163, CD206, CD209, FIZZ1, or Ym1/2, or any combination thereof. In some embodiments, the macrophage antigen comprises CD206.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyristoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the PEG is PEG 2000 or PEG 3400. In some embodiments, the lipid-immune cell targeting group conjugate is present in the lipid blend in a range of 0.001 to 0.5 mole percent (e.g., 0.002-0.2 mole percent).

In some embodiments, the lipid blend further comprises one or more of a structural lipid (e.g., a sterol), a neutral phospholipid, and a free PEG-lipid.

In some embodiments, the ionizable cationic lipid is present in the lipid blend in a range of 30-70 (e.g., 40-60) mole percent. In some embodiments, the sterol is present in the lipid blend in a range of 20-70 (e.g., 30-50) mole percent. In some embodiments, the sterol is cholesterol. In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin. In some embodiments, the neutral phospholipid is present in the lipid blend in a range of 5-15 mole percent.

In some embodiments, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylglycerol (PEG-DOG), PEG-dimyristoyl-glycerol (PEG-DMG), PEG-dipalmitoyl-glycerol (PEG-DPG), PEG-dilinolenoyl-glycero-phosphatidyl ethanolamine (PEG-DLPE), PEG-dimyristoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid. In some embodiments, the free PEG-lipid comprises a diacylphosphatidylethanolamine comprising Dipalmitoyl (C16) chain or Distearoyl (C18) chain, and optionally the free PEG-lipid comprises PEG-DPG and PEG-DMG. In some embodiments, the free PEG-lipid is present in the lipid blend in a range of 1-4 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.01 to 0.1. In some embodiments, the LNP has a zeta potential of from about +0 mV to about +10 mV at pH 5.5, or from about −5 mV to about 0 mV at pH 7.4.

In some embodiments, the nucleic acid is DNA or RNA. In some embodiments, the RNA is an mRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes polypeptide capable of reprogramming an M2 macrophage to an M1 macrophage.

In some embodiments, the immune cell targeting group comprises an antibody, and the antibody is a Fab or an immunoglobulin single variable domain (e.g., a V_{HH}, such as a Nanobody®). Nanobody® is a registered trademark of Ablynx N.V. In some embodiments, the immune cell targeting group comprises a Fab, F(ab′)2, Fab′-SH, Fv, or scFv fragment. In some embodiments, the immune cell targeting group comprises a Fab that is engineered to knock out the natural interchain disulfide bond at the C-terminus. In some embodiments, the Fab comprises a heavy chain fragment that comprises C233S substitution, and a light chain fragment that comprises C214S substitution, numbering according to Kabat. In some embodiments, the immune cell targeting group comprises a Fab that has a non-natural interchain disulfide bond (e.g., an engineered, buried interchain disulfide bond). In some embodiments, the Fab comprises F174C substitution in the heavy chain fragment, and S176C substitution in the light chain fragment, numbering according to Kabat. In some embodiments, the immune cell targeting group comprises a Fab that comprises a cysteine at the C-terminus of the heavy or light chain fragment. In some embodiments, the Fab further comprises one or more amino acids between the heavy chain fragment of the Fab and the C-terminal cysteine.

In some embodiments, the immune cell targeting group comprises an immunoglobulin single variable domain. In some embodiments, the immunoglobulin single variable domain comprises a cysteine at the C-terminus. In some embodiments, the immunoglobulin single variable domain comprises a VHH domain and further comprises a spacer comprising one or more amino acids between the VHH domain and the C-terminal cysteine. In some embodiments, the immune cell targeting group comprises two or more V_{HH} domains. In some embodiments, the two or more V_{HH} domains are linked by an amino acid linker. In some embodiments, the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds. In some embodiments, the immune cell targeting group comprises a V_{HH} domain linked to an antibody CH1 domain, and wherein the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat.

In some embodiments, the LNP is for delivering a nucleic acid into an immune cell, and wherein the LNP binds a first macrophage antigen, and also binds a second macrophage antigen. In some embodiments, the LNP comprises two conjugates, wherein the first conjugate comprises an antibody that binds the first macrophage antigen, and the second conjugate comprises an antibody that binds the second macrophage antigen. In some embodiments, the LNP comprises one conjugate, and the conjugate comprises a bispecific antibody that binds both the first macrophage antigen and the second macrophage antigen. In some embodiments, the bispecific antibody is an immunoglobulin single variable domain or Fab-ScFv. In some embodiments, the LNP binds to a first antigen on the surface of the first type of immune cell, and also binds to a second antigen on the surface of the second type of immune cell. In some embodiments, the first type of immune cell is a first macrophage, and the second type of immune cell is a second macrophage, a T-cell, or an NK cell. In some embodiments, the LNP comprises two conjugates, and the first conjugate comprises a first antibody that binds to the first antigen of the first type of immune cell, and the second conjugate comprises a second antibody that binds to the second antigen of the second type of immune cell. In some embodiments, the LNP comprises one conjugate, and the conjugate comprises a bispecific antibody, and the bispecific antibody binds to both the first antigen on the first type of immune cell, and the second antigen on the second type of immune cells. In some embodiments, the bispecific antibody is an immunoglobulin single variable domain or a Fab-ScFv. In some embodiments, the LNP is for delivering a nucleic acid into an immune cell, and wherein the immune cell targeting group comprises a single antibody that binds to CD206. In some embodiments, the LNP is for delivering a nucleic acid into both a macrophage and a T-cell or both a macrophage and an NK cell, wherein the immune cell targeting group binds to both (i) CD206 and (ii) one of CD3, CD7, CD8, and CD56.

In one aspect, provided is an LNP comprising a lipid blend for targeted delivery of a nucleic acid into a macrophage, the lipid blend comprising: a lipid-macrophage targeting group conjugate comprising the compound of Formula (II-m): [Lipid]-[optional linker]-[macrophage targeting group]; and a nucleic acid, wherein the nucleic acid is encapsulated in the LNP.

In some embodiments, the macrophage is an M2 macrophage. In some embodiments, the macrophage targeting group binds CD206. In some embodiments, the nucleic acid is mRNA, and the mRNA encodes polypeptide capable of reprogramming an M2 macrophage to an M1 macrophage. In some embodiments, the LNP further comprises an ionizable cationic lipid. In some embodiments, the ionizable cationic lipid is selected from the lipids described herein.

In some aspect, provided is a method of targeting the delivery of a nucleic acid to an immune cell of a subject. In some embodiments, the method comprises contacting the immune cell with the LNP described herein, wherein the LNP comprises the nucleic acid.

In some aspect, provided is a method of expressing a polypeptide of interest in a targeted immune cell of a subject. In some embodiments, the method comprises contacting the immune cell with the LNP described herein, wherein the LNP comprises a nucleic acid encoding the polypeptide.

In some aspect, provided is a method of modulating cellular function of a target immune cell of a subject. In some embodiments, the method comprises administering to the subject the LNP described herein, wherein the LNP comprises a nucleic acid modulates the cellular function of the immune cell.

In some aspect, provided is a method of treating, ameliorating, or preventing a symptom of a disorder or disease in a subject in need thereof. In some embodiments, the method comprises administering to the subject an LNP described herein for delivering a nucleic acid into an immune cell of the subject, wherein the LNP comprises the nucleic acid.

In some embodiments, the disorder is an immune disorder, an inflammatory disorder, or cancer. In some embodiments, the nucleic acid encodes an antigen for use in a therapeutic or prophylactic vaccine for treating or preventing cancer. In some embodiments, the antibody is a human or humanized antibody.

In some embodiments, the free PEG-lipid comprises a diacylphosphatidylethanolamines comprising dimyristoyl (C14) chain, Dipalmitoyl (C16) chain or Distearoyl (C18) chain. In some embodiments, the free PEG-lipid is present in the lipid blend in a range of 0.5-2.5 mole percent. In some embodiments, the RNA is an mRNA, tRNA, siRNA, gRNA, or microRNA. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker.

In some embodiments, no more than 5% non-immune cells are transfected by the LNP. In some embodiments, half-life of the nucleic acid delivered by the LNP or a polypeptide encoded by the nucleic acid delivered by the LNP is at least 10% longer than half-life of nucleic acid delivered by a reference LNP or a polypeptide encoded by the nucleic acid delivered by the reference LNP. In some embodiments, at least 10% immune cells are transfected by the LNP. In some embodiments, expression level of the nucleic acid delivered by the LNP is at least 10% higher than expression level of nucleic acid delivered by a reference LNP.

In some embodiments, the nucleic acid is DNA or RNA. In some embodiments, the RNA is an mRNA, tRNA, siRNA, gRNA (guide RNA), circRNA (circular RNA), ribozymes, decoy RNA, or microRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell.

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. The undesired immune cells may be immune cells not bound by the immune cell targeting group. The undesired immune cells may be immune cells other than macrophages, for instance wherein the undesired immune cells are immune cells other than M2a macrophages, M2b macrophages, and/or M2c macrophages. The undesired immune cells may be immune cells other than B cells. The undesired immune cells may be immune cells other than NK cells. The undesired immune cells may be immune cells other than T cells, for example CD4+ T cells and/or CD8+ T cells. The undesired immune cells may be immune cells other than NK cells and T cells, for example NK cells and CD4+ T cells and/or CD8+ T cells. In some embodiments, the immune cells are monocytes. In some embodiments, the immune cells are dendritic cells. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP. Immune cells that are meant to be the destination of the delivery may be immune cells bound by the immune cell targeting group. Immune cells that are meant to be the destination of the delivery may be macrophages, for example M2a macrophages, M2b macrophages, and/or M2c macrophages. Immune cells that are meant to be the destination of the delivery may be B cells. Immune cells that are meant to be the destination of the delivery may be NK cells. Immune cells that are meant to be the destination of the delivery may be T cells, for example CD4+ T cells and/or CD8+ T cells. Immune cells that are meant to be the destination of the delivery may be NK cells and T cells, for example NK cells and CD4+ T cells and/or CD8+ T cells. In some embodiments, the immune cells are monocytes. In some embodiments, the immune cells are dendritic cells.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In some embodiments, the antibody is an immunoglobulin single variable (ISV) domain, and the ISV domain is a V_{HH}. In some embodiments, the free PEG lipid comprises a PEG having a molecular weight of at least 2000 daltons. In some embodiments, the PEG has a molecular weight of about 3000 to 5000 daltons. In some embodiments, the antibody is a Fab. In some embodiments, the Fab binds CD206, and the free PEG lipid in the LNP comprises a PEG having a molecular weight of about 2000 daltons. In some embodiments, the Fab is an anti-CD206 antibody, and the free PEG lipid in the LNP comprises a PEG having a molecular weight of about 3000 to 3500 daltons.

Various aspects and embodiments of the invention are described in further detail below.

### BRIEF DESCRIPTION OF THE DRAWINGS

FIG. 1A, FIG. 1B, and FIG. 1C depict the structure (FIG. 1A), proton NMR spectrum (FIG. 1B), and LC-CAD chromatogram (FIG. 1C) of purified lipid 40.

FIG. 2A, FIG. 2B, and FIG. 2C depict the structure (FIG. 2A), proton NMR spectrum (FIG. 2B), and LC-CAD chromatogram (FIG. 2C) of purified lipid 41.

FIG. 3A, FIG. 3B, and FIG. 3C depict the structure (FIG. 3A), proton NMR spectrum (FIG. 3B), and LC-CAD chromatogram (FIG. 3C) of purified lipid 42.

FIG. 4A, FIG. 4B, FIG. 4C, and FIG. 4D depict the structure (FIG. 4A), proton NMR spectrum (FIG. 4B), HPLC-ELSD chromatogram (FIG. 4C), and LC-CAD chromatogram (FIG. 4D) of purified lipid 43.

FIG. 5A, FIG. 5B, and FIG. 5C depict the structure (FIG. 5A), proton NMR spectrum (FIG. 5B), and LC-CAD chromatogram (FIG. 5C) of purified lipid 46.

FIG. 6A, FIG. 6B, FIG. 6C, and FIG. 6D depict the structure (FIG. 6A), proton NMR spectrum (FIG. 6B), HPLC-ELSD chromatogram (FIG. 6C), and LC-CAD chromatogram (FIG. 6D) of purified lipid 52.

FIG. 7A, FIG. 7B, FIG. 7C, and FIG. 7D depict physiochemical characterization results of parent LNPs comprising lipids 15, 26, 25A, 27, 28, 40, or ALC-0315. FIG. 7A depicts hydrodynamic diameter (DLS) pre (“Parent LNPs”) and post (“FT LNPs”) 1× freeze-thaw cycle. FIG. 7B depicts polydispersity index (DLS) pre (“Parent LNPs”) and post (“FT LNPs”) 1× freeze-thaw (FT) cycle. FIG. 7C depicts charge (zeta potential) of LNPs at pH 5.5 or pH 7.4. FIG. 7D depicts total and dye accessible RNA (Ribogreen Assay) of LNPs.

FIG. 8A and FIG. 8B depict physiochemical characterization of parent (“Parent LNPs”) and aCD206 targeted LNPs (“Targeted LNPs”)), where LNPs comprise lipids 15, 26, 25A, 27, 28, 40, or ALC-0315. FIG. 8A depicts hydrodynamic diameter (DLS). FIG. 8B depicts polydispersity index (DLS).

FIG. 9A, FIG. 9B, FIG. 9C, and FIG. 9D depict physiochemical characterization of parent LNPs (DLS), comprising lipids 15A, 17A, 18A, 19A, 21A, 20A, 46, or 40. FIG. 9A depicts hydrodynamic diameter (DLS) of pre (“Zav”) and post (“FT_Zav”) 1× freeze-thaw cycle. FIG. 9B. depicts polydispersity index (DLS) pre (“PDI_4C”) and post (“PDI_FT”) 1× freeze-thaw cycle. FIG. 9C depicts charge (zeta potential) of LNPs, at pH 5.5 or pH 7.4. FIG. 9D depicts total and dye accessible RNA (Ribogreen Assay) of LNPs.

FIG. 10A, FIG. 10B, and FIG. 10C depict results of LNPs binding and GFP protein expression using LNPs comprising comparator lipid ALC-0315; αCD206 targeted LNPs comprising lipid 15, 15A, 26, 40, 27, or 28; parent (non-targeted) LNPs comprising lipid 15; and buffer (PBS) control. FIG. 10A depicts % GFP+ Macrophages. FIG. 10B depicts % DiI+ Macrophages. FIG. 10C depicts mean fluorescence intensity (MFI) of GFP+ Macrophages.

FIG. 11A, FIG. 11B, FIG. 11C, and FIG. 11D depict results of Parent and αCD206 targeted LNPs binding and GFP protein expression using LNPs comprising lipid 15, 40, 17A, 18A, 19A, 21A, 20A, or 46, and PBS buffer control (“No LNP”) in macrophages derived from Human PBMC Donor 108. FIG. 11A depicts % GFP+ Macrophages. FIG. 11B depicts % DiI+ Macrophages. FIG. 11C depicts mean fluorescence intensity (MFI) of GFP+ Macrophages. FIG. 11D depicts mean fluorescence intensity (MFI) of DiI+ Macrophages.

FIG. 12A, FIG. 12B, FIG. 12C, and FIG. 12D depict results of Parent and αCD206 targeted LNPs binding and GFP protein expression using LNPs comprising lipid 15, 40, 17A, 18A, 19A, 21A, 20A, or 46, and PBS buffer control (“No LNP”) in macrophages derived from Human PBMC Donor 282. FIG. 12A depicts % GFP+ Macrophages. FIG. 12B depicts % DiI+ Macrophages. FIG. 12C depicts mean fluorescence intensity (MFI) of GFP+ Macrophages. FIG. 12D depicts mean fluorescence intensity (MFI) of DiI+ Macrophages.

FIG. 13A, FIG. 13B, FIG. 13C, and FIG. 13D depict the physicochemical characterization of mCherry-mRNA Lipid 40 LNPs and 1.5 mole % and 3.5 mole % DSG-PEG lipid. FIG. 13A depicts the hydrodynamic diameter (DLS) of parent LNPs pre- and post-1× freeze-thaw cycle. FIG. 13B depicts the polydispersity Index (DLS) of parent LNPs pre- and post-1× freeze-thaw cycle. FIG. 13C depicts the charge (Zeta Potential) of parent LNPs at pH 5.5 and pH 7.4. FIG. 13D depicts the total and dye accessible RNA (Ribogreen Assay) of parent LNPs.

FIG. 14A, FIG. 14B, FIG. 14C, and FIG. 14D depict the physicochemical characterization of mCherry-mRNA Lipid 40 LNPs and 3.5 mole % DPG-PEG and DSG-PEG lipids. FIG. 14A depicts the LNP diameter pre- and post-1× freeze-thaw cycle. FIG. 14B depicts the polydispersity Index (DLS) of LNPs pre- and post-1× freeze-thaw cycle. FIG. 14C depicts the charge (Zeta Potential) of LNPs at pH 5.5 and pH 7.4. FIG. 14D depicts the total and dye accessible RNA (Ribogreen Assay) of LNPs.

FIG. 15A, FIG. 15B, FIG. 15C, and FIG. 15D depict mCherry expression and LNP uptake (DiI label) in M2 Macrophages using mCherry-mRNA LNPs based on Lipid 40 and 1.5 mole % and 3.5 mole % DSG-PEG lipid. LNP and buffer (PBS) control. FIG. 15A depicts % mCherry+ Macrophages. FIG. 15B depicts Mean Fluorescence Intensity (MFI) of mCherry+ Macrophages. FIG. 15C depicts % DiI+ Macrophages. FIG. 15D depicts Mean Fluorescence Intensity (MFI) of DiI+ Macrophages.

FIG. 16A, FIG. 16B, FIG. 16C, and FIG. 16D depict mCherry expression and LNP uptake (DiI label) in M2 Macrophages using mCherry/DiI-mRNA LNPs based on Lipid 40 and 3.5 mole % DSG-PEG or 3.5 mole % DPG-PEG lipid. LNP and buffer (PBS) control. FIG. 16A depicts % mCherry+ Macrophages. FIG. 16B depicts Mean Fluorescence Intensity (MFI) of mCherry+ Macrophages. FIG. 16C depicts % DiI+ Macrophages. FIG. 16D depicts Mean Fluorescence Intensity (MFI) of DiI+ Macrophages.

FIG. 17 depicts hemagglutinin (HA) expression 20 hours post HA-mRNA LNPs based on Lipid 40, 46, ALC-0315, CL-1191 and SM-102 Ionizable Lipids dosed in vitro (1 ug/1E6 cells) to human dendritic cells. HA expression in human dendritic cells, quantified as Mean Fluorescence Intensity (MFI) of Alexa-647 conjugated secondary Ab, 20 hours post-HA-mRNA LNP dose of 1 ug/1E6 cells: Relative Expression of LNPs based on Lipid 40, Lipid 46, and ALC-0315, SM-102, and benchmark control (CL-1191) ionizable lipids.

FIG. 18 depicts hemagglutinin (HA) expression 20 hours post HA-mRNA LNPs based on Lipid 40, 46, and CL-1191 Ionizable Lipids dosed in vitro (1 ug/1E6 cells) to human Skeletal Muscle Cells. HA expression in human Skeletal Muscle cells, quantified as Mean Fluorescence Intensity (MFI) of Alexa-647 conjugated secondary Ab, 20 hours post-HA-mRNA LNP dose of 1 ug/1E6 cells: Relative Expression of LNPs based on Lipid 40, Lipid 46, and benchmark control (CL-1191) ionizable lipids.

FIG. 19A, FIG. 19B, and FIG. 19C depict results of C57BL/6 mice engrafted with MC-38 tumor (n=4) dosed (1.5 mg/kg) with Lipid 40 LNPs formulated with 1.5 or 3.5 mole % DSG-PEG. FIG. 19A depicts lipid concentration (ng/g) in blood 10 minutes, 6 hour and 24 hours post dose. FIG. 19B depicts percent of injected lipid dose in blood 10 minutes, 6 hour and 24 hours post dose. FIG. 19C depicts lipid concentration in blood, liver, spleen, lung and tumor tissue 24 hours post dose.

FIG. 20A, FIG. 20B, and FIG. 20C depict results of C57BL/6 mice engrafted with MC-38 tumor (n=4) dosed (1.0 mg/kg) with Lipid 40 LNPs formulated with 3.5 mole % DPG-PEG or DSG-PEG. FIG. 20A depicts lipid concentration (ng/g) in blood 10 minutes, 6 hour and 24 hour post dose. FIG. 20B depicts percent of injected lipid dose in blood 10 minutes, 6 hour and 24 hour post dose. FIG. 20C depicts lipid concentration in blood, liver, spleen, lung and tumor tissue 24 hours post dose.

FIG. 21A and FIG. 21B depict results of C57BL/6 mice engrafted with MC-38 tumor (n=4) dosed (1.5 mg/kg) with Lipid 40 mCherry-LNPs with 1.5 or 3.5 mole % DSG-PEG. FIG. 21A depicts mCherry expression monitored (FACS) in tumor, liver, spleen and lung tissues 24 hour post dose in macrophages. FIG. 21B depicts mCherry expression monitored (FACS) in tumor, liver, spleen and lung tissues 24 hour post dose in monocytes.

FIG. 22A and FIG. 22B depict results of C57BL/6 mice engrafted with MC-38 tumor (n=4) dosed (1.5 mg/kg) with Lipid 40 mCherry-LNPs with 1.5 mole % DSG-PEG. FIG. 22A depicts mCherry expression monitored (FACS) in relevant cell types 24 hours post dose in liver. FIG. 22B depicts mCherry expression monitored (FACS) in relevant cell types 24 hours post dose in spleen.

FIG. 23A, FIG. 23B, FIG. 23C, FIG. 23D, FIG. 23E, and FIG. 23F depict results of C57BL/6 mice engrafted with MC-38 tumor (n=4) dosed (1.0 mg/kg) with Lipid 40 mCherry-LNPs with 3.5 mole % DSG-PEG and 3.5 mole % DPG-PEG. FIG. 23A depicts mCherry expression monitored (FACS) 24 hours post dose in liver macrophages. FIG. 23B depicts mCherry expression monitored (FACS) 24 hours post dose in liver monocytes. FIG. 23C depicts mCherry expression monitored (FACS) 24 hours post dose in spleen macrophages. FIG. 23D depicts mCherry expression monitored (FACS) 24 hours post dose in spleen monocytes. FIG. 23E depicts mCherry expression monitored (FACS) 24 hours post dose in lung macrophages. FIG. 23F depicts mCherry expression monitored (FACS) 24 hours post dose in lung monocytes.

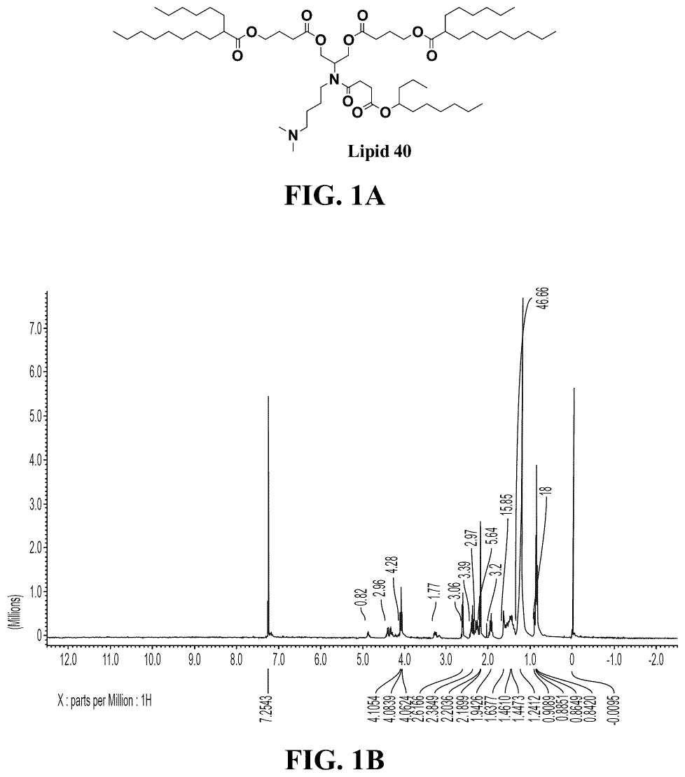

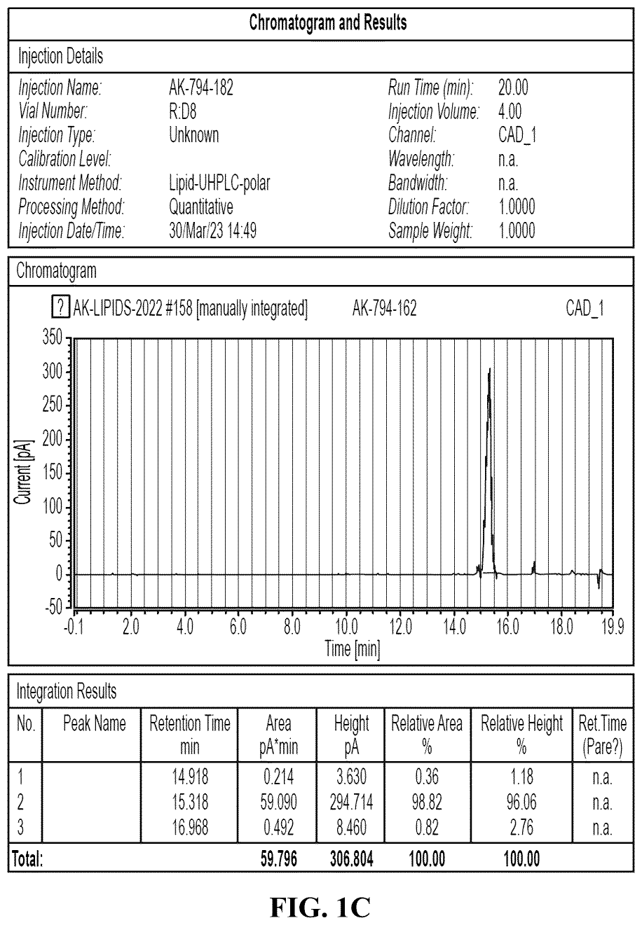

### DETAILED DESCRIPTION

The invention provides ionizable cationic lipids, lipid-immune cell targeting group conjugates, and lipid nanoparticle compositions comprising such ionizable cationic lipids and/or lipid-immune cell (e.g., macrophage, monocytes, or dendritic cells) targeting group conjugates, medical kits containing such lipids and/or conjugates, and methods of making and using, such lipids and conjugates.

The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts. In addition, when a chemical group is a diradical, for example, it is understood a that the chemical groups can be bonded to their adjacent atoms in the remainder of the structure in one or both orientations, for example, —OC(O)— is interchangeable with —C(O)O— or —OC(S)— is interchangeable with —C(S)O—.

The terms “a” and “an” as used herein mean “one or more” and include the plural unless the context is inappropriate. In some embodiments, “one or more” is 1 or 2. In some embodiments, “one or more” is 1, 2, or 3. In some embodiments, “one or more” is 1, 2, 3, or 4. In some embodiments, “one or more” is 1, 2, 3, 4, or 5. In some embodiments, “one or more” is 1, 2, 3, 4, 5, or more.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, or C₁-C₆alkyl, respectively. In some embodiments, alkyl is optionally substituted. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. In some embodiments, alkylene is optionally substituted. An exemplary alkylene group is —CH2CH2-.

The term “haloalkyl” refers to an alkyl group that is substituted with at least one halogen. For example, —CH₂F, —CHF₂, —CF₃, —CH₂CF₃, —CF₂CF₃, and the like.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

The term “oxo” is art-recognized and refers to a “═O” substituent. For example, a cyclopentane substituted with an oxo group is cyclopentanone.

The term “morpholinyl” refers to a substituent having the structure of:

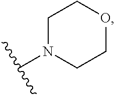 [SMILES: *N1CCOCC1]

which is optionally substituted.

The term “piperidinyl” refers to a substituent having a structure of:

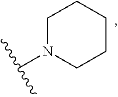 [SMILES: *N1CCCCC1]

which is optionally substituted.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, “optionally substituted” is equivalent to “unsubstituted or substituted.” In some embodiments, “optionally substituted” indicates that the designated atom or group is optionally substituted with one or more substituents independently selected from optional substituents provided herein. In some embodiments, optional substituent may be selected from the group consisting of: C₁₋₆alkyl, cyano, halogen, —O—C₁₋₆alkyl, C₁₋₆haloalkyl, C₃₋₇cycloalkyl, 3- to 7-membered heterocyclyl, 5- to 6-membered heteroaryl, and phenyl. In some embodiments, optional substituent is alkyl, cyano, halogen, halo, azide, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl, or heteroaryl. In some embodiments, optional substituent is —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13}, or —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

The term “haloalkyl” refers to an alkyl group that is substituted with at least one halogen. For example, —CH₂F, —CHF₂, —CF₃, —CH₂CF₃, —CF₂CF₃, and the like.

The term “cycloalkyl” refers to a monovalent saturated cyclic, bicyclic, bridged cyclic (e.g., adamantyl), or spirocyclic hydrocarbon group of 3-12, 3-10, 3-8, 4-8, or 4-6 carbons, referred to herein, e.g., as “C₄₋₈cycloalkyl,” derived from a cycloalkane. In some embodiments, cycloalkyl is optionally substituted. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. In some embodiments, heterocyclyl is optionally substituted. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems (e.g., fused, spiro, bridged bicyclic). A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isoxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “aryl” is art-recognized and refers to a carbocyclic aromatic group. In some embodiments, aryl is optionally substituted. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6- to 10-membered ring structure. In some embodiments, the aryl group is a C₆-C₁₄ aryl.

The term “heteroaryl” is art-recognized and refers to aromatic groups that include at least one ring heteroatom. In some embodiments, heteroaryl is optionally substituted. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula —N(R¹⁰)(R¹¹), wherein R¹⁰ and R¹¹ each independently represent hydrogen, alkyl, cycloalkyl, heterocyclyl, alkenyl, aryl, aralkyl, or (CH₂)ₘ—R¹²; or R¹⁰ and R¹¹, taken together with the N atom to which they are attached complete a heterocycle having from 4 to 8 atoms in the ring structure; R¹² represents an aryl, a cycloalkyl, a cycloalkenyl, a heterocycle or a polycycle; and m is zero or an integer in the range of 1 to 8. In certain embodiments, R¹⁰ and R¹¹ each independently represent hydrogen, alkyl, alkenyl, or —(CH₂)ₘ—R¹².

The terms “alkoxyl” or “alkoxy” are art-recognized and refer to an alkyl group, as defined above, having an oxygen radical attached thereto. In some embodiments, alkoxyl is optionally substituted. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, O-alkynyl, —O—(CH₂)ₘ—R¹², where m and R¹² are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol “ [symbol] ” denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with 3H and 14C) are useful in compound and/or substrate tissue distribution assays. Tritiated (i.e., 3H) and carbon-14 (i.e., 14C) isotopes are particularly preferred for their ease of preparation and detectability. Further, substitution with heavier isotopes such as deuterium (i.e., 2H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable excipient” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Remington's The Science and Practice of Pharmacy, 21st Edition, A. R. Gennaro; Lippincott, Williams & Wilkins, Baltimore, MD, 2006.

As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

Abbreviations as used herein include diisopropylethylamine (DIPEA); 4-dimethylaminopyridine (DMAP); tetrabutylammonium iodide (TBAI); 1-ethyl-3-(3-dimethylaminopropyl)carbodiimide (EDC); benzotriazol-1-yl-oxytripyrrolidinophosphonium hexafluorophosphate (PyBOP), 9-Fluorenylmethoxycarbonyl (Fmoc), tetrabutyldimethylsilyl chloride (TBDMSCl), hydrogen fluoride (HF), phenyl (Ph), bis(trimethylsilyl)amine (HNDS), dimethylformamide (DMF); methylene chloride (DCM); tetrahydrofuran (THF); high-performance liquid chromatography (HPLC); mass spectrometry (MS), evaporative light scattering detector (ELSD), electrospray (ES)); nuclear magnetic resonance spectroscopy (NMR).

As used herein, the term “effective amount” refers to the amount of a compound (e.g., a nucleic acid, e.g., an mRNA) sufficient to effect beneficial or desired results. An effective amount can be administered in one or more administrations, applications or dosages and is not intended to be limited to a particular formulation or administration route. The term effective amount can be considered to include therapeutically and/or prophylactically effective amounts of a compound.

The phrase “therapeutically effective amount” as used herein means that amount of a compound (e.g., a nucleic acid, e.g., an mRNA), material, or composition comprising a compound (e.g., a nucleic acid, e.g., an mRNA) which is effective for producing some desired therapeutic effect in at least a sub-population of cells in a mammal, for example, a human, or a subject (e.g., a human subject) at a reasonable benefit/risk ratio applicable to any medical treatment.

The phrase “prophylactically effective amount” as used herein means that amount of a compound (e.g., a nucleic acid, e.g., an mRNA), material, or composition comprising a compound (e.g., a nucleic acid, e.g., an mRNA) which is effective for producing some desired prophylactic effect in at least a sub-population of cells in a mammal, for example, a human, or a subject (e.g., a human subject) by reducing, minimizing or eliminating the risk of developing a condition or the reducing or minimizing severity of a condition at a reasonable benefit/risk ratio applicable to any medical treatment.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As used herein, unless otherwise indicated, the term “antibody” means any antigen-binding molecule or molecular complex comprising at least one complementarity determining region (CDR) that specifically binds to or interacts with a particular antigen. It is understood the term encompasses an intact antibody (e.g., an intact monoclonal antibody), or a fragment thereof, such as an Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified or engineered. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). The term also encompasses an immunoglobulin single variable domain (e.g., a V_{HH}). The numbering of amino acid residues in antibodies disclosed herein is according to Kabat, unless otherwise explicitly stated.

As used here, an “antibody that binds to X” (i.e., X being a particular antigen), or “an anti-X antibody”, is an antibody that specifically recognizes the antigen X.

As used herein, a “buried interchain disulfide bond” or an “interchain buried disulfide bond” refers to a disulfide bond on a polypeptide which is not readily accessible to water soluble reducing agents, or is effectively “buried” in the hydrophobic regions of the polypeptide, such that it is unavailable to both reducing agents and for conjugation to other hydrophilic PEGs. Buried interchain disulfide bonds are further described in WO2017096361A1, which is incorporated by reference in its entirety.

As used herein, specificity of the targeted delivery by an LNP is defined by the ratio between % of a desired immune cell type that receives the delivered nucleic acid (e.g., on-target delivery), and % of an undesired immune cell type that is not meant to be the destination of the delivery, but receives the delivered nucleic acid (e.g., off-target delivery). For example, the specificity is higher when more desired immune cells receive the delivered nucleic acid, while less undesired immune cells receive the delivered nucleic acid. Specificity of the targeted delivery by an LNP can also be defined the ratio of amount of nucleic acid being delivered to the desired immune cells (e.g., on-target delivery) and amount of nucleic acid being delivered to the undesired immune cells (e.g., off-target delivery). Specificity of the delivery can be determined using any suitable method. As a non-limiting example, expression level of the nucleic acid in the desired immune cell type can be measured and compared to that of a different immune cell type that is not meant to be the destination of the delivery.

As used herein, in some embodiments, a reference LNP is an LNP that does not have the immune cell targeting group but is otherwise the same as the tested LNP. In some other embodiments, a reference LNP is an LNP that has a different ionizable cationic lipid but is otherwise the same as the tested LNP. In some embodiments, a reference LNP comprises D-Lin-MC3-DMA as the ionizable cationic lipid which is different from the ionizable cationic lipid in a tested LNP, but is otherwise the same as the tested LNP.

As used herein, a humanized antibody is an antibody which is wholly or partially of non-human origin and whose protein sequence has been modified to replace certain amino acids, for instance that occur at the corresponding position(s) in the framework regions of the VH and VL domains in a sequence of antibody from a human being, to increase its similarity to antibodies produced naturally in humans, in order to avoid or minimize an immune response in humans. For example, using techniques of genetic engineering, the variable domains of a non-human antibodies of interest may be combined with the constant domains of human antibodies. The constant domains of a humanized antibody are most of the time human CH and CL domains.

As used herein, the term “structural lipid” refers to sterols and also to lipids containing sterol moieties.

The “percent identity” between two sequences is a function of the number of identical positions shared by the sequences (i.e., % identity=number of identical positions/total number of positions×100), taking into account the number of gaps, and the length of each gap, which need to be introduced for optimal alignment of the two sequences. The percent identity between two amino acid sequences may be determined using the algorithm of E. Meyers and W. Miller (Comput. Appl. Biosci. 4: 11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (Mol. Biol. 48:444-453 (1970)) algorithm which has been incorporated into the GAP program in the GCG software package (available at www gcg com), using either a Blossum 62 matrix or a PAM250 matrix, and a gap weight of 16, 14, 12, 10, 8, 6, or 4 and a length weight of 1, 2, 3, 4, 5, or 6.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### Immunoglobulin Single Variable Domain

In some embodiments, the immune cell targeting group of the LNPs as described herein comprise an immunoglobulin single variable domain, such as a V_{HH}.

The term “immunoglobulin single variable domain” (ISV), interchangeably used with “single variable domain,” defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain. Hence, the antigen binding site of an immunoglobulin single variable domain is formed by no more than three CDRs.

As such, the single variable domain may be a light chain variable domain sequence (e.g., a V_{L}-sequence) or a suitable fragment thereof, or a heavy chain variable domain sequence (e.g., a V_{H}-sequence or V_{HH} sequence) or a suitable fragment thereof, as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISV) can for example be a heavy chain ISV, such as a V_{H}, V_{HH}, including a camelized V_{H} or humanized V_{HH}. In one embodiment, it is a V_{HH}, including a camelized V_{H} or humanized V_{HH}. Heavy chain ISVs can be derived from a conventional four-chain antibody or from a heavy chain antibody.

For example, the immunoglobulin single variable domain may be a (single) domain antibody (or an amino acid sequence that is suitable for use as a single domain antibody), a “dAb” or dAb (or an amino acid sequence that is suitable for use as a dAb) or a V_{HH}; other single variable domains, or any suitable fragment of any one thereof.

In particular, the immunoglobulin single variable domain may be a V_{HH} (e.g., a humanized V_{HH} or camelized V_{H}) or a suitable fragment thereof.

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. 1993 (Nature 363: 446-448). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L}domains”). For a further description of V_{HH}'s, reference is made to the review article by Muyldermans 2001 (Reviews in Molecular Biotechnology 74: 277-302).

For the term “dAb's” and “domain antibody”, reference is for example made to Ward et al. 1989 (Nature 341: 544), to Holt et al. 2003 (Trends Biotechnol. 21: 484); as well as to for example WO 2004/068820, WO 2006/030220, WO 2006/003388 and other published patent applications of Domantis Ltd. It should also be noted that, although less preferred in the context of the present invention because they are not of mammalian origin, single variable domains can be derived from certain species of shark (for example, the so-called “IgNAR domains”, see for example WO 2005/18629).

Typically, the generation of immunoglobulins involves the immunization of experimental animals, fusion of immunoglobulin producing cells to create hybridomas and screening for the desired specificities. Alternatively, immunoglobulins can be generated by screening of naïve, immune or synthetic libraries, e.g., by phage display.

The generation of immunoglobulin sequences, such as VHHs, has been described extensively in various publications, among which WO 1994/04678, Hamers-Casterman et al. 1993 (Nature 363: 446-448) and Muyldermans et al. 2001 (Reviews in Molecular Biotechnology 74: 277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be used herein. Also, fully human, humanized or chimeric sequences can be used in the method described herein. For example, camelid immunoglobulin sequences and humanized camelid immunoglobulin sequences, or camelized domain antibodies, e.g., camelized dAb as described by Ward et al. 1989 (Nature 341: 544), WO 1994/04678, and Davis and Riechmann (1994, Febs Lett., 339:285-290; and 1996, Prot. Eng., 9:531-537) can be used herein. Moreover, the ISVs are fused forming a multivalent and/or multispecific construct (for multivalent and multispecific polypeptides containing one or more V_{HH} domains and their preparation, reference is also made to Conrath et al. 2001 (J. Biol. Chem., Vol. 276, 10. 7346-7350) as well as to for example WO 1996/34103 and WO 1999/23221).

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the prior art (e.g., WO 2008/020079). Again, it should be noted that such humanized V_{HH}s can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the description in the prior art (e.g., Davies and Riechman 1994, FEBS 339: 285; 1995, Biotechnol. 13: 475; 1996, Prot. Eng. 9: 531; and Riechman 1999, J. Immunol. Methods 231: 25). Such “camelizing” substitutions are inserted at amino acid positions that form and/or are present at the V_{H}-V_{L} interface, and/or at the so-called Camelidae hallmark residues, as defined herein (see for example WO 1994/04678 and Davies and Riechmann (1994 and 1996, supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a V_{L}-sequence) and/or from a heavy chain variable domain (e.g., a V_{H}-sequence or V_{HH} sequence). In one particular aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISV sequence described herein may contain one or more of hallmark residues (as defined herein), such that the ISV sequence is a V_{HH}, (e.g., a humanized V_{HH} or camelized V_{H}). Non-limiting examples of (suitable combinations of) such framework sequences will become clear from the further disclosure herein.

The total number of amino acid residues in a V_{H} domain and a V_{HH} domain will usually be in the range of from 110 to 120, often between 112 and 115. It should however be noted that smaller and longer sequences may also be suitable for the purposes described herein.

However, it should be noted that the ISVs described herein is not limited as to the origin of the ISV sequence (or of the nucleotide sequence used to express it), nor as to the way that the ISV sequence or nucleotide sequence is (or has been) generated or obtained. Thus, the ISV sequences may be naturally occurring sequences (from any suitable species) or synthetic or semi-synthetic sequences. In a specific but non-limiting aspect, the ISV sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, V_{HH} sequences (including (partially) humanized V_{HH} sequences and camelized V_{H} sequences) can be characterized by the presence of one or more “Hallmark residues” (as described herein) in one or more of the framework sequences (again as further described herein). Thus, generally, a V_{HH}, can be defined as an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a V_{HH} can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a V_{HH} can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table A below.

TABLE A
Hallmark Residues in V_{HH}^{S}

| Position | Human V_{H}³ | Hallmark Residues |
|---|---|---|
| 11 | L, V; | L, S, V, M, W, F, T, Q, E, A, R, |
| | predominantly L | G, K, Y, N, P, I; preferably L |
| 37 | V, I, F; | F⁽¹⁾, Y, V, L, A, H, S, I, W, C, N, |
| | usually V | G, D, T, P, preferably F⁽¹⁾ or Y |
| 44⁽⁸⁾ | G | E⁽³⁾, Q⁽³⁾, G⁽²⁾, D, A, K, R, L, P, S, V, |
| | | H, T, N, W, M, I; preferably G⁽²⁾, E⁽³⁾ |
| | | or Q⁽³⁾;most preferably G⁽²⁾ or Q⁽³⁾. |
| 45⁽⁸⁾ | L | L⁽²⁾, R⁽³⁾, P, H, F, G, Q, S, E, T, Y, C, |
| | | I, D, V; preferably L⁽²⁾ or R⁽³⁾ |
| 47⁽⁸⁾ | W, Y | F⁽¹⁾, L⁽¹⁾ or W⁽²⁾ G, I, S, A, V, M, R, Y, |
| | | E, P, T, C, H, K, Q, N, D; preferably |
| | | W⁽²⁾, L⁽¹⁾ or F⁽¹⁾ |
| 83 | R or K; | R, K⁽⁵⁾, T, E⁽⁵⁾, Q, N, S, I, V, G, M, L, |
| | usually R | A, D, Y, H; preferably K or R; most |
| | | preferably K |
| 84 | A, T, D; | P⁽⁵⁾, S, H, L, A, V, I, T, F, D, R, Y, N, |
| | predominantly A | Q, G, E; preferably P |
| 103 | W | W⁽⁴⁾, R⁽⁶⁾, G, S, K, A, M, Y, L, F, T, N, |
| | | V, Q, P⁽⁶⁾, E, C; preferably W |
| 104 | G | G, A, S, T, D, P, N, E, C, L; |
| | | preferably G |
| 108 | L, M or T; | Q, L⁽⁷⁾, R, P, E, K, S, T, M, A, H; |
| | predominantly L | preferably Q or L⁽⁷⁾ |

Notes:
In particular, but not exclusively, in combination with KERE (SEQ ID NO: 103) or KQRE (SEQ ID NO: 104) at positions 43-46.
Usually as GLEW (SEQ ID NO: 105) at positions 44-47.
Usually as KERE (SEQ ID NO: 103) or KQRE (SEQ ID NO: 104) at positions 43-46, e.g., as KEREL (SEQ ID NO: 106), KEREF (SEQ ID NO: 107), KQREL (SEQ ID NO: 108), KQREF (SEQ ID NO: 109), KEREG (SEQ ID NO: 110), KQREW (SEQ ID NO: 111) or KQREG (SEQ ID NO: 112) at positions 43-47. Alternatively, also sequences such as TERE (SEQ ID NO: 113) (for example TEREL (SEQ ID NO: 114)), TQRE (SEQ ID NO: 115) (for example TQREL (SEQ ID NO: 116)), KECE (SEQ ID NO: 117) (for example KECEL (SEQ ID NO: 118) or KECER (SEQ ID NO: 119)), KQCE (SEQ ID NO: 120) (for example KQCEL (SEQ ID NO: 121)), RERE (SEQ ID NO: 122) (for example REREG (SEQ ID NO: 123)), RQRE (SEQ ID NO: 124) (for example RQREL (SEQ ID NO: 125), RQREF (SEQ ID NO: 126) or RQREW (SEQ ID NO: 127)), QERE (SEQ ID NO: 128) (for example QEREG (SEQ ID NO: 129)), QQRE (SEQ ID NO: 130), (for example QQREW (SEQ ID NO: 131), QQREL (SEQ ID NO: 132) or QQREF (SEQ ID NO: 133)), KGRE (SEQ ID NO: 134) (for example KGREG (SEQ ID NO: 135)), KDRE (SEQ ID NO: 136) (for example KDREV (SEQ ID NO: 137)) are possible. Some other possible, but less preferred sequences include for example DECKL (SEQ ID NO: 138) and NVCEL (SEQ ID NO: 139).
With both GLEW (SEQ ID NO: 105) at positions 44-47 and KERE (SEQ ID NO: 103) or KQRE (SEQ ID NO: 104) at positions 43-46.
Often as KP or EP at positions 83-84 of naturally occurring V_{HH} domains.
In particular, but not exclusively, in combination with GLEW (SEQ ID NO: 105) at positions 44-47.
With the proviso that when positions 44-47 are GLEW (SEQ ID NO: 105), position 108 is always Q in (non-humanized) V_{HH} sequences that also contain a W at 103.
The GLEW group also contains GLEW-like sequences at positions 44-47, such as for example GVEW (SEQ ID NO: 140), EPEW (SEQ ID NO: 141), GLER (SEQ ID NO: 142), DQEW (SEQ ID NO: 143), DLEW (SEQ ID NO: 144), GIEW (SEQ ID NO: 145), ELEW (SEQ ID NO: 146), GPEW (SEQ ID NO: 147), EWLP (SEQ ID NO: 148), GPER (SEQ ID NO: 149), GLER (SEQ ID NO: 142) and ELEW (SEQ ID NO: 146).

In one embodiment, the immunoglobulin single variable domain has certain amino acid substitutions in the framework regions effective in preventing or reducing binding of so-called “pre-existing antibodies” to the polypeptides. ISVs in which (i) the amino acid residue at position 112 is one of K or Q; and/or (ii) the amino acid residue at position 89 is T; and/or (iii) the amino acid residue at position 89 is L and the amino acid residue at position 110 is one of K or Q; and (iv) in each of cases (i) to (iii), the amino acid at position 11 is preferably V have been described in WO2015/173325.

### Polypeptides

The immunoglobulin single variable domains may form part of a protein or polypeptide, which may comprise or essentially consist of one or more (at least one) immunoglobulin single variable domains and which may optionally further comprise one or more further amino acid sequences (all optionally linked via one or more suitable linkers). The term “immunoglobulin single variable domain” may also encompass such polypeptides. The one or more immunoglobulin single variable domains may be used as a binding unit in such a protein or polypeptide, which may optionally contain one or more further amino acids that can serve as a binding unit, so as to provide a monovalent, multivalent or multispecific polypeptide of the invention, respectively (for multivalent and multispecific polypeptides containing one or more VHH domains and their preparation, reference is also made to Conrath et al. 2001 (J. Biol. Chem. 276: 7346), as well as to for example WO 1996/34103, WO 1999/23221 and WO 2010/115998).

The polypeptides may comprise or essentially consist of one immunoglobulin single variable domain, as outlined above. Such polypeptides are also referred to herein as monovalent polypeptides.

The term “multivalent” indicates the presence of multiple ISVs in a polypeptide. In one embodiment, the polypeptide is “bivalent”, i.e., comprises or consists of two ISVs. In one embodiment, the polypeptide is “trivalent”, i.e., comprises or consists of three ISVs. In another embodiment, the polypeptide is “tetravalent”, i.e. comprises or consists of four ISVDs. The polypeptide can thus be “bivalent”, “trivalent”, “tetravalent”, “pentavalent”, “hexavalent”, “heptavalent”, “octavalent”, “nonavalent”, etc., i.e., the polypeptide comprises or consists of two, three, four, five, six, seven, eight, nine, etc., ISVs, respectively. In one embodiment the multivalent ISV polypeptide is trivalent. In another embodiment the multivalent ISV polypeptide is tetravalent. In still another embodiment, the multivalent ISV polypeptide is pentavalent.

In one embodiment, the multivalent ISV polypeptide can also be multispecific. The term “multispecific” refers to binding to multiple different target molecules (also referred to as antigens). The multivalent ISV polypeptide can thus be “bispecific”, “trispecific”, “tetraspecific”, etc., i.e., can bind to two, three, four, etc., different target molecules, respectively.

For example, the polypeptide may be bispecific-trivalent, such as a polypeptide comprising or consisting of three ISVs, wherein two ISVs bind to a first target and one ISV binds to a second target different from the first target. In another example, the polypeptide may be trispecific-tetravalent, such as a polypeptide comprising or consisting of four ISVs, wherein one ISV binds to a first target, two ISVs bind to a second target different from the first target and one ISV binds to a third target different from the first and the second target. In still another example, the polypeptide may be trispecific-pentavalent, such as a polypeptide comprising or consisting of five ISVs, wherein two ISVs bind to a first target, two ISVs bind to a second target different from the first target and one ISV binds to a third target different from the first and the second target.

In one embodiment, the multivalent ISV polypeptide can also be multiparatopic. The term “multiparatopic” refers to binding to multiple different epitopes on the same target molecules (also referred to as antigens). The multivalent ISV polypeptide can thus be “biparatopic”, “triparatopic”, etc., i.e., can bind to two, three, etc., different epitopes on the same target molecules, respectively.

In another aspect, the polypeptide of the invention that comprises or essentially consists of one or more immunoglobulin single variable domains (or suitable fragments thereof), may further comprise one or more other groups, residues, moieties or binding units. Such further groups, residues, moieties, binding units or amino acid sequences may or may not provide further functionality to the immunoglobulin single variable domain (and/or to the polypeptide in which it is present) and may or may not modify the properties of the immunoglobulin single variable domain.

For example, such further groups, residues, moieties or binding units may be one or more additional amino acids, such that the compound, construct or polypeptide is a (fusion) protein or (fusion) polypeptide. In a preferred but non-limiting aspect, said one or more other groups, residues, moieties or binding units are immunoglobulins. Even more preferably, said one or more other groups, residues, moieties or binding units are chosen from the group consisting of domain antibodies, amino acids that are suitable for use as a domain antibody, single domain antibodies, amino acids that are suitable for use as a single domain antibody, “dAb”s, amino acids that are suitable for use as a dAb, or V_{HH}s.

Alternatively, such groups, residues, moieties or binding units may for example be chemical groups, residues, moieties, which may or may not by themselves be biologically and/or pharmacologically active. For example, and without limitation, such groups may be linked to the one or more immunoglobulin single variable domain so as to provide a “derivative” of the immunoglobulin single variable domain.

In another embodiment, said further residues may be effective in preventing or reducing binding of so-called “pre-existing antibodies” to the polypeptides. For this purpose, the polypeptides and constructs may contain a C-terminal extension (X)n (SEQ ID NO: 150) (in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I), for which reference is made to WO 2012/175741. Accordingly, the polypeptide may further comprise a C-terminal extension (X)n (SEQ ID NO: 151), in which n is 1 to 5, such as 1, 2, 3, 4 or 5, and in which X is a naturally occurring amino acid, preferably no cysteine.

In the polypeptides described above, the one or more immunoglobulin single variable domains and the one or more groups, residues, moieties or binding units may be linked directly to each other and/or via one or more suitable linkers or spacers. For example, when the one or more groups, residues, moieties or binding units are amino acids, the linkers may also be an amino acid, so that the resulting polypeptide is a fusion protein or fusion polypeptide.

As used herein, the term “linker” denotes a peptide that fuses together two or more ISVs into a single molecule. The use of linkers to connect two or more (poly)peptides is well known in the art. Further exemplary peptidic linkers are shown in Table B. One often used class of peptidic linker are known as the “Gly-Ser” or “GS” linkers. These are linkers that essentially consist of glycine (G) and serine (S) residues, and usually comprise one or more repeats of a peptide motif such as the GGGGS (SEQ ID NO:154) motif (for example, having the formula (Gly-Gly-Gly-Gly-Ser)n (SEQ ID NO: 152) in which n may be 1, 2, 3, 4, 5, 6, 7 or more). Some often-used examples of such GS linkers are 9GS linkers (GGGGSGGGS, SEQ ID NO: 157), 15GS linkers (n=3) and 35GS linkers (n=7). Reference is for example made to Chen et al. 2013 (Adv. Drug Deliv. Rev. 65(10): 1357-1369) and Klein et al. 2014 (Protein Eng. Des. Sel. 27 (10): 325-330).

TABLE B
Linker sequences (“ID” refers to the SEQ ID NO as used herein)

| Name | ID | Amino acid sequence |
|---|---|---|
| 3 A linker | 153 | AAA |
| 5GS linker | 154 | GGGGS |
| 7GS linker | 155 | SGGSGGS |
| 8GS linker | 156 | GGGGSGGS |
| 9GS linker | 157 | GGGGSGGGS |
| 10GS linker | 158 | GGGGSGGGGS |
| 15GS linker | 159 | GGGGSGGGGSGGGGS |
| 18GS linker | 160 | GGGGSGGGGSGGGGSGGS |
| 20GS linker | 161 | GGGGSGGGGSGGGGSGGGGS |
| 25GS linker | 162 | GGGGSGGGGSGGGGSGGGGSGGGGS |
| 30GS linker | 163 | GGGGSGGGGSGGGGSGGGGSGGGGSGGGGS |
| 35GS linker | 164 | GGGGSGGGGSGGGGSGGGGSGGGGSGGGGSGGGGS |
| 40GS linker | 165 | GGGGSGGGGSGGGGSGGGGSGGGGSGGGGSGGGGSGGGGS |
| G1 hinge | 166 | EPKSCDKTHTCPPCP |
| 9GS-G1 hinge | 167 | GGGGSGGGSEPKSCDKTHTCPPCP |
| Llama upper long | 168 | EPKTPKPQPAAA |
| hinge region | | |
| G3 hinge | 169 | ELKTPLGDTTHTCPRCPEPKSCDTPPPCPRCPEPKSCDTPPPCPRCPEPKSCDTPPPCPRCP |

In one aspect, the disclosure also relates to such amino acid sequences and/or V_{HH}s that can bind to and/or are directed against CD8 and that comprise CDR sequences that are generally as further defined herein, to suitable fragments thereof, as well as to polypeptides that comprise or essentially consist of one or more of such V_{HH}s and/or suitable fragments. In some aspect, the disclosure relates to V_{HH}s with SEQ ID NO: 77. In particular, the disclosure in some specific aspects provides:
I) amino acid sequences that are directed against CD8 and that have at least 80%, preferably at least 85%, such as 90% or 95% or more sequence identity with SEQ ID NO: 77;
II) amino acid sequences that cross-block the binding of the amino acid sequence of SEQ ID NO: 77 to CD8 and/or that compete with at least the amino acid sequence of SEQ ID NO: 77 for binding to CD8;

Such amino acid sequences may be as further described herein (and may for example be V_{HH}s); as well as polypeptides of the disclosure that comprise one or more of such amino acid sequences (which may be as further described herein), and particularly bispecific (or multispecific) polypeptides as described herein, and nucleic acid sequences that encode such amino acid sequences and polypeptides. Such amino acid sequences and polypeptides do not include any naturally occurring ligands.

In some embodiments, the CD8 is derived from a mammalian animal, such as a human being. In one specific, but non-limiting aspect, the disclosure relates to an amino acid sequence directed against CD8, that comprises:
a) the amino acid sequence of SEQ ID NO: 77;
b) amino acid sequences that have at least 80% amino acid identity with a SEQ ID NO: 77, or
c) amino acid sequences that have 3, 2, or 1 amino acid difference with SEQ ID NO: 77;
or any suitable combination thereof.

In some embodiments, disclosed is a V_{HH} against CD8, which consist of 4 framework regions (FR1 to FR4 respectively) and 3 complementarity determining regions (CDR1 to CDR3 respectively). In some embodiments, in such a V_{HH}:
(I) CDR1 comprises or essentially consists of an amino acid sequence of GSTFSDYG (SEQ ID NO: 100),
or amino acid sequences that have at least 80%, at least 90%, at least 95%, at least 99% or more sequence identity with GSTFSDYG (SEQ ID NO: 100), in which (1) any amino acid substitution is a conservative amino acid substitution; and/or (2) said amino acid sequence only contains amino acids substitutions, and no amino acid deletions or insertions, compared to GSTFSDYG (SEQ ID NO: 100);
and/or from the group consisting of amino acids sequences that have 2 or only 1 amino acid difference(s) with GSTFSDYG (SEQ ID NO: 100), in which
any amino acid substitution is a conservative amino acid substitution; and/or
said amino acid sequence only contains amino acid substitutions, and no amino acid deletions or insertions, compared to GSTFSDYG (SEQ ID NO: 100).
(II) CDR2 comprises or essentially consists of an amino acid sequence of IDWNGEHT (SEQ ID NO: 101),
or amino acid sequences that have at least 80%, at least 90%, at least 95%, at least 99% or more sequence identity with IDWNGEHT (SEQ ID NO: 101), in which (1) any amino acid substitution is a conservative amino acid substitution; and/or (2) said amino acid sequence only contains amino acids substitutions, and no amino acid deletions or insertions, compared to IDWNGEHT (SEQ ID NO: 101);
and/or from the group consisting of amino acids sequences that have 2 or only 1 amino acid difference(s) with IDWNGEHT (SEQ ID NO: 101), in which
any amino acid substitution is a conservative amino acid substitution; and/or
said amino acid sequence only contains amino acid substitutions, and no amino acid deletions or insertions, compared to IDWNGEHT (SEQ ID NO: 101).
(III) CDR3 comprises or essentially consists of an amino acid sequence of AADALPYTVRKYNY (SEQ ID NO: 102),
or amino acid sequences that have at least 80%, at least 90%, at least 95%, at least 99% or more sequence identity with AADALPYTVRKYNY (SEQ ID NO: 102), in which (1) any amino acid substitution is a conservative amino acid substitution; and/or (2) said amino acid sequence only contains amino acids substitutions, and no amino acid deletions or insertions, compared to AADALPYTVRKYNY (SEQ ID NO: 102);
and/or from the group consisting of amino acids sequences that have 2 or only 1 amino acid difference(s) with AADALPYTVRKYNY (SEQ ID NO: 102), in which
any amino acid substitution is a conservative amino acid substitution; and/or
said amino acid sequence only contains amino acid substitutions, and no amino acid deletions or insertions, compared to AADALPYTVRKYNY (SEQ ID NO: 102).
CD8 V_{HH}s as disclosed herein may comprise one, two or all three of the CDRs explicitly listed above. In some embodiments, the CD8 V_{HH} comprises:

CDR1:
(SEQ ID NO: 100)
GSTFSDYG,
based on IMGT designation;
CDR2:
(SEQ ID NO: 101)
IDWNGEHT,
based on IMGT designation; and
CDR3:
(SEQ ID NO: 102)
AADALPYTVRKYNY,
based on IMGT designation.

In the V_{HH}s of the disclosure that comprise the combinations of CDRs mentioned above, each CDR can be replaced by a CDR chosen from the group consisting of amino acid sequences that have at least 80%, preferably at least 90%, more preferably at least 95%, even more preferably at least 99% sequence identity with the mentioned CDRs; in which:
(1) any amino acid substitution is preferably a conservative amino acid substitution; and/or
(2) said amino acid sequence preferably only contains amino acid substitutions, and no amino acid deletions or insertions, compared to the above amino acid sequence(s);
and/or chosen from the group consisting of amino acid sequences that have 3, 2 or only 1 (as indicated in the preceding paragraph) “amino acid difference(s)” with the mentioned CDR(s) one of the above amino acid sequences, in which:
(1) any amino acid substitution is preferably a conservative amino acid substitution; and/or
(2) said amino acid sequence preferably only contains amino acid substitutions, and no amino acid deletions or insertions, compared to the above amino acid sequence(s).

In one embodiment, the CD8 V_{HH} is BDSn:

(SEQ ID NO: 77)
Anti-CD8 BDSn Nb sequence
(CDR1, CDR2, CDR3 underlined based
on IMGT designation):
EVQLVESGGGLVQAGGSLRLSCAASGSTFSDYGVGWF
RQAPGKGREFVADIDWNGEHTSYADSVKGRFATSRDN
AKNTAYLQMNSLKPEDTAVYYCAADALPYTVRKYNYW
GQGTQVTVSSGGCGGHHHHHH

In some embodiments, a CD8 V_{HH} of the present disclosure binds to CD8 with a dissociation constant (KD) of 10⁻⁵ to 10⁻¹² moles/liter (M) or less, and preferably 10⁻⁷ to 10-12 moles/liter (M) or less and more preferably 10⁻⁸ to 10⁻¹²moles/liter (M), and/or with an association constant (KA) of at least 10⁷ M⁻¹, preferably at least 10⁸ M⁻¹, more preferably at least 10⁹ M⁻¹, such as at least 10¹² M⁻¹; and in particular with a KD less than 500 nM, preferably less than 200 nM, more preferably less than 10 nM, such as less than 5 nM. The KD and KA values of the V_{HH} of the disclosure against vWF can be determined in a manner known per se, for example using the assay described herein. More generally, the V_{HH}s described herein preferably have a dissociation constant with respect to vWF that is as described in this paragraph.

Generally, it should be noted that the term V_{HH} as used herein in its broadest sense is not limited to a specific biological source or to a specific method of preparation. For example, as will be discussed in more detail below, the V_{HH}s can be obtained (1) by isolating the VHH domain of a naturally occurring heavy chain antibody; (2) by expression of a nucleotide sequence encoding a naturally occurring VHH domain; (3) by “humanization” (as described below) of a naturally occurring VHH domain or by expression of a nucleic acid encoding a such humanized VHH domain; (4) by “camelization” (as described below) of a naturally occurring VH domain from any animal species, in particular a species of mammal, such as from a human being, or by expression of a nucleic acid encoding such a camelized VH domain; (5) by “camelisation” of a “domain antibody” or “Dab” as described by Ward et al (supra), or by expression of a nucleic acid encoding such a camelized VH domain; (6) using synthetic or semi-synthetic techniques for preparing proteins, polypeptides or other amino acid sequences; (7) by preparing a nucleic acid encoding a VHH using techniques for nucleic acid synthesis, followed by expression of the nucleic acid thus obtained; and/or (8) by any combination of the foregoing. Suitable methods and techniques for performing the foregoing will be clear to the skilled person based on the disclosure herein and for example include the methods and techniques described in more detail hereinbelow.

In some embodiments, the CD8 V_{HH}s of the present disclosure do not have an amino acid sequence that is exactly the same as (i.e. as a degree of sequence identity of 100% with) the amino acid sequence of a naturally occurring VH domain, such as the amino acid sequence of a naturally occurring VH domain from a mammal, and in particular from a human being.

One class of CD8 V_{HH}s of the disclosure comprises V_{HH}s with an amino acid sequence that corresponds to the amino acid sequence of a naturally occurring VHH domain, but that has been “humanized”, i.e. by replacing one or more amino acid residues in the amino acid sequence of said naturally occurring VHH sequence by one or more of the amino acid residues that occur at the corresponding position(s) in a VH domain from a conventional 4-chain antibody from a human being (e.g., indicated above). It should be noted that such humanized CD8 V_{HH}s of the present disclosure can be obtained in any suitable manner known per se (i.e. as indicated under points (1)-(8) above) and thus are not strictly limited to polypeptides that have been obtained using a polypeptide that comprises a naturally occurring VHH domain as a starting material.

Another class of CD8 V_{HH}s of the present disclosure comprises V_{HH}s with an amino acid sequence that corresponds to the amino acid sequence of a naturally occurring VH domain that has been “camelized”, i.e. by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. This can be performed in a manner known per se, which will be clear to the skilled person, for example on the basis of the further description below. Reference is also made to WO 94/04678. Such camelization may preferentially occur at amino acid positions which are present at the VH-VL interface and at the so-called Camelidae hallmark residues (see for example also WO 94/04678), as also mentioned below. In some embodiments, the VH domain or sequence that is used as a starting material or starting point for generating or designing the camelized V_{HH} is a VH sequence from a mammal, e.g., VH sequence of a human being. It should be noted that such camelized V_{HH}s of the present disclosure can be obtained in any suitable manner known per se and thus are not strictly limited to polypeptides that have been obtained using a polypeptide that comprises a naturally occurring VH domain as a starting material.

For example, both “humanization” and “camelization” can be performed by providing a nucleotide sequence that encodes such a naturally occurring VHH domain or VH domain, respectively, and then changing, in a manner known per se, one or more codons in said nucleotide sequence such that the new nucleotide sequence encodes a humanized or camelized VHH of the present disclosure, respectively, and then expressing the nucleotide sequence thus obtained in a manner known per se so as to provide the desired VHH. Alternatively, based on the amino acid sequence of a naturally occurring VHH domain or VH domain, respectively, the amino acid sequence of the desired humanized or camelized VHH of the present disclosure, respectively, can be designed and then synthesized de novo using techniques for peptide synthesis known per se. Also, based on the amino acid sequence or nucleotide sequence of a naturally occurring VHH domain or VH domain, respectively, a nucleotide sequence encoding the desired humanized or camelized VHH can be designed and then synthesized de novo using techniques for nucleic acid synthesis known per se, after which the nucleotide sequence thus obtained can be expressed in a manner known per se so as to provide the desired VHH.

Other suitable ways and techniques for obtaining VHHs and/or nucleotide sequences and/or nucleic acids encoding the same, starting from (the amino acid sequence of) naturally occurring VH domains or preferably VHH domains and/or from nucleotide sequences and/or nucleic acid sequences encoding the same will be clear from the skilled person, and may for example comprising combining one or more amino acid sequences and/or nucleotide sequences from naturally occurring VH domains (such as one or more FR's and/or CDR's) with one or more one or more amino acid sequences and/or nucleotide sequences from naturally occurring VHH domains (such an one or more FR's or CDR's), in a suitable manner so as to provide (a nucleotide sequence or nucleic acid encoding) a VHH. Also provided are compounds and constructs, and in particular proteins and polypeptides that comprise or essentially consists of at least one such amino acid sequence and/or VHH of the disclosure (or suitable fragments thereof), and optionally further comprises one or more other groups, residues, moieties or binding units. In some embodiments, such further groups, residues, moieties, binding units or amino acid sequences may or may not provide further functionality to the amino acid sequence and/or VHH (and/or to the compound or construct in which it is present) and may or may not modify the properties of the amino acid sequence and/or VHH.

The disclosure also encompasses any polypeptide of the present disclosure that has been glycosylated at one or more amino acid positions, usually depending on the host used to express the polypeptide. A polypeptide can comprise an amino acid sequence of a CD8 VHH of the present disclosure, which is fused at its amino terminal end, at its carboxy terminal end, or both at its amino terminal end and at its carboxy terminal end with at least one further amino acid sequence. Such further amino acid sequence may comprise at least one further VHH, so as to provide a polypeptide that comprises at least two, such as three, four or five, VHHs, in which said VHHs may optionally be linked via one or more linker sequences (as defined herein). Polypeptides of comprising CD8 VHH of the present disclosure and one or more another multivalent polypeptides. In a multivalent polypeptide, the two or more VHHs may be the same or different. For example, the two or more VHHs in a multivalent polypeptide:
may be directed against the same antigen, i.e. against the same parts or epitopes of said antigen or against two or more different parts or epitopes of said antigen; and/or:
may be directed against the different antigens;
or a combination thereof.

Thus, a bivalent polypeptide, for example:
may comprise two identical VHH;
may comprise a first VHH directed against a first part or epitope of an antigen and a second VHH directed against the same part or epitope of said antigen or against another part or epitope of said antigen;
   or may comprise a first VHH directed against a first antigen and a second VHH directed against a second antigen different from said first antigen;
   whereas a trivalent Polypeptide of the Invention for example:
may comprise three identical or different VHHs directed against the same or different parts or epitopes of the same antigen;
may comprise two identical or different VHHs directed against the same or different parts or epitopes on a first antigen and a third VHH directed against a second antigen different from said first antigen; or
may comprise a first VHH directed against a first antigen, a second VHH directed against a second antigen different from said first antigen, and a third VHH directed against a third antigen different from said first and second antigen.

The CD8 VHHs and polypeptides as disclosed herein can also be introduced and expressed in one or more cells, tissues or organs of a multicellular organism, for example for prophylactic and/or therapeutic purposes (e.g., as a gene therapy). For this purpose, the nucleotide sequences encoding the CD8 VHHs or polypeptides as disclosed herein can be introduced into the cells or tissues in any suitable way, for example as such (e.g., using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patent by administering a nucleic acid of the invention or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the invention and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y.). Giordano, Nature F Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. No. 5,580,859; 1 U.S. Pat. No. 5,589,5466; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

Accordingly, nucleic acid sequences encoding the CD8 VHHs as described herein, and expression construct and host cells comprising the nucleic acid sequence are also provided.

Also disclosed are methods of using CD8 VHHs and polypeptides of the present disclosure.

In some embodiments, a polypeptide comprising a CD8 VHH can be used in the lipid nanoparticles of the present disclosure for delivering a nucleic acid into an immune cell, as described herein. In some embodiments, CD8 VHHs and polypeptides of the present disclosure can be used to treat a condition or a disease in a subject in need thereof. In some embodiments, such conditions or diseases include, but are not limited to, cancer, infections, immune disorders, autoimmune diseases.

In some embodiments, a polypeptide comprising a CD8 VHH can be used in an imaging agent. In some embodiments, the imaging agent allows for the detection of human CD8 which is a specific biomarker found on the surface of a subset of T-cell for diagnostic imaging of the immune system. Imaging of CD8 allows for the in vivo detection of T-cell localization. Changes in T-cell localization can reflect the progression of an immune response and can occur over time as a result of various therapeutic treatments or even disease states. In some embodiments, it is used for imaging T-cell localization for immunotherapy.

In addition, CD8 plays a role in activating downstream signaling pathways that are important for the activation of cytolytic T cells that function to clear viral pathogens and provide immunity to tumors. CD8 positive T cells can recognize short peptides presented within the MHCI protein of antigen presenting cells. In some embodiments, a polypeptide comprising a CD8 VHH can potentiate signaling through the T cell receptor and enhance the ability of a subject to clear viral pathogens and respond to tumor antigens. Thus, in some embodiments, the antigen binding constructs provided herein can be agonists and can activate the CD8 target.

### II. Ionizable Cationic Lipids

Provided herein are ionizable cationic lipids that can be used to produce lipid nanoparticle compositions to facilitate the delivery of a payload (e.g., a nucleic acid, such as a DNA or RNA, such as an mRNA) disposed therein to cells, e.g., mammalian cells, e.g., human cells, e.g., immune cells. The ionizable cationic lipids have been designed to enable intracellular delivery of a nucleic acid, e.g., mRNA, to the cytosolic compartment of a target cell type and rapidly degrade into non-toxic components. The complex functionalities of the ionizable cationic lipids are facilitated by the interplay between the chemistry and geometry of the ionizable lipid head group, the hydrophobic “acyl-tail” groups and the linkers connecting the head group and the acyl tail groups. Typically, the pKₐ of the ionizable amine head group is designed to be in the range of 6-8, such as between 6.2-7.4, or between 6.7-7.2, such that it remains strongly cationic under acidic formulation conditions (e.g., pH 4-pH 5.5), neutral or slightly anionic in physiological pH (7.4) and cationic in the early and late endosomal compartments (e.g., pH 5.5-pH 7). The acyl-tail groups play a key role in fusion of the lipid nanoparticle with endosomal membranes and membrane destabilization through structural perturbation. The three-dimensional structure of the acyl-tail (determined by its length, and degree and site of unsaturation) along with the relative sizes of the head group and tail group are thought to play a role in promoting membrane fusion, and hence lipid nanoparticle endosomal escape (a key requirement for cytosolic delivery of a nucleic acid payload). The linker connecting the head group and acyl tail groups is designed to degrade by physiologically prevalent enzymes (e.g., esterases, or proteases) or by acid catalyzed hydrolysis.

In one aspect, the present invention provides a compound represented by Formula (I):

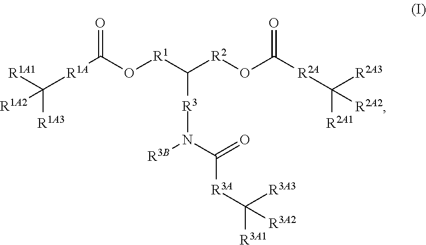

or a salt thereof, wherein:
R¹ and R² are each C₁₋₃ alkylene;
R³ is C₁₋₃ alkylene or a bond;
R^{1A} and R^{2A} are each a bond or C₁₋₁₀ alkylene;
R^{3A} is a bond or C₁₋₃ alkylene;
R^{1A1}, R^{2A1}, R^{3A1}, and R^{3A2} are each H;
R^{1A2} and R^{2A2} are each H, —(CH₂)₀₋₅C(O)OR^{a1}, or —(CH₂)₀₋₅OC(O)R^{a2};
R^{1A3} and R^{2A3} are each H, —(CH₂)₀₋₅C(O)OR^{a1}, or —(CH₂)₀₋₅OC(O)R^{a2};
R^{3A3} is —C(O)OR^{a1};
R^{a1} and R^{a2} are each independently C₁₋₂₀ alkyl;
R^{3B} is

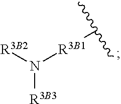

R^{3B1} is C₄₋₆ alkylene; and
R^{3B2} and R^{3B3} are each C₁₋₃ alkyl.

Any of the variables or substitutents provided herein is unsubstituted or substituted with one or more substituents. In some embodiments, any of the variables or substituents provided herein is optionally substituted. In some embodiments, any of the variables or substituents provided herein is optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R^{s11}, —NR^{s12}C(O)R^{s13}, and —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

In some embodiments, R¹ and R² are each C₁₋₃ alkylene. In some embodiments, R¹ and R² are each methylene. In some embodiments, R³ is C₁₋₃ alkylene or a bond. In some embodiments, R³ is a bond. In some embodiments, R¹ and R² are each methylene, and R³ is a bond.

In some embodiments, R^{1A} and R^{2A} are each a bond or C₁₋₁₀ alkylene. In some embodiments, R^{3A} is a bond or C₁₋₃ alkylene. In some embodiments, R^{1A} and R^{2A} are each a bond, —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, or —(CH₂)₁₀—. In some embodiments, R^{1A} and R^{2A} are each a bond, —(CH₂)₂—, —(CH₂)₅—, —(CH₂)₇—, or —(CH₂)₉—. In some embodiments, R^{3A} is a bond, —CH₂—, or —(CH₂)₂—. In some embodiments, R^{3A} is —CH₂—.

In some embodiments, R^{1A1}, R^{2A1}, R^{3A1}, and R^{3A2} are each H. In some embodiments, R^{1A2} and R^{2A2} are each H, —(CH₂)₀₋₅C(O)OR^{a1}, or —(CH₂)₀₋₅OC(O)R². In some embodiments, R^{1A3} and R^{2A3} are each H, —(CH₂)₀₋₅C(O)OR^{a1}, or —(CH₂)₀₋₅OC(O)R^{a2}. In some embodiments, R^{3A3} is —C(O)OR^{a1}.

In some embodiments, R^{1A2} and R^{2A2} are each —OC(O)(C₁₋₁₅ alkyl), —C(O)O(C₁₋₁₅ alkyl), —OC(O)CH(C₁₋₁₀ alkyl)(C₁₋₁₀ alkyl), —C(O)OCH(C₁₋₁₀ alkyl)(C₁₋₁₀ alkyl), —(CH₂)C(O)O(C₁₋₁₀ alkyl), or —(CH₂)OC(O)(C₁₋₁₀ alkyl). In some embodiments, R^{1A2} and R^{2A2} are each —OC(O)(C₁₋₁₀ alkyl), —C(O)O(C₁₋₁₀ alkyl), —OC(O)CH(C₆ alkyl)(C₈ alkyl), —C(O)OCH(C₂₋₃ alkyl)(C₅₋₆ alkyl), or —(CH₂)C(O)O(C₁₀ alkyl). In some embodiments, R^{1A2} and R^{2A2} are each

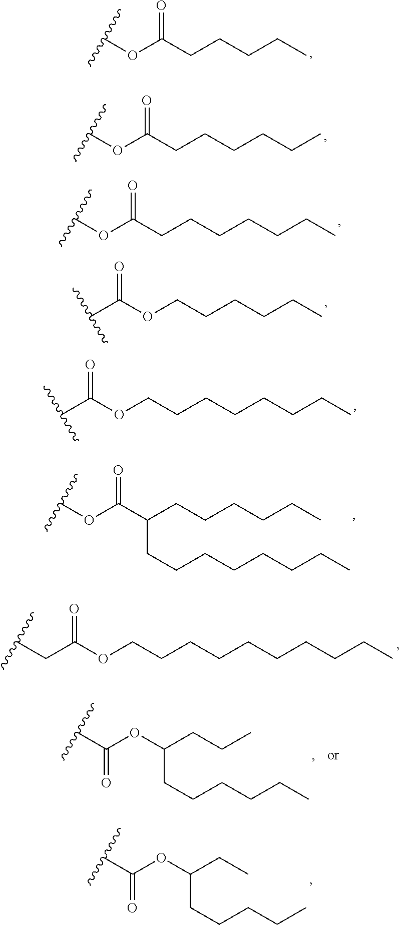

each of which is optionally substituted.

In some embodiments, R^{1A3} and R^{2A3} are each H, —OC(O)(C₁₋₁₅ alkyl), or —C(O)O(C₁₋₁₅ alkyl). In some embodiments, R^{1A3} and R^{2A3} are each H, —OC(O)(C₅₋₁₀ alkyl), —C(O)O(C₆₋₁₀ alkyl), or —(CH₂)C(O)O(C₁₀ alkyl). In some embodiments, R^{1A3} and R^{2A3} are each H,

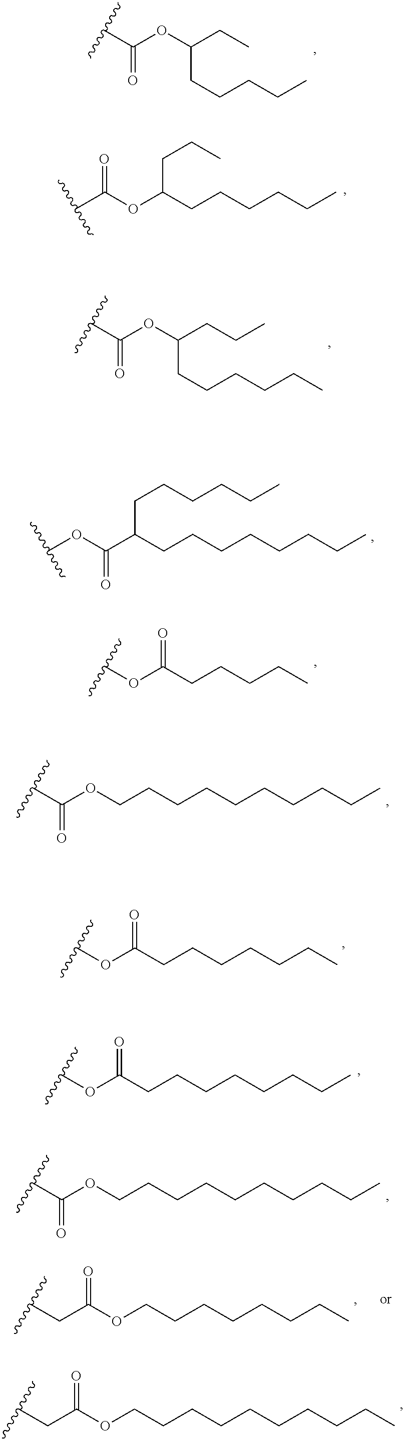

each of which is optionally substituted.

In some embodiments, R^{3A3} is —C(O)OCH(C₁₋₅ alkyl)(C₁₋₁₀ alkyl). In some embodiments, R^{3A3} is —C(O)OCH(C₃ alkyl)(C₆ alkyl). In some embodiments, R^{3A3} is

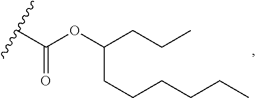 [SMILES: *C(=O)OC(CCC)CCCCCC]

which is optionally substituted.

In some embodiments, R^{3B1} is C₄₋₆ alkylene. In some embodiments, R^{3B2} and R^{3B3} are each C₁₋₃ alkyl. In some embodiments, R^{3B1} is —(CH₂)₄—. In some embodiments, R^{3B1} is —(CH₂)₅—. In some embodiments, R^{3B1} is —(CH₂)₆—. In some embodiments, R^{3B2} and R^{3B3} are each methyl. In some embodiments, R^{3B2} and R^{3B3} are each ethyl. In some embodiments,

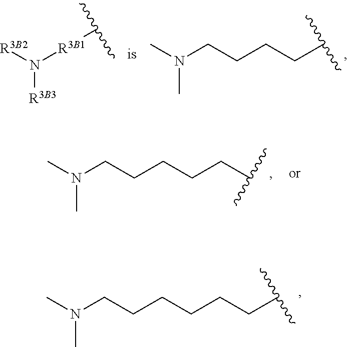

each of which is optionally substituted.

In some embodiments, R^{3B1} is unsubstituted or substituted. In some embodiments, R^{3B1} is optionally substituted. In some embodiments, R^{3B1} is unsubstituted. In some embodiments, R^{3B1} is not substituted with oxo.

In some embodiments, R^{3B} is

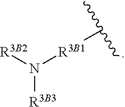

In some embodiments, R^{3B} is H. In some embodiments, R^{3B} is unsubstituted or substituted. In some embodiments, R^{3B} is unsubstituted.

In some embodiments, R^{3B2} and R^{3B3} are each independently and optionally substituted. In some embodiments, R^{3B2} and R^{3B3} are each independently H or C₁₋₆ alkyl optionally substituted with one or more substituents each independently selected from the group consisting of —OH and —O—(C₁₋₆ alkyl). In some embodiments, R^{3B2} and R^{3B3} are each independently H or C₁₋₆ alkyl optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13}, and —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted. In some embodiments, R^{3B2} and R^{3B3} are each independently H, methyl, ethyl, propyl, butyl, or pentyl, each of which is optionally substituted with one or more substituents each independently selected from the group consisting of —OH and —O—(C₁₋₆ alkyl). In some embodiments, R^{3B2} and R^{3B3} are each independently methyl or ethyl, each optionally substituted with one or more —OH. In some embodiments, R^{3B2} and R^{3B3} are each methyl or each ethyl, each optionally substituted with one or more —OH. In some embodiments, R^{3B2} and R^{3B3} are each unsubstituted methyl.

In one aspect, the present invention provides a compound represented by Formula (Ia):

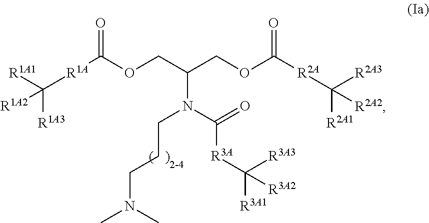

or a salt thereof, wherein R^{1A}, R^{2A}, R^{3A}, R^{1A1}, R^{1A2}, R^{1A3}, R^{2A1}, R^{2A2}, R^{2A3}, R^{3A1}, R^{3A2}, and R^{3A3} are as defined for Formula (I) or any variation or embodiment thereof.

In some embodiments, provided is a compound of Formula (II):

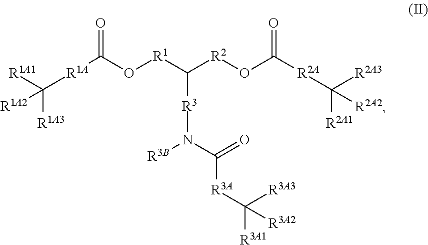

or a salt thereof.

In some embodiments, R¹, R², and R³ are each independently a bond or C₁₋₃ alkylene. In some embodiments, R^{1A}, R^{2A}, and R^{A} are each independently a bond or C₁₋₁₀ alkylene. In some embodiments, R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}. In some embodiments, R^{a1} and R^{a2} are each independently C₁₋₂₀ alkyl or C₁₋₂₀ alkenyl. In some embodiments, R^{3B} is

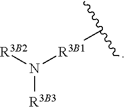

In some embodiments, R^{3B1} is C₄₋₆ alkylene. In some embodiments, R^{3B2} and R^{3B3} are each independently H or C₁₋₆ alkyl.

In some embodiments, R¹, R², and R³ are each independently a bond or C₁₋₃ alkylene. In some embodiments, R¹, R², and R³ are each independently a bond or methylene. In some embodiments, R¹ and R² are each methylene and R³ is a bond. In some embodiments, R¹, R², and R³ are each methylene. In some embodiments, R¹, R², and R³ are each independently unsubstituted or substituted. In some embodiments, R¹, R², and R³ are unsubstituted.

In some embodiments, R^{1A}, R^{2A}, and R^{3A} are each independently a bond or C₁₋₁₀ alkylene. In some embodiments, R^{1A}, R^{2A}, and R^{3A} are each independently a bond or —(CH₂)₁₋₁₀—. In some embodiments, R^{1A} and R^{2A} are each independently a bond, —CH₂—, —(CH₂)₂—, —(CH₂)₃—, —(CH₂)₄—, —(CH₂)₅—, —(CH₂)₆—, —(CH₂)₇—, or —(CH₂)₈—. In some embodiments, R^{1A} and R^{2A} are each a bond, each —CH₂—, each —(CH₂)₂—, each —(CH₂)₃—, each —(CH₂)₄—, each —(CH₂)₅—, each —(CH₂)₆—, each —(CH₂)₇—, or each —(CH₂)₈—. In some embodiments, R^{1A} and R^{2A} are each independently a bond, —(CH₂)₂—, —(CH₂)₄—, —(CH₂)₆—, —(CH₂)₇—, or —(CH₂)₈—. In some embodiments, R^{1A} and R^{2A} are each a bond, each —(CH₂)₂—, each —(CH₂)₄—, each —(CH₂)₆—, each —(CH₂)₇—, or each —(CH₂)₈—. In some embodiments, R^{3A} is a bond, —CH₂—, —(CH₂)₂—, or —(CH₂)₇—. In some embodiments, R^{1A}, R^{2A}, and R^{3A} are each independently unsubstituted or substituted. In some embodiments, R^{1A}, R^{2A}, and R^{3A} are unsubstituted.

In some embodiments, R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}. In some embodiments, R^{1A1}, R^{1A2}, R^{1A3}, R^{2A1}, R^{2A2}, R^{2A3}, R^{3A1}, R^{3A2}, and R^{3A3} are each independently H, C₁₋₁₅ alkyl, —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), —(CH₂)₀₋₄OC(O)CH(C₁₋₁₀ alkyl)(C₁₋₁₅ alkyl), —(CH₂)₀₋₄C(O)OCH₂(C₁₋₁₅ alkyl), or —(CH₂)₀₋₄OC(O)CH₂(C₁₋₁₅ alkyl). In some embodiments, R^{1A1}, R^{1A2}, R^{1A3}, R^{2A1}, R^{2A2}, R^{2A3}, R^{3A1}, R^{3A2}, R^{3A3}, R¹, R², R³, R^{1A}, R^{2A}, and R^{3A} are each independently unsubstituted or substituted. In some embodiments, R^{1A1}, R^{1A2}, R^{1A3}, R^{2A1}, R^{2A2}, R^{2A3}, R^{3A1}, R^{3A2}, R^{3A3}, R¹, R², R³, R^{1A}, R^{2A}, and R^{3A} are each unsubstituted. In some embodiments, R^{1A1}, R^{1A2}, R^{1A3}, R^{2A1}, R^{2A2}, R^{2A3}, R^{3A1}, R^{3A2}, and R^{3A3} are each independently unsubstituted or substituted. In some embodiments, R^{1A1}, R^{1A2}, R^{1A3}, R^{2A1}, R^{2A2}, R^{2A3}, R^{3A1}, R^{3A2}, and R^{3A3} are each unsubstituted. In some embodiments, R¹, R², R³, R^{1A}, R^{2A}, and R^{3A} are each independently unsubstituted or substituted. In some embodiments, R¹, R², R³, R^{1A}, R^{2A}, and R^{3A} are each unsubstituted. In some embodiments, R¹, R², and R³ are each unsubstituted.

In some embodiments, R^{3B1} is unsubstituted. In some embodiments, R^{3B1} is not substituted with oxo.

In some embodiments, R^{1A1} and R^{2A1} are each independently —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H. In some embodiments, R^{1A2} and R^{2A1} are each —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H. In some embodiments, R^{1A1} and R^{2A1} are each

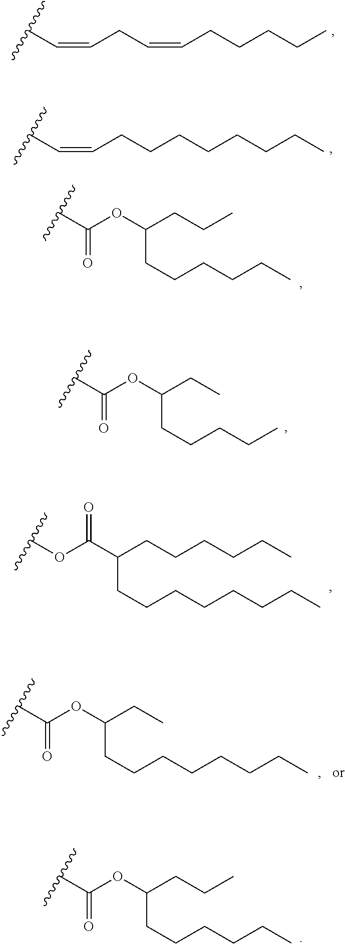

In some embodiments, R^{1A1} and R^{2A1} are each

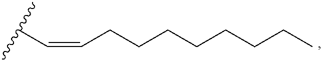 [SMILES: */C=C\CCCCCCCC]

In some embodiments, R^{1A2}, R^{1A3}, R^{2A2}, and R^{2A3} are each H.

In some embodiments, R^{1A1} and R^{2A1} are each C₁₋₁₅ alkyl; R^{1A2} and R^{2A2} are each C₁₋₁₅ alkyl; and R^{1A3} and R^{2A3} are each H. In some embodiments, R^{1A1} and R^{2A1} are each

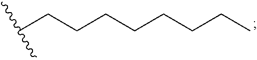 [SMILES: *CCCCCCCC ; ";"]

and R^{1A2} and R^{2A2} are each

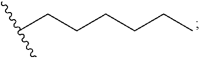 [SMILES: *CCCCCC ; ";"]

In some embodiments, R^{1A3} and R^{2A3} are each H. In some embodiments, R^{1A} and R^{2A} are each a bond.

In some embodiments, R^{1A1} and R^{2A1} are each —(CH₂)₀₋₄OC(O)CH₂(C₁₋₁₅ alkyl); R^{2A1} and R^{2A2} are each —(CH₂)₀₋₄C(O)OCH₂(C₁₋₁₅ alkyl); and R^{1A3} and R^{2A3} are each H. In some embodiments, R^{1A1} and R^{2A1} are each

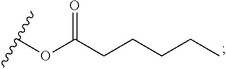 [SMILES: *OC(=O)CCCCC]

and R^{2A1} and R^{2A2} are each

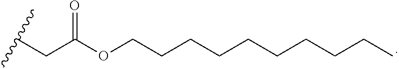 [SMILES: *CC(=O)OCCCCCCCCCC]

In some embodiments, R^{1A3} and R^{2A3} are each H. In some embodiments, R^{1A} and R^{2A} are each a bond.

In some embodiments, R^{1A1} and R^{2A1} are each —C(O)OCH₂(C₁₋₁₅ alkyl); R^{1A2} and R^{2A2} are each —(CH₂)₀₋₄C(O)OCH₂(C₁₋₁₅ alkyl); and R^{1A3} and R^{2A3} are each H. In some embodiments, R^{1A1} and R^{2A1} are each

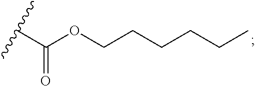 [SMILES: *C(=O)OCCCCCC]

and R^{1A2} and R^{2A2} are each

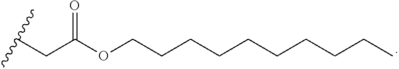 [SMILES: *CC(=O)OCCCCCCCCCC]

In some embodiments, R^{1A1} and R^{2A1} are each

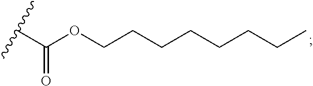 [SMILES: *C(=O)OCCCCCCCC]

and R^{2A1} and R^{2A2} are each

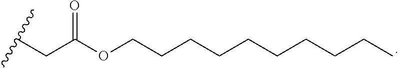 [SMILES: *CC(=O)OCCCCCCCCCC]

In some embodiments, R^{1A3} and R^{2A3} are each H. In some embodiments, R^{1A} and R^{2A} are each a bond.

In some embodiments, R^{3A1}, R^{3A2}, and R^{3A3} are each independently H, C₁₋₁₅ alkyl, —(CH₂)₀₋₄C(O)OCH(C₁₋₅ alkyl)(C₁₋₁₀ alkyl), —(CH₂)₀₋₄OC(O)CH(C₁₋₅ alkyl)(C₁₋₁₀ alkyl), —(CH₂)₀₋₄C(O)OCH₂(C₁₋₁₀ alkyl), or —(CH₂)₀₋₄OC(O)CH₂(C₁₋₁₀ alkyl).

In some embodiments, R^{3A1} and R^{3A2} are each independently C₁₋₁₅ alkyl; and R^{3A3} is H. In some embodiments, R^{3A1} and R^{3A2} are each independently ethyl, propyl, butyl, pentyl, hexyl, or heptyl. In some embodiments, R^{3A1} and R^{3A2} are each independently ethyl,

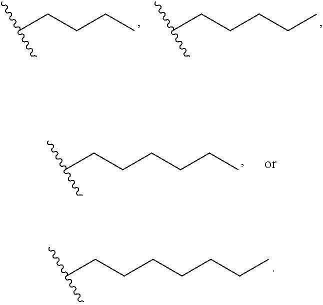

In some embodiments, R^{3A3} is H. In some embodiments, R^{3A} is a bond.

In some embodiments, R^{3A1} is C₁₋₁₅ alkyl; and R^{3A2} and R^{3A3} are each H. In some embodiments, R^{3A1} is

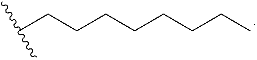 [SMILES: *CCCCCCCC ; "."]

In some embodiments, R^{3A2} and R^{3A3} are each H. In some embodiments, R^{3A} is a bond.

In some embodiments, R^{3A1} is —C(O)OCH(C₁₋₅ alkyl)(C₁₋₁₀ alkyl); and R^{3A2} and R^{3A3} are each H. In some embodiments, R^{3A1} is

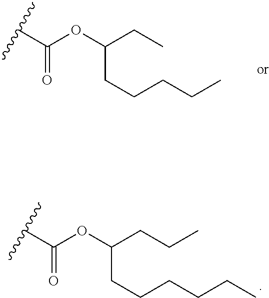 [SMILES: *C(=O)OC(CC)CCCCC ; *C(=O)OC(CCC)CCCCCC]

In some embodiments, R^{3A1} is

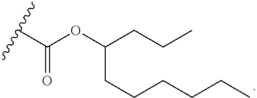 [SMILES: *C(=O)OC(CCC)CCCCCC]

In some embodiments, R^{3A} is ethylene or —(CH₂)₂—. In some embodiments, R^{3A2} and R^{3A3} are each H.

In some embodiments, R^{3A1} is —(CH₂)₀₋₄OC(O)CH₂(C₁₋₁₀ alkyl); R^{3A2} is —(CH₂)₀₋₄(O)OCH₂(C₁₋₁₀ alkyl); and R^{3A3} is H. In some embodiments, R^{3A1} is

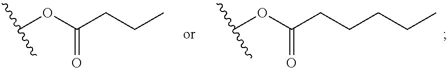

### and R^{3A2} is

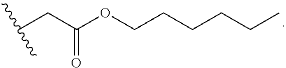 [SMILES: *CC(=O)OCCCCCC]

In some embodiments, R^{3A3} is H. In some embodiments, R^{3A} is a bond.

In some embodiments, R^{3A1} is —(CH₂)₀₋₄C(O)OCH₂(C₁₋₁₀ alkyl); R^{3A2} is —(CH₂)₀₋₄C(O)OCH₂(C₁₋₁₀ alkyl); and R^{3A3} is H. In some embodiments, R^{3A1} is

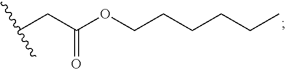 [SMILES: *CC(=O)OCCCCCC]

### and R^{3A2} is

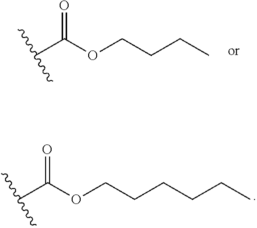

In some embodiments, R^{3A3} is H. In some embodiments, R^{3A} is a bond.

In some embodiments, R^{3A1}, R^{3A2}, and R^{3A3} are each H.

R^{a1} and R^{a2} are each independently C₁₋₂₀ alkyl or C₁₋₂₀ alkenyl. In some embodiments, R^{a1} and R^{a2} are each independently —(CH₂)₀₋₁₅CH₃ or —CH(C₁₋₁₀ alkyl)C₁₋₁₅ alkyl). In some embodiments, R^{a1} and R^{a2} are each independently

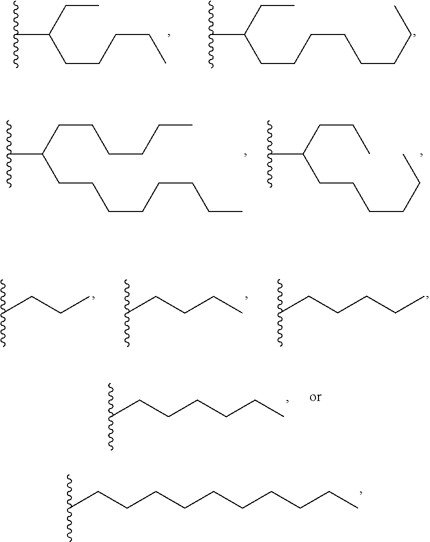

each of which is optionally substituted. In some embodiments, R^{a1} and R^{a2} are each independently unsubstituted or substituted. In some embodiments, R^{a1} and R^{a2} are unsubstituted.

In some embodiments, R^{3B} is

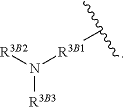

In some embodiments, R^{3B} is H. In some embodiments, R^{3B} is unsubstituted or substituted. In some embodiments, R^{3B} is unsubstituted.

In some embodiments, R^{3B1} is C₄₋₆ alkylene. In some embodiments, R^{3B1} is unsubstituted or substituted. In some embodiments, R^{3B1} is optionally substituted.

In some embodiments, R^{3B2} and R^{3B3} are each independently and optionally substituted. In some embodiments, R^{3B2} and R^{3B3} are each independently H or C₁₋₆ alkyl optionally substituted with one or more substituents each independently selected from the group consisting of —OH and —O—(C₁₋₆ alkyl). In some embodiments, R^{3B2} and R^{3B3} are each independently H or C₁₋₆ alkyl optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13}, and —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s5} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted. In some embodiments, R^{3B2} and R^{3B3} are each independently H, methyl, ethyl, propyl, butyl, or pentyl, each of which is optionally substituted with one or more substituents each independently selected from the group consisting of —OH and —O—(C₁₋₆ alkyl). In some embodiments, R^{3B2} and R^{3B3} are each independently methyl or ethyl, each optionally substituted with one or more —OH. In some embodiments, R^{3B2} and R^{3B3} are each methyl or each ethyl, each optionally substituted with one or more —OH. In some embodiments, R^{3B2} and R^{3B3} are each unsubstituted methyl.

In some embodiments,

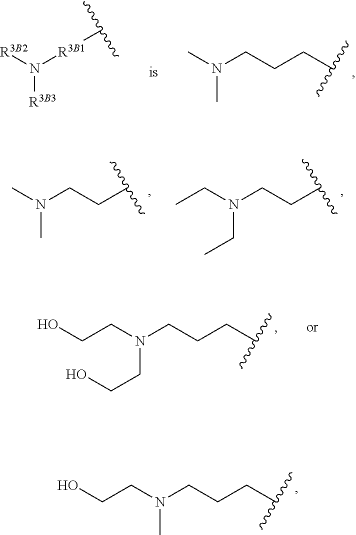

each of which is optionally substituted.

### III. Lipid-Immune Cell Targeting Group Conjugates

As discussed herein, the LNPs may be targeted to a particular cell type, e.g., an immune cell, e.g., a macrophages, monocytes, or dendritic cells. This can be accomplished by using one or more of the lipids described herein. Furthermore, targeting can be enhanced by including a targeting group at a solvent accessible surface of an LNP particle. For example, targeting groups may include a member of a specific binding pair, e.g., an antibody-antigen pair, a ligand-receptor pair, etc. In certain embodiments, the targeting group is an antibody. Targeting can be implemented, for example, by using lipid-immune cell targeting group conjugates described herein.

Optionally, the targeting moiety is an antibody fragment without an Fc component. Previous attempts to target circulating immune cells with LNPs have employed full antibodies (WO 2016/189532 A1). Liposomes or lipid based particles with conjugated full antibodies clear more quickly from the circulation due to engagement of the Fc, reducing their potential for reaching the target cell of interest (Harding et al. (1997) Biochim Biophys. Acta 1327, 181-192; Sapra et al. (2004) Clin Cancer Res 10, 1100-1111; Aragnol et al., (1986) Proc Natl Acad Sci USA 83, 2699-2703). Liposomes targeted with antibody fragments retain their long circulating properties, like those targeted to EGFR (Mamot et al., (2005) Cancer Res 65, 11631-11638), ErbB2 (Park et al. (2002) Clin Cancer Res 8, 1172-1181), or EphA2 (Kamoun et al., 2019 Nat. Biomed. Eng 3, 264-280). In addition, lipid based carriers can be prepared using a micellar insertion process that allows for the nearly quantitative incorporation of the antibody conjugation following its separate manufacturing (Nellis et al. (2005) Biotechnol Prog 21, 221-232), compared to a highly inefficient insertion when conjugating full IgGs (Ishida et al. (1999) FEBS Lett. 460, 129-133) or the need to complete conjugation directly on an intact LNP (WO 2016/189532 A1). scFv, Fab, or VHH fragments can also be directly conjugated to activated PEG-lipids to make insertable conjugates.

In some embodiments, PEG-(lipid) is equivalent to (lipid)-PEG.

In certain embodiments, a targeting group may be a surface-bound antibody or surface bound antigen binding fragment thereof, which can permit tuning of cell targeting specificity. This is especially useful since highly specific antibodies can be raised against an epitope of interest for the desired targeting site. In one embodiment, multiple different antibodies can be incorporated into, and presented at the surface of an LNP, where each antibody binds to different epitopes on the same antigen or different epitopes on different antigens. Such approaches can increase the avidity and specificity of targeting interactions to a particular target cell.

A targeting group or combination of targeting groups can be selected based on the desired localization, function, or structural features of a given target cell. For example, in order to target a T-cell, T-cell population or T-cell subpopulation, one or more antibodies or antigen binding fragments or antigen binding derivatives thereof may be selected that target a T-cell, such as via a T-cell surface antigen. Exemplary T-cell surface antigens include, but are not limited to, for example, CD2, CD3, CD4, CD5, CD7, CD8, CD28, CD39, CD69, CD103, CD137, CD45, T-cell receptor (TCR) β, TCR-a, TCR-a/b, TCR-g/d, PD1, CTLA4, TIM3, LAG3, CD18, IL-2 receptor, CD11a, GL7, TLR2, TLR4, TLR5 and IL-15 receptor. In order to target an NK cell, or NK cell population, one or more antibodies, antigen binding fragments or antigen binding derivatives thereof may be selected that target an NK cell such as via a NK cell surface antigen. Exemplary NK cell surface antigens include, but are not limited to, CD48, CD56, CD85a, CD85c, CD85d, CD85e, CD85f, CD85i, CD85j, CD158b2, CD161, CD244, CD16a, CD16b, IL-2 receptor, CD27, CD28, CD48, CD69, CD70, CD86, CD112, CD122, CD155, CD161, CD244, CD266, CD314/NKG2D, CD336/NKP44, CD337/NKP30. In order to target a B cell or B cell population, one or more antibodies, antigen binding fragments or antigen binding derivatives thereof may be selected that target a B cell such as via a B cell antigen. Exemplary B cell antigens include, but are not limited to, CD19 for all B cells except plasma cells, CD19, CD25, and CD30 for activated B cells, CD27, CD38, CD78, CD138, and CD319 for plasma cells, CD20, CD27, CD40, CD80 and PDL-2 for memory cells, Notch2, CD1, CD21, and CD27 for marginal zone B cells, CD21, CD22, and CD23 for follicular B cells, and CD1, CD5, CD21, CD24, and TLR4 for regulatory B cells.

In order to target a macrophage, macrophage population or macrophage subpopulation, one or more antibodies or antigen binding fragments or antigen binding derivatives thereof may be selected that target a macrophage, such as via a macrophage surface antigen. In some embodiments, the antigen is a M1 macrophage specific antigen. In some embodiments, the antigen is a M2 macrophage specific antigen. Exemplary macrophage surface antigens include, but are not limited to, for example, CDIIB, CD80, CD86, HLA, CD68, CD163, CD206. In some embodiments, tumor macrophages are targeted, and the antigen is CD206.

In order to target a monocyte, monocyte population or monocyte subpopulation, one or more antibodies or antigen binding fragments or antigen binding derivatives thereof may be selected that target a monocyte, such as via a monocyte surface antigen. Exemplary monocyte surface antigens include, but are not limited to, for example, CD14, CCR2, CCR5, CD62L, HLA, CD68, CXCR1, CXCR3, and CD11c.

In order to target a dendritic cell, dendritic cell population or dendritic subpopulation, one or more antibodies or antigen binding fragments or antigen binding derivatives thereof may be selected that target a dendritic cell, such as via a dendritic surface antigen. Exemplary dendritic surface antigens include, but are not limited to, for example, DEC205 (see Katakowski, 2016:24(1):146-155, Molecular Therapy).

In certain embodiments, targeting can be implemented, for example, by using lipid-immune cell targeting group conjugates described herein. Exemplary lipid-immune cell targeting group conjugates can include compounds of Formula (II),

[Lipid]-[optional linker]-[immune cell targeting group, e.g., macrophage targeting molecule, e.g., anti-CDIIB antibody, anti-CD80 antibody, anti-CD86 antibody, anti-CD68 antibody, anti-CD163 antibody, and/or anti-CD206 antibody.] (Formula II).

In some embodiments, the immune cell targeting group is a polypeptide, and the lipid is conjugated to the N-terminus, C-terminus, or anywhere in the middle part of the polypeptide.

In certain embodiments, the targeting group or targeting molecule is a T-cell targeting agent, for example, an antibody, that binds to a T-cell antigen selected from the group consisting of CD2, CD3, CD4, CD5, CD7, CD8, CD28, CD137, CD45, T-cell receptor (TCR)β, TCR-a, TCR-a/b, TCR-g/d, PD1, CTLA4, TIM3, LAG3, CD18, IL-2 receptor, CD11a, TLR2, TLR4, TLR5, IL-7 receptor, or IL-15 receptor. In certain embodiments, the T cell antigen may be CD2, and the targeting group can be, for example, an anti-CD2 antibody. In certain embodiments, the T cell antigen may be CD3, and the targeting group can be, for example, an anti-CD3 antibody. In certain embodiments, the T cell antigen may be CD4, and the targeting group can be, for example, an anti-CD4 antibody. In certain embodiments, the T cell antigen may be CD5, and the targeting group can be, for example, an anti-CD5 antibody. In certain embodiments, the T cell antigen may be CD7, and the targeting group can be, for example, an anti-CD7 antibody. In certain embodiments, the T cell antigen may be CD8, and the targeting group can be, for example, an anti-CD8 antibody. In certain embodiments, the T cell antigen may be TCR β, and the targeting group can be, for example, an anti-TCR β antibody. In some embodiments, the antibody is a human or humanized antibody.

An exemplary CD2 binding agent can be an antibody selected from the group consisting of 9.6 (https://academic.oup.com/intimm/article/10/12/1863/744536), 9-1 (https://academic.oup.com/intimm/article/10/12/1863/744536), TS2/18.1.1 (ATCC HB-195), Lo-CD2b (ATCC PTA-802), Lo-CD2a/BTI-322 (U.S. Pat. No. 6,849,258B1), Sipilzumab/MEDI-507 (U.S. Pat. No. 6,849,258B1/en), 35.1 (ATCC HB-222), OKT11 (ATCC CRL-8027), RPA-2.1 (PCT Publication WO2020023559A1), AF1856 (R&D Systems), MAB18562 (R&D Systems), MAB18561 (R&D Systems), MAB1856 (R&D Systems), PAB30359 (Abnova Corporation), 10299-1 (Abnova Corporation), and antigen binding fragments thereof. In certain embodiments, the binding agent comprises a heavy chain variable domain (V_{H}) and a light chain variable domain (V_{L}) of an antibody selected from the group consisting of AF1856 (R&D Systems), MAB18562 (R&D Systems), MAB18561 (R&D Systems), MAB1856 (R&D Systems), PAB30359 (Abnova Corporation), and 10299-1 (Abnova Corporation). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of AF1856 (R&D Systems), MAB18562 (R&D Systems), MAB18561 (R&D Systems), MAB1856 (R&D Systems), PAB30359 (Abnova Corporation), and 10299-1 (Abnova Corporation).

An exemplary CD2 binding agent can also be selected from antibodies or antibody fragments employing CDRs of clones 9.6, 9-1, TS2/18.1.1, Lo-CD2b, Lo-CD2a, BTI-322, sipilzumab, 35.1, OKT11, RPA-2.1, SQB-3.21, LT2, TS1/8, UT329, 4F22, OX-34, UQ2/42, MU3, U7.4, NFN-76, or MOM-181-4-F(E).

An exemplary CD3 binding agent (CD3γ/δ/ε, CD3γ, CD3δ, CD3γ/ε, CD3δ/ε, or CD3ε) can be an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), 16669-1-AP (CD3δ, Invitrogen) and antigen binding fragments thereof. In certain embodiments, the binding agent comprises a V_{H} domain and a V_{L} domain of an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3δ, Invitrogen). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3δ, Invitrogen).

An exemplary CD3 binding agent can also be selected from antibodies or antibody fragments employing CDRs of clones hsp34, OKT-3, UCHT1, 38.1, HIT3a, RFT8, SK7, BC3, SP34-2, HU291, TRX4, Catumaxomab, teplizumab, 3-106, 3-114, 3-148, 3-190, 3-271, 3-550, 4-10, 4-48, H2C, F12Q, I2C, SP7, 3F3A1, CD3-12, 301, RIV9, JB38-29, JE17-74, GT0013, 4E2, 7A4, 4D10A6, SPV-T3b, M2AB, ICO-90, 30A1 or Hu38E4.v1 (US Patent Application 20200299409A1), REGN5458 (US Patent Application 20200024356A1), Blinatumomab (https://go.drugbank.com/drugs/DB09052/polypeptide_sequences.fasta). In some embodiments, the conjugate comprises a Fab, wherein the Fab comprises (a) a heavy chain fragment comprising the amino acid sequence of SEQ ID NO: 1 and a light chain fragment comprising the amino acid sequence of SEQ ID NO: 2 or 3.

An exemplary CD4 binding agent can be an antibody selected from the group consisting of Ibalizumab (https://www.genome.jp/dbget-bin/www_bget?D09575), AF1856 (R&D Systems), MAB554 (R&D Systems), BF0174 (Affinity Biosciences), PAB31115 (Abnova Corporation), CAL4 (Abcam), and antigen binding fragments thereof. In certain embodiments, the binding agent comprises a V_{H} domain and a V_{L} domain of an antibody selected from the group consisting of AF1856 (R&D Systems), MAB554 (R&D Systems), BF0174 (Affinity Biosciences), PAB31115 (Abnova Corporation), and CAL4 (Abcam). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of AF1856 (R&D Systems), MAB554 (R&D Systems), BF0174 (Affinity Biosciences), PAB31115 (Abnova Corporation), and CAL4 (Abcam).

An exemplary CD4 binding agent can also be selected from antibodies or antibody fragments employing CDRs of clones Ibalizumab, OKT4, RPA-T4, S3.5, SK3, N1UG0, RIV6, OTI18E3, MEM-241, B486A1, RFT-4g, 7E14, MDX.2, MEM-115, MEM-16, ICO-86, Edu-2, or ibalizumab.

An exemplary CD5 binding agent can be an antibody selected from the group consisting of He3, MAB1636 (R&D Systems), AF1636 (R&D Systems), MAB115 (R&D Systems), C5/473+CD5/54/F6 (Abcam), CD5/54/F6 (Abcam), 65152 (Proteintech), and antigen binding fragments thereof. In some embodiments, the binding agent comprises a V_{H} domain and a V_{L} of an antibody selected from the group consisting of MAB1636 (R&D Systems), AF1636 (R&D Systems), MAB115 (R&D Systems), C5/473+CD5/54/F6 (Abcam), CD5/54/F6 (Abcam), and 65152 (Proteintech). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of MAB1636 (R&D Systems), AF1636 (R&D Systems), MAB115 (R&D Systems), C5/473+CD5/54/F6 (Abcam), CD5/54/F6 (Abcam), and 65152 (Proteintech).

An exemplary CD5 binding agent can also be selected from antibodies or antibody fragments employing CDRs of clones of zolimomab, 5D7, L17F12, and UCHT2, 1D8, 3I21, 4H10, 8J23, 5O4, 4H2, 5G2, 8G8, 6M4, 2E3, 4E24, 4F10, 7J9, 7P9, 8E24, 6L18, 7H7, 1E7, 8J21, 7I11, 8M9, 1P21, 2H11, 3M22, 5M6, 5H8, 7I19, 1A2, 8E15, 8C10, 3P16, 4F3, 5M24, 5O24, 7B16, 1E8, 2H16, BLa1, 1804, DK23, Cris1, MEM-32, H65, 4C7, OX-19, Leu-1, 53-7.3, 4H8E6, T101, EP2952, D-9, H-3, HK231, N-20, Y2/178, H-300, CD5/54/F6, Q-20, CC17, MOM-18539-S(P), or MOM-18885-S(P).

An exemplary CD7 binding agent can be an antibody selected from the group consisting of MAB7579 (R&D Systems), AF7579 (R&D Systems), EPR22065 (Abcam), 1G10D8 (Proteintech), NBP2-32097 (Novus Biologicals), NBP2-38440 (Novus Biologicals), and antigen binding fragments thereof. In certain embodiments, the binding agent comprises a V_{H} domain and a V_{L} of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals).

An exemplary CD7 binding agent can also be selected from antibodies or antibody fragments employing CDRs of clones TH-69, 3Afl1, T3-3A1, 124-1D1, 3A1f, CD7-6B7, or VHH6.

An exemplary CD8 (CD8α, CD8α/α, CD8α/β or CD8β) binding agent can be an antibody selected from the group consisting of 2.43 (Invitrogen), Du CD8-1 (CD8α, Invitrogen), 9358-CD (CD8α/β, R&D Systems), MAB 116 (CD8α, R&D Systems), ab4055 (CD8α, Abcam), C8/144B (CD8α, Novus Biologicals), YTS105.18 (CD8α, Novus Biologicals), TRX2 (https://patents.justia.com/patent/20170198045), and antigen binding fragments thereof. In certain embodiments, the binding agent comprises a V_{H} domain and a V_{L} domain of an antibody selected from the group consisting of 2.43 (Invitrogen), 51.1 (ATCC HB-230), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), and YTS105.18 (CD8α, Novus Biologicals). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of 2.43 (Invitrogen), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), and YTS105.18 (CD8α, Novus Biologicals).

An exemplary CD8 binding agent can also be selected from antibodies or antibody fragments employing CDRs of clones OKT-8, 51.1, S6F1, TRX2, and UCHT4, SP16, 3B5, C8-144B, HIT8a, RAVB3, LT8, 17D8, MEM-31, MEM-87, RIV11, DK-25, YTC141.1HL, or YTC182.20. In some embodiments, the conjugate comprises a Fab, wherein the Fab comprises a heavy chain fragment comprising the amino acid sequence of SEQ ID NO: 6 and a light chain fragment comprising the amino acid sequence of SEQ ID NO: 7.

An exemplary CD137 binding agent can be selected from antibodies or antibody fragments employing CDRs of clones 4B4-1, P566, or Urelumab. An exemplary CD28 binding agent can be selected from antibodies or antibody fragments employing CDRs of clone TAB08. An exemplary CD45 binding agent can be selected from antibodies or antibody fragments employing CDRs of clones BC8, 9.4, 4B2, Tu116, or GAP8.3. An exemplary CD18 binding agent can be selected from antibodies or antibody fragments employing CDRs of clones 1B4, TS1/18, MEM-48, YFC118-3, TA-4, MEM-148, or R3-3, 24. An exemplary CD11a binding agent can be selected from antibodies or antibody fragments employing CDRs of clone MHN24 or Efalizumab. An exemplary IL-2 receptor binding agent can be selected from of antibodies or antibody fragments employing CDRs of clones YTH 906.9HL, IL2R.1, BC96, B-B10, 216, MEM-181, ITYV, MEM-140, ICO-105, Daclizumab, or from the group consisting of IL2 or fragments of IL2. An exemplary IL-15R binding agent can be selected from antibodies or antibody fragments employing CDRs of clones JM7A4, or OTI3D5, or from the group consisting of IL15 or fragments of IL15. An exemplary TLR2 binding agent can be selected from antibodies or antibody fragments employing CDRs of clones JM22-41, TL2.1, 11G7, or TLR2.45. An exemplary TLR4 binding agent can be selected from antibodies or antibody fragments employing CDRs of clones HTA125, or 76B357-1. An exemplary TLR5 binding agent can be selected from antibodies or antibody fragments employing CDRs of clones 85B152-5, or 9D759-2. An exemplary GL7 binding agent can be selected from antibodies or antibody fragments employing CDRs of clone GL7.

An exemplary PD1 binding agent can be selected from antibodies or antibody fragments employing CDRs of clones MIH4, J116, J150, OTIB11, OTI17B10, OTI3A1, or OTI16D4. In addition, exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

An exemplary CTLA-4 binding agent can be selected from antibodies or antibody fragments employing CDRs of clones ER4.7G.11 [7G11], OTI9G4, OTI9F3, OTI3A5, A3.4H2.H12, 14D3, OTI3A12, OTI1A11, OTI1E8, OTI3B11, OTI3D2, OTI10C8, OTI2E9, OTI6F1, OTI7D3, OTI85B, OTI12C6. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

An exemplary TCR β binding agent can be an antibody selected from the group consisting of H57-597 (Invitrogen), 8A3 (Novus Biologicals), R73 (TCRα/β, Abcam), E6Z3S (TRBC1/TCRβ, Cell Signaling Technology), and antigen binding fragments thereof. In certain embodiments, the binding agent comprises a V_{H} domain and a V_{L} of an antibody selected from the group consisting of H57-597 (Invitrogen), 8A3 (Novus Biologicals), R73 (TCRα/β, Abcam), and E6Z3S (TRBC1/TCRβ, Cell Signaling Technology). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262: 732-745), or any other CDR determination method known in the art, of the V_{H} and V_{L} sequences of an antibody selected from the group consisting of H57-597 (Invitrogen), 8A3 (Novus Biologicals), R73 (TCRα/β, Abcam), and E6Z3S (TRBC1/TCRβ, Cell Signaling Technology).

An exemplary CD137 binding agent can be selected from antibodies or antibody fragments employing CDRs of clones 4B4-1, P566, or Urelumab.

In some embodiments, the immune cell targeting group comprises an antibody selected from the group consisting of a Fab, F(ab′)2, Fab′-SH, Fv, and scFv fragment. In some embodiments, the antibody is a human or humanized antibody. In some embodiments, the immune cell targeting group comprises a Fab or an immunoglobulin single variable domain, such as a VHH. In some embodiments, the immune cell targeting group comprises a Fab that does not comprise a natural interchain disulfide bond. For example, in some embodiments, the Fab comprises a heavy chain fragment that comprises a C233S substitution, and/or a light chain fragment that comprises a C214S substitution, numbering according to Kabat. In some embodiments, the immune cell targeting group comprises a Fab that comprises one or more non-native interchain disulfide bonds. In some embodiments, the interchain disulfide bonds are between two non-native cysteine residues on the light chain fragment and heavy chain fragment, respectively. For example, in some embodiments, the Fab comprises a heavy chain fragment that comprises F174C substitution, and/or a light chain fragment that comprises S176C substitution, numbering according to Kabat. In some embodiments, the Fab comprises a heavy chain fragment that comprises F174C and C233S substitutions, and/or alight chain fragment that comprises S176C and C214S substitutions, numbering according to Kabat. In some embodiments, the immune cell targeting group comprises a C-terminal cysteine residue. In some embodiments, the immune cell targeting group comprises a Fab that comprises a cysteine at the C-terminus of the heavy or light chain fragment. In some embodiments, the Fab further comprises one or more amino acids between the heavy chain of the Fab and the C-terminal cysteine. For example, in some embodiments, the Fab comprises two or more amino acids derived from an antibody hinge region (e.g., a partial hinge sequence) between the C-terminus of the Fab and the C-terminal cysteine. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker. In some embodiments, the Fab antibody is a DS Fab, a NoDS Fab, a bDS Fab, a bDS Fab-ScFv, as demonstrated in FIG. 47.

In some embodiments, the immune cell targeting group comprises an immunoglobulin single variable domain, such as a V_{HH}. In some embodiments, the V_{HH} comprises a cysteine at the C-terminus. In some embodiments, the V_{HH} further comprises a spacer comprising one or more amino acids between the V_{HH} domain and the C-terminal cysteine. In some embodiments, the spacer comprises one or more glycine residues, e.g., two glycine residues. In some embodiments, the immune cell targeting group comprises two or more V_{HH} domains. In some embodiments, the two or more V_{HH} domains are linked by an amino acid linker. In some embodiments, the amino acid linker comprises one or more glycine and/or serine residues (e.g., one or more repeats of the sequence GGGGS). In some embodiments, the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds (e.g., interchain disulfide bonds). In some embodiments, the immune cell targeting group comprises a V_{HH} domain linked to an antibody CH1 domain, and wherein the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat. In some embodiments, the antibody is a ScFv, a V_{HH}, a 2×V_{HH}, a V_{HH}-CH1/empty Vk, or a V_{HH}1-CH1/V_{HH-}2-Nb bDS, as demonstrated in FIG. 31.

An exemplary targeting moiety may have an amino sequence as set forth below:

Anti-CD3 hSP34-Fab sequences:
hSP34 heavy chain (HC) sequence (SEQ ID NO: 1):
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYIS
YWAYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSSDKTHTC
hSP34-mlam light chain (LC) sequence (mouse lambda)
(SEQ ID NO: 2):
QTVVTQEPSLTVSPGGTVTLTCGSSTGAVTSGNYPNWVQQKPGQAPRGLIGGTKF
LAPGTPARFSGSLLGGKAALTLSGVQPEDEAEYYCVLWYSNRWVFGGGTKLTVLG
QPKSSPSVTLFPPSSEELETNKATLVCTITDFYPGVVTVDWKVDGTPVTQGMETT
QPSKQSNNKYMASSYLTLTARAWERHSSYSCQVTHEGHTVEKSLSRADSS
SP34-hlam LC (human lambda) (SEQ ID NO: 3):
QTVVTQEPSLTVSPGGTVTLTCGSSTGAVTSGNYPNWVQQKPGQAPRGLIGGTKF
LAPGTPARFSGSLLGGKAALTLSGVQPEDEAEYYCVLWYSNRWVFGGGTKLTVLS
QPKAAPSVTLFPPSSEELQANKATLVCLVSDFYPGAVTVAWKADGSPVKVGVETT
KPSKQSNNKYAASSYLSLTPEQWKSHRSYSCRVTHEGSTVEKTVAPAESS
Anti-CD3 Hu291-Fab sequences:
Hu291 HC (SEQ ID NO: 4):
QVQLVQSGAEVKKPGASVKVSCKASGYTFISYTMHWVRQAPGQGLEWMGYINPRS
GYTHYNQKLKDKATLTADKSASTAYMELSSLRSEDTAVYYCARSAYYDYDGFAYW
GQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEP
KSSDKTHTC
Hu 291 LC (SEQ ID NO: 5):
MDMRVPAQLLGLLLLWLPGAKCDIQMTQSPSSLSASVGDRVTITCSASSSVSYMN
WYQQKPGKAPKRLIYDTSKLASGVPSRFSGSGSGTDFTLTISSLQPEDFATYYCQ
QWSSNPPTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQ
GLSSPVTKSFNRGES
Anti-CD8 TRX2-Fab sequences:
TRX2 HC (SEQ ID NO: 6):
QVQLVESGGGVVQPGRSLRLSCAASGFTFSDFGMNWVRQAPGKGLEWVALIYYDG
SNKFYADSVKGRFTISRDNSKNTLYLQMNSLRAEDTAVYYCAKPHYDGYYHFFDS
WGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVE
PKSSDKTHTC
TRX2 LC (SEQ ID NO: 7):
DIQMTQSPSSLSASVGDRVTITCKGSQDINNYLAWYQQKPGKAPKLLIYNTDILH
TGVPSRFSGSGSGTDFTFTISSLQPEDIATYYCYQYNNGYTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGES
Anti-CD8 OKT8-Fab sequences:
OKT8 HC (SEQ ID NO: 8):
QVQLVQSGAEDKKPGASVKVSCKASGFNIKDTYIHWVRQAPGQGLEWMGRIDPAN
DNTLYASKFQGRVTITADTSSNTAYMELSSLRSEDTAVYYCGRGYGYYVFDHWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
SDKTHTC
OKT8 LC (SEQ ID NO: 9):
DIVMTQSPSSLSASVGDRVTITCRTSRSISQYLAWYQEKPGKAPKLLIYSGSTLQ
SGVPSRFSGSGSGTDFTLTISSLQPEDFATYYCQQHNENPLTFGQGTKVEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGES
Anti-CD4 Ibalizumab-Fab sequences:
Ibalizumab HC (SEQ ID NO: 10):
QVQLQQSGPEVVKPGASVKMSCKASGYTFTSYVIHWVRQKPGQGLDWIGYINPYN
DGTDYDEKFKGKATLTSDTSTSTAYMELSSLRSEDTAVYYCAREKDNYATGAWFA
YWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSSDKTHTC
Ibalizumab LC (SEQ ID NO: 11):
DIVMTQSPDSLAVSLGERVTMNCKSSQSLLYSTNQKNYLAWYQQKPGQSPKLLIY
WASTRESGVPDRFSGSGSGTDFTLTISSVQAEDVAVYYCQQYYSYRTFGGGTKLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGES
anti-CD5 He3-Fab sequences:
He3 HC (SEQ ID NO: 12):
EIQLVQSGGGLVKPGGSVRISCAASGYTFTNYGMNWVRQAPGKGLEWMGWINTHT
GEPTYADSFKGRFTFSLDDSKNTAYLQINSLRAEDTAVYFCTRRGYDWYFDVWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
SDKTHTC
He3 LC (SEQ ID NO: 13):
DIQMTQSPSSLSASVGDRVTITCRASQDINSYLSWFQQKPGKAPKTLIYRANRLE
SGVPSRFSGSGSGTDYTLTISSLQYEDFGIYYCQQYDESPWTFGGGTKLEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGES
anti-CD7 TH-69-Fab sequences:
TH-69 HC (SEQ ID NO: 14):
EVQLVESGGGLVKPGGSLKLSCAASGLTFSSYAMSWVRQTPEKRLEWVASISSGG
FTYYPDSVKGRFTISRDNARNILYLQMSSLRSEDTAMYYCARDEVRGYLDVWGAG
TTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSC
DKTHTC
TH-69 LC (SEQ ID NO: 15):
DIQMTQTTSSLSASLGDRVTISCSASQGISNYLNWYQQKPDGTVKLLIYYTSSLH
SGVPSRFSGSGSGTDYSLTISNLEPEDIATYYCQQYSKLPYTFGGGTKLEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
anti-CD2 TS2/18.1-Fab sequences:
TS2/18.1 HC (SEQ ID NO: 16):
EVQLVESGGGLVMPGGSLKLSCAASGFAFSSYDMSWVRQTPEKRLEWVAYISGGG
FTYYPDTVKGRFTLSRDNAKNTLYLQMSSLKSEDTAMYYCARQGANWELVYWGQG
TLVTVSAASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSS
DKTHTC
TS2/18.1 LC (SEQ ID NO: 17):
DIVMTQSPATLSVTPGDRVFLSCRASQSISDFLHWYQQKSHESPRLLIKYASQSI
SGIPSRFSGSGSGSDFTLSINSVEPEDVGVYFCQNGHNFPPTFGGGTKLEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGES
anti-CD2 9.6-Fab sequences:
9.6 HC (SEQ ID NO: 18):
QVQLQQPGAELVRPGSSVKLSCKASGYTFTRYWIHWVKQRPIQGLEWIGNIDPSD
SETHYNQKFKDKATLTVDKSSGTAYMQLSSLTSEDSAVYYCATEDLYYAMEYWGQ
GTSVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
SDKTHTC
9.6 LC (SEQ ID NO: 19):
NIMMTQSPSSLAVSAGEKVTMTCKSSQSVLYSSNQKNYLAWYQQKPGQSPKLLIY
WASTRESGVPDRFTGSGSGTDFTLTISSVQPEDLAVYYCHQYLSSHTFGGGTKLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGES
anti-CD2 9-1-Fab sequences:
9-1 HC (SEQ ID NO: 20):
QVQLQQPGTELVRPGSSVKLSCKASGYTFTSYWVNWVKQRPDQGLEWIGRIDPYD
SETHYNQKFTDKAISTIDTSSNTAYMQLSTLTSDASAVYYCSRSPRDSSTNLADW
GQGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEP
KSSDKTHTC
9-1 LC (SEQ ID NO: 21):
DIVMTQSPATLSVTPGDRVSLSCRASQSISDYLHWYQQKSHESPRLLIKYASQSI
SGIPSRFSGSGSGSDFTLSINSVEPEDVGVYYCQNGHSFPLTFGAGTKLELRRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGES
mutOKT8-Fab sequences:
mutOKT8 HC (SEQ ID NO: 22):
QVQLVQSGAEDKKPGASVKVSCKASGFNIKDTYIHWVRQAPGQGLEWMGRIDPAN
DNTLYASKFQGRVTITADTSSNTAYMELSSLRSEDTAVYYCGRGAGAYVFDHWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
SDKTHTC
mutOKT8 LC (SEQ ID NO: 23):
DIVMTQSPSSLSASVGDRVTITCRTSRSISAALAWYQEKPGKAPKLLIYSGSTLQ
SGVPSRFSGSGSGTDFTLTISSLQPEDFATYYCQQHNENPLTFGQGTKVEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGES.
Anti-CD56 A1 Fab sequence
A1 bDS HC (SEQ ID NO: 26):
QVQLQQSGPGLVKPSQTLSLTCAISGDSVSSNSAAWNWIRQSPSNWIRQSPSGLE
WLGRTYYRSKWYNDYAVSVKSRITINPDTSKNQFSLQLNSVTPEDTAVYYCAREN
IAAWTWAFDIWGQGTMVTVSSASTKGPSVFPLAPSSKSTSGGTAALGCLVKDYFP
EPVTVSWNSGALTSGVHTCPAVLQSSGLYSLSSVVTVPSSSLGTQTYICNVNHKP
SNTKVDKKVEPKSSDKTHTCGGHHHHHH
A1 bDS LC (SEQ ID NO: 27):
EIVMTQSPGTLSLSPGERATLSCRASQSVSSSYLAWYQQKPGLAPRLLIYDTSLR
ATDIPDRFSGSGSGTAFTLTISRLEPEDFAVYYCQQYGSSPTFGQGTKVEIKRTV
AAPSVFIFPPSDEQLKSGTASVVCLLNNFYPREAKVQWKVDNALQSGNSQESVTE
QDSKDSTYSLCSTLTLSKADYEKHKVYACEVTHQGLSSPVTKSFNRGES
Anti-CD56 A2 Fab sequence
A2 bDS HC (SEQ ID NO: 28):
EVQLVQSGAEVKKPGSSVKVSCKASGGTFTGYYMHWVRQAPGQGLEWMGWINPNS
GGTNYAQKFQGRVTMTRDTSISTAYMELSRLRSDDTAVYYCARDLSSGYSGYFDY
WGQGTLVTVSSASTKGPSVFPLAPSSKSTSGGTAALGCLVKDYFPEPVTVSWNSG
ALTSGVHTCPAVLQSSGLYSLSSVVTVPSSSLGTQTYICNVNHKPSNTKVDKKVE
PKSSDKTHTCGGHHHHHH
A2 bDS LC (SEQ ID NO: 29):
DVVMTQSPLSLPVTPGEPASISCRSSQSLLHSNGYNYLNWYLQKPGQSPQLLIYL
GSNRASGVPDRFSGSGSGTDFTLKISRVEGEDVGDYYCMQALQSPFTFGQGTKLE
IKRTVAAPSVFIFPPSDEQLKSGTASVVCLLNNFYPREAKVQWKVDNALQSGNSQ
ESVTEQDSKDSTYSLCSTLTLSKADYEKHKVYACEVTHQGLSSPVTKSFNRGES
Anti-CD56 A3 Fab sequence
A3 bDS HC (SEQ ID NO: 30):
EVQLVQSGAEVKKPGSSVKVSCKASGGTFTGYYMHWVRQAPGQGLEWMGWINPNS
GGTNYAQKFQGRVTMTRDTSISTAYMELSRLRSDDTAVYYCARDLSSGYSGYFDY
WGQGTLVTVSSASTKGPSVFPLAPSSKSTSGGTAALGCLVKDYFPEPVTVSWNSG
ALTSGVHTCPAVLQSSGLYSLSSVVTVPSSSLGTQTYICNVNHKPSNTKVDKKVE
PKSSDKTHTCGGHHHHHH
A3 bDS LC (SEQ ID NO: 31):
DVVMTQSPLSLPVTPGEPASISCRSSQSLLHSNGYNFLDWYLQKPGQSPQLLIYL
GSNRASGVPDRFSGSGSGTDFTLKISRVEADDVGVYYCMQSLQTPWTFGHGTKVE
IKRTVAAPSVFIFPPSDEQLKSGTASVVCLLNNFYPREAKVQWKVDNALQSGNSQ
ESVTEQDSKDSTYSLCSTLTLSKADYEKHKVYACEVTHQGLSSPVTKSFNRGES
Anti-CD56 Lorvotuzumab Fab sequence
Lorvotuzumab bDS HC (SEQ ID NO: 32):
QVQLVESGGG VVQPGRSLRL SCAASGFTFS SFGMHWVRQA
PGKGLEWVAYISSGSFTIYY ADSVKGRFTI SRDNSKNTLY
LQMNSLRAED TAVYYCARMR KGYAMDYWGQ
GTLVTVSSASTKGPSVFPLAPSSKSTSGGTAALGCLVKDYFPEPVTVSWNSGALT
SGVHTCPAVLQSSGLYSLSSVVTVPSSSLGTQTYICNVNHKPSNTKVDKKVEPKS
SDKTHTCHHHHHH
Lorvotuzumab bDS LC (SEQ ID NO: 33):
DVVMTQSPLSLPVTLGQPASISCRSSQIIIHSDGNTYLEWFQQRPGQSPRRLIYK
VSNRFSGVPDRFSGSGSGTDFTLKISRVEAEDVGVYYCFQGSHVPHTFGQGTKVE
IKRTVAAPSVFIFPPSDEQLKSGTASVVCLLNNFYPREAKVQWKVDNALQSGNSQ
ESVTEQDSKDSTYSLCSTLTLSKADYEKHKVYACEVTHQGLSSPVTKSFNRGES
Anti-CD2 RPA-2.10v1 Fab sequence
RPA-2.10v1 bDS HC (SEQ ID NO: 34):
EVKLVESGGGLVKPGGSLKLSCAASGFTFSSYDMSWVRQTPEKRLEWVASISGGG
FLYYLDSVKGRFTISRDNARNILYLHMTSLRSEDTAMYYCARSSYGEIMDYWGQG
TSVTVSSASTKGPSVFPLAPSSKSTSGGTAALGCLVKDYFPEPVTVSWNSGALTS
GVHTCPAVLQSSGLYSLSSVVTVPSSSLGTQTYICNVNHKPSNTKVDKKVEPKSS
DKTHTCHHHHHH
RPA-2.10v1 bDS LC (SEQ ID NO: 35):
DILLTQSPAILSVSPGERVSFSCRASQRIGTSIHWYQQRTTGSPRLLIKYASESI
SGIPSRFSGSGSGTDFTLSINSVESEDVADYYCQQSHGWPFTFGGGTKLEIERTV
AAPSVFIFPPSDEQLKSGTASVVCLLNNFYPREAKVQWKVDNALQSGNSQESVTE
QDSKDSTYSLCSTLTLSKADYEKHKVYACEVTHQGLSSPVTKSFNRGES
Anti-CD137 4B4-1 Fab sequence
4B4-1 bDS HC (SEQ ID NO: 36):
QVQLQQPGAELVKPGASVKLSCKASGYTFSSYWMHWVKQRPGQVLEWIGEINPGN
GHTNYNEKFKSKATLTVDKSSSTAYMQLSSLTSEDSAVYYCARSFTTARGFAYWG
QGTLVTVSASTKGPSVFPLAPSSKSTSGGTAALGCLVKDYFPEPVTVSWNSGALT
SGVHTCPAVLQSSGLYSLSSVVTVPSSSLGTQTYICNVNHKPSNTKVDKKVEPKS
SDKTHTCHHHHHH
4B4-1 bDS LC (SEQ ID NO: 37):
DIVMTQSPATQSVTPGDRVSLSCRASQTISDYLHWYQQKSHESPRLLIKYASQSI
SGIPSRFSGSGSGSDFTLSINSVEPEDVGVYYCQDGHSFPPTFGGGTKLEIKRTV
AAPSVFIFPPSDEQLKSGTASVVCLLNNFYPREAKVQWKVDNALQSGNSQESVTE
QDSKDSTYSLCSTLTLSKADYEKHKVYACEVTHQGLSSPVTKSFNRGES
hSP34-hlam NoDS HC (SEQ ID NO: 38):
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYIS
YWAYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSSDKTHTC
hSP34-hlam NoDS LC (SEQ ID NO: 39):
QTVVTQEPSLTVSPGGTVTLTCGSSTGAVTSGNYPNWVQQKPGQAPRGLIGGTKF
LAPGTPARFSGSLLGGKAALTLSGVQPEDEAEYYCVLWYSNRWVFGGGTKLTVLS
QPKAAPSVTLFPPSSEELQANKATLVCLVSDFYPGAVTVAWKADGSPVKVGVETT
KPSKQSNNKYAASSYLSLTPEQWKSHRSYSCRVTHEGSTVEKTVAPAESS
hSP34-hlam DS HC (SEQ ID NO: 40):
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYIS
YWAYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTC
hSP34-hlam DS LC (SEQ ID NO: 41):
QTVVTQEPSLTVSPGGTVTLTCGSSTGAVTSGNYPNWVQQKPGQAPRGLIGGTKF
LAPGTPARFSGSLLGGKAALTLSGVQPEDEAEYYCVLWYSNRWVFGGGTKLTVLS
QPKAAPSVTLFPPSSEELQANKATLVCLVSDFYPGAVTVAWKADGSPVKVGVETT
KPSKQSNNKYAASSYLSLTPEQWKSHRSYSCRVTHEGSTVEKTVAPAECS
Anti-CD2 TS2/18.1 DS Fab
TS2/18.1 DS HC (SEQ ID NO: 42):
EVQLVESGGGLVMPGGSLKLSCAASGFAFSSYDMSWVRQTPEKRLEWVAYISGGG
FTYYPDTVKGRFTLSRDNAKNTLYLQMSSLKSEDTAMYYCARQGANWELVYWGQG
TLVTVSAASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSC
DKTHTC
TS2/18.1 DS LC (SEQ ID NO: 43):
DIVMTQSPATLSVTPGDRVFLSCRASQSISDFLHWYQQKSHESPRLLIKYASQSI
SGIPSRFSGSGSGSDFTLSINSVEPEDVGVYFCQNGHNFPPTFGGGTKLEIKRTV
AAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Anti-CD2 9.6 DS Fab
9.6 DS HC (SEQ ID NO: 44):
QVQLQQPGAELVRPGSSVKLSCKASGYTFTRYWIHWVKQRPIQGLEWIGNIDPSD
SETHYNQKFKDKATLTVDKSSGTAYMQLSSLTSEDSAVYYCATEDLYYAMEYWGQ
GTSVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTC
9.6 DS LC (SEQ ID NO: 45):
NIMMTQSPSSLAVSAGEKVTMTCKSSQSVLYSSNQKNYLAWYQQKPGQSPKLLIY
WASTRESGVPDRFTGSGSGTDFTLTISSVQPEDLAVYYCHQYLSSHTFGGGTKLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
hSP34-hlam bDS HC (SEQ ID NO: 46):
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYIS
YWAYWGQGTLVTVSSASTKGPSVFPLAPSSKSTSGGTAALGCLVKDYFPEPVTVS
WNSGALTSGVHTCPAVLQSSGLYSLSSVVTVPSSSLGTQTYICNVNHKPSNTKVD
KKVEPKSSDKTHTCHHHHHH
hSP34-hlam bDS LC (SEQ ID NO: 47):
QTVVTQEPSLTVSPGGTVTLTCGSSTGAVTSGNYPNWVQQKPGQAPRGLIGGTKF
LAPGTPARFSGSLLGGKAALTLSGVQPEDEAEYYCVLWYSNRWVFGGGTKLTVLS
QPKAAPSVTLFPPSSEELQANKATLVCLVSDFYPGAVTVAWKADGSPVKVGVETT
KPSKQSNNKYAACSYLSLTPEQWKSHRSYSCRVTHEGSTVEKTVAPAESS
Anti-CD3 TR66 bDS Fab sequence
TR66 bDS HC (SEQ ID NO: 48):
QVQLQQSGAELARPGASVKMSCKTSGYTFTRYTMHWVKQRPGQGLEWIGYINPSR
GYTNYNQKFKDKATLTTDKSSSTAYMQLSSLTSEDSAVYYCARYYDDNYSLDYWG
QGTTLTVSSASTKGPSVFPLAPSSKSTSGGTAALGCLVKDYFPEPVTVSWNSGAL
TSGVHTCPAVLQSSGLYSLSSVVTVPSSSLGTQTYICNVNHKPSNTKVDKKVEPK
SSDKTHTCHHHHHH
TR66 bDS LC (SEQ ID NO: 49):
QIVLTQSPSSLSASLGEKVTMTCRASSSVSYMNWYQQKPGTSPKRWIYDTSKVAS
GVPDRFSGSGSGTSYSLTISSMEAEDAATYYCQQWSSNPLTFGAGTKLELKRTVA
APSVFIFPPSDEQLKSGTASVVCLLNNFYPREAKVQWKVDNALQSGNSQESVTEQ
DSKDSTYSLCSTLTLSKADYEKHKVYACEVTHQGLSSPVTKSFNRGES
Anti-CD3 TRX4 bDS Fab sequence
TRX4 bDS HC (SEQ ID NO: 50):
EVQLLESGGGLVQPGGSLRLSCAASGFTFSSFPMAWVRQAPGKGLEWVSTISTSG
GRTYYRDSVKGRFTISRDNSKNTLYLQMNSLRAEDTAVYYCAKFRQYSGGFDYWG
QGTLVTVSSASTKGPSVFPLAPSSKSTSGGTAALGCLVKDYFPEPVTVSWNSGAL
TSGVHTCPAVLQSSGLYSLSSVVTVPSSSLGTQTYICNVNHKPSNTKVDKKVEPK
SSDKTHTCHHHHHH
TRX4 bDS LC (SEQ ID NO: 51):
DIQLTQPNSVSTSLGSTVKLSCTLSSGNIENNYVHWYQLYEGRSPTTMIYDDDKR
PDGVPDRFSGSIDRSSNSAFLTIHNVAIEDEAIYFCHSYVSSFNVFGGGTKLTVL
GQPKANPTVTLFPPSSEELQANKATLVCLISDFYPGAVTVAWKADGSPVKAGVET
TKPSKQSNNKYAACSYLSLTPEQWKSHRSYSCQVTHEGSTVEKTVAPTESS
Anti-CD3 HzUCHT1 bDS Fab sequence
HzUCHT1(Y59T) bDS HC (SEQ ID NO: 52):
EVQLVESGGGLVQPGGSLRLSCAASGYSFTGYTMNWVRQAPGKGLEWVALINPTK
GVSTYNQKFKDRFTISVDKSKNTAYLQMNSLRAEDTAVYYCARSGYYGDSDWYFD
VWGQGTLVTVSSASTKGPSVFPLAPSSKSTSGGTAALGCLVKDYFPEPVTVSWNS
GALTSGVHTCPAVLQSSGLYSLSSVVTVPSSSLGTQTYICNVNHKPSNTKVDKKV
EPKSSDKTHTCHHHHHH
HzUCHT1 bDS LC (SEQ ID NO: 53):
DIQMTQSPSSLSASVGDRVTITCRASQDIRNYLNWYQQKPGKAPKLLIYYTSRLE
SGVPSRFSGSGSGTDYTLTISSLQPEDFATYYCQQGNTLPWTFGQGTKVEIKRTV
AAPSVFIFPPSDEQLKSGTASVVCLLNNFYPREAKVQWKVDNALQSGNSQESVTE
QDSKDSTYSLCSTLTLSKADYEKHKVYACEVTHQGLSSPVTKSFNRGES
Anti-CD3 Teplizumab bDS Fab sequence
Teplizumab bDS HC (SEQ ID NO: 54):
QVQLVQSGGGVVQPGRSLRLSCKASGYTFTRYTMHWVRQAPGKGLEWIGYINPSR
GYTNYNQKVKDRFTISRDNSKNTAFLQMDSLRPEDTGVYFCARYYDDHYCLDYWG
QGTPVTVSSASTKGPSVFPLAPSSKSTSGGTAALGCLVKDYFPEPVTVSWNSGAL
TSGVHTCPAVLQSSGLYSLSSVVTVPSSSLGTQTYICNVNHKPSNTKVDKKVEPK
SSDKTHTCHHHHHH
Teplizumab bDS LC (SEQ ID NO: 55):
DIQMTQSPSSLSASVGDRVTITCSASSSVSYMNWYQQTPGKAPKRWIYDTSKLAS
GVPSRFSGSGSGTDYTFTISSLQPEDIATYYCQQWSSNPFTFGQGTKLQITRTVA
APSVFIFPPSDEQLKSGTASVVCLLNNFYPREAKVQWKVDNALQSGNSQESVTEQ
DSKDSTYSLCSTLTLSKADYEKHKVYACEVTHQGLSSPVTKSFNRGES
Anti-CD8 TRX2 bDS Fab sequence
TRX2 bDS HC (SEQ ID NO: 56):
QVQLVESGGGVVQPGRSLRLSCAASGFTFSDFGMNWVRQAPGKGLEWVALIYYDG
SNKFYADSVKGRFTISRDNSKNTLYLQMNSLRAEDTAVYYCAKPHYDGYYHFFDS
WGQGTLVTVSSASTKGPSVFPLAPSSKSTSGGTAALGCLVKDYFPEPVTVSWNSG
ALTSGVHTCPAVLQSSGLYSLSSVVTVPSSSLGTQTYICNVNHKPSNTKVDKKVE
PKSSDKTHTC
TRX2 bDS LC (SEQ ID NO: 57):
DIQMTQSPSSLSASVGDRVTITCKGSQDINNYLAWYQQKPGKAPKLLIYNTDILH
TGVPSRFSGSGSGTDFTFTISSLQPEDIATYYCYQYNNGYTFGQGTKVEIKRTVA
APSVFIFPPSDEQLKSGTASVVCLLNNFYPREAKVQWKVDNALQSGNSQESVTEQ
DSKDSTYSLCSTLTLSKADYEKHKVYACEVTHQGLSSPVTKSFNRGES
Anti-CD2 Lo-CD2b bDS Fab sequence
Lo-CD2b bDS HC (SEQ ID NO: 58):
EVQLVESGGGLVQPGASLKLSCVASGFTFSDYWMSWVRQTPGKPMEWIGHIKYDG
SYTNYAPSLKNRFTISRDNAKTTLYLQMSNVRSEDSATYYCAREAPGAASYWGQG
TLVTVSSASTKGPSVFPLAPSSKSTSGGTAALGCLVKDYFPEPVTVSWNSGALTS
GVHTCPAVLQSSGLYSLSSVVTVPSSSLGTQTYICNVNHKPSNTKVDKKVEPKSS
DKTHTC
Lo-CD2b bDS LC (SEQ ID NO: 59):
DVVLTQTPVAQPVTLGDQASISCRSSQSLVHSNGNTYLEWFLQKPGQSPQLLIYK
VSNRFSGVPDRFIGSGSGSDFTLKISRVEPEDWGVYYCFQGTHDPYTFGAGTKLE
LKRTVAAPSVFIFPPSDEQLKSGTASVVCLLNNFYPREAKVQWKVDNALQSGNSQ
ESVTEQDSKDSTYSLCSTLTLSKADYEKHKVYACEVTHQGLSSPVTKSFNRGES
Anti-CD2 35.1 bDS Fab sequence
35.1 bDS HC (SEQ ID NO: 60):
EVQLQQSGAELVKPGASVKLSCRTSGFNIKDTYIHWVKQRPEQGLKWIGRIDPAN
GNTKYDPKFQDKATVTADTSSNTAYLQLSSLTSEDTAVYYCVTYAYDGNWYFDVW
GAGTAVTVSSASTKGPSVFPLAPSSKSTSGGTAALGCLVKDYFPEPVTVSWNSGA
LTSGVHTCPAVLQSSGLYSLSSVVTVPSSSLGTQTYICNVNHKPSNTKVDKKVEP
KSSDKTHTC
35.1 bDS LC (SEQ ID NO: 61):
DIKMTQSPSSMYVSLGERVTITCKASQDINSFLSWFQQKPGKSPKTLIYRANRLV
DGVPSRFSGSGSGQDYSLTISSLEYEDMEIYYCLQYDEFPYTFGGGTKLEMKRTV
AAPSVFIFPPSDEQLKSGTASVVCLLNNFYPREAKVQWKVDNALQSGNSQESVTE
QDSKDSTYSLCSTLTLSKADYEKHKVYACEVTHQGLSSPVTKSFNRGES
Anti-CD2 OKT11 bDS Fab sequence
OKT11 bDS HC (SEQ ID NO: 62):
QVQLQQPGAELVRPGTSVKLSCKASGYTFTSYWMHWIKQRPEQGLEWIGRIDPYD
SETHYNEKFKDKAILSVDKSSSTAYIQLSSLTSDDSAVYYCSRRDAKYDGYALDY
WGQGTSVTVSSASTKGPSVFPLAPSSKSTSGGTAALGCLVKDYFPEPVTVSWNSG
ALTSGVHTCPAVLQSSGLYSLSSVVTVPSSSLGTQTYICNVNHKPSNTKVDKKVE
PKSSDKTHTC
OKT11 bDS LC (SEQ ID NO: 63):
DIVMTQAAPSVPVTPGESVSISCRSSKTLLHSNGNTYLYWFLQRPGQSPQVLIYR
MSNLASGVPNRFSGSGSETTFTLRISRVEAEDVGIYYCMQHLEYPYTFGGGTKLE
IERTVAAPSVFIFPPSDEQLKSGTASVVCLLNNFYPREAKVQWKVDNALQSGNSQ
ESVTEQDSKDSTYSLCSTLTLSKADYEKHKVYACEVTHQGLSSPVTKSFNRGES
Anti-CD11a HzMHM24 bDS Fab sequence
HzMHM24 bDS HC (SEQ ID NO: 64):
EVQLVESGGGLVQPGGSLRLSCAASGYSFTGHWMNWVRQAPGKGLEWVGMIHPSD
SETRYNQKFKDRFTISVDKSKNTLYLQMNSLRAEDTAVYYCARGIYFYGTTYFDY
WGQGTLVTVSSASTKGPSVFPLAPSSKSTSGGTAALGCLVKDYFPEPVTVSWNSG
ALTSGVHTCPAVLQSSGLYSLSSVVTVPSSSLGTQTYICNVNHKPSNTKVDKKVE
PKSSDKTHTCHHHHHH
HzMHM24 bDS LC (SEQ ID NO: 65):
DIQMTQSPSSLSASVGDRVTITCRASKTISKYLAWYQQKPGKAPKLLIYSGSTLQ
SGVPSRFSGSGSGTDFTLTISSLQPEDFATYYCQQHNEYPLTFGQGTKVEIKRTV
AAPSVFIFPPSDEQLKSGTASVVCLLNNFYPREAKVQWKVDNALQSGNSQESVTE
QDSKDSTYSLCSTLTLSKADYEKHKVYACEVTHQGLSSPVTKSFNRGES
Anti-CD18 h1B4 bDS Fab sequence
h1B4 bDS HC (SEQ ID NO: 66):
EVQLVESGGDLVQPGRSLRLSCAASGFTFSDYYMSWVRQAPGKGLEWVAAIDNDG
GSISYPDTVKGRFTISRDNAKNSLYLQMNSLRVEDTALYYCARQGRLRRDYFDYW
GQGTLVTVSTASTKGPSVFPLAPSSKSTSGGTAALGCLVKDYFPEPVTVSWNSGA
LTSGVHTCPAVLQSSGLYSLSSVVTVPSSSLGTQTYICNVNHKPSNTKVDKKVEP
KSSDKTHTCHHHHHH
h1B4 bDS LC (SEQ ID NO: 67):
DIQMTQSPSSLSASVGDRVTITCRASESVDSYGNSFMHWYQQKPGKAPKLLIYRA
SNLESGVPSRFSGSGSGTDFTFTISSLQPEDIATYYCQQSNEDPLTFGQGTKLEI
KRTVAAPSVFIFPPSDEQLKSGTASVVCLLNNFYPREAKVQWKVDNALQSGNSQE
SVTEQDSKDSTYSLCSTLTLSKADYEKHKVYACEVTHQGLSSPVTKSFNRGES
Anti-CD18 Erlizumab bDS Fab sequence
Erlizumab bDS HC (SEQ ID NO: 68):
EVQLVESGGGLVQPGGSLRLSCATSGYTFTEYTMHWMRQAPGKGLEWVAGINPKN
GGTSHNQRFMDRFTISVDKSTSTAYMQMNSLRAEDTAVYYCARWRGLNYGFDVRY
FDVWGQGTLVTVSSASTKGPSVFPLAPSSKSTSGGTAALGCLVKDYFPEPVTVSW
NSGALTSGVHTCPAVLQSSGLYSLSSVVTVPSSSLGTQTYICNVNHKPSNTKVDK
KVEPKSSDKTHTCHHHHHH
Erlizumab bDS LC (SEQ ID NO: 69):
DIQMTQSPSSLSASVGDRVTITCRASQDINNYLNWYQQKPGKAPKLLIYYTSTLH
SGVPSRFSGSGSGTDYTLTISSLQPEDFATYYCQQGNTLPPTFGQGTKVEIKRTV
AAPSVFIFPPSDEQLKSGTASVVCLLNNFYPREAKVQWKVDNALQSGNSQESVTE
QDSKDSTYSLCSTLTLSKADYEKHKVYACEVTHQGLSSPVTKSFNRGES
Anti-CD4/CD8 Ibalizumab/TRX2 bDS Fab-ScFv sequence
Ibalizumab/TRX2 bDS Fab-ScFv HC (SEQ ID NO: 70):
QVQLQQSGPEVVKPGASVKMSCKASGYTFTSYVIHWVRQKPGQGLDWIGYINPYN
DGTDYDEKFKGKATLTSDTSTSTAYMELSSLRSEDTAVYYCAREKDNYATGAWFA
YWGQGTLVTVSSASTKGPSVFPLAPSSKSTSGGTAALGCLVKDYFPEPVTVSWNS
GALTSGVHTCPAVLQSSGLYSLSSVVTVPSSSLGTQTYICNVNHKPSNTKVDKKV
EPKSSDKTHTCHHHHHH
Ibalizumab/TRX2 bDS Fab-ScFv LC (SEQ ID NO: 71):
DIVMTQSPDSLAVSLGERVTMNCKSSQSLLYSTNQKNYLAWYQQKPGQSPKLLIY
WASTRESGVPDRFSGSGSGTDFTLTISSVQAEDVAVYYCQQYYSYRTFGGGTKLE
IKRTVAAPSVFIFPPSDEQLKSGTASVVCLLNNFYPREAKVQWKVDNALQSGNSQ
ESVTEQDSKDSTYSLCSTLTLSKADYEKHKVYACEVTHQGLSSPVTKSFNRGESG
GGGSGGGGSGGGGSQVQLVESGGGVVQPGRSLRLSCAASGFTFSDFGMNWVRQAP
GKGLEWVALIYYDGSNKFYADSVKGRFTISRDNSKNTLYLQMNSLRAEDTAVYYC
AKPHYDGYYHFFDSWGQGTLVTVSSGGGGSGGGGSGGGGSGGGGSDIQMTQSPSS
LSASVGDRVTITCKGSQDINNYLAWYQQKPGKAPKLLIYNTDILHTGVPSRFSGS
GSGTDFTFTISSLQPEDIATYYCYQYNNGYTFGQGTKVEIK
Anti-CD4 Ibalizumab NoDS Fab sequence
Ibalizumab NoDS LC (SEQ ID NO: 72):
QVQLQQSGPEVVKPGASVKMSCKASGYTFTSYVIHWVRQKPGQGLDWIGYINPYN
DGTDYDEKFKGKATLTSDTSTSTAYMELSSLRSEDTAVYYCAREKDNYATGAWFA
YWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKV
EPKSSDKTHTC
Ibalizumab NoDS HC (SEQ ID NO: 73):
DIVMTQSPDSLAVSLGERVTMNCKSSQSLLYSTNQKNYLAWYQQKPGQSPKLLIY
WASTRESGVPDRFSGSGSGTDFTLTISSVQAEDVAVYYCQQYYSYRTFGGGTKLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGES
Anti-CD4 OKT4 bDS Fab sequence
OKT4 bDS LC (SEQ ID NO: 74):
EVQLVESGGGLVQPGGSLRLSCAASGFTFSNYAMSWVRQAPGKRLEWVSAISDHS
TNTYYPDSVKGRFTISRDNAKNTLYLQMNSLRAEDTAVYYCARKYGGDYDPFDYW
GQGTLVTVSSASTKGPSVFPLAPSSKSTSGGTAALGCLVKDYFPEPVTVSWNSGA
LTSGVHTCPAVLQSSGLYSLSSVVTVPSSSLGTQTYICNVNHKPSNTKVDKKVEP
KSSDKTHTCHHHHHH
OKT4 bDS HC (SEQ ID NO: 75):
DIQMTQSPSSLSASVGDRVTITCQASQDINNYIAWYQHKPGKGPKLLIHYTSTLQ
PGIPSRFSGSGSGRDYTLTISSLQPEDFATYYCLQYDNLLFTFGGGTKVEIKRTV
AAPSVFIFPPSDEQLKSGTASVVCLLNNFYPREAKVQWKVDNALQSGNSQESVTE
QDSKDSTYSLCSTLTLSKADYEKHKVYACEVTHQGLSSPVTKSFNRGES
Anti-CD4 T023200008 Nb sequence (SEQ ID NO: 76)
CDR1, CDR2, CDR3 underlined based on IMGT designation:
EVQLVESGGGSVQPGGSLTLSCGTSGRTFNVMGWFRQAPGKEREFVAAVRWSSTG
IYYTQYADSVKSRFTISRDNAKNTVYLEMNSLKPEDTAVYYCAADTYNSNPARWD
GYDFRGQGTLVTVSSGGCGGHHHHHH
Anti-CD8 BDSn Nb sequence (SEQ ID NO: 77)
CDR1, CDR2, CDR3 underlined based on IMGT designation:
EVQLVESGGGLVQAGGSLRLSCAASGSTFSDYGVGWFRQAPGKGREFVADIDWNG
EHTSYADSVKGRFATSRDNAKNTAYLQMNSLKPEDTAVYYCAADALPYTVRKYNY
WGQGTQVTVSSGGCGGHHHHHH
Anti-CD3 T0170117G03-A Nb sequence (SEQ ID NO: 78)
EVQLVESGGGPVQAGGSLRLSCAASGRTYRGYSMGWFRQAPGKEREFVAAIVWSG
GNTYYEDSVKGRFTISRDNAKNIMYLQMTSLKPEDSATYYCAAKIRPYIFKIAGQ
YDYWGQGTLVTVSSAGGGSGGHHHHHHC
Anti-CD3 T0170060E11 Nb sequence (SEQ ID NO: 79)
EVQLVESGGGLVQPGGSLRLSCAASGDIYKSFDMGWYRQAPGKQRDLVAVIGSRG
NNRGRTNYADSVKGRFTISRDGTGNTVYLLMNKLRPEDTAIYYCNTAPLVAGRPW
GRGTLVTVSSGGGSGGHHHHHHC
Anti-CD7 V1 Nb sequence (SEQ ID NO: 80)
DVQLQESGGGLVQAGGSLRLSCAVSGYPYSSYCMGWFRQAPGKEREGVAAIDSDG
RTRYADSVKGRFTISQDNAKNTLYLQMNRMKPEDTAMYYCAARFGPMGCVDLSTL
SFGHWGQGTQVTVSITGGGCHHHHHHHH
Anti-TCR T017000700 Nb sequence (SEQ ID NO: 81)
CDR1, CDR2, CDR3 underlined based on IMGT designation:
EVQLVESGGGVVQPGGSLRLSCVASGYVHKINEYGWYRQAPGKEREKVAHISIGD
QTDYADSAKGRFTISRDESKNTVYLQMNSLRPEDTAAYYCRALSRIWPYDYWGQG
TLVTVSSGGCGGHHHHHH
Anti-CD28 28CD065G01 Nb sequence (SEQ ID NO: 82)
EVQLVESGGGLVQPGGSLRLSCAASGSIFRLHTMEWYRRTPETQREWVATITSGG
TTNYPDSVKGRFTISRDDTKKTVYLQMNSLKPEDTAVYYCHAVATEDAGFPPSNY
WGQGTLVTVSSGGCGGHHHHHH
Anti-CD3 T0170061C09 Nb sequence (SEQ ID NO: 83)
EVQLVESGGGPVQAGGSLRLSCAASGRTYRGYSMGWFRQAPGREREFVAAIVWSD
GNTYYEDSVKGRFTISRDNAKNTMYLQMTSLKPEDSATYYCAAKIRPYIFKIAGQ
YDYWGQGTLVTVSSGGCGGHHHHHH
Anti-CD3 12D2 bDS Fab sequence
12D2 bDS HC (SEQ ID NO: 84):
EVKLVESGGGLVQPGRSLRLSCAASGFNFYAYWMGWVRQAPGKGLEWIGEIKKDG
TTINYTPSLKDRFTISRDNAQNTLYLQMTKLGSEDTALYYCAREERDGYFDYWGQ
GVMVTVSSASTKGPSVFPLAPSSKSTSGGTAALGCLVKDYFPEPVTVSWNSGALT
SGVHTCPAVLQSSGLYSLSSVVTVPSSSLGTQTYICNVNHKPSNTKVDKKVEPKS
SDKTHTCGGHHHHHH
12D2 bDS LC (SEQ ID NO: 85):
QFVLTQPNSVSTNLGSTVKLSCKRSTGNIGSNYVNWYQQHEGRSPTTMIYRDDKR
PDGVPDRFSGSIDRSSNSALLTINNVQTEDEADYFCQSYSSGIVFGGGTKLTVLS
QPKAAPSVTLFPPSSEELQANKATLVCLVSDFYPGAVTVAWKADGSPVKVGVETT
KPSKQSNNKYAACSYLSLTPEQWKSHRSYSCRVTHEGSTVEKTVAPAESS
Anti-CD28 8G8A Fab sequence
8G8A bDS HC (SEQ ID NO: 86):
EVQLQQSGPELVKPGASVKMSCKASGYTFTSYVIQWVKQKPGQGLEWIGSINPYN
DYTKYNEKFKGKATLTSDKSSITAYMEFSLTSEDSALYCARWGDGNYWGRGTLTV
SSASTKGPSVFPLAPSSKSTSGGTAALGCLVKDYFPEPVTVSWNSGALTSGVHTC
PAVLQSSGLYSLSSVVTVPSSSLGTQTYICNVNHKPSNTKVDKKVEPKSSDKTHT
CGGHHHHHH
8G8A bDS LC (SEQ ID NO: 87):
DIEMTQSPAIMSASLGERVTMTCTASSSVSSSYFHWYQKPGSSPKLCIYSTSNLA
SGVPPRFSGSGSTSYSLTISMEAEDAATYFCHQYHRSPTFGGGTKLETKRTVAAP
SVFIFPPSDEQLKSGTASVVCLLNNFYPREAKVQWKVDNALQSGNSQESVTEQDS
KDSTYSLCSTLTLSKADYEKHKVYACEVTHQGLSSPVTKSFNRGES
Anti-CD28 2E12 Fab sequence
2E12 bDS HC (SEQ ID NO: 88):
QVQLKESGPGLVAPSQSLSITCTVSGFSLTGYGVNWVRQPPGKGLEWLGMIWGDG
STDYNSALKSRLSITKDNSKSQVFLKMNSLQTDDTARYYCARDGYSNFHYYVMDY
WGQGTSVTVSSASTKGPSVFPLAPSSKSTSGGTAALGCLVKDYFPEPVTVSWNSG
ALTSGVHTCPAVLQSSGLYSLSSVVTVPSSSLGTQTYICNVNHKPSNTKVDKKVE
PKSSDKTHTCGGHHHHHH
2E12 bDS LC (SEQ ID NO: 89):
DIVLTQSPASLAVSLGQRATISCRASESVEYYVTSLMQWYQQKPGQPPKLLISAA
SNVESGVPARFSGSGSGTDFSLNIHPVEEDDIAMYFCQQSRKVPWTFGGGTKLEI
KRRTVAAPSVFIFPPSDEQLKSGTASVVCLLNNFYPREAKVQWKVDNALQSGNSQ
ESVTEQDSKDSTYSLCSTLTLSKADYEKHKVYACEVTHQGLSSPVTKSFNRGES
Anti-CD28 CD28.9.3 Fab sequence
CD28.9.3 bDS HC (SEQ ID NO: 90):
QVKLQQSGPGLVTPSQSLSITCTVSGFSLSDYGVHWVRQSPGQGLEWLGVIWAGG
GTNYNSALMSRKSISKDNSKSQVFLKMNSLQADDTAVYYCARDKGYSYYYSMDYW
GQGTTVTVSSASTKGPSVFPLAPSSKSTSGGTAALGCLVKDYFPEPVTVSWNSGA
LTSGVHTCPAVLQSSGLYSLSSVVTVPSSSLGTQTYICNVNHKPSNTKVDKKVEP
KSSDKTHTCGGHHHHHH
CD28.9.3 bDS LC (SEQ ID NO: 91):
DIVLTQSPAS LAVSLGQRAT ISCRASESVEYYVTSLMQWY QQKPGQPPKL
LIFAASNVES GVPARFSGSG SGTNFSLNIHPVDEDDVAMY FCQQSRKVPY
TFGGGTKLEIKRTVAAPSVFIFPPSDEQLKSGTASVVCLLNNFYPREAKVQWKVD
NALQSGNSQESVTEQDSKDSTYSLCSTLTLSKADYEKHKVYACEVTHQGLSSPVT
KSFNRGES
Anti-CD28 HzTN228 Fab sequence
HzTN228 bDS HC (SEQ ID NO: 92):
QVQLQESGPGLVKPSETLSLTCAVSGFSLTSYGVHWIRQPGKGLEWLGVIWPGTN
FNSALMSRLTISEDTSKNQVSLKLSSVTAADTAVYCARDRAYGNYLYAMDYWGQG
TLVTVSSASTKGPSVFPLAPSSKSTSGGTAALGCLVKDYFPEPVTVSWNSGALTS
GVHTCPAVLQSSGLYSLSSVVTVPSSSLGTQTYICNVNHKPSNTKVDKKVEPKSS
DKTHTCGGHHHHHH
HzTN228 bDS LC (SEQ ID NO: 93):
DIQMTQSPSLSASVGDRVTITCRASESVEYVTSLMQWYQKPGKAPKLLIYAASNV
DSGVPSRFSGSGTDFTLTISLQPEDIATYCQSRKVPFTFGGGTKVEIKRTVAAPS
VFIFPPSDEQLKSGTASVVCLLNNFYPREAKVQWKVDNALQSGNSQESVTEQDSK
DSTYSLCSTLTLSKADYEKHKVYACEVTHQGLSSPVTKSFNRGES
Anti-CD28 TGN2122.C Fab sequence
TGN2122.C bDS HC (SEQ ID NO: 94):
QVQLVQSGAEVKKPGASVKVSCKASGYTFTDYKIHWVRQAPGQGLEWIGYIYPYS
GSSDYNQKFKSRATLTVDNSISTAYMELSRLRSDDTAVYYCARGGDAMDYWGQGT
LVTVSSASTKGPSVFPLAPSSKSTSGGTAALGCLVKDYFPEPVTVSWNSGALTSG
VHTCPAVLQSSGLYSLSSVVTVPSSSLGTQTYICNVNHKPSNTKVDKKVEPKSSD
KTHTCGGHHHHHH
TGN2122.C bDS LC (SEQ ID NO: 95):
DIQMTQSPSSLSASVGDRVTITCGASENIYGALNWYQRKPGKAPKLLIYGATNLA
DGVPSRFSGSGSGRDYTLTISSLQPEDFATYFCQNILGTWTFGGGTKVEIKRTVA
APSVFIFPPSDEQLKSGTASVVCLLNNFYPREAKVQWKVDNALQSGNSQESVTEQ
DSKDSTYSLCSTLTLSKADYEKHKVYACEVTHQGLSSPVTKSFNRGES
Anti-CD28 TGN2122.H Fab sequence
TGN2122.H bDS HC (SEQ ID NO: 96):
EVQLVESGGGLVQPGGSLRLSCAASGFTFNIYYMSWVRQAPGKGLELVAAINPDG
GNTYYPDTVKGRFTISRDNAKNSLYLQMNSLRAEDTAVYYCARYGGPGFDSWGQG
TLVTVSSASTKGPSVFPLAPSSKSTSGGTAALGCLVKDYFPEPVTVSWNSGALTS
GVHTCPAVLQSSGLYSLSSVVTVPSSSLGTQTYICNVNHKPSNTKVDKKVEPKSS
DKTHTCGGHHHHHH
TGN2122.H bDS LC (SEQ ID NO: 97):
ENVLTQSPATLSLSPGERATLSCSASSSVSYMHWYQQKPGQAPRLWIYDTSKLAS
GIPARFSGSGSRNDYTLTISSLEPEDFAVYYCFPGSGFPFMYTFGGGTKVEIKRT
VAAPSVFIFPPSDEQLKSGTASVVCLLNNFYPREAKVQWKVDNALQSGNSQESVT
EQDSKDSTYSLCSTLTLSKADYEKHKVYACEVTHQGLSSPVTKSFNRGES
Anti-CD8 TRX2 ScFv sequence (SEQ ID NO: 98):
QVQLVESGGGVVQPGRSLRLSCAASGFTFSDFGMNWVRQAPGKGLEWVALIYYDG
SNKFYADSVKGRFTISRDNSKNTLYLQMNSLRAEDTAVYYCAKPHYDGYYHFFDS
WGQGTLVTVSSGGGGSGGGGSGGGGSGGGGSDIQMTQSPSSLSASVGDRVTITCK
GSQDINNYLAWYQQKPGKAPKLLIYNTDILHTGVPSRFSGSGSGTDFTFTISSLQ
PEDIATYYCYQYNNGYTFGQGTKVEIKGGGSGGCGGHHHHHH
V1 VHH-CH1 bDS HC (SEQ ID NO: 99):
DVQLQESGGGLVQAGGSLRLSCAVSGYPYSSYCMGWFRQAPGKEREGVAAIDSDG
RTRYADSVKGRFTISQDNAKNTLYLQMNRMKPEDTAMYYCAARFGPMGCVDLSTL
SFGHWGQGTQVTVSITASTKGPSVFPLAPSSKSTSGGTAALGCLVKDYFPEPVTV
SWNSGALTSGVHTCPAVLQSSGLYSLSSVVTVPSSSLGTQTYICNVNHKPSNTKV
DKKVEPKSSDKTHTCGGHHHHHH

In some embodiments, the targeting moiety comprises a polypeptide sequence as disclosed herein. In some embodiments, the targeting moiety comprises all six CDRs of a polypeptide sequence as disclosed herein. In some embodiments, the targeting moiety comprises CDR1, CDR2, and CDR3 of an immunoglobulin single variable domain (ISVD) as disclosed herein. In further embodiments, the targeting moiety binds to the same epitope on the targeting molecule that a polypeptide sequence as disclosed herein binds to. In further embodiments, the targeting moiety competes with a polypeptide sequence as disclosed herein to bind to the same epitope on the targeting molecule.

In certain embodiments, the targeting group or immune cell targeting group (e.g., macrophage targeting agent) may be covalently coupled to a lipid via a polyethylene glycol (PEG) containing linker.

In other embodiments, the lipid used to create a conjugate may be selected from distearoyl-phosphatidylethanolamine (DSPE):

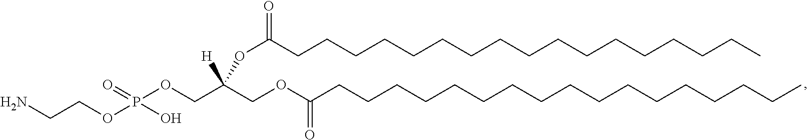 [SMILES: CCCCCCCCCCCCCCCCCC(=O)OC[C@H](COP(=O)(O)OCCN)OC(=O)CCCCCCCCCCCCCCCCC]

dipalmitoyl-phosphatidylethanolamine (DPPE):

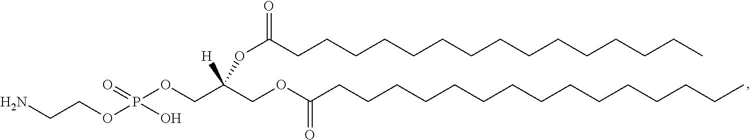 [SMILES: CCCCCCCCCCCCCCCC(=O)OC[C@H](COP(=O)(O)OCCN)OC(=O)CCCCCCCCCCCCCCC]

dimyristoyl-phosphatidylethanolamine (DMPE):

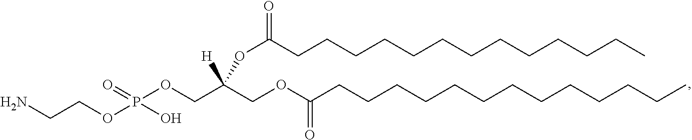 [SMILES: CCCCCCCCCCCCCC(=O)OC[C@H](COP(=O)(O)OCCN)OC(=O)CCCCCCCCCCCCC]

distearoyl-glycero-phosphoglycerol (DSPG):

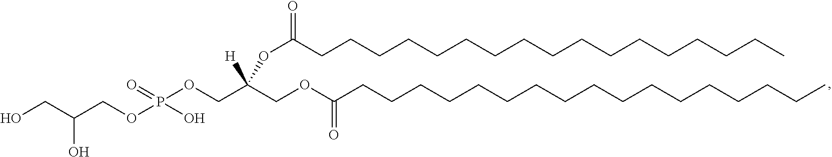 [SMILES: CCCCCCCCCCCCCCCCCC(=O)OC[C@H](COP(=O)(O)OCC(O)CO)OC(=O)CCCCCCCCCCCCCCCCC]

dimyristoyl-glycerol (DMG):

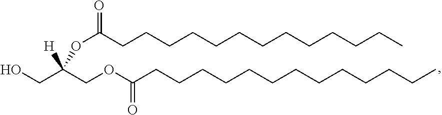 [SMILES: CCCCCCCCCCCCCC(=O)OC[C@H](CO)OC(=O)CCCCCCCCCCCCC]

distearoylglycerol (DSG):

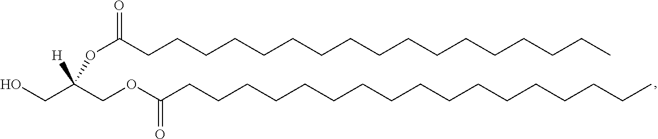 [SMILES: CCCCCCCCCCCCCCCCCC(=O)OC[C@H](CO)OC(=O)CCCCCCCCCCCCCCCCC]

and N-palmitoyl-sphingosine (C16-ceramide)

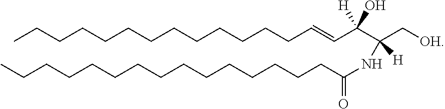 [SMILES: CCCCCCCCCCCCC/C=C/[C@@H](O)[C@H](CO)NC(=O)CCCCCCCCCCCCCCC]

The immune cell targeting group can be covalently coupled to a lipid either directly or via a linker, for example, a polyethylene glycol (PEG) containing linker. In certain embodiments, the PEG is PEG 1000, PEG 2000, PEG 3400, PEG 3000, PEG 3450, PEG 4000, or PEG 5000. In certain, embodiments, the PEG is PEG 2000.

In some embodiments, the lipid-immune cell targeting group conjugate is present in the lipid blend in a range of 0.001-0.5 mole percent, 0.001-0.3 mole percent, 0.002-0.2 mole percent, 0.01-0.1 mole percent, 0.1-0.3 mole percent, or 0.1-0.2 mole percent.

In certain embodiments, the lipid immune-cell targeting agent conjugate comprises DSPE, a PEG component and a targeting antibody. In certain embodiments, the antibody is a T-cell targeting agent, for example, an anti-CD2 antibody, an anti-CD3 antibody, an anti-CD4 antibody, an anti-CD5 antibody, an anti-CD7 antibody, an anti CD8 antibody, or an anti-TCR β antibody.

An exemplary lipid-immune cell targeting group conjugate comprises DSPE and PEG 2000, for example, as described in Nellis et al. (2005) BIOTECHNOL. PROG. 21, 205-220. An exemplary conjugate comprises the structure of Formula (III), where the scFv represents an engineered antibody binding site that binds to a target of interest. In certain embodiments, the engineered antibody binding site binds to any of the targets described hereinabove. In certain embodiments, the engineered antibody binding site can be, for example, an engineered anti-CD3 antibody or an engineered anti-CD8 antibody. In certain embodiments, the engineered antibody binding site can be, for example, an engineered anti-CD2 antibody or an engineered anti-CD7 antibody.

An example of a compound of Formula (III) is as shown below:

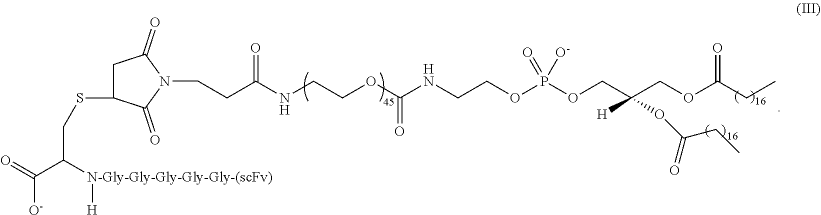

It is contemplated that the scFv in Formula (III) may be replaced with an intact antibody or an antigen fragment thereof (e.g., a Fab).

Another example of a compound of Formula (IV) is as shown below:

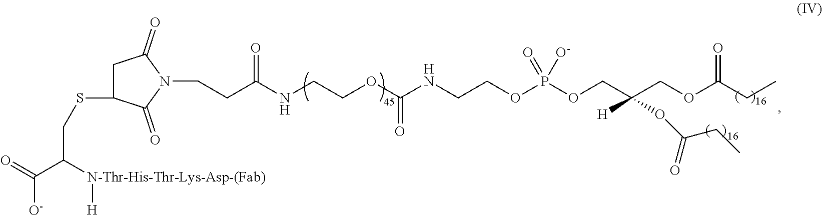

the production of which is described in Nellis et al. (2005) supra, or U.S. Pat. No. 7,022,336. It is contemplated that the Fab in Formula (IV) may be replaced with an intact antibody or an antigen fragment thereof (e.g., an (Fab′)₂ fragment) or an engineering antibody binding site (e.g., an scFv).

Other lipid immune cell target group conjugates are described, for example, in U.S. Pat. No. 7,022,336, where the targeting group may be replaced with a targeting group of interest, for example, a targeting group that binds a T-cell or NK cell surface antigen as described hereinabove.

In certain embodiments, the lipid component of an exemplary conjugate of Formula (II) can be any of the lipids described herein. In some embodiments, the lipid component of a conjugate of Formula (II) is based on an ionizable, cationic lipid described herein, for example, an ionizable, cationic lipid of Formula (I), Formula (Ia), Formula (Ib), or a slat thereof. For example, an exemplary ionizable, cationic lipid can be selected from Table 1, or a salt thereof.

In certain embodiments, the conjugate based on a lipid of the present disclosure may include:

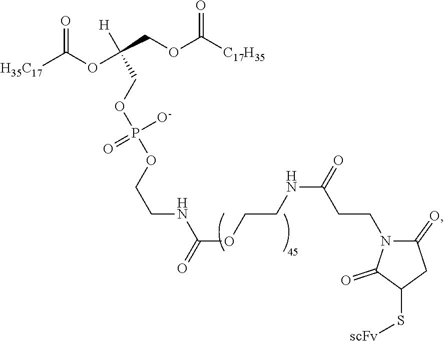

where scFv represents an engineered antibody binding site that binds a target described hereinabove, e.g., CD2, CD3, CD7, or CD8.

In certain embodiments, the lipid blend may further comprise free PEG-lipid so as to reduce the amount of non-specific binding via the targeting group. The free PEG-lipid can be the same or different from the PEG-lipid included in the conjugate. In certain embodiments, the free PEG-lipid is selected from the group consisting of PEG-distearoyl-phosphatidylethanolamine (PEG-DSPE) or PEG-dimyristoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl[methoxy(polyethylene glycol)](PEG-ceramide), DSPE-PEG-cysteine, or a derivative thereof, all with average PEG lengths between 2000-5000, with 2000, 3400, or 5000. A final composition may comprise a mixture of two or more of these pegylated lipids. In certain embodiments, the LNP composition comprises a mixture of PEG-lipids with myristoyl and stearic acyl chains. In certain embodiments, the LNP composition comprises a mixture of PEG-lipids with palmitoyl and stearoyl acyl chains.

In certain embodiments, the derivative of the PEG-lipid has a methyoxy, hydroxyl or a carboxylic acid end group at the PEG terminus.

The lipid-immune cell targeting group conjugate can be incorporated into LNPs as described below, for example, in LNPs comprising, for example, an ionizable cationic lipid, a sterol, a neutral phospholipid and a PEG-lipid. It is contemplated that, in certain embodiments, the LNPs comprising the lipid-immune cell targeting group can comprise an ionizable cationic lipid described herein or a cationic lipid described, for example, in U.S. Pat. Nos. 10,221,127, 10,653,780 or U.S. Published application No. US2018/0085474, US2016/0317676, International Publication No. WO2009/086558, or Miao et al. (2019) NATURE BIOTECH 37:1174-1185, or Jayaraman et al. (2012) ANGEW CHEM INT. 51: 8529-8533.

In some embodiments, the cationic lipid can be selected from an ionizable cationic lipid set forth in Table 1, or a salt thereof.

TABLE 1
Lipids Structures

| | |
|---|---|
| 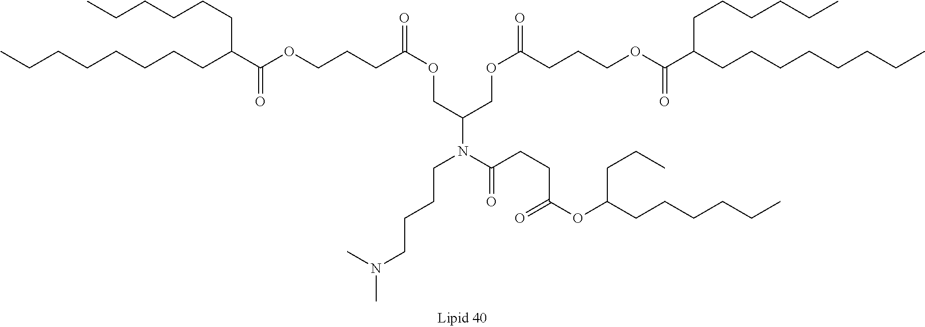 [SMILES: CCCCCCCCC(CCCCCC)C(=O)OCCCC(=O)OCC(COC(=O)CCCOC(=O)C(CCCCCC)CCCCCCCC)N(CCCCN(C)C)C(=O)CCC(=O)OC(CCC)CCCCCC] | (Lipid 40) |
| 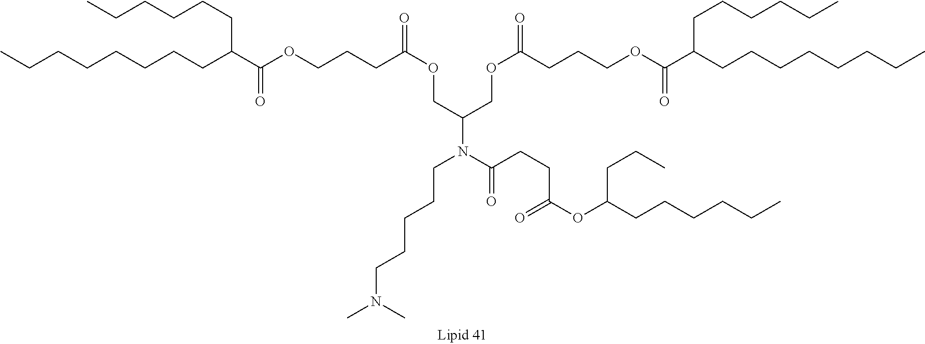 [SMILES: CCCCCCCCC(CCCCCC)C(=O)OCCCC(=O)OCC(COC(=O)CCCOC(=O)C(CCCCCC)CCCCCCCC)N(CCCCCN(C)C)C(=O)CCC(=O)OC(CCC)CCCCCC] | (Lipid 41) |
| 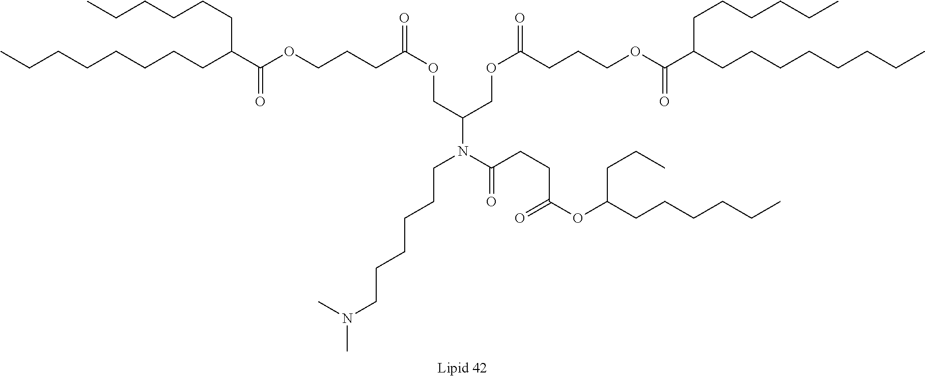 [SMILES: CCCCCCCCC(CCCCCC)C(=O)OCCCC(=O)OCC(COC(=O)CCCOC(=O)C(CCCCCC)CCCCCCCC)N(CCCCCCN(C)C)C(=O)CCC(=O)OC(CCC)CCCCCC] | (Lipid 42) |
| 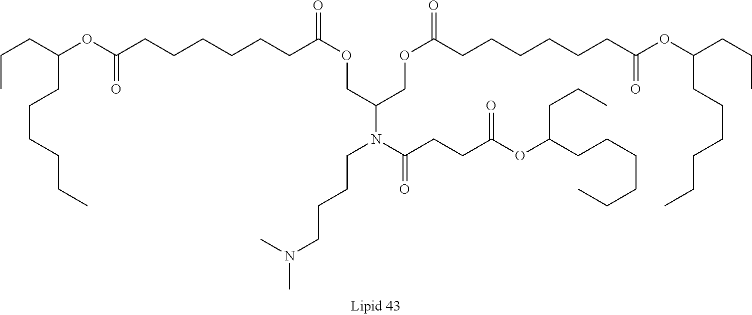 [SMILES: CCCCCCC(CCC)OC(=O)CCCCCCC(=O)OCC(COC(=O)CCCCCCC(=O)OC(CCC)CCCCCC)N(CCCCN(C)C)C(=O)CCC(=O)OC(CCC)CCCCCC] | (Lipid 43) |
| 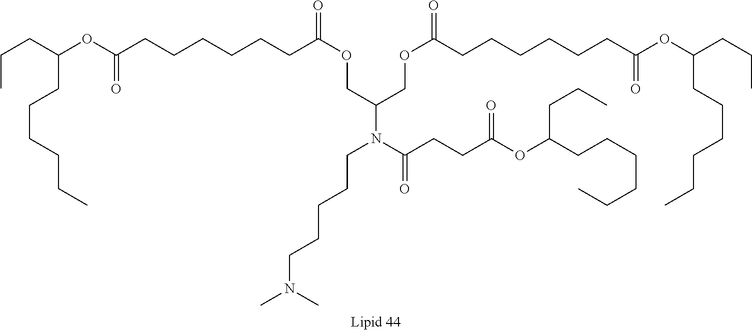 [SMILES: CCCCCCC(CCC)OC(=O)CCCCCCC(=O)OCC(COC(=O)CCCCCCC(=O)OC(CCC)CCCCCC)N(CCCCCN(C)C)C(=O)CCC(=O)OC(CCC)CCCCCC] | (Lipid 44) |
| 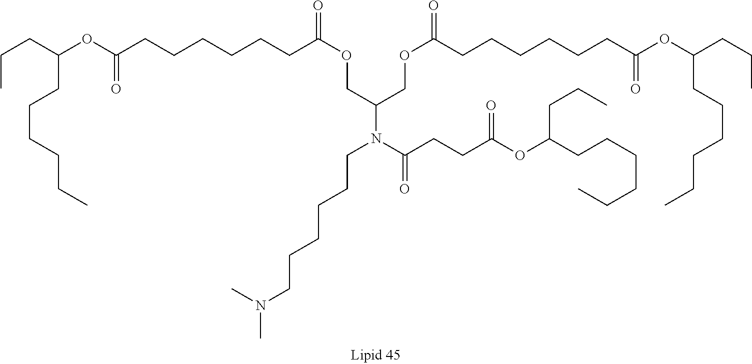 [SMILES: CCCCCCC(CCC)OC(=O)CCCCCCC(=O)OCC(COC(=O)CCCCCCC(=O)OC(CCC)CCCCCC)N(CCCCCCN(C)C)C(=O)CCC(=O)OC(CCC)CCCCCC] | (Lipid 45) |
| 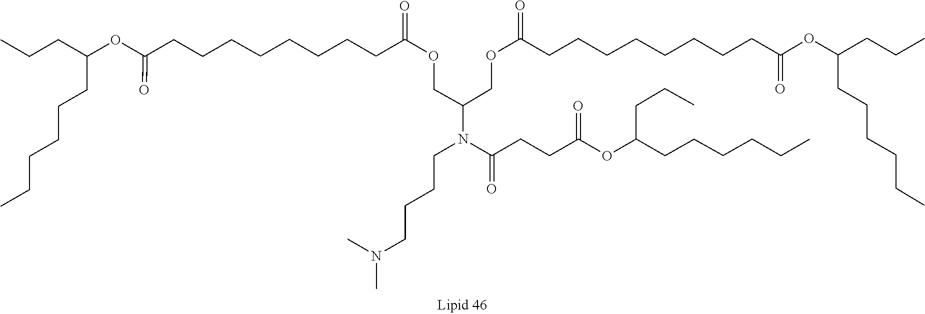 [SMILES: CCCCCCC(CCC)OC(=O)CCCCCCCCC(=O)OCC(COC(=O)CCCCCCCCC(=O)OC(CCC)CCCCCC)N(CCCCN(C)C)C(=O)CCC(=O)OC(CCC)CCCCCC] | (Lipid 46) |
| 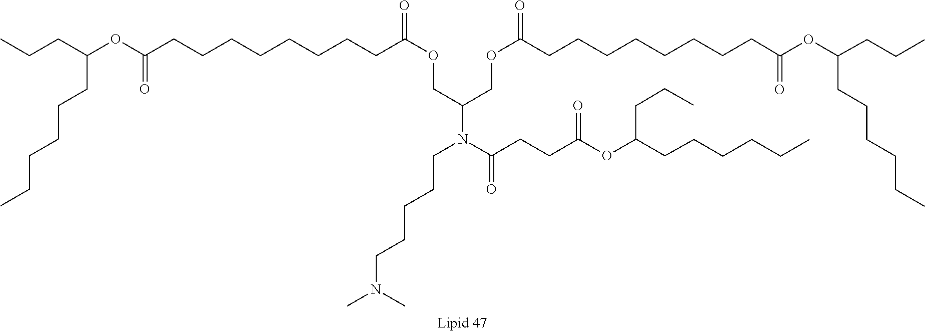 [SMILES: CCCCCCC(CCC)OC(=O)CCCCCCCCC(=O)OCC(COC(=O)CCCCCCCCC(=O)OC(CCC)CCCCCC)N(CCCCCN(C)C)C(=O)CCC(=O)OC(CCC)CCCCCC] | (Lipid 47) |
| 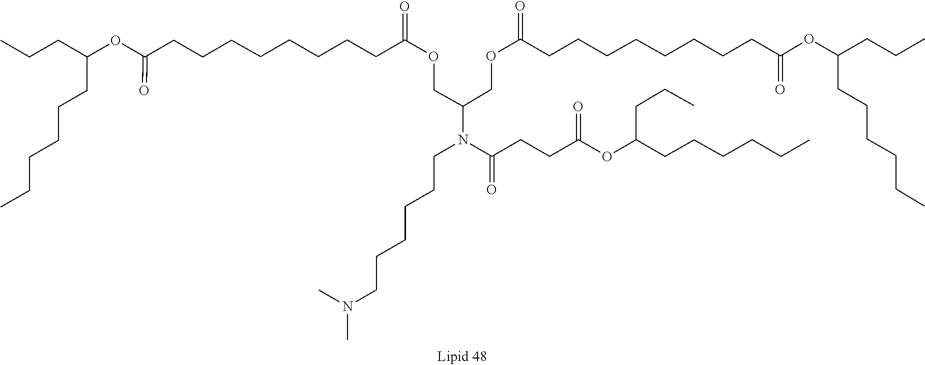 [SMILES: CCCCCCC(CCC)OC(=O)CCCCCCCCC(=O)OCC(COC(=O)CCCCCCCCC(=O)OC(CCC)CCCCCC)N(CCCCCCN(C)C)C(=O)CCC(=O)OC(CCC)CCCCCC] | (Lipid 48) |
| 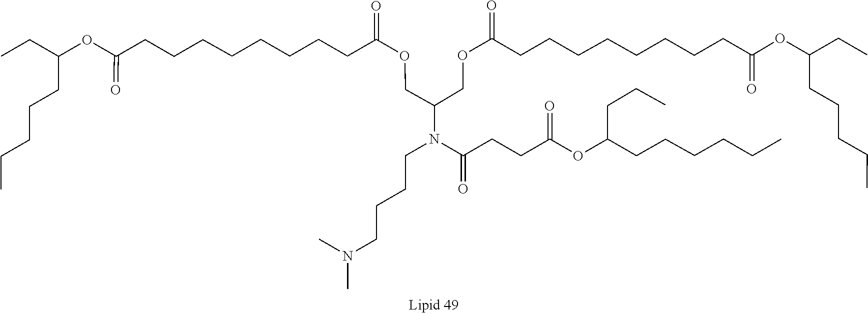 [SMILES: CCCCCCC(CCC)OC(=O)CCC(=O)N(CCCCN(C)C)C(COC(=O)CCCCCCCCC(=O)OC(CC)CCCCC)COC(=O)CCCCCCCCC(=O)OC(CC)CCCCC] | (Lipid 49) |
| 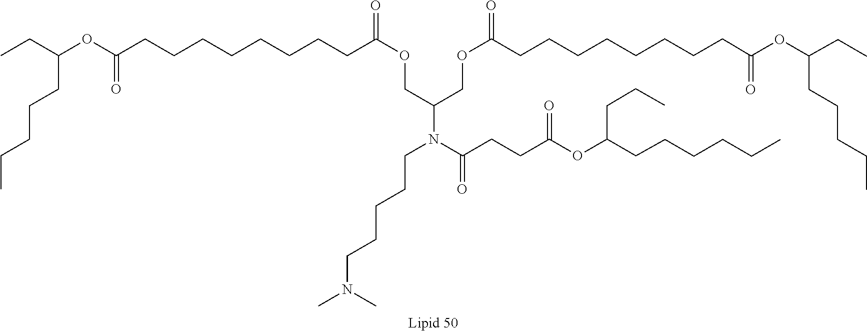 [SMILES: CCCCCCC(CCC)OC(=O)CCC(=O)N(CCCCCN(C)C)C(COC(=O)CCCCCCCCC(=O)OC(CC)CCCCC)COC(=O)CCCCCCCCC(=O)OC(CC)CCCCC] | (Lipid 50) |
| 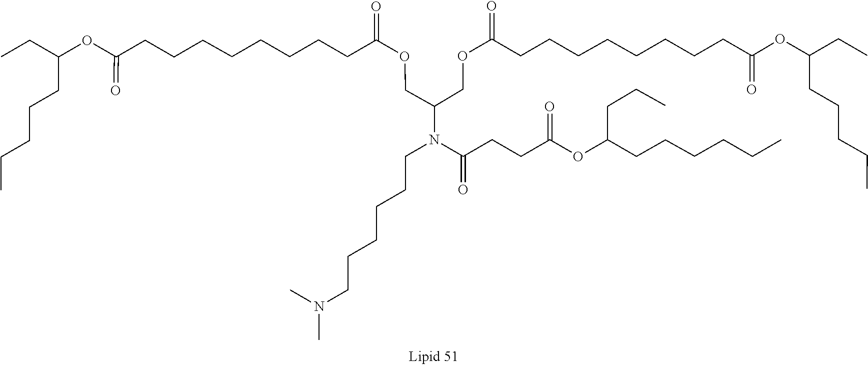 [SMILES: CCCCCCC(CCC)OC(=O)CCC(=O)N(CCCCCCN(C)C)C(COC(=O)CCCCCCCCC(=O)OC(CC)CCCCC)COC(=O)CCCCCCCCC(=O)OC(CC)CCCCC] | (Lipid 51) |
| 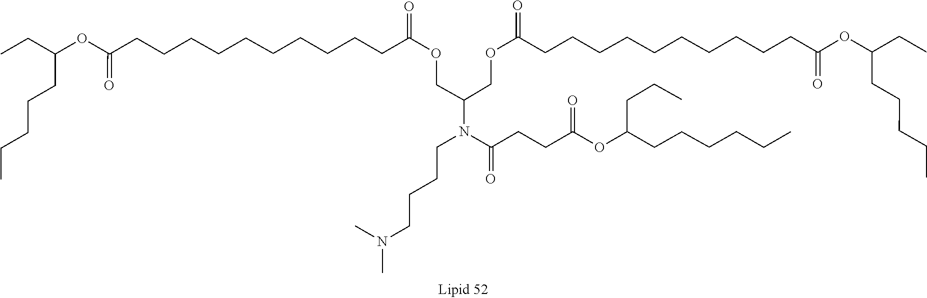 [SMILES: CCCCCCC(CCC)OC(=O)CCC(=O)N(CCCCN(C)C)C(COC(=O)CCCCCCCCCCC(=O)OC(CC)CCCCC)COC(=O)CCCCCCCCCCC(=O)OC(CC)CCCCC] | (Lipid 52) |
| 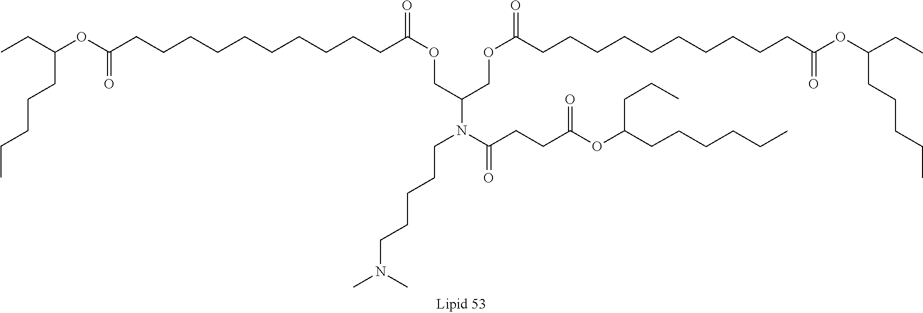 [SMILES: CCCCCCC(CCC)OC(=O)CCC(=O)N(CCCCCN(C)C)C(COC(=O)CCCCCCCCCCC(=O)OC(CC)CCCCC)COC(=O)CCCCCCCCCCC(=O)OC(CC)CCCCC] | (Lipid 53) |
| 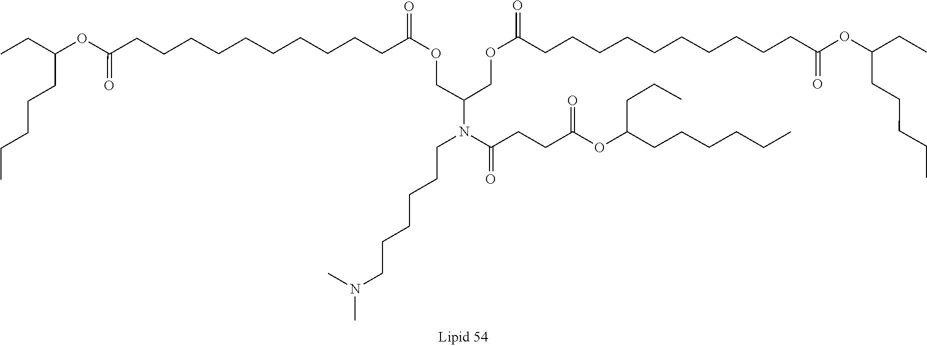 [SMILES: CCCCCCC(CCC)OC(=O)CCC(=O)N(CCCCCCN(C)C)C(COC(=O)CCCCCCCCCCC(=O)OC(CC)CCCCC)COC(=O)CCCCCCCCCCC(=O)OC(CC)CCCCC] | (Lipid 54) |
| 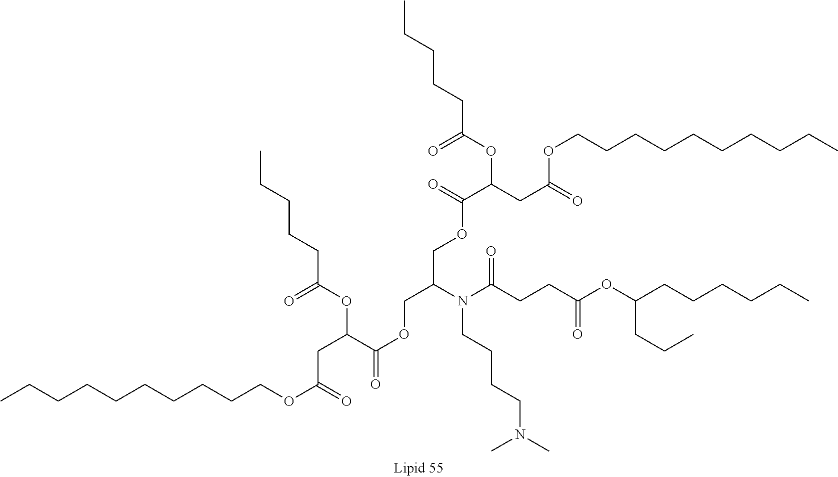 [SMILES: CCCCCCCCCCOC(=O)CC(OC(=O)CCCCC)C(=O)OCC(COC(=O)C(CC(=O)OCCCCCCCCCC)OC(=O)CCCCC)N(CCCCN(C)C)C(=O)CCC(=O)OC(CCC)CCCCCC] | (Lipid 55) |
| 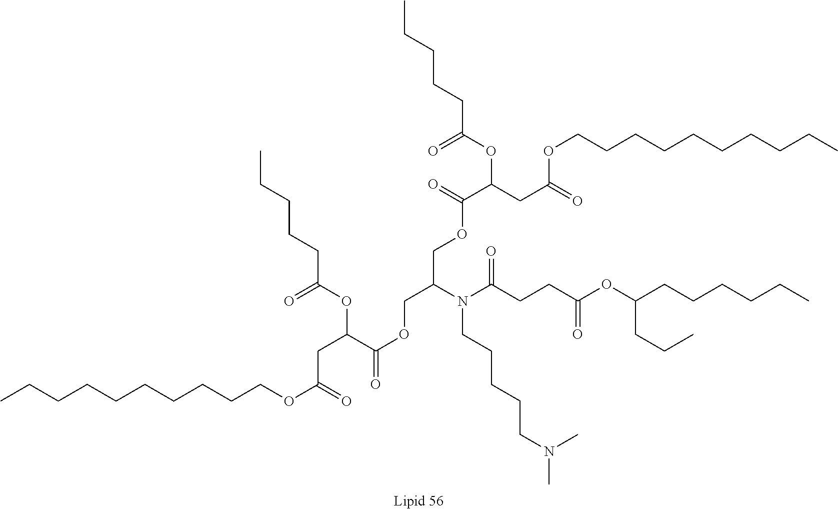 [SMILES: CCCCCCCCCCOC(=O)CC(OC(=O)CCCCC)C(=O)OCC(COC(=O)C(CC(=O)OCCCCCCCCCC)OC(=O)CCCCC)N(CCCCCN(C)C)C(=O)CCC(=O)OC(CCC)CCCCCC] | (Lipid 56) |
| 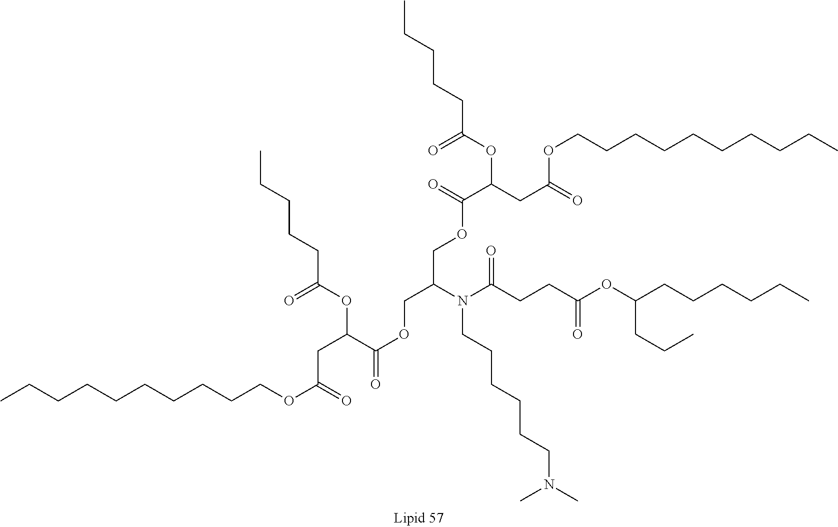 [SMILES: CCCCCCCCCCOC(=O)CC(OC(=O)CCCCC)C(=O)OCC(COC(=O)C(CC(=O)OCCCCCCCCCC)OC(=O)CCCCC)N(CCCCCCN(C)C)C(=O)CCC(=O)OC(CCC)CCCCCC] | (Lipid 57) |
| 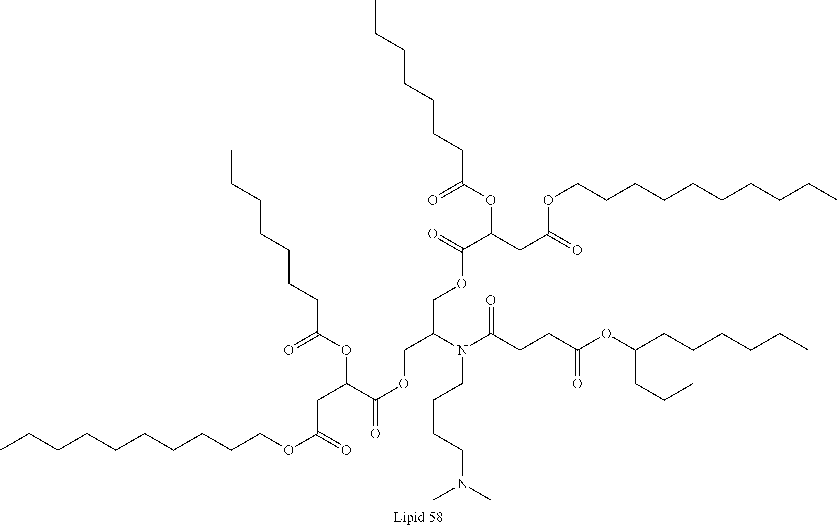 [SMILES: CCCCCCCCCCOC(=O)CC(OC(=O)CCCCCCC)C(=O)OCC(COC(=O)C(CC(=O)OCCCCCCCCCC)OC(=O)CCCCCCC)N(CCCCN(C)C)C(=O)CCC(=O)OC(CCC)CCCCCC] | (Lipid 58) |
| 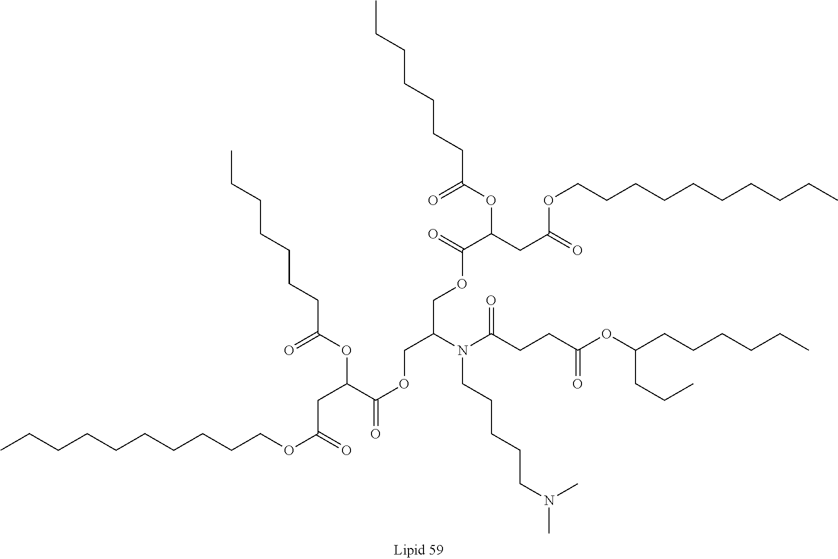 [SMILES: CCCCCCCCCCOC(=O)CC(OC(=O)CCCCCCC)C(=O)OCC(COC(=O)C(CC(=O)OCCCCCCCCCC)OC(=O)CCCCCCC)N(CCCCCN(C)C)C(=O)CCC(=O)OC(CCC)CCCCCC] | (Lipid 59) |
| 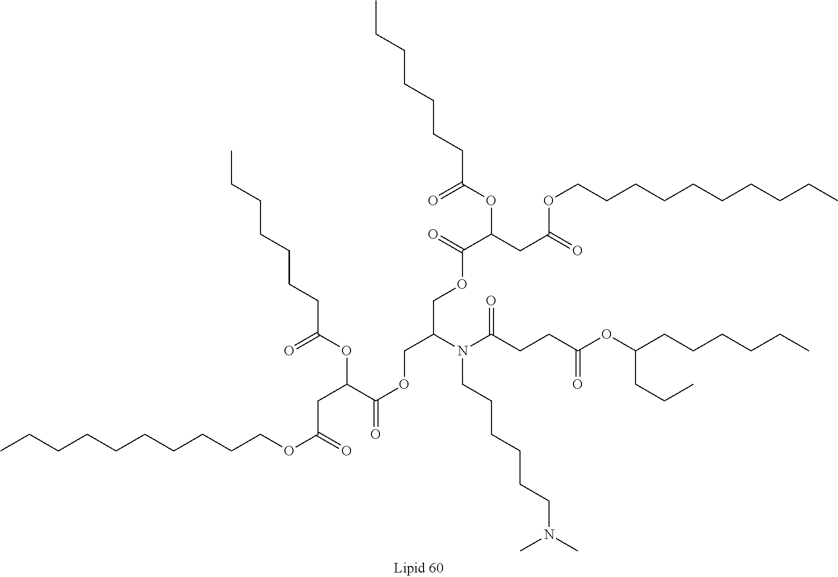 [SMILES: CCCCCCCCCCOC(=O)CC(OC(=O)CCCCCCC)C(=O)OCC(COC(=O)C(CC(=O)OCCCCCCCCCC)OC(=O)CCCCCCC)N(CCCCCCN(C)C)C(=O)CCC(=O)OC(CCC)CCCCCC] | (Lipid 60) |
| 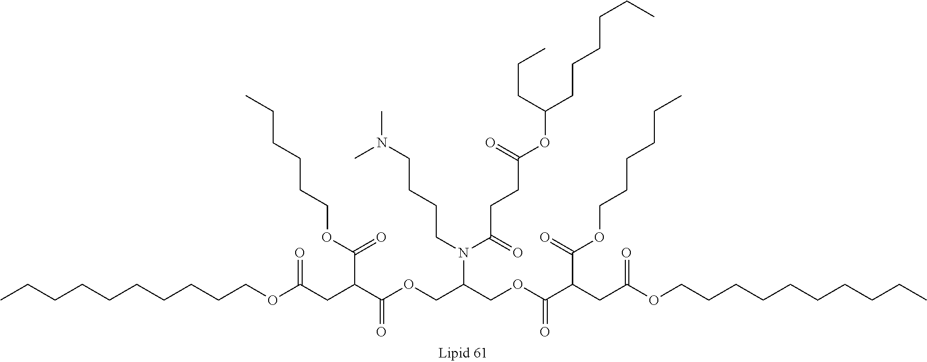 [SMILES: CCCCCCCCCCOC(=O)CC(C(=O)OCCCCCC)C(=O)OCC(COC(=O)C(CC(=O)OCCCCCCCCCC)C(=O)OCCCCCC)N(CCCCN(C)C)C(=O)CCC(=O)OC(CCC)CCCCCC] | (Lipid 61) |
| 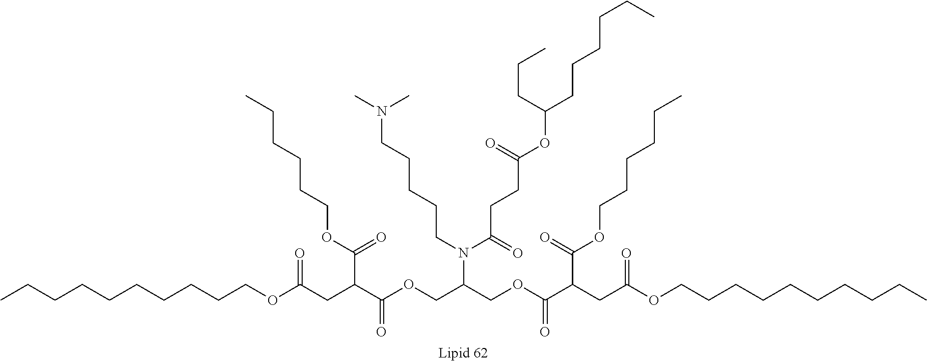 [SMILES: CCCCCCCCCCOC(=O)CC(C(=O)OCCCCCC)C(=O)OCC(COC(=O)C(CC(=O)OCCCCCCCCCC)C(=O)OCCCCCC)N(CCCCCN(C)C)C(=O)CCC(=O)OC(CCC)CCCCCC] | (Lipid 62) |
| 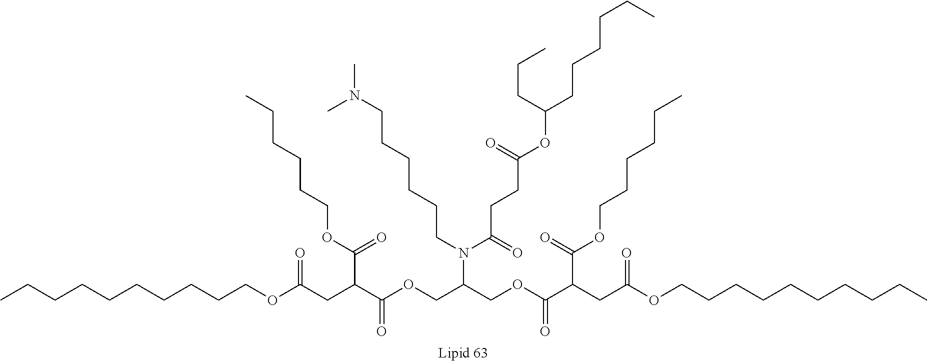 [SMILES: CCCCCCCCCCOC(=O)CC(C(=O)OCCCCCC)C(=O)OCC(COC(=O)C(CC(=O)OCCCCCCCCCC)C(=O)OCCCCCC)N(CCCCCCN(C)C)C(=O)CCC(=O)OC(CCC)CCCCCC] | (Lipid 63) |
| 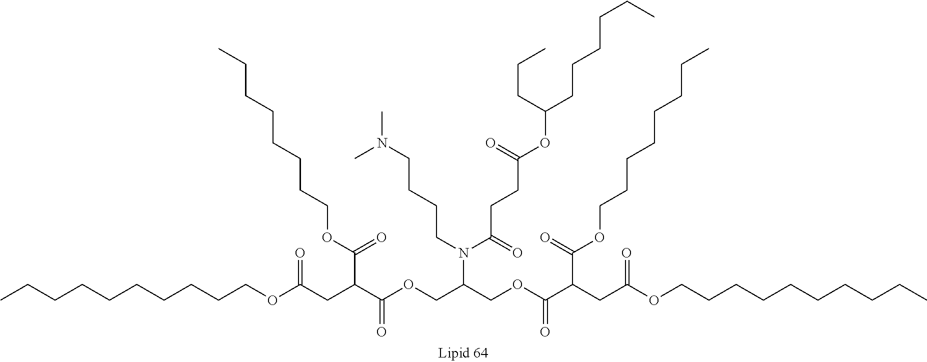 [SMILES: CCCCCCCCCCOC(=O)CC(C(=O)OCCCCCCCC)C(=O)OCC(COC(=O)C(CC(=O)OCCCCCCCCCC)C(=O)OCCCCCCCC)N(CCCCN(C)C)C(=O)CCC(=O)OC(CCC)CCCCCC] | (Lipid 64) |
| 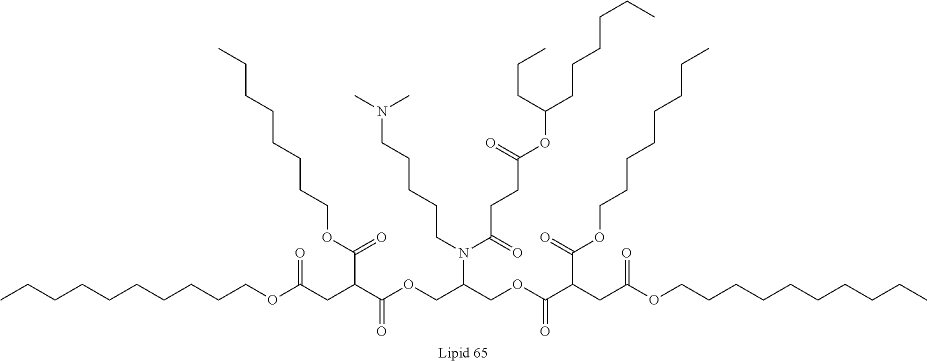 [SMILES: CCCCCCCCCCOC(=O)CC(C(=O)OCCCCCCCC)C(=O)OCC(COC(=O)C(CC(=O)OCCCCCCCCCC)C(=O)OCCCCCCCC)N(CCCCCN(C)C)C(=O)CCC(=O)OC(CCC)CCCCCC] | (Lipid 65) |
| 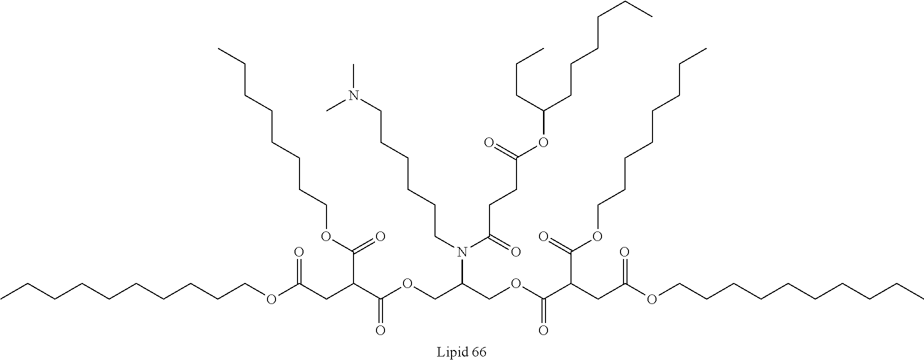 [SMILES: CCCCCCCCCCOC(=O)CC(C(=O)OCCCCCCCC)C(=O)OCC(COC(=O)C(CC(=O)OCCCCCCCCCC)C(=O)OCCCCCCCC)N(CCCCCCN(C)C)C(=O)CCC(=O)OC(CCC)CCCCCC] | (Lipid 66) |

In some embodiments, the cationic lipid is lipid 40, lipid 41, lipid 42, lipid 43, lipid 46, or lipid 52, or a salt thereof. In some embodiments, the cationic lipid is lipid 40.

Any variation or embodiment of R¹, R², R³, R^{1A}, R^{2A}, R^{3A}, R^{1A1}, R^{1A2}, R^{1A3}, R^{2A1}, R^{2A2}, R^{2A3}, R^{3A1}, R^{3A2}, R^{3A3}, R^{a1}, R^{a2}, R^{3B}, R^{3B1}, R^{3B2}, R^{3B3}, R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, or R^{s15} provided herein can be combined with every other variation or embodiment of R¹, R², R³, R^{1A}, R^{2A}, R^{3A}, R^{1A1}, R^{1A2}, R^{A3}, R^{2A1}, R^{2A2}, R^{2A3}, R^{3A1}, R^{3A2}, R^{3A3}, R^{a1}, R^{a2}, R^{3B}, R^{3B1}, R^{3B2}, R^{3B3}, R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, or R^{s15}, as if each combination had been individually and specifically described.

The LNPs can be formulated using the methods and other components described below in the following sections.

### IV. Lipid Nanoparticle Compositions

The invention provides a lipid nanoparticle (LNP) composition comprising a lipid blend that comprises an ionizable cationic lipid described herein and/or a lipid-immune cell targeting agent conjugate described herein. In certain embodiments, the lipid blend may comprise an ionizable, cationic lipid described herein and one or more of a sterol, a neutral phospholipid, a PEG-lipid, and a lipid-immune cell targeting group conjugate.

In certain embodiments, the ionizable, cationic lipid described herein may be present in the lipid blend in a range of 30-70 mole percent, 30-60 mole percent 30-50 mole percent, 40-70 mole percent, 40-60 mole percent, 40-50 mole percent, 50-70 mole percent, 50-60 mole percent, or of about 30 mole percent, about 35 mole percent, about 40 mole percent, about 45 mole percent, about 50 mole percent, about 55 mole percent, about 60 mole percent, about 65 mole percent, or about 70 mole percent.

### Sterol

In certain embodiments, the lipid blend of the lipid nanoparticle may comprise a sterol component, for example, one or more sterols selected from the group consisting of cholesterol, fecosterol, β-sitosterol, ergosterol, campesterol, stigmasterol, stigmastanol, brassicasterol. In certain embodiments, the sterol is cholesterol.

The sterol (e.g., cholesterol) may be present in the lipid blend in a range of 20-70 mole percent, 20-60 mole percent, 20-50 mole percent, 30-70 mole percent, 30-60 mole percent, 30-50 mole percent, 40-70 mole percent, 40-60 mole percent, 40-50 mole percent, 50-70 mole percent, 50-60 mole percent, or about 20 mole percent, about 25 mole percent, about 30 mole percent, about 35 mole percent, about 40 mole percent, about 45 mole percent, about 50 mole percent, about 55 mole percent, about 60 mole percent or about 65 mole percent.

### Neutral Phospholipid

In certain embodiments, the lipid blend of the lipid nanoparticle may comprise one or more neutral phospholipids. The neutral phospholipid can be selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), 1,2-dioleoyl-sn-glycero-3-phosphocholine (DOPC), sphingomyelin (SM).

Other neutral phospholipids can be selected from the group consisting of distearoyl-phosphatidylethanolamine (DSPE), dimyristoyl-phosphatidylethanolamine (DMPE), distearoyl-glycero-phosphocholine (DSPC), hydrogenated soy phosphatidylcholine (HSPC), dioleoyl-glycero-phosphoethanolamine (DOPE), dilinolenoyl-glycero-phosphocholine (DLPC), dimyristoyl-glycero-phosphocholine (DMPC), dioleoyl-glycero-phosphocholine (DOPC), dipalmitoyl-glycero-phosphocholine (DPPC), diundecanoyl-glycero-phosphocholine (DUPC), palmitoyl-oleoyl-glycero-phosphocholine (POPC), dioctadecenyl-glycero-phosphocholine, oleoyl-cholesterylhemisuccinoyl-glycero-phosphocholine, hexadecyl-glycero-phosphocholine, dilinolenoyl-glycero-phosphocholine, diarachidonoyl-glycero-3-phosphocholine, didocosahexaenoyl-glycero-phosphocholine, or sphingomyelin.

The neutral phospholipid may be present in the lipid blend in a range of 1-10 mole percent, 1-15 mole percent, 1-12 mole percent, 1-10 mole percent, 3-15 mole percent, 3-12 mole percent, 3-10 mole percent, 4-15 mole percent, 4-12 mole percent, 4-10 mole percent, 4-8 mole percent, 5-15 mole percent, 5-12 mole percent, 5-10 mole percent, 6-15 mole percent, 6-12 mole percent, 6-10 more percent, or about 1 mole percent, about 2 mole percent, about 3 mole percent, about 4 mole percent, about 5 mole percent, about 6 mole percent, about 7 mole percent, about 8 mole percent, about 9 mole percent, about 10 mole percent, about 11 mole percent, about 12 mole percent, about 13 mole percent, about 14 mole percent, or about 15 mole percent.

### PEG-Lipid

The lipid blend of the lipid nanoparticle may include one or more PEG or PEG-modified lipids. Such species may be alternately referred to as PEGylated lipids. A PEG lipid is a lipid modified with polyethylene glycol. As noted above, free PEG-lipids can be included in the lipid blend to reduce or eliminate non-specific binding via a targeting group when a lipid-immune cell targeting group is included in the lipid blend.

A PEG lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylglycerol (PEG-DOG), PEG-dimyristoyl-glycerol (PEG-DMG), PEG-dipalmitoyl-glycerol (PEG-DPG), PEG-dilinolenoyl-glycero-phosphatidyl ethanolamine (PEG-DLPE), PEG-dimyristoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

In certain embodiments, the blend may comprise a free PEG-lipid that can be selected from the group consisting of PEG-distearoylglycerol (PEG-DSG), PEG-diacylglycerol (PEG-DAG, e.g., PEG-DMG, PEG-DPG, and PEG-DSG), PEG-dimyristoyl-glycerol (PEG-DMG), PEG-distearoyl-phosphatidylethanolamine (PEG-DSPE) and PEG-dimyristoyl-phosphatidylethanolamine (PEG-DMPE). In some embodiments, the free PEG-lipid comprises a diacylphosphatidylcholines comprising Dipalmitoyl (C16) chain or Distearoyl (C18) chain.

The PEG-lipid may be present in the lipid blend in a range of 1-10 mole percent, 1-8 mole percent, 1-7 mole percent, 1-6 mole percent, 1-5 mole percent, 1-4 mole percent, 1-3 mole percent, 2-8 mole percent, 2-7 mole percent, 2-6 mole percent, 2-5 mole percent, 2-4 mole percent, 2-3 mole percent, or about 1 mole percent, about 2 mole percent, about 3 mole percent, about 4 mole percent, or about 5 mole percent. In some embodiments, the PEG-lipid is a free PEG-lipid.

In some embodiments, the PEG-lipid may be present in the lipid blend in the range of 0.01-10 mole percent, 0.01-5 mole percent, 0.01-4 mole percent, 0.01-3 mole percent, 0.01-2 mole percent, 0.01-1 mole percent, 0.1-10 mole percent, 0.1-5 mole percent, 0.1-4 mole percent, 0.1-3 mole percent, 0.1-2 mole percent, 0.1-1 mole percent, 0.5-10 mole percent, 0.5-5 mole percent, 0.5-4 mole percent, 0.5-3 mole percent, 0.5-2 mole percent, 0.5-1 mole percent, 1-2 mole percent, 3-4 mole percent, 4-5 mole percent, 5-6 mole percent, or 1.25-1.75 mole percent. In some embodiments, the PET-lipid may be about 0.5 mole percent, about 1 mole percent, about 1.5 mole percent, about 2 mole percent, about 2.5 mole percent, about 3 mole percent, about 3.5 mole percent, about 4 mole percent, about 4.5 mole percent, about 5 mole percent, or about 5.5 mole percent of the lipid blend. In some embodiments, the PEG-lipid is a free PEG-lipid.

In some embodiments, the lipid anchor length of PEG-lipid is C14 (as in PEG-DMG). In some embodiments, the lipid anchor length of PEG-lipid is C16 (as in DPG). In some embodiments, the lipid anchor length of PEG-lipid is C18 (as in PEG-DSG). In some embodiments, the back bone or head group of PEG-lipid is diacyl glycerol or phosphoethanolamine. In some embodiments, the PEG-lipid is a free PEG-lipid.

A LNP of the present disclosure may comprise one or more free PEG-lipid that is not conjugated to an immune cell targeting group, and a PEG-lipid that is conjugated to immune cell targeting group. In some embodiments, the free PEG-lipid comprises the same or a different lipid as the lipid in the lipid-immune cell targeting group conjugate.

### Immune Cell Targeting Group Conjugate

In certain embodiments, the lipid blend can also include a lipid-immune cell targeting group conjugate.

The lipid-immune cell targeting group conjugate may be present in the lipid blend in a range of 0.001-0.5 mol percent, 0.001-0.1 mole percent, 0.01-0.5 mole percent, 0.05-0.5 mole percent, 0.1-0.5 mole percent, 0.1-0.3 mole percent, 0.1-0.2 mole percent, 0.2-0.3 mole percent, of about 0.01 mole percent, about 0.05 mole percent, about 0.1 mole percent, about 0.15 mole percent, about 0.2 mole percent, about 0.25 mole percent, about 0.3 mole percent, about 0.35 mole percent, about 0.4 mole percent, about 0.45 mole percent, or about 0.5 mole percent.

In addition to the lipids present in the lipid blend, the LNP compositions may further comprise a payload, for example, a payload described hereinbelow. In certain embodiments, the payload is a nucleic acid, for example, DNA or RNA, for example, an mRNA, transfer RNA (tRNA), a microRNA, or small interfering RNA (siRNA).

In certain embodiments, the number of the nucleotides in the nucleic acid is from about 400 to about 6000.

### Production of Lipid Nanoparticles

In some embodiments, the LNPs are produced by using either rapid mixing by an orbital vortexer or by microfluidic mixing. Orbital vortexer mixing is accomplished by rapid addition of lipids solution in ethanol to the aqueous solution of a nucleic acid of interest followed immediately by vortexing at 2,500 rpm. In some embodiments, the LNPs are produced using a microfluidic mixing step. In some embodiments, microfluidic mixing is achieved mixing the aqueous and organic streams at a controlled flow rates in a microfluidic channel using, e.g., a NanoAssemblr device and microfluidic chips featuring optimized mixing chamber geometry (Precision Nanosystems, Vancouver, BC). In some embodiments, the LNPs are produced using a microfluidic mixing step to rapidly mix the ethanolic lipid solution and aqueous nucleic acid solution, resulting in encapsulation of the nucleic acid in the solid lipid nanoparticles. The nanoparticle suspension is then buffer exchanged into an all aqueous buffer using membrane filtration device of choice for ethanol removal and nanoparticle maturation.

In certain embodiments, the resulting LNP compositions comprise a lipid blend comprising, for example, from about 40 mole percent to about 60 mole percent of one or more ionizable cationic lipids described herein, from about 35 mole percent to about 50 mole percent of one or more sterols, from about 5 mole percent to about 15 mole percent of one or more neutral lipids, and from about 0.5 mole percent to about 5 mole percent of one or more PEG-lipids.

### Physical Properties of Lipid Nanoparticles

The characteristics of an LNP composition may depend on the components, their absolute or relative amounts, contained in a lipid nanoparticle (LNP) composition. Characteristics may also vary depending on the method and conditions of preparation of the LNP composition.

LNP compositions may be characterized by a variety of methods. For example, microscopy (e.g., transmission electron microscopy or scanning electron microscopy) may be used to examine the morphology and size distribution of an LNP composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of an LNP composition, such as particle size, polydispersity index, and zeta potential. RNA encapsulated efficiency is determined by a combination of methods relying on RNA binding dyes (ribogreen, cybergreen to determine dye accessible RNA fraction) and LNP de-formulation followed by HPLC analysis for total RNA content.

In some embodiments, the LNP may have a mean diameter in the range of 1-250 nm, 1-200 nm, 1-150 nm, 1-100 nm, 50-250 nm, 50-200 nm, 50-150 nm, 50-100 nm, 75-250 nm, 75-200 nm, 75-150 nm, 75-100 nm, 100-250 nm, 100-200 nm, 100-150 nm. In certain embodiments, the LNP compositions may have a mean diameter of about 1 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the LNP has a mean diameter of about 100 nm.

In some embodiments, LNPs comprising an ionizable cationic lipid described herein, prepared and characterized using methods described herein, show average diameter change after a freeze-thaw of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change after a freeze-thaw of less than 30%. In some embodiments, the freeze-thaw and diameter measurements are conducted with 10% sucrose in MES pH 6.5 buffer.

In some embodiments, LNPs comprising an ionizable cationic lipid described herein, prepared and characterized using methods described herein, show average diameter change upon targeting antibody insertion of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change upon targeting antibody insertion of less than 15%. In some embodiments, the diameter change upon targeting antibody insertion is measured in pH 6.5 MES using a 37° C. incubation for 4 hours.

In some embodiments, LNPs comprising an ionizable cationic lipid described herein, prepared and characterized using methods described herein, have average LNP diameter of less than 50, 60, 70, 80, 90, 100, 110, 120, 130, 140, 150, 160, 170, 180, 190, or 200 nm. In some embodiments, LNPs comprising an ionizable cationic lipid described herein, prepared and characterized using methods described herein, have average LNP diameter of less than 100 nm.

Alternatively or in addition, the LNP compositions may have a polydispersity index in a range from 0.05-1, 0.05-0.75, 0.05-0.5, 0.05-0.4, 0.05-0.3, 0.05-0.2, 0.08-1, 0.08-0.75, 0.08-0.5, 0.08-0.4, 0.08-0.3, 0.08-0.2, 0.1-1, 0.1-0.75, 0.1-0.5, 0.1-0.4, 0.1-0.3, 0.1-0.2. In certain embodiments, the polydispersity index is in the range of 0.1-0.25, 0.1-0.2, 0.1-0.19, 0.1-0.18, 0.1-0.17, 0.1-0.16, or 0.1-0.15.

In some embodiments, the LNP compositions or LNPs comprising an ionizable cationic lipid described herein, prepared and characterized using methods described herein, have polydispersity of less than 0.4, 0.3, 0.25, 0.2, 0.15, 0.1, or 0.05. In some embodiments, LNPs comprising an ionizable cationic lipid described herein, prepared and characterized using methods described herein, have polydispersity of less than 0.25.

Alternatively or in addition, the LNP compositions may have a zeta potential of about −30 mV to about +30 mV. In certain embodiments, the LNP composition has a zeta potential of about −10 mV to about +20 mV. The zeta potential may vary as a function of pH. As a result, in certain embodiments, the LNP compositions may have a zeta potential of about 0 mV to about +30 mV or about +10 mV to +30 mV or about +20 mV to about +30 mV at pH 5.5 or pH 5, and/or a zeta potential of about −30 mV to about +5 mV or about −20 mV to about +15 mV at pH 7.4.

In some embodiments, the LNP compositions or LNPs comprising an ionizable cationic lipid described herein, prepared and characterized using methods described herein, have Zeta Potential at pH 7.4 greater than −10, −9, −8, −7, −6, −5.5, −5, −4.5, −4, −3.5, −3, −2.5, −2, −1.5, −1, or −0.5 mV. In some embodiments, the LNP compositions LNPs comprising an ionizable cationic lipid described herein, prepared and characterized using methods described herein, have Zeta Potential at pH 7.4 greater than −10 mV. In some embodiments, the LNP compositions LNPs comprising an ionizable cationic lipid described herein, prepared and characterized using methods described herein, have Zeta Potential at pH 7.4 greater than −1 mV. In some embodiments, the LNP compositions LNPs comprising an ionizable cationic lipid described herein, prepared and characterized using methods described herein, have Zeta Potential at pH 5.5 greater than −1, 0, 1, 2, 3, 4, 4.5, 5, 7.5, 10, 12.5, 15, 17.5, 20, 22.5, or 25 mV. In some embodiments, the LNP compositions LNPs comprising an ionizable cationic lipid described herein, prepared and characterized using methods described herein, have Zeta Potential at pH 5.5 greater than 5 mV. In some embodiments, the LNP compositions LNPs comprising an ionizable cationic lipid described herein, prepared and characterized using methods described herein, have Zeta Potential at pH 5.5 greater than 15 mV.

### Selective Organ Delivery

In some embodiments, the LNP described herein has high liver avoidance. In some embodiments, the LNP comprises about 1.5 mol % of free PEG-lipid. In some embodiments, the LNP comprises about 1 to about 2 mol % of free PEG-lipid. In some embodiments, the free PEG-lipid is DSG-PEG. In some embodiments, the LNP comprising about 3.5 mol % of DSG-PEG has high liver targeting.

In some embodiments, liver avoidance is measured with imaging (e.g., ex vivo luciferase imaging). In some embodiments, liver avoidance is measured as a non-liver/liver ratio. In some embodiments, the non-liver/liver ratio is the level of LNP accumulation, cargo delivery, or cargo expression in a non-liver organ (e.g., spleen) relative to that in the liver. In some embodiments, liver avoidance is measured certain period of time (e.g., 24 hours) after dosing the subject with the LNP. In some embodiments, the non-liver/liver ratio is about 1 to about 1.5. In some embodiments, the non-liver/liver ratio is about 1.25.

In some embodiments, the LNP described herein has high liver targeting. In some embodiments, the LNP comprises about 3.5 mol % of free PEG-lipid. In some embodiments, the LNP comprises about 3 to about 4 mol % of free PEG-lipid. In some embodiments, the free PEG-lipid is DSG-PEG. In some embodiments, the free PEG-lipid is DPG-PEG. In some embodiments, the LNP comprising about 3.5 mol % of DSG-PEG or about 3.5 mol % of DPG-PEG has high liver targeting.

In some embodiments, liver targeting is measured with imaging (e.g., ex vivo luciferase imaging). In some embodiments, liver targeting is measured as a liver/non-liver ratio. In some embodiments, the liver/non-liver ratio is the level of LNP accumulation, cargo delivery, or cargo expression in the liver relative to that in a non-liver organ (e.g., spleen). In some embodiments, liver targeting is measured certain period of time (e.g., 24 hours) after dosing the subject with the LNP. In some embodiments, the liver/non-liver ratio is about 1 to about 2, or about 1.5 to about 2. In some embodiments, the liver/non-liver ratio is about 1.6 or about 1.8.

### V. Payloads

The LNP compositions may comprise an agent, for example, a nucleic acid molecule for delivery to a cell (e.g., an immune cell) or tissue, for example, a cell (e.g., an immune cell) or tissue in a subject.

The LNP compositions of the present invention may include a nucleic acid, for example, a DNA or RNA, such as an mRNA, tRNA, microRNA, siRNA, gRNA (guide RNA), circRNA (circular RNA), ribozymes, decoy RNA or dicer substrate siRNA. It is contemplated that nucleic acids can contain naturally occurring components, such as, naturally occurring bases, sugars or linkage groups (e.g., phosphodiester linkage groups) or may contain non-naturally occurring components or modifications, (e.g., thioester linkage groups). For example, the nucleic acid can be synthesized to contain base, sugar, and/or linker modifications known to those skilled in the art. Furthermore, the nucleic acids can be linear or circular, or have any desired configuration. The LNP compositions can include multiple nucleic acid molecules, for example, multiple RNA molecules, which can be the same or different.

In certain embodiments, the payload is an mRNA. In certain embodiments, a particular LNP composition may comprise a number of mRNA molecules that can be the same or different. In certain embodiments, one or more LNP compositions including one or more different mRNAs may be combined, and/or simultaneously contacted, with a cell. It is contemplated that an mRNA may include one or more of a stem loop, a chain terminating nucleoside, a polyA sequence, a polyadenylation signal, and/or a 5′ cap structure. The mRNA may encode a receptor, such as a chimeric antigen receptor (CAR), for use in for example, an immune disorder, inflammatory disorder or cancer. In addition, the mRNA may encode an antigen for use in a therapeutic or prophylactic vaccine, for example, for treating or preventing an infection by a pathogen, for example, a microbial or viral pathogen, or for reducing or ameliorating the side effects caused directly or indirectly by such an infection.

In certain embodiments, the LNP composition may include one or more other components including, but not limited to, one or more pharmaceutically acceptable excipients, small hydrophobic molecules, therapeutic agents, carbohydrates, polymers, permeability enhancing molecules, and surface altering agents.

In some embodiments, the wt/wt ratio of the lipid component to the payload (e.g., mRNA) in the resulting LNP composition is from about 1:1 to about 50:1. In certain embodiments, the wt/wt ratio of the lipid component to the payload (e.g., mRNA) in the resulting composition is from about 5:1 to about 50:1. In certain embodiments, the wt/wt ratio is from about 5:1 to about 40:1. In certain embodiments, the wt/wt ratio is from about 10:1 to about 40:1. In certain embodiments, the wt/wt ratio is from about 15:1 to about 25:1.

In certain embodiments, the encapsulation efficiency of the payload (e.g., mRNA) in the lipid nanoparticles is at least 50%. In certain embodiments, the encapsulation efficiency is at least 80%, at least 90%, or greater than 90%.

In some embodiments, LNPs comprising an ionizable cationic lipid described herein, prepared and characterized using methods described herein, exhibit encapsulation efficiency of greater than 50, 55, 60, 65, 70, 75, 80, 82.5, 85, 87.5, 90, 92.5, 95, 97.5, or 99%. In some embodiments, LNPs comprising an ionizable cationic lipid described herein, prepared and characterized using methods described herein, exhibit encapsulation efficiency of greater than 87.5%. In some embodiments, LNPs comprising an ionizable cationic lipid described herein, prepared and characterized using methods described herein, exhibit dye accessible RNA of less than 50, 45, 40, 35, 30, 25, 20, 17.5, 15, 12.5, 10, 7.5, 5, 2.5, or 1%. In some embodiments, LNPs comprising an ionizable cationic lipid described herein, prepared and characterized using methods described herein, exhibit dye accessible RNA of less than 12.5%.

In some embodiments, LNPs comprising an ionizable cationic lipid described herein, prepared and characterized using methods described herein, exhibit total mRNA recovery of greater than 50, 55, 60, 65, 70, 75, 80, 85, 90, or 95%. In some embodiments, LNPs comprising an ionizable cationic lipid described herein, prepared and characterized using methods described herein, exhibit total mRNA recovery of greater than 80%.

### RNA Payload

In certain embodiments, the RNA payload is an mRNA, tRNA, microRNA, or siRNA payload.

In certain embodiments, the lipid nanoparticle compositions are optimized for the delivery of RNA, e.g., mRNA, to a target cell for translation within the cell. An mRNA may be a naturally or non-naturally occurring mRNA. An mRNA may include one or more modified nucleobases, nucleosides, or nucleotides.

The nucleobases may be selected from the non-limiting group consisting of adenine, guanine, uracil, cytosine, 7-methylguanine, 5-methylcytosine, 5-hydroxymethylcytosine, thymine, pseudouracil, dihydrouracil, N1-methylpseudouracil, hypoxanthine, and xanthine. In some embodiments, nucleobase is N1-methylpseudouracil.

A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications.

A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein.

An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. In certain embodiments, one or more or all uridine bases may be N1-methylpseudouridines.

In certain embodiments, an mRNA may include a 5′ cap structure, a chain terminating nucleotide, a stem loop, a polyA sequence, and/or a polyadenylation signal.

A cap structure or cap species is a compound including two nucleoside moieties joined by a linker and may be selected from a naturally occurring cap, a non-naturally occurring cap or a cap analog. A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m7Gpppm7G, m73′dGpppG, and m27 02′GppppG.

Alternatively or in addition, an mRNA may include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

Alternatively or in addition, an mRNA may include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

Alternatively or in addition, an mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA.

An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell. In some embodiments, the mRNA may encode an antibody, enzyme, growth factor, hormone, cytokine, viral protein (e.g., a viral capsid protein), antigen, vaccine, or receptor. In some embodiments, the mRNA may encode an engineered receptor such as a CAR or an antigen for use in a therapeutic vaccine (e.g., a cancer vaccine) or a prophylactic vaccine (e.g., a vaccine for minimizing the risk or severity of an infection by a microbial or viral pathogen). In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

A lipid composition may be designed for one or more specific applications or targets. For example, an LNP composition may be designed to deliver mRNA to a particular cell, tissue, organ, or system or group thereof in a mammal's body, such as the renal system. Physiochemical properties of LNP compositions may be altered in order to increase selectivity for particular target site within a subject. For instance, particle sizes may be adjusted based on the fenestration sizes of different organs. The mRNA included in an LNP composition may also depend on the desired delivery target or targets. For example, an mRNA may be selected for a particular indication, condition, disease, or disorder and/or for delivery to a particular cell, tissue, organ, or system or group thereof (e.g., localized or specific delivery).

The amount of mRNA in a lipid composition may depend on the size, sequence, and other characteristics of the mRNA. The amount of mRNA in an LNP may also depend on the size, composition, desired target, and other characteristics of the LNP composition. The relative amounts of mRNA and other elements (e.g., lipids) may also vary. The amount of mRNA in an LNP composition may, for example, be measured using absorption spectroscopy (e.g., ultraviolet-visible spectroscopy).

In some embodiments, the one or more mRNAs, lipids, and polymers and amounts thereof may be selected to provide a specific N:P ratio (the ratio of positively-chargeable lipid or polymer amine (N=nitrogen) groups to negatively-charged nucleic acid phosphate (P) groups). The N:P ratio of the composition refers to the molar ratio of nitrogen atoms in one or more lipids to the number of phosphate groups in an mRNA. In general, a lower N:P ratio is preferred. A N:P ratio may be dependent on a specific lipid and its pKa. In certain embodiments, the mRNA and LNP composition, and/or their relative amounts may be selected to provide an N:P ratio from about 1:1 to about 30:1, or from about 1:1 to about 20:1. In certain embodiments, the N:P ratio can be, for example, 1:1, 2:1, 3:1, 4:1, 5:1, 6:1, 7:1, or 8:1. In certain embodiments, the N:P ratio may be from about 2:1 to about 5:1. In certain embodiments, the N:P ratio may be about 4:1. In other embodiments, the N:P ratio is from about 4:1 to about 8:1. For example, the N:P ratio may be about 4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 6.0:1, about 6.5:1, or about 7.0:1.

The amount of mRNA in a nanoparticle composition may depend on the size, sequence, and other characteristics of the mRNA. The amount of mRNA in a nanoparticle composition may also depend on the size, composition, desired target, and other characteristics of the nanoparticle composition. The relative amounts of mRNA and other elements (e.g., lipids) may also vary. In some embodiments, the wt/wt ratio of the lipid component to an mRNA in a nanoparticle composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of the lipid component to an mRNA may be from about 10:1 to about 40:1. The amount of mRNA in a nanoparticle composition may, for example, be measured using absorption spectroscopy (e.g., ultraviolet-visible spectroscopy).

The efficiency of encapsulation of an mRNA describes the amount of mRNA that is encapsulated or otherwise associated with a lipid composition after preparation, relative to the initial amount provided. The encapsulation efficiency is desirably high (e.g., close to 100%). The encapsulation efficiency may be measured, for example, by comparing the amount of mRNA in a solution containing the lipid composition before and after breaking up the LNP composition with one or more organic solvents or detergents. Fluorescence may be used to measure the amount of free mRNA in a solution. For the LNP compositions of the invention, the encapsulation efficiency of an mRNA may be at least 50%, for example 50%, 55%, 60%, 65%, 70%, 75%, 80%, 85%, 90%, 91%, 92%, 93%, 94%, 95%, 96%, 97%, 98%, 99%, or 100%. In certain embodiments, the encapsulation efficiency may be at least 80%.

### VI. Formulation and Mode of Delivery

LNP compositions of the invention may be formulated in whole or in part as a pharmaceutical composition. The pharmaceutical compositions may further include one or more pharmaceutically acceptable excipients or accessory ingredients such as those described herein. General guidelines for the formulation and manufacture of pharmaceutical compositions and agents are available, for example, in Remington's (2006) supra. Conventional excipients and accessory ingredients may be used in any pharmaceutical composition of the invention, except insofar as any conventional excipient or accessory ingredient may be incompatible with one or more components of an LNP composition of the invention. An excipient or accessory ingredient may be incompatible with a component of an LNP composition if its combination with the component may result in any undesirable biological effect or otherwise deleterious effect.

In some embodiments, one or more excipients or accessory ingredients may make up greater than 50% of the total mass or volume of a pharmaceutical composition including an LNP composition of the invention. For example, the one or more excipients or accessory ingredients may make up 30%, 40%, 50%, 60%, 70%, 80%, 90%, or more of a pharmaceutical composition. In certain embodiments, the excipient is approved for use in humans and for veterinary use, for example, by United States Food and Drug Administration. In certain embodiments, the excipient is pharmaceutical grade. In certain embodiments, an excipient meets the standards of the United States Pharmacopoeia (USP), the European Pharmacopoeia (EP), the British Pharmacopoeia, and/or the International Pharmacopoeia.

Relative amounts of the one or more lipids or LNPs, one or more pharmaceutically acceptable excipients, and/or any additional ingredients in a pharmaceutical composition will vary, depending upon the identity, size, and/or condition of the subject treated and further depending upon the route by which the composition is to be administered.

Lipid compositions and/or pharmaceutical compositions including one or more LNP compositions may be administered to any subject, including a human patient that may benefit from a therapeutic effect provided by the delivery of a nucleic acid, e.g., an RNA (e.g., mRNA, tRNA or siRNA) to one or more particular cells, tissues, organs, or systems or groups thereof, such as the renal system. Although the descriptions provided herein of LNP compositions and pharmaceutical compositions including LNP compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is understood.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., the payload).

Pharmaceutical compositions of the invention may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

### Other Components

In addition, it is contemplated that the pharmaceutical compositions may include one or more components in addition to those described hereinabove.

The pharmaceutical compositions may also include one or more permeability enhancer molecules, carbohydrates, polymers, therapeutic agents, surface altering agents, or other components. A permeability enhancer molecule may be a molecule described, for example, in U.S. patent application publication No. 2005/0222064. Carbohydrates may include simple sugars (e.g., glucose) and polysaccharides (e.g., glycogen and derivatives and analogs thereof).

The pharmaceutical compositions may also comprise a surface altering agent, including for example, anionic proteins (e.g., bovine serum albumin), surfactants (e.g., cationic surfactants such as dimethyldioctadecyl-ammonium bromide), sugars or sugar derivatives (e.g., cyclodextrin), polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within and/or upon the surface of a composition described herein.

In addition to these components, a pharmaceutical composition comprising an LNP composition of the invention may include any substance useful in pharmaceutical compositions. For example, the pharmaceutical composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see, e.g., Remington's (2006) supra).

Dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In certain embodiments, the lipid nanoparticle compositions and formulations thereof are adapted for administration intravenously, intramuscularly, intradermally, subcutaneously, intra-arterially, intra-tumor, or by inhalation. In certain embodiments, a dose of about 0.001 mg/kg to about 10 mg/kg is administered to a subject. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

The specific therapeutically effective, prophylactically effective, or otherwise appropriate dose level (e.g., for imaging) for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

### VII. Methods

The present disclosure provides methods of delivering a payload to a target cell or tissue, for example, a target cell or tissue in a subject, and LNPs or pharmaceutical compositions comprising the LNPs for use in such methods. Any disclosure herein of a method of, e.g., treating a disease or disorder or, e.g., delivering a nucleic acid to a cell or, e.g., producing a polypeptide of interest in a cell should be interpreted also as a disclosure of an LNP or pharmaceutical composition comprising said LNP for use in such methods.

In certain embodiments, the invention provides a method of producing a polypeptide of interest (e.g., a protein of interest) in a mammalian cell, and LNPs or pharmaceutical compositions comprising the LNPs for use in such methods. Methods of producing polypeptides in such a cell involve contacting a cell with an LNP composition comprising an RNA of interest (e.g., an mRNA encoding the polypeptide of interest (e.g., a protein of interest)). Upon contacting the cell with the LNP composition, the mRNA may be taken up and translated in the cell to produce the polypeptide of interest.

In general, the step of contacting a mammalian cell with an LNP composition including an mRNA encoding a polypeptide of interest may be performed in vivo, ex vivo, or in vitro. The amount of an LNP composition contacted with a cell, and/or the amount of mRNA therein, may depend on the type of cell or tissue being contacted, the means of administration, the physiochemical characteristics of the LNP composition and the mRNA (e.g., size, charge, and chemical composition) therein, and other factors. In general, an effective amount of the LNP composition will allow for efficient polypeptide production in the cell. Metrics for efficiency may include polypeptide translation (indicated by polypeptide expression), level of mRNA degradation, and immune response indicators.

The step of contacting an LNP composition including an mRNA with a cell may involve or cause transfection where the LNP composition may fuse with the membrane of cell to permit the delivery of the mRNA into the cell. Upon introduction into the cytoplasm of the cell, the mRNA is then translated into a protein or peptide via the protein synthesis machinery within the cytoplasm of the cell.

In certain embodiments, the LNP compositions described herein may be used to deliver therapeutic or prophylactic agents to a subject. For example, an mRNA included in an LNP composition may encode a polypeptide and produce the therapeutic or prophylactic polypeptide upon contacting and/or entry (e.g., transfection) into a cell. In certain embodiments, an mRNA included in an LNP composition of the invention may encode a polypeptide that may improve or increase the immunity of a subject.

In certain embodiments, contacting a cell with an LNP composition including an mRNA may reduce the innate immune response of a cell to an exogenous nucleic acid. A cell may be contacted with a first LNP composition including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times.

Additionally, efficiency of polypeptide production in the cell may be optionally determined, and the cell may be re-contacted with the first and/or second composition repeatedly until a target protein production efficiency is achieved.

The present disclosure provides methods of delivering a nucleic acid (e.g., an mRNA) to a mammalian cell or tissue, for example, a mammalian cell or tissue in a subject. Delivery of an mRNA to such a cell or tissue involves administering an LNP composition including the mRNA to a subject, for example, by injection, e.g., via intramuscular injection or intravascular delivery into the subject. After administration, the LNP can target and/or contact a cell, for example, an immune cell, such as a T-cell. Upon contacting the cell with the LNP composition, a translatable mRNA may be translated in the cell to produce a polypeptide of interest.

In certain embodiments, an LNP composition of the invention may target a particular type or class of cells. This targeting may be facilitated using the lipids described herein to form LNPs, which may also include a targeting group for targeting cells of interest. In certain, embodiments, specific delivery may result in a greater than 2 fold, 5 fold, 10 fold, 15 fold, or 20 fold increase in the amount of mRNA to the targeted destination (e.g., cells that express or express at high levels the receptor of interest which binds to the immune cell targeting group of the LNPs) as compared to another destinations (e.g., cells that either do not express or only express at low levels the receptor of interest).

LNP compositions of the invention may be useful for treating a disease, disorder, or condition characterized by missing or aberrant protein or polypeptide activity. Upon delivery of an mRNA encoding the missing or aberrant polypeptide to a cell, translation of the mRNA may produce the polypeptide, thereby reducing or eliminating an issue caused by the absence of or aberrant activity caused by the polypeptide. Because translation may occur rapidly, the methods and compositions of the invention may be useful in the treatment of acute diseases, disorders, or conditions such as sepsis, stroke, and myocardial infarction. An mRNA included in an LNP composition of the invention may also be capable of altering the rate of transcription of a given species, thereby affecting gene expression.

Diseases, disorders, and/or conditions characterized by dysfunctional or aberrant protein or polypeptide activity for which a composition of the invention may be administered include, but are not limited to, cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis. The present disclosure provides a method for treating such diseases, disorders, and/or conditions in a subject by administering an LNP composition including an mRNA and a lipid component including KL10, a phospholipid (optionally unsaturated), a PEG lipid, and a structural lipid, wherein the m RNA encodes a polypeptide that antagonizes or otherwise overcomes an aberrant protein activity present in the cell of the subject.

The therapeutic and/or prophylactic compositions described herein may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject, the purpose of the administration, the particular composition, the mode of administration, and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

A LNP composition including one or more mRNAs may be administered by a variety of routes, for example, orally, intravenously, intramuscularly, intra-arterially, intramedullary, intrathecally, subcutaneously, intraventricularly, trans- or intra-dermally, intradermally, rectally, intravaginally, intraperitoneally, topically, mucosally, nasally, intratumorally. In certain embodiments, an LNP composition may be administered intravenously, intramuscularly, intradermally, intra-arterially, intratumorally, or subcutaneously. However, the present disclosure encompasses the delivery of LNP compositions of the invention by any appropriate route taking into consideration likely advances in the sciences of drug delivery. In general, the most appropriate route of administration will depend upon a variety of factors including the nature of the LNP composition including one or more mRNAs (e.g., its stability in various bodily environments such as the bloodstream and gastrointestinal tract), the condition of the patient (e.g., whether the patient is able to tolerate particular routes of administration), etc.

LNP compositions including one or more mRNAs may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more LNP compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

In some embodiments, no more than 1%, no more than 2%, no more than 3%, no more than 4%, no more than 5%, no more than 6%, no more than 7%, no more than 8%, no more than 9%, no more than 10%, no more than 15%, no more than 20%, no more than 25%, no more than 30%, no more than 35%, no more than 40%, no more than 45%, or no more than 50% of cells that are not meant to be the destination of the delivery are transfected by the LNP. In some embodiments, the cells that are not meant to be the destination of the delivery are subject's non-immune cells. In some embodiments, the cells that are not meant to be the destination of the delivery are cells not targeted by the method. In some embodiments, the cells that are not meant to be the destination of the delivery are subject's cells not targeted by the method.

In some embodiments, the half-life of the nucleic acid delivered by the LNP described herein to the immune cell or a polypeptide encoded by the nucleic acid delivered by the LNP and expressed in the immune cell is at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, or at least 5 times longer than the half-life of the nucleic acid delivered by a reference LNP to the immune cells or a polypeptide encoded by the nucleic acid delivered by the reference LNP and expressed in the immune cell.

In some embodiments, the composition of the LNP differs from the composition of the reference LNP in the type of ionizable cationic lipid, relative amount of ionizable cationic lipid, length of the lipid anchor in PEG lipid, back bone or head group of the PEG lipid, relative amount of PEG lipid, or type of immune cell targeting group, or any combination thereof. In some embodiments, the composition of the LNP differs from the composition of the reference LNP only in the type of ionizable cationic lipid. In some embodiments, the composition of the LNP differs from the composition of the reference LNP only in the amount of PEG lipid. In some embodiments, the reference LNP comprises cationic Lipid DLin-KC3-DMA, but otherwise as the same as a tested LNP. In some embodiments, the reference LNP comprises cationic Lipid DLin-KC2-DMA, but otherwise as the same as a tested LNP. In some embodiments, the reference LNP comprises cationic Lipid ALC-0315, but otherwise as the same as a tested LNP. In some embodiments, the reference LNP comprises cationic Lipid SM-102, but otherwise as the same as a tested LNP. In some embodiments, PEG lipid is a free PEG lipid.

In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the immune cells are transfected by the LNP. In some embodiments, the immune cells are subject's immune cells. In some embodiments, the immune cells are immune cells targeted by the method. In some embodiments, the immune cells are subject's immune cells targeted by the method. In some embodiments, the immune cells are macrophages, for instance M2a macrophages, M2b macrophages, and/or M2c macrophages. In some embodiments, the immune cells are B cells. In some embodiments, the immune cells are NK cells. In some embodiments, the immune cells are T cells, for example CD4+ T cells and/or CD8+ T cells. In some embodiments, the immune cells are NK cells and T cells, for example NK cells and CD4+ T cells and/or CD8+ T cells. In some embodiments, the immune cells are monocytes. In some embodiments, the immune cells are dendritic cells.

In some embodiments, the expression level of the nucleic acid delivered by the LNP is at least at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, at least 5 times, at least 6 times, at least 7 times, at least 8 times, at least 9 times, or at least 10 times higher than the expression level of the nucleic acid delivered by a reference LNP. In some embodiments, the expression level is measured and compared with a method described herein. In some embodiments, the expression level is measured by the ratio of cells expressing the encoded polypeptide. In some embodiments, the expression level is measured with FACS. In some embodiments, the expression level is measured by the average amount of the encoded polypeptide expressed in cells. In some embodiments, the expression level is measured as mean fluorescence intensity. In some embodiments, the expression level is measured by the amount of the encoded polypeptide or other materials secreted by cells.

In another aspect, provided herein are methods of targeting the delivery of a nucleic acid to an immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the compound of the following formula: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, an aspect of the disclosure relates to an LNP or a pharmaceutical composition containing thereof, as disclosed herein, for use in a method of targeting the delivery of a nucleic acid to an immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP). In some embodiments, the LNP is an LNP as described herein in the present disclosure.

In some embodiments, the LNP provides at least one of the following benefits:
(i) increased specificity of targeted delivery to the immune cell compared to a reference LNP;
(ii) increased half-life of the nucleic acid or a polypeptide encoded by the nucleic acid in the immune cell compared to a reference LNP;
(iii) increased transfection rate compared to a reference LNP; and
(iv) a low level of dye accessible mRNA (<15%) and high RNA encapsulation efficiencies, wherein at least 80% mRNA was recovered in final formulation relative to the total RNA used in LNP batch preparation.

In some aspect, provided are methods of expressing a polypeptide of interest in a targeted immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition containing thereof, as disclosed herein, for use in a method of expressing a polypeptide of interest in a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP; and
(v) a low level of dye accessible mRNA (<15%) and high RNA encapsulation efficiencies, wherein at least 80% mRNA was recovered in final formulation relative to the total RNA used in LNP batch preparation.

In some aspects, provided are methods of modulating cellular function of a target immune cell of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the immune cell. In some embodiments, an aspect of the disclosure relates to an LNP or a pharmaceutical composition containing thereof, as disclosed herein, for use in a method of modulating cellular function of a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP;
(v) the LNP can be administered at a lower dose compared to a reference LNP to reach the same biologic effect in the immune cell; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the modulation of cell function comprises reprogramming the immune cells to initiate an immune response. In some embodiments, the modulation of cell function comprises modulating antigen specificity of the immune cell.

In some aspect, provided are methods of treating, ameliorating, or preventing a symptom of a disorder or disease in a subject in need thereof. In some embodiments, the method comprises administering to the subject a lipid nanoparticle (LNP) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the nucleic acid modulates the immune response of the immune cell, therefore to treat or ameliorate the symptom. In some embodiments, an aspect of the disclosure relates to an LNP or a pharmaceutical composition containing thereof, as disclosed herein, for use in a method of treating, ameliorating, or preventing a symptom of a disorder or disease in a subject in need thereof. A disease or disorder may be as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:
(i) increased specificity of delivery of the nucleic acid into the immune cell compared to a reference LNP;
(ii) increased half-life of the nucleic acid or a polypeptide encoded by the nucleic acid in the immune cell compared to a reference LNP;
(iii) increased transfection rate compared to a reference LNP;
(iv) the LNP can be administered at a lower dose compared to a reference LNP to reach the same treatment efficacy;
(v) increased level of gain of function by an immune cell compared to a reference LNP; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the disorder is an immune disorder, an inflammatory disorder, or cancer. In some embodiments, the nucleic acid encodes an antigen for use in a therapeutic or prophylactic vaccine for treating or preventing an infection by a pathogen.

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In some aspects, provided are methods of targeting the delivery of a nucleic acid to an immune cell of a subject. In some embodiments, the method comprises contacting the immune cell with a lipid nanoparticle (LNP) provided herein. In some embodiments, the method is for targeting NK cells. In some embodiments, the immune cell targeting group binds to CD56. In some embodiments, the method is for targeting both T cells and NK cells simultaneously. In some embodiments, the immune cell targeting group binds to CD7, CD8, or both CD7 and CD8. In some embodiments, the method is for targeting both CD4+ and CD8+ T cells simultaneously. In some embodiments, the immune cell targeting group comprises a polypeptide that binds to CD3 or CD7.

In some aspects, provided are methods of expressing a polypeptide of interest in a targeted immune cell of a subject. In some embodiments, the method comprises contacting the immune cell with a lipid nanoparticle (LNP) provided herein.

In some aspect, provided are method of modulating cellular function of a target immune cell of a subject. In some embodiments, the method comprises administering to the subject a lipid nanoparticle (LNP) provided herein.

In some aspects, provided are method of treating, ameliorating, or preventing a symptom of a disorder or disease in a subject in need thereof. In some embodiments, the method comprises administering to the subject a lipid nanoparticle (LNP) provided herein.

In some aspects, provided are methods of treating a disease or disorder related to CD8 in a subject. In some embodiments, the method comprises administering a pharmaceutical composition described herein to the subject. In some embodiments, the disease or disorder is cancer.

LNPs disclosed in the present disclosure and as claimed are suitable for the methods described above.

### VIII. Kits for Use in Medical Applications

Another aspect of the invention provides a kit for treating a disorder. The kit comprises: an ionizable cationic lipid, a lipid-immune cell targeting group conjugate, a lipid nanoparticle composition comprising an ionizable cationic lipid and/or a lipid-immune cell targeting group conjugate with or without an encapsulated payload (e.g., an mRNA), and instructions for treating a medical disorder, such as, cancer or a microbial or viral infection.

### Enumerated Embodiments

The following enumerated embodiments are representative of some aspects of the invention. It will be understood that reference to an embodiment number refers to all subembodiments, unless specified otherwise. For example, “embodiment 20” refers to subembodiments 20A to 20F, unless specified otherwise.

1. A compound of Formula (I):

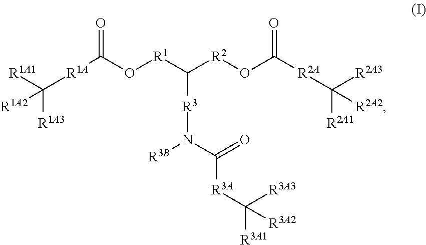

or a salt thereof, wherein:
R¹ and R² are each C₁₋₃ alkylene;
R³ is C₁₋₃ alkylene or a bond;
R^{1A} and R^{2A} are each a bond or C₁₋₁₀ alkylene;
R^{3A} is a bond or C₁₋₃ alkylene;
R^{1A1}, R^{2A1}, R^{3A1}, and R^{3A2} are each H;
R^{1A2} and R^{2A2} are each H, —(CH₂)₀₋₅C(O)OR^{a1}, or —(CH₂)₀₋₅OC(O)R^{a2};
R^{1A3} and R^{2A3} are each H, —(CH₂)₀₋₅C(O)OR^{a1}, or —(CH₂)₀₋₅OC(O)R^{a2};
R^{3A3} is —C(O)OR^{a1};
R^{a1} and R^{a2} are each independently C₁₋₂₀ alkyl;
R^{3B} is

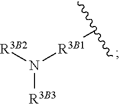

R^{3B1} is C₄₋₆ alkylene; and
R^{3B2} and R^{3B3} are each C₁₋₃ alkyl.

2. The compound of embodiment 1, or a salt thereof, wherein R¹ and R² are each methylene.

3. The compound of embodiment 1 or 2, or a salt thereof, wherein R^{1A} and R^{2A} are each a bond, —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, or —(CH₂)₁₀—.

4. The compound of embodiment 3, or a salt thereof, wherein R^{1A} and R^{2A} are each a bond, —(CH₂)₂—, —(CH₂)₅—, —(CH₂)₇—, or —(CH₂)₉—.

5. The compound of any one of embodiments 1 to 4, or a salt thereof, wherein R^{3A} is a bond, —CH₂—, or —(CH₂)₂—.

6. The compound of embodiment 5, or a salt thereof, wherein R^{3A} is —CH₂—.

7. The compound of any one of embodiments 1 to 6, or a salt thereof, wherein R^{1A2} and R^{2A2} are each —OC(O)(C₁₋₁₅ alkyl), —C(O)O(C₁₋₁₅ alkyl), —OC(O)CH(C₁₋₁₀ alkyl)(C₁₋₁₀ alkyl), —C(O)OCH(C₁₋₁₀ alkyl)(C₁₋₁₀ alkyl), —(CH₂)C(O)O(C₁₋₁₀ alkyl), or —(CH₂)OC(O)(C₁₋₁₀ alkyl).

8. The compound of embodiment 7, or a salt thereof, wherein R^{1A2} and R^{2A2} are each —OC(O)(C₁₋₁₀ alkyl), —C(O)O(C₁₋₁₀ alkyl), —OC(O)CH(C₆ alkyl)(C₈ alkyl), —C(O)OCH(C₂₋₃ alkyl)(C₅₋₆ alkyl), or —(CH₂)C(O)O(C₁₀ alkyl).

9. The compound of embodiment 8, or a salt thereof, wherein R^{1A2} and R^{2A2} are each

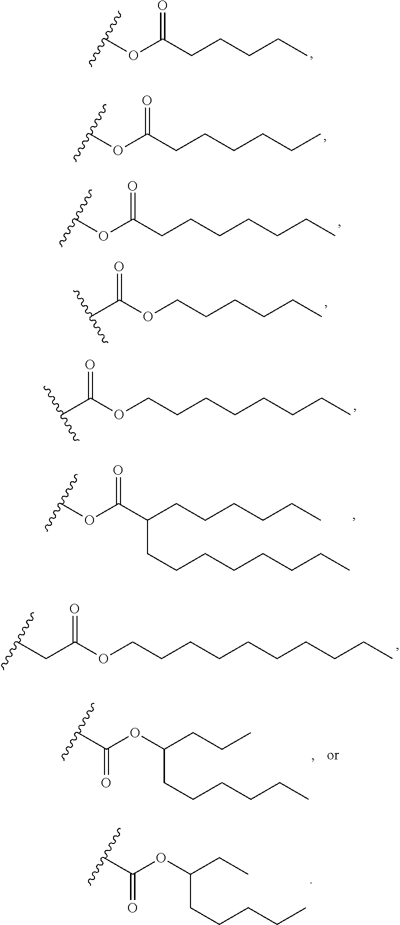

10. The compound of any one of embodiments 1 to 9, or a salt thereof, wherein R^{1A3} and R^{2A3} are each H, —OC(O)(C₁₋₁₅ alkyl), or —C(O)O(C₁₋₁₅ alkyl).

11. The compound of embodiment 10, or a salt thereof, wherein R^{1A3} and R^{2A3} are each H, —OC(O)(C₅₋₁₀ alkyl), —C(O)O(C₆₋₁₀ alkyl), or —(CH₂)C(O)O(C₁₀ alkyl).

12. The compound of embodiment 11, or a salt thereof, wherein R^{1A3} and R^{2A3} are each H,

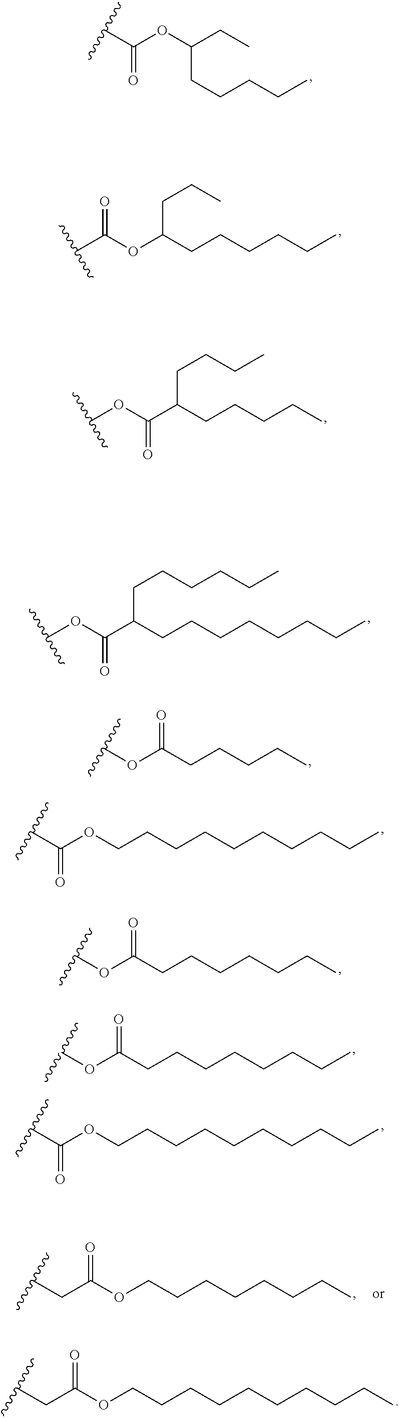

13. The compound ofany one of embodiments 1 to 12, or a salt thereof, wherein R^{3A3} is —C(O)OCH(C₁₋₅ alkyl)(C₁₋₁₀ alkyl).

14. The compound of embodiment 13, or a salt thereof, wherein R^{3A3} is —C(O)OCH(C₃ alkyl)(C₆ alkyl).

15. The compound of embodiment 14, or a salt thereof, wherein R^{3A3} is

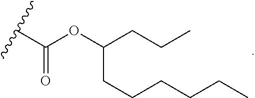 [SMILES: *C(=O)OC(CCC)CCCCCC]

16. The compound of any one of embodiments 1 to 15, or a salt thereof, wherein R^{3B1} is —(CH₂)₄—.

17. The compound of any one of embodiments 1 to 16, or a salt thereof, wherein R^{3B2} and R^{3B3} are each methyl.

18. The compound of any one of embodiments 1 to 15 and 17, or a salt thereof, wherein

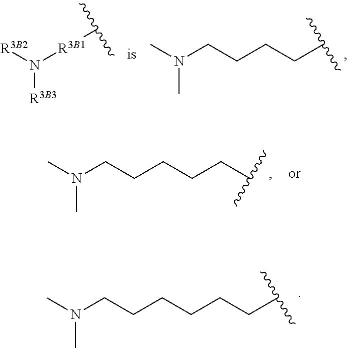

19. The compound of embodiment 1, or a salt thereof, wherein the compound is selected from Table 1.

20A. The compound of embodiment 1, or a salt thereof, wherein the compound is

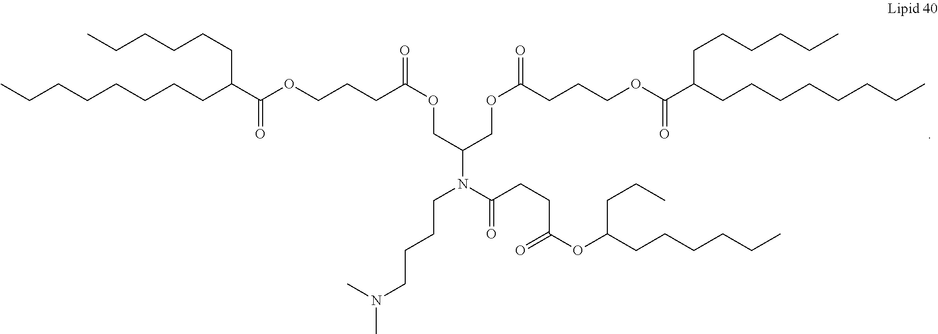 [SMILES: CCCCCCCCC(CCCCCC)C(=O)OCCCC(=O)OCC(COC(=O)CCCOC(=O)C(CCCCCC)CCCCCCCC)N(CCCCN(C)C)C(=O)CCC(=O)OC(CCC)CCCCCC]

20B. The compound of embodiment 1, or a salt thereof, wherein the compound is

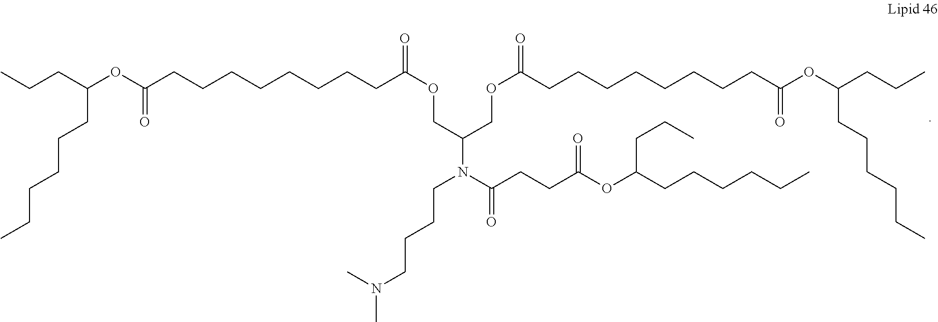 [SMILES: CCCCCCC(CCC)OC(=O)CCCCCCCCC(=O)OCC(COC(=O)CCCCCCCCC(=O)OC(CCC)CCCCCC)N(CCCCN(C)C)C(=O)CCC(=O)OC(CCC)CCCCCC]

20C. The compound of embodiment 1, or a salt thereof, wherein the compound is

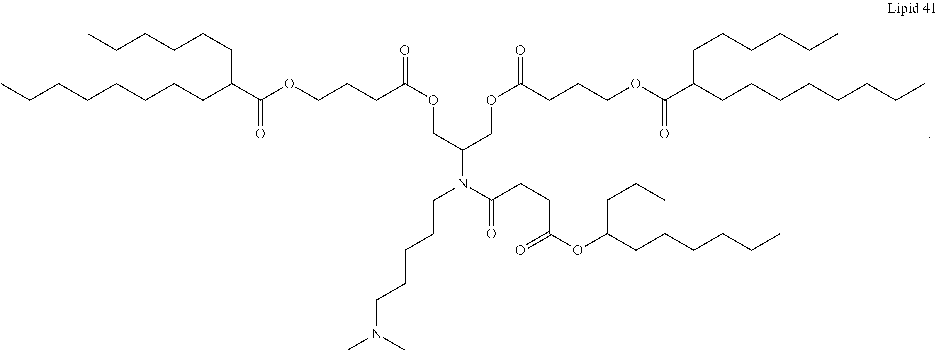 [SMILES: CCCCCCCCC(CCCCCC)C(=O)OCCCC(=O)OCC(COC(=O)CCCOC(=O)C(CCCCCC)CCCCCCCC)N(CCCCCN(C)C)C(=O)CCC(=O)OC(CCC)CCCCCC]

20D. The compound of embodiment 1 or a salt thereof wherein the compound is

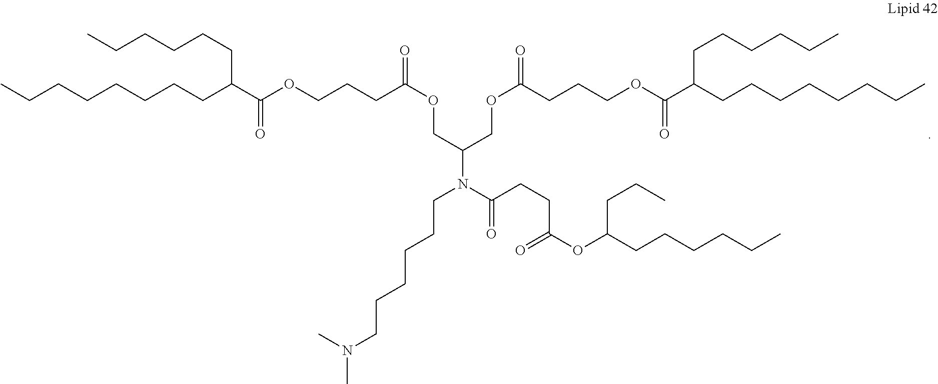 [SMILES: CCCCCCCCC(CCCCCC)C(=O)OCCCC(=O)OCC(COC(=O)CCCOC(=O)C(CCCCCC)CCCCCCCC)N(CCCCCCN(C)C)C(=O)CCC(=O)OC(CCC)CCCCCC]

20E. The compound of embodiment 1, or a salt thereof, wherein the compound is

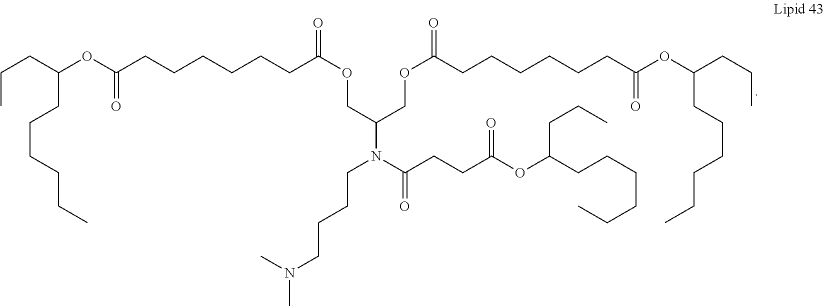 [SMILES: CCCCCCC(CCC)OC(=O)CCCCCCC(=O)OCC(COC(=O)CCCCCCC(=O)OC(CCC)CCCCCC)N(CCCCN(C)C)C(=O)CCC(=O)OC(CCC)CCCCCC]

20F. The compound of embodiment 1, or a salt thereof, wherein the compound is

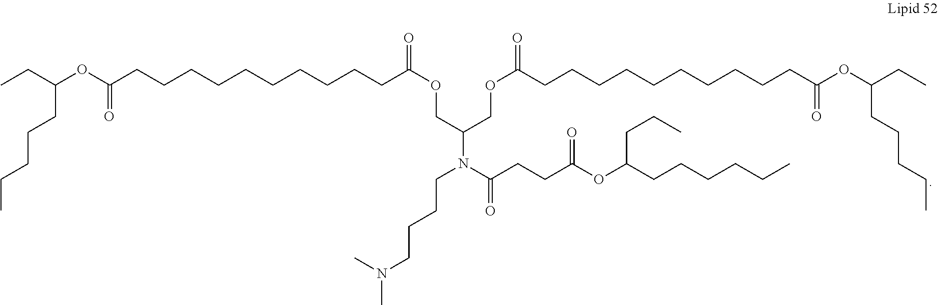 [SMILES: CCCCCCC(CCC)OC(=O)CCC(=O)N(CCCCN(C)C)C(COC(=O)CCCCCCCCCCC(=O)OC(CC)CCCCC)COC(=O)CCCCCCCCCCC(=O)OC(CC)CCCCC]

21. A lipid nanoparticle (LNP) comprising a lipid blend for targeted delivery of a nucleic acid into an immune cell, the lipid blend comprising:
(a) a lipid-immune cell targeting group conjugate comprising the compound of Formula (II): [Lipid]-[optional linker]-[immune cell targeting group],
(b) an ionizable cationic lipid, and
(c) a nucleic acid, wherein the nucleic acid is encapsulated in the LNP.

22. The LNP of embodiment 21, wherein the ionizable cationic lipid comprises the compound of any one of embodiments 1 to 20.

23. The LNP of embodiment 21 or 22, wherein the immune cell targeting group comprises an antibody that binds a macrophage antigen, a monocyte antigen, and/or a dendritic antigen.

24. The LNP of embodiment 23, wherein the immune cell targeting group comprises an antibody that binds a macrophage antigen.

25. The LNP of embodiment 23 or 24, wherein the macrophage comprises an M1 macrophage, an M2 macrophage, or both.

26. The LNP of any one of embodiments 23 to 25, wherein the macrophage comprises an M2a macrophage, an M2b macrophage, an M2c macrophage, or any combination thereof.

27. The LNP of embodiment 23, wherein the macrophage antigen comprises CDIIB, CD68, CD80, CD86, TRL-2, TRL-4, iNOS, MHC-IL, CD163, CD206, CD209, FIZZ1, or Ym1/2, or any combination thereof.

28. The LNP of embodiment 27, wherein the macrophage antigen comprises CD206.

29. The LNP of embodiment 23, wherein the immune cell targeting group comprises an antibody that binds a monocyte antigen.

30. The LNP of embodiment 29, wherein the monocyte antigen comprises CD14, CCR2, CCR5, CD62L, HLA, CD68, CXCR1, CXCR3, CD11c, or any combination thereof.

31. The LNP of any one of embodiments 21 to 30, wherein the immune cell targeting group is covalently coupled to a lipid in the lipid blend via a polyethylene glycol (PEG) containing linker.

32. The LNP of embodiment 31, wherein the lipid covalently coupled to the immune cell targeting group via a PEG containing linker is distearoylglycerol (DSG), distearoyl-phosphatidylethanolamine (DSPE), dimyristoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.

33. The LNP of embodiment 31 or 32, wherein the PEG is PEG 2000 or PEG 3400.

34. The LNP of any one of embodiments 21 to 33, wherein the lipid-immune cell targeting group conjugate is present in the lipid blend in a range of 0.001 to 0.5 mole percent (e.g., 0.002-0.2 mole percent).

35. The LNP of any one of embodiments 21 to 34, wherein the lipid blend further comprises one or more of a structural lipid (e.g., a sterol), a neutral phospholipid, and a free PEG-lipid.

36. The LNP of any one of embodiments 21 to 35, wherein the ionizable cationic lipid is present in the lipid blend in a range of 30-70 (e.g., 40-60) mole percent.

37. The LNP of embodiment 35, wherein the sterol is present in the lipid blend in a range of 20-70 (e.g., 30-50) mole percent.

38. The LNP of embodiment 35 or 37, wherein the sterol is cholesterol.

39. The LNP of any one of embodiments 35 to 38, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin.

40. The LNP of any one of embodiments 35 to 39, wherein the neutral phospholipid is present in the lipid blend in a range of 5-15 mole percent.

41A. The LNP of any one of embodiments 35 to 40, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylglycerol (PEG-DOG), PEG-dimyristoyl-glycerol (PEG-DMG), PEG-dipalmitoyl-glycerol (PEG-DPG), PEG-dilinolenoyl-glycero-phosphatidyl ethanolamine (PEG-DLPE), PEG-dimyristoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

41B. The LNP of any one of embodiments 35 to 40, wherein the free PEG-lipid is DSG-PEG.

41C. The LNP of any one of embodiments 35 to 40, wherein the free PEG-lipid is DPG-PEG.

42. The LNP of any one of embodiments 35 to 40, wherein the free PEG-lipid comprises a diacylphosphatidylethanolamine comprising Dipalmitoyl (C16) chain or Distearoyl (C18) chain, and optionally the free PEG-lipid comprises PEG-DPG and PEG-DMG.

43A. The LNP of any one of embodiments 35 to 42, wherein the free PEG-lipid is present in the lipid blend in a range of 1-4 mole percent.

43B. The LNP of any one of embodiments 35 to 42, wherein the free PEG-lipid is present in the lipid blend in a range of about 1 to about 2 mole percent.

43C. The LNP of any one of embodiments 35 to 42, wherein the free PEG-lipid is present in the lipid blend in a concentration of about 1.5 mole percent.

43D. The LNP of any one of embodiments 35 to 42, wherein the free PEG-lipid is present in the lipid blend in a range of about 3 to about 4 mole percent.

43E. The LNP of any one of embodiments 35 to 42, wherein the free PEG-lipid is present in the lipid blend in a concentration of about 3.5 mole percent.

44. The LNP of any one of embodiments 35 to 43, wherein the free PEG-lipid comprises the same or a different lipid as the lipid in the lipid-immune cell targeting group conjugate.

45. The LNP of any one of embodiments 21 to 44, wherein the LNP has a mean diameter in the range of 50-200 nm.

46. The LNP of embodiment 45, where the LNP has a mean diameter of about 100 nm.

47. The LNP of any one of embodiments 21 to 46, wherein the LNP has a polydispersity index in a range from 0.01 to 0.1.

48. The LNP of any one of embodiments 21 to 47, wherein the LNP has a zeta potential of from about +0 mV to about +10 mV at pH 5.5, or from about −5 mV to about 0 mV at pH 7.4.

49. The LNP of any one of embodiments 21 to 48, wherein the nucleic acid is DNA or RNA.

50. The LNP of embodiment 49, wherein the RNA is an mRNA.

51. The LNP of embodiment 50, wherein the mRNA encodes a receptor, a growth factor, a hormone, a cytokine, an antibody, an antigen, an enzyme, or a vaccine.

52. The LNP of embodiment 50, wherein the mRNA encodes a polypeptide capable of regulating immune response in the immune cell.

53. The LNP of embodiment 50, wherein the mRNA encodes a polypeptide capable of reprogramming the immune cell.

54. The LNP of embodiment 53, wherein the mRNA encodes polypeptide capable of reprogramming an M2 macrophage to an M1 macrophage.

55. The LNP of any one of embodiments 21 to 54, wherein the immune cell targeting group comprises an antibody, and the antibody is a Fab or an immunoglobulin single variable domain (e.g., a V_{HH}).

56. The LNP of any one of embodiments 21 to 54, wherein the immune cell targeting group comprises a Fab, F(ab′)2, Fab′-SH, Fv, or scFv fragment.

57. The LNP of embodiment 55 or embodiment 56, wherein the immune cell targeting group comprises a Fab that is engineered to knock out the natural interchain disulfide bond at the C-terminus.

58. The LNP of embodiment 57, wherein the Fab comprises a heavy chain fragment that comprises C233S substitution, and a light chain fragment that comprises C214S substitution, numbering according to Kabat.

59. The LNP of any one of embodiments 56 to 58, wherein the immune cell targeting group comprises a Fab that has a non-natural interchain disulfide bond (e.g., an engineered, buried interchain disulfide bond).

60. The LNP of embodiment 59, wherein the Fab comprises F174C substitution in the heavy chain fragment, and S176C substitution in the light chain fragment, numbering according to Kabat.

61. The LNP of embodiments 56 to 60, wherein the immune cell targeting group comprises a Fab that comprises a cysteine at the C-terminus of the heavy or light chain fragment.

62. The LNP of embodiment 61, wherein the Fab further comprises one or more amino acids between the heavy chain fragment of the Fab and the C-terminal cysteine.

63. The LNP of embodiment 55, wherein the immune cell targeting group comprises an immunoglobulin single variable domain.

64. The LNP of embodiment 55 or 63, wherein the immunoglobulin single variable domain comprises a cysteine at the C-terminus.

65. The LNP of embodiment 64, wherein the immunoglobulin single variable domain comprises a VHH domain and further comprises a spacer comprising one or more amino acids between the VHH domain and the C-terminal cysteine.

66. The LNP of any one of embodiments 56 and 63 to 65, wherein the immune cell targeting group comprises two or more V_{HH} domains.

67. The LNP of embodiment 66, wherein the two or more V_{HH} domains are linked by an amino acid linker.

68. The LNP of embodiment 66, wherein the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds.

69. The LNP of any one of embodiments 55 and 63 to 65, wherein the immune cell targeting group comprises a V_{HH} domain linked to an antibody CH1 domain, and wherein the antibody CH1 domain is linked to an antibody light chain constant domain by one or more disulfide bonds.

70. The LNP of embodiment 68 or 69, wherein the CH1 domain comprises F174C and C233S substitutions, and the light chain constant domain comprises S176C and C214S substitutions, numbering according to Kabat.

71. The LNP of any one of embodiments 21 to 70, wherein the LNP comprises:
(a) the ionizable cationic lipid;
(b) the conjugate comprising the compound of the following formula:

[Lipid]-[optional linker]-[immune cell targeting group];
(c) a sterol or other structural lipid;
(d) a neutral phospholipid;
(e) a free Polyethylene glycol (PEG) lipid; and
(f) the nucleic acid.

72. The LNP of any one of embodiments 21 to 71, wherein the LNP is for delivering a nucleic acid into an immune cell, and wherein the immune cell is a macrophage, and the immune cell targeting group comprises an antibody that binds CD206.

73. The LNP of any one of embodiments 21 to 71, wherein the LNP is for delivering a nucleic acid into an immune cell, and wherein the immune cell targeting group comprises an antibody that binds CD206, and the free PEG lipid is DMG-PEG or PEG-DPG.

74. The LNP of any one of embodiments 21 to 73, wherein the immune cell targeting group comprises an antibody, and the antibody is a Fab or an immunoglobulin single variable domain.

75. The LNP of embodiment 73, wherein the Fab is engineered to knock out the natural interchain disulfide at the C-terminus.

76. The LNP of embodiment 75, wherein the Fab comprises a heavy chain fragment that comprises C233S substitutions, and a light chain fragment that comprises C214S substitutions.

77. The LNP of embodiment 75, wherein the Fab comprises a non-natural interchain disulfide.

78. The LNP of embodiment 75, wherein the Fab comprises F174C substitution in the heavy chain fragment, and S176C substitution in the light chain fragment.

79. The LNP of embodiment 74, wherein the antibody is an immunoglobulin single variable (ISV) domain, and the ISV domain is a V_{HH}.

80. The LNP of embodiment 79, wherein the free PEG lipid comprises a PEG having a molecular weight of at least 2000 daltons.

81. The LNP of embodiment 80, wherein the PEG has a molecular weight of about 3000 to 5000 daltons.

82. The LNP of embodiment 74, wherein the antibody is a Fab.

83. The LNP of embodiment 82, wherein the Fab binds CD206, and the free PEG lipid in the LNP comprises a PEG having a molecular weight of about 2000 daltons.

84. The LNP of embodiment 82, wherein the Fab is an anti-CD206 antibody, and the free PEG lipid in the LNP comprises a PEG having a molecular weight of about 3000 to 3500 daltons.

85. The LNP of embodiment 74, wherein the immune cell targeting group comprises two or more V_{HH} domains.

86. The LNP of embodiment 85, wherein the two or more V_{HH} domains are linked by an amino acid linker.

87. The LNP of embodiment 86, wherein the immune cell targeting group comprises a first V_{HH} domain linked to an antibody CH1 domain and a second V_{HH} domain linked to an antibody light chain constant domain.

88. The LNP of any one of embodiments 21 to 71, wherein the LNP is for delivering a nucleic acid into an immune cell, and wherein the LNP binds a first macrophage antigen, and also binds a second macrophage antigen.

89. The LNP of embodiment 88, wherein the LNP comprises two conjugates, wherein the first conjugate comprises an antibody that binds the first macrophage antigen, and the second conjugate comprises an antibody that binds the second macrophage antigen.

90. The LNP of embodiment 88, wherein the LNP comprises one conjugate, and the conjugate comprises a bispecific antibody that binds both the first macrophage antigen and the second macrophage antigen.

91. The LNP of embodiment 90, wherein the bispecific antibody is an immunoglobulin single variable domain or Fab-ScFv.

92. The LNP of any one of embodiments 21 to 71, wherein the LNP binds to a first antigen on the surface of the first type of immune cell, and also binds to a second antigen on the surface of the second type of immune cell.

93. The LNP of embodiment 92, wherein the first type of immune cell is a first macrophage, and the second type of immune cell is a second macrophage, a T-cell, or an NK cell.

94. The LNP of embodiment 92 or 93, wherein the LNP comprises two conjugates, and the first conjugate comprises a first antibody that binds to the first antigen of the first type of immune cell, and the second conjugate comprises a second antibody that binds to the second antigen of the second type of immune cell.

95. The LNP of embodiment 92 or 93, wherein the LNP comprises one conjugate, and the conjugate comprises a bispecific antibody, and the bispecific antibody binds to both the first antigen on the first type of immune cell, and the second antigen on the second type of immune cells.

96. The LNP of any one of embodiments 21 to 71, wherein the bispecific antibody is an immunoglobulin single variable domain or a Fab-ScFv.

97. The LNP of any one of embodiments 21 to 71, wherein the LNP is for delivering a nucleic acid into an immune cell, and wherein the immune cell targeting group comprises a single antibody that binds to CD206.

98. The LNP of any one of embodiments 21 to 71, wherein the LNP is for delivering a nucleic acid into both a macrophage and a T-cell or both a macrophage and an NK cell, wherein the immune cell targeting group binds to both (i) CD206 and (ii) one of CD3, CD7, CD8, and CD56.

99. The LNP of any one of embodiments 72 to 98, wherein the LNP has a mean diameter in the range of 50-200 nm.

100. The LNP of embodiment 99, where the LNP has a mean diameter of about 100 nm.

101. The LNP of any one of embodiments 72 to 98, wherein the LNP has a polydispersity index in a range from 0.01 to 0.1.

102. The LNP of any one of embodiments 72 to 101, wherein the LNP has a zeta potential of from about +0 mV to about +10 mV at pH 5.5, or from about −5 mV to about 0 mV at pH 7.4.

103. The LNP of any one of embodiments 72 to 102, wherein the nucleic acid is DNA or RNA.

104. The LNP of embodiment 103, wherein the RNA is an mRNA.

105. The LNP of embodiment 104, wherein the mRNA encodes a receptor, a growth factor, a hormone, a cytokine, an antibody, an antigen, an enzyme, or a vaccine.

106. The LNP of embodiment 104, wherein the mRNA encodes a polypeptide capable of regulating immune response in the immune cell.

107. The LNP of embodiment 106, wherein the mRNA encodes a polypeptide capable of reprogramming the immune cell.

108. The LNP of embodiment 107, wherein the mRNA encodes polypeptide capable of reprogramming an M2 macrophage to an M1 macrophage.

109. The LNP of any one of embodiments 21 to 71, wherein the LNP is for delivering a nucleic acid into an immune cell, and wherein the immune cell targeting group comprises a Fab lacking the native interchain disulfide bond.

110. The LNP of embodiment 109, wherein the Fab is engineered to replace one or both cysteines on the native constant light chain and the native constant heavy chain that form the native interchain disulfide with a non-cysteine amino acid, therefor to remove the native interchain disulfide bond in the Fab.

111. A lipid nanoparticle (LNP) comprising a lipid blend for targeted delivery of a nucleic acid into a macrophage, the lipid blend comprising:
(a) a lipid-macrophage targeting group conjugate comprising the compound of Formula (II-m): [Lipid]-[optional linker]-[macrophage targeting group]; and
(b) a nucleic acid, wherein the nucleic acid is encapsulated in the LNP.

112. The LNP of embodiment 111, wherein the macrophage is an M2 macrophage.

113. The LNP of embodiment 111 or 112, wherein the macrophage targeting group binds CD206.

114. The LNP of any one of embodiments 111 to 113, wherein the nucleic acid is mRNA, and the mRNA encodes polypeptide capable of reprogramming an M2 macrophage to an M1 macrophage.

115. The LNP of any one of embodiments 111 to 114, wherein the LNP further comprises an ionizable cationic lipid.

116. The LNP of embodiment 115, wherein the ionizable cationic lipid comprises the compound of any one of embodiments 1 to 20.

117. A method of targeting the delivery of a nucleic acid to an immune cell of a subject, comprising contacting the immune cell with the LNP of any one of embodiments 21 to 116, wherein the LNP comprises the nucleic acid.

118. A method of expressing a polypeptide of interest in a targeted immune cell of a subject, comprising contacting the immune cell with the LNP of any one of embodiments 21 to 116, wherein the LNP comprises a nucleic acid encoding the polypeptide.

119. A method of modulating cellular function of a target immune cell of a subject, comprising administering to the subject the LNP of any one of embodiments 21 to 116, wherein the LNP comprises a nucleic acid modulates the cellular function of the immune cell.

120. A method of treating, ameliorating, or preventing a symptom of a disorder or disease in a subject in need thereof, comprising administering to the subject an LNP for delivering a nucleic acid into an immune cell of the subject, wherein the LNP is any one of embodiments 21 to 116, wherein the LNP comprises the nucleic acid.

121. The method of embodiment 120, wherein the disorder is an immune disorder, an inflammatory disorder, or cancer.

122. The method of embodiment 120, wherein the nucleic acid encodes an antigen for use in a therapeutic or prophylactic vaccine for treating or preventing cancer.

123. The method of any one of embodiments 117 to 122, wherein the immune cell targeting group comprises an antibody that binds a macrophage antigen.

124. The method of embodiment 123, wherein the macrophage antigen comprises CDIIB, CD68, CD80, CD86, TRL-2, TRL-4, iNOS, MHC-IL, CD163, CD206, CD209, FIZZ1, or Ym1/2, or any combination thereof.

125. The method of any one of embodiments 117 to 124, wherein the antibody is a human or humanized antibody.

126. The method of any one of embodiments 117 to 125, wherein the immune cell targeting group is covalently coupled to a lipid in the lipid blend via a polyethylene glycol (PEG) containing linker.

127. The method of embodiment 126, wherein the lipid covalently coupled to the immune cell targeting group via a PEG containing linker is distearoylglycerol (DSG), distearoyl-phosphatidylethanolamine (DSPE), dimyristoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.

128. The method of embodiment 126 or 127, wherein the PEG is PEG 2000.

129. The method of any one of embodiments 117 to 128, wherein the lipid-immune cell targeting group conjugate is present in the lipid blend in a range of 0.002-0.2 mole percent.

130. The method of any one of embodiments 117 to 129, wherein the ionizable cationic lipid is present in the lipid blend in a range of 40-60 mole percent.

131. The method of embodiment 117 to 130, wherein the sterol is cholesterol.

132. The method of any one of embodiments 117 to 131, wherein the sterol is present in the lipid blend in a range of 30-50 mole percent.

133. The method of claim 117 to 132, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), sphingomyelin (SM).

134. The method of embodiment 117 to 133, wherein the neutral phospholipid is present in the lipid blend in a range of 5-15 mole percent.

135. The method of any one of embodiments 117 to 134, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylglycerol (PEG-DOG), PEG-dimyristoyl-glycerol (PEG-DMG), PEG-dipalmitoyl-glycerol (PEG-DPG), PEG-dilinolenoyl-glycero-phosphatidyl ethanolamine (PEG-DLPE), PEG-dimyristoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

136. The method of embodiments 117 to 134, wherein the free PEG-lipid comprises a diacylphosphatidylethanolamines comprising dimyristoyl (C14) chain, Dipalmitoyl (C16) chain or Distearoyl (C18) chain.

137. The method of any one of embodiments 117 to 136, wherein the free PEG-lipid is present in the lipid blend in a range of 0.5-2.5 mole percent.

138. The method of any one of embodiments 117 to 137, wherein the free PEG-lipid comprises the same or a different lipid as the lipid in the lipid-immune cell targeting group conjugate.

139. The method of embodiments 117 to 138, wherein the LNP has a mean diameter in the range of 50-200 nm.

140. The method of embodiment 139, where the LNP has a mean diameter of about 100 nm.

141. The method of embodiments 117 to 140, wherein the LNP has a polydispersity index in a range from 0.01 to 0.1.

142. The method of embodiments 117 to 141, wherein the LNP has a zeta potential of from about +0 mV to about +10 mV at pH 5.5, or from about −5 mV to about 0 mV at pH 7.4.

143. The method of embodiments 117 to 142, wherein the nucleic acid is DNA or RNA.

144. The method of embodiment 143, wherein the RNA is an mRNA, tRNA, siRNA, gRNA, or microRNA.

145. The method of embodiment 144, wherein the mRNA encodes a receptor, a growth factor, a hormone, a cytokine, an antibody, an antigen, an enzyme, or a vaccine.

146. The method of embodiment 144, wherein the mRNA encodes a polypeptide capable of regulating immune response in the immune cell.

147. The method of embodiment 144, wherein the mRNA encodes a polypeptide capable of reprogramming the immune cell.

148. The method of embodiment 144, wherein the mRNA encodes polypeptide capable of reprogramming an M2 macrophage to an M1 macrophage.

149. The method of any one of embodiments 117 to 148, wherein the immune cell targeting group comprises an antibody, and the antibody is a Fab or an immunoglobulin single variable domain.

150. The method of any one of embodiments 117 to 149, wherein the immune cell targeting group comprises an antibody fragment selected from the group consisting of a Fab, F(ab′)2, Fab′-SH, Fv, and scFv fragment.

151. The method of embodiment 142 or 150, wherein the immune cell targeting group comprises a Fab that comprises one or more interchain disulfide bonds.

152. The method of embodiment 151, wherein the Fab comprises a heavy chain fragment that comprises F174C and C233S substitutions, and a light chain fragment that comprises S176C and C214S substitutions, numbering according to Kabat.

153. The method of any one of embodiments 149 to 152, wherein the immune cell targeting group comprises a Fab that comprises a cysteine at the C-terminus of the heavy or light chain fragment.

154. The method of embodiment 153, wherein the Fab further comprises one or more amino acids between the heavy chain fragment of the Fab and the C-terminal cysteine.

155. The method of any one of embodiments 149 to 154, wherein the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker.

156. The method of embodiment 149, wherein the immune cell targeting group comprises an immunoglobulin single variable domain.

157. The method of embodiment 149 or 156, wherein the immunoglobulin single variable domain comprises a cysteine at the C-terminus.

158. The method of embodiment 157, wherein the immunoglobulin single variable domain comprises a V_{HH} domain and further comprises a spacer comprising one or more amino acids between the V_{HH} domain and the C-terminal cysteine.

159. The method of any one of embodiments 149 and 156 to 158, wherein the immune cell targeting group comprises two or more V_{HH} domains.

160. The method of embodiment 159, wherein the two or more V_{HH} domains are linked by an amino acid linker.

161. The method of embodiment 159, wherein the immune cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds.

162. The method of any one of embodiments 149 and 156 to 158, wherein the immune cell targeting group comprises a V_{HH} domain linked to an antibody CH1 domain, and wherein the antibody CH1 domain is linked to an antibody light chain constant domain by one or more disulfide bonds.

163. The method of embodiment 161 or 162, wherein the CH1 domain comprises F174C and C233S substitutions, and the light chain constant domain comprises S176C and C214S substitutions, numbering according to Kabat.

164. The method of any one of embodiments 117 to 163, wherein no more than 5% non-immune cells are transfected by the LNP.

165. The method of any one of embodiments 117 to 164, wherein half-life of the nucleic acid delivered by the LNP or a polypeptide encoded by the nucleic acid delivered by the LNP is at least 10% longer than half-life of nucleic acid delivered by a reference LNP or a polypeptide encoded by the nucleic acid delivered by the reference LNP.

166. The method of any one of embodiments 117 to 165, wherein at least 10% immune cells are transfected by the LNP.

167. The method of any one of embodiments 117 to 166, wherein expression level of the nucleic acid delivered by the LNP is at least 10% higher than expression level of nucleic acid delivered by a reference LNP.

168. A method of targeting delivery of a nucleic acid to a non-liver cell, the method comprising contacting the non-liver cell with an LNP of any one of embodiments 21 to 116, wherein the LNP comprises about 1 to about 2 mol % of free PEG-lipid.

169. The method of embodiment 168, wherein the LNP comprises about 1.5 mol % of free PEG-lipid.

170. A method of targeting delivery of a nucleic acid to a liver cell, the method comprising contacting the liver cell with an LNP of any one of embodiments 21 to 116, wherein the LNP comprises about 2 to about 4 mol % of free PEG-lipid.

171. The method of embodiment 170, wherein the LNP comprises about 3.5 mol % of free PEG-lipid.

### EXAMPLES

The invention now being generally described, will be more readily understood by reference to the following examples, which are included merely for purposes of illustration of certain aspects and embodiments of the present invention, and are not intended to limit the invention.

### Example 1. Preparation of Ionizable Cationic Lipids

This Example describes the synthesis of various cationic lipids.

### Synthesis of Head Group Intermediates 8, 8′, 9 and 9′

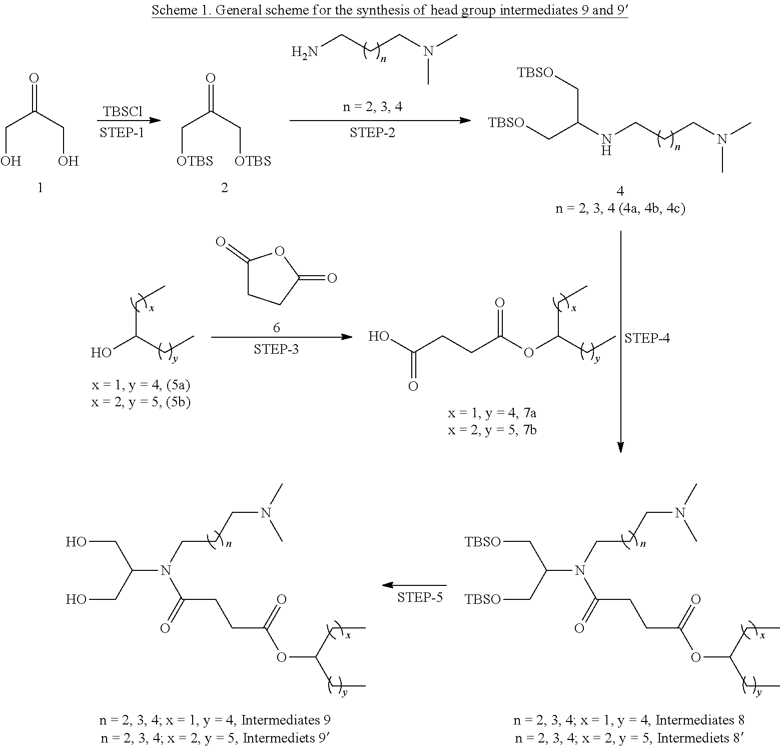

### Synthesis of Intermediates 9′a, 9′b, and 9′c

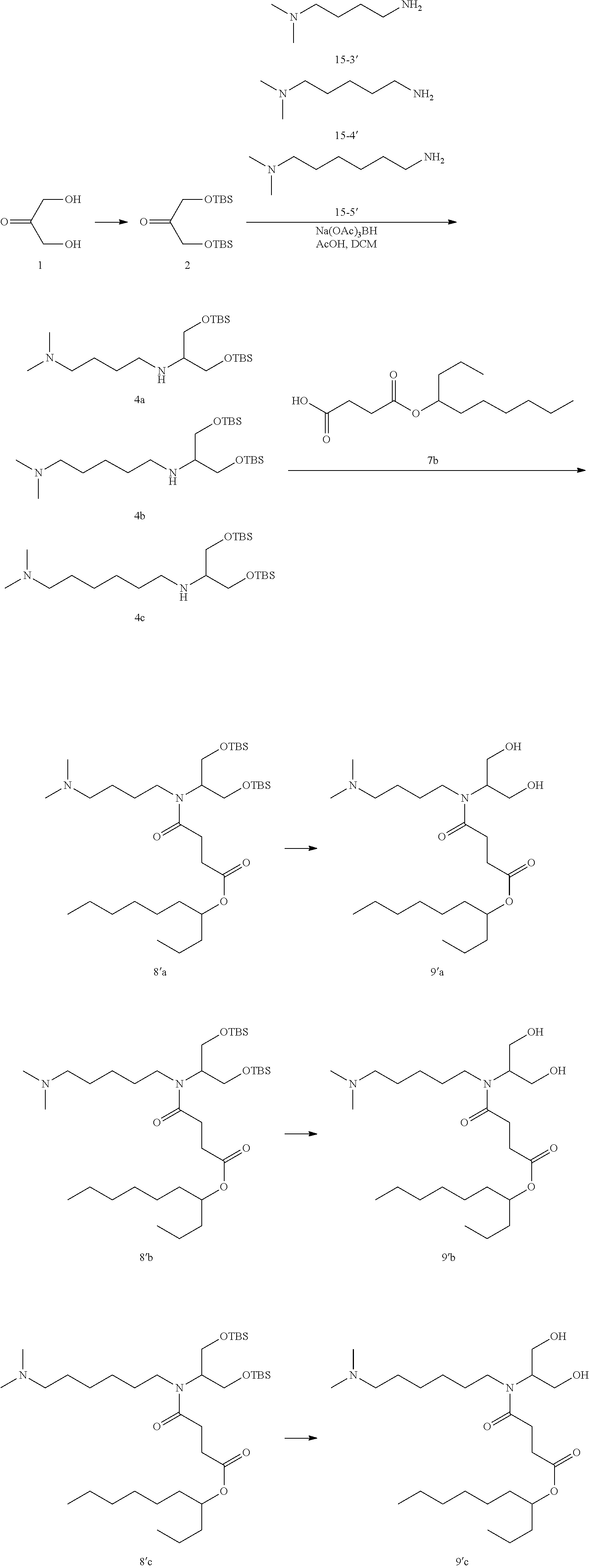

Protection of starting material dihydroxyacetone 1 (111 mmol, 10 g, 1 eq.) using tert-butyltrimethylsilyl chloride TBSCl (332.9 mmol, 50.2 g, 3.0 eq), TEA (554.9 mmol, 56.15 g, 5 eq,) and DMAP (22.1 mmol, 2.71 g, 0.2 eq.) in 100 mL DCM at room temperature for 16 hours yielded protected intermediate 2. Crude intermediate 2 (45 g) was purified using ISCO column chromatography on silica column eluting with Hexane and ethyl acetate to obtain 20 g of purified intermediate 2.

Intermediate 2 (6 g, 18.8 mmol, 1 eq.) was reductively aminated using N,N-dimethylaminobutyl amine 15-3′, (37.7 mmol, 4.38 g, 2.0 eq.), Acetic acid (37.7 mmol, 2.26 mL, 2.0 eq.), and Na(OAc)₃BH (22.6 mmol, 4.79 g, 1.2 eq.), in 60 mL of dichloromethane at room temperature for 6 hours. Crude product was purified by filter column chromatography on silica column eluting with DCM and (10% MeOH in DCM+1% NH₄OH) to obtain desired product yielding 5.6 g (66%) pure intermediate 4a based on TLC.

Intermediate 2 (3 g, 9.4 mmol, 1 eq.) was reductively aminated using N,N-dimethylaminobutyl amine 15-4′, (18.8 mmol, 2.45 g, 2.0 eq.), Acetic acid (18.8 mmol, 1.13 mL, 2.0 eq.), and Na(OAc)₃BH (11.3 mmol, 2.39 g, 1.2 eq.), in 30 mL of dichloromethane at room temperature for 3 hours. Crude product was purified by filter column chromatography on silica column eluting with DCM and (10% MeOH in DCM+1% NH₄OH) to obtain desired product yielding 2.9 g (71%) pure intermediate 4b based on product mass and proton NMR.

Intermediate 2 (1 g, 3.1 mmol, 1 eq.) was reductively aminated using N,N-dimethylaminobutyl amine 15-5′, (37.7 mmol, 0.86 g, 2.0 eq.), Acetic acid (37.7 mmol, 1.13 mL, 2.0 eq.), and Na(OAc)₃BH (11.3 mmol, 2.39 g, 1.2 eq.), in 30 mL of dichloromethane at room temperature for 3 hours. Crude product was purified by filter column chromatography on silica column eluting with DCM and (10% MeOH in DCM+1% NH₄OH) to obtain desired product yielding 1.1 g (78%) of pure intermediate 4c based on product mass and proton NMR.

Intermediate 4a (5.2 g, 12.4 mmol, 1 eq.) was reacted with previously produced intermediate 7b (4.81 g, 1.5 eq, 18.6 mmol) using EDCI (3.57 g, 1.5 eq, 18.6 mmol), DIPEA (2.41 g, 1.5 eq, 18.6 mmol) and DMAP (0.75 g, 0.5 eq, 6.2 mmol) in DCM (60 mL) at room temperature for 4 hours. Crude product was purified by filter column chromatography on silica column eluting with DCM and (10% MeOH in DCM+1% NH₄OH) to obtain desired product yielding 3.4 g (36%) of pure intermediate 8′a based on product mass and proton NMR.

Intermediate 4b (3.1 g, 7.1 mmol, 1 eq.) was reacted with previously produced intermediate 7b (2.77 g, 1.5 eq, 10.7 mmol) using EDCI (2.05 g, 1.5 eq, 10.7 mmol), DIPEA (1.38 g, 1.5 eq, 10.7 mmol) and DMAP (0.43 g, 0.5 eq, 3.5 mmol) in DCM (30 mL) at room temperature for 4 hours. Crude product was purified by filter column chromatography on silica column eluting with DCM and (10% MeOH in DCM+1% NH₄OH) to obtain desired product yielding 2.7 g (56%) of pure intermediate 8′b based on product mass and proton NMR.

Intermediate 4c (1.1 g, 1.6 mmol, 1 eq.) was reacted with previously produced intermediate 7b (0.9 g, 1.5 eq, 2.4 mmol) using EDCI (0.7 g, 1.5 eq, 3.6 mmol), DIPEA (0.47 g, 1.5 eq, 3.6 mmol) and DMAP (0.15 g, 0.5 eq, 1.2 mmol) in DCM (10 mL) at room temperature for 4 hours. Crude product was purified by filter column chromatography on silica column eluting with DCM and (10% MeOH in DCM+1% NH₄OH) to obtain desired product yielding 0.8 g (47%) of pure intermediate 8′c based on product mass and proton NMR.

Intermediate 8′a (300 mg, 0.45 mmol, 1 eq.) was deprotected in HF-Pyridine (0.41 mL, 10.0 eq, 4.5 mmol) and THF (20 mL) at room temperature for 2 hours to obtain dihydroxyl intermediate 9′a. Crude product was used in subsequent reactions (as described below).

Intermediate 8′b (300 mg, 0.44 mmol, 1 eq.) was deprotected in HF-Pyridine (0.40 mL, 10.0 eq, 4.4 mmol) and THF (4 mL) at room temperature for 2 hours to obtain dihydroxyl intermediate 9′b. Crude product was used in subsequent reactions (as described below).

Intermediate 8′c (300 mg, 0.45 mmol, 1 eq.) was deprotected in HF-Pyridine (0.41 mL, 10.0 eq, 4.5 mmol) and THF (4 mL) at room temperature for 2 hours to obtain dihydroxyl intermediate 9′c. Crude product was used in subsequent reactions (as described below).

### Synthesis of Intermediate 14-34′

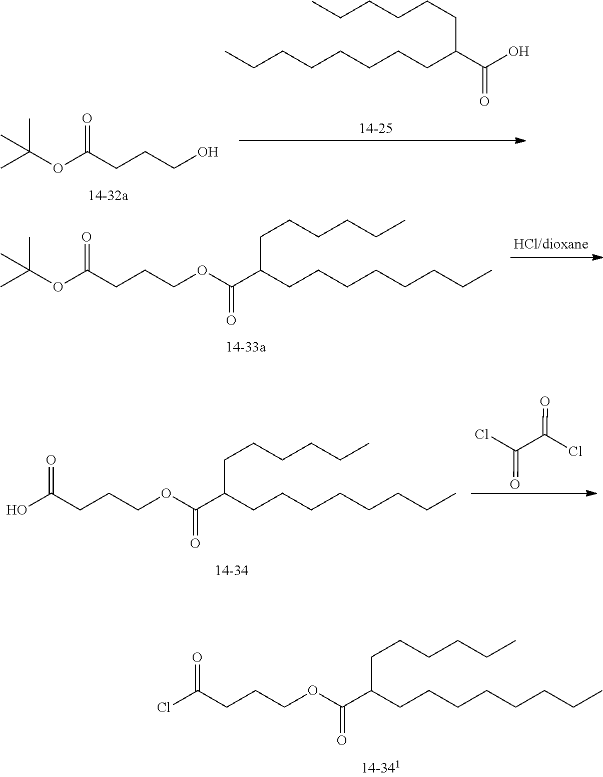 [SMILES: CCCCCCCCC(CCCCCC)C(=O)O ; CC(C)(C)OC(=O)CCCO ; CCCCCCCCC(CCCCCC)C(=O)OCCCC(=O)OC(C)(C)C ; CCCCCCCCC(CCCCCC)C(=O)OCCCC(=O)O ; O=C(Cl)C(=O)Cl ; CCCCCCCCC(CCCCCC)C(=O)OCCCC(=O)Cl]

Starting material 14-32a (4.82 g, 30.09 mmol, 1.0 eq.) was esterified with 7-carboxyhexanoic acid 14-25 (11.30 g, 44.07 mmol, 1.45 eq.) using EDCI (8.84 g, 46.11 mmol, 1.53 eq), DIPEA (8 mL, 45.93 mmol, 1.53 eq) and DMAP (0.796 g, 6.51 mmol, 0.22 eq) in DCM (50 mL) at room temperature overnight to obtain protected intermediate 14-33a. Crude product was purified on Silica gel column using hexanes/ethyl acetate (6/4) mixture as eluent to yield pure compound 14-33a (7.75 g, 65%) based on product mass and NMR.

Protected intermediate 14-33a (4.38 g, 10.98 mmol, 1.0 eq.) was deprotected using 4N HCl in dioxane (24 mL) at room temperature overnight. Crude product was purified by column chromatography on silica column eluting with Hexanes/Ethyl acetate to obtain free acid intermediate 14-34 (3 g, 80%) based on ¹H NMR. Intermediate 14-34 (1.86 g, 5.4 mmol) was converted to the corresponding acid chloride 14-34′ using oxalyl chloride (1.59 mL, 3.4 eq, 18.4 mmol) and DMF (200 μL) in toluene (12.0 mL) for 2 hours at room temperature. Crude product was used for the synthesis of lipids 40, 41 and 42 as described below.

### Synthesis of Lipid 40

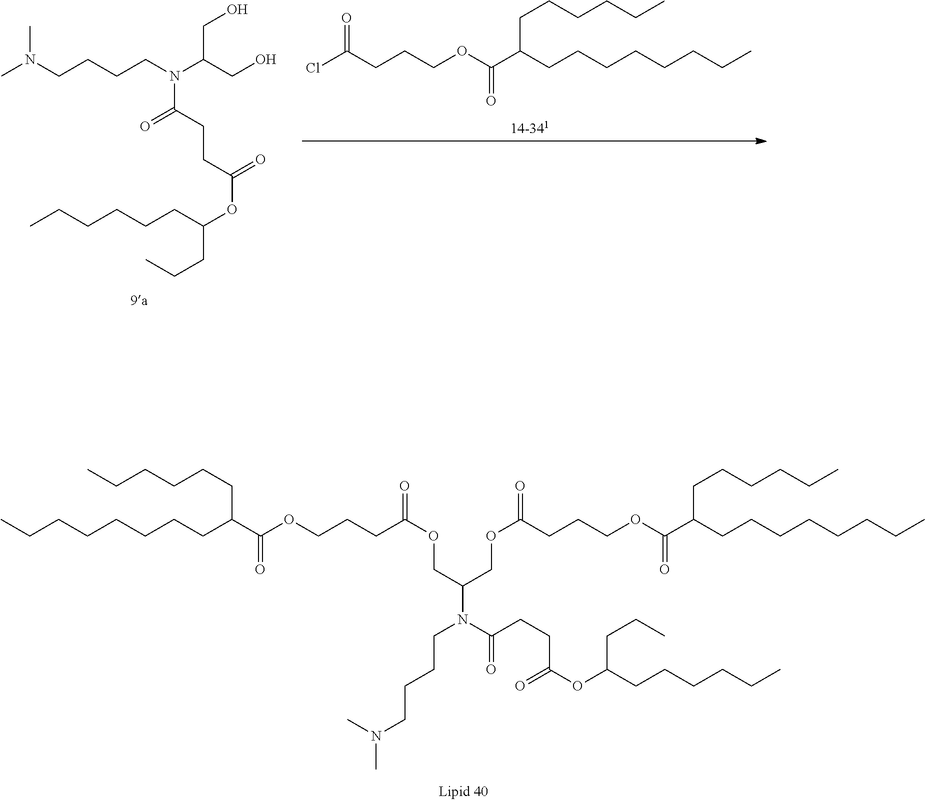 [SMILES: CCCCCCC(CCC)OC(=O)CCC(=O)N(CCCCN(C)C)C(CO)CO ; CCCCCCCCC(CCCCCC)C(=O)OCCCC(=O)Cl ; CCCCCCCCC(CCCCCC)C(=O)OCCCC(=O)OCC(COC(=O)CCCOC(=O)C(CCCCCC)CCCCCCCC)N(CCCCN(C)C)C(=O)CCC(=O)OC(CCC)CCCCCC]

Diol intermediate 9′a (0.39 g, 0.9 mmol, 1 eq.) was reacted with crude acid chloride 14-34′ (1.86 g, 6.0 eq, 5.4 mmol) using TEA (1.27 mL, 10.0 eq, 9.05 mmol) in toluene (10.0 mL), at room temperature overnight to obtain crude lipid 40. Crude product was purified on ISCO column chromatography on silica column eluting with DCM and 10% MeOH in DCM to obtain pure lipid 40 (480 mg, 49%) characterized by mass spectrometry, ¹H NMR, and LC-CAD (>99%).

### Synthesis of Lipid 41

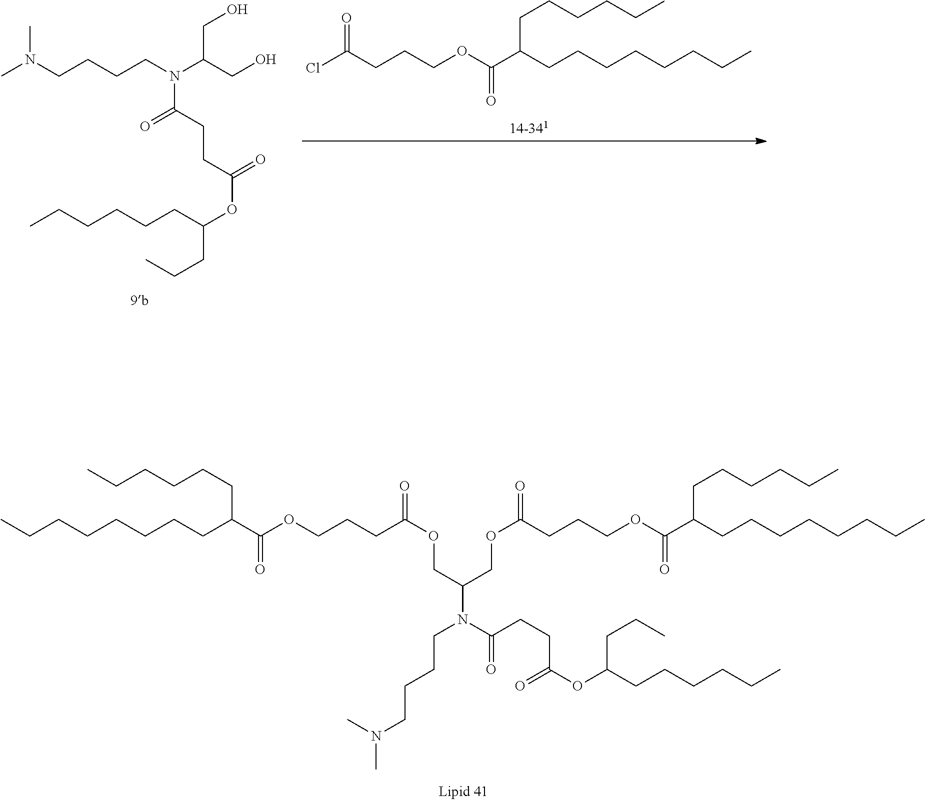 [SMILES: CCCCCCC(CCC)OC(=O)CCC(=O)N(CCCCN(C)C)C(CO)CO ; CCCCCCCCC(CCCCCC)C(=O)OCCCC(=O)Cl ; CCCCCCCCC(CCCCCC)C(=O)OCCCC(=O)OCC(COC(=O)CCCOC(=O)C(CCCCCC)CCCCCCCC)N(CCCCN(C)C)C(=O)CCC(=O)OC(CCC)CCCCCC]

Diol intermediate 9′b (0.195 g, 0.43 mmol, 1 eq.) was reacted with crude acid chloride 14-34′ (0.90 g, 6.0 eq, 2.6 mmol) using TEA (0.61 mL, 10.0 eq, 4.3 mmol) in toluene (5.0 mL), at room temperature overnight to obtain crude lipid 41. Crude product was purified on ISCO column chromatography on silica column eluting with DCM and 10% MeOH in DCM to obtain pure lipid 41 (245 mg, 51%) characterized by mass spectrometry, ¹H NMR, and LC-CAD (>99%).

### Synthesis of Lipid 42

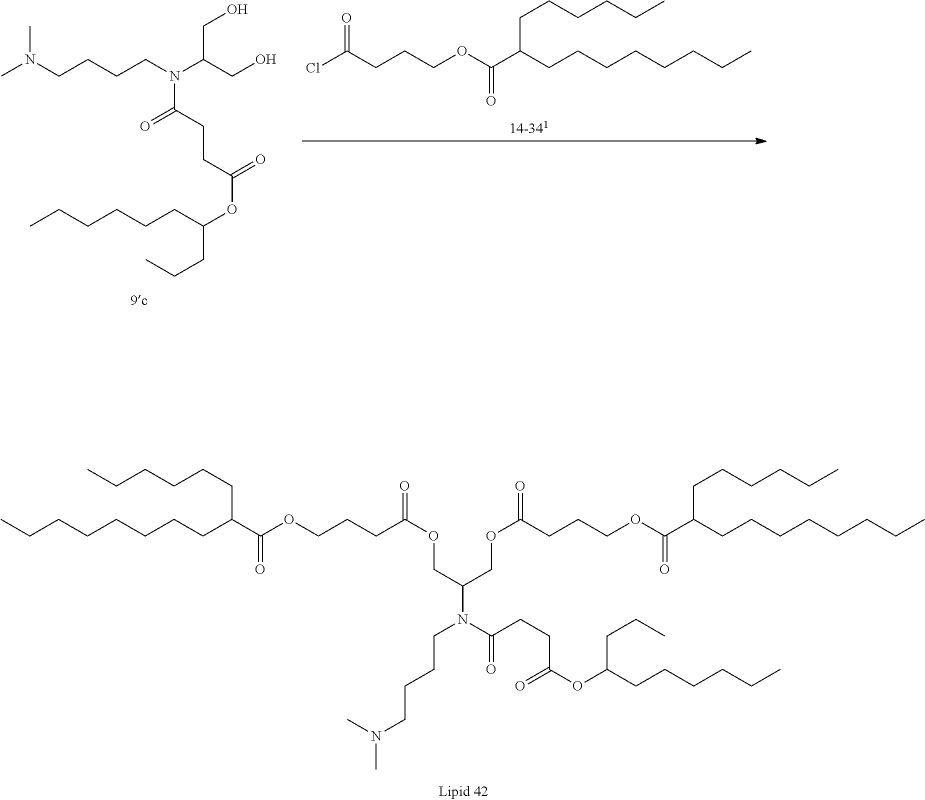 [SMILES: CCCCCCC(CCC)OC(=O)CCC(=O)N(CCCCN(C)C)C(CO)CO ; CCCCCCCCC(CCCCCC)C(=O)OCCCC(=O)Cl ; CCCCCCCCC(CCCCCC)C(=O)OCCCC(=O)OCC(COC(=O)CCCOC(=O)C(CCCCCC)CCCCCCCC)N(CCCCN(C)C)C(=O)CCC(=O)OC(CCC)CCCCCC]

Diol intermediate 9′c (0.195 g, 0.43 mmol, 1 eq.) was reacted with crude acid chloride 14-34′ (0.90 g, 6.0 eq, 2.6 mmol) using TEA (0.61 mL, 10.0 eq, 4.3 mmol) in toluene (5.0 mL), at room temperature overnight to obtain crude lipid 41. Crude product was purified on ISCO column chromatography on silica column eluting with DCM and 10% MeOH in DCM to obtain pure lipid 42 (320 mg, 67%) characterized by mass spectrometry, ¹H NMR, and LC-CAD (>99%).

### Synthesis of Tail Group Intermediate 10

Starting material 10a (3.0 g, (1.0 eq, 13.0 mmol)) was reacted with 8-(tert-Butoxy)-8-oxooctanoic acid (3.1 g, 1.5 eq, 19.5 mmol) using EDCI (3.75 g, 1.5 eq, 19.5 mmol), DMAP (728 mg, 0.5 eq, 6.5 mmol), and DIPEA (3.4 mL, 1.5 eq, 19.5 mmol) in DCM (20.0 mL) at room temperature, overnight to obtain protected intermediate 10b. Crude product was purified by Silica column chromatography eluting with Hexanes:Ethyl Acetate to obtain pure Intermediate 10b (4.39 g, 91%) characterized by mass spectrometry and ¹H NMR.

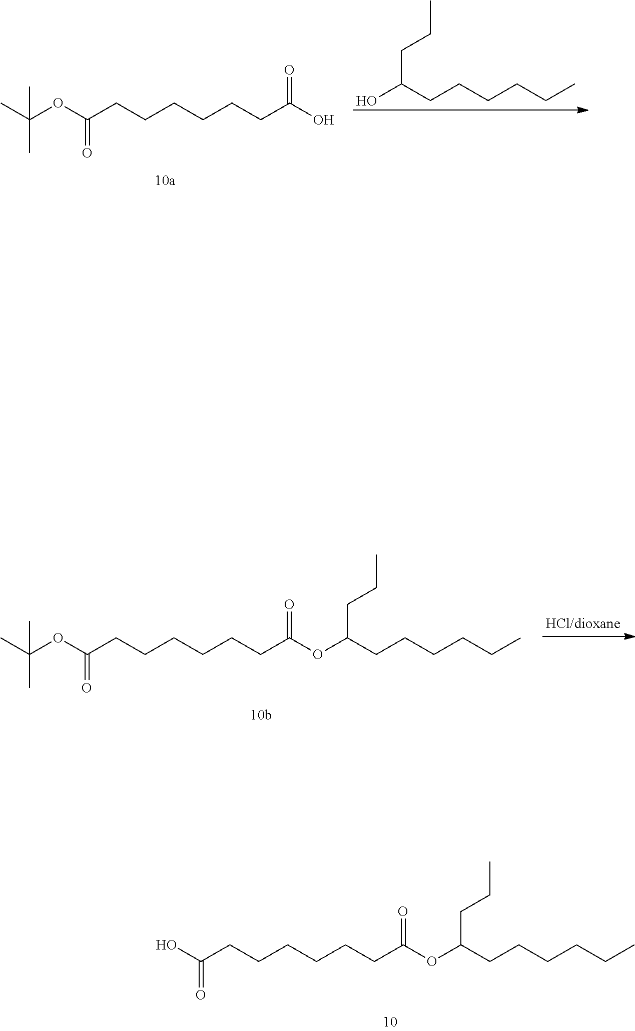 [SMILES: CC(C)(C)OC(=O)CCCCCCC(=O)O ; CCCCCCC(O)CCC ; CCCCCCC(CCC)OC(=O)CCCCCCC(=O)OC(C)(C)C ; CCCCCCC(CCC)OC(=O)CCCCCCC(=O)O]

Intermediate 10b (4.39 g, 1.0 eq, 11.8 mmol) was deprotected in 4.OM HCl in Dioxane/DCM (25 mL/10 mL) at room temperature, overnight to obtain acid intermediate 10. Crude product was purified by silica column chromatography with DCM:10% MeOH in DCM+1% NH₄OH (2×) to obtain 3.24 g (92%) characterized by ¹H NMR and Mass Spectrometry.

### Synthesis of Tail Group Intermediate 14-22

Protected starting material 14-19 (4.0 g, 1.0 eq, 15.5 mmol) was reacted with 3-hydroxydecanoyl 14-20 (3.7 g, 1.5 eq, 23.25 mmol) using EDCI (4.5 g, 1.5 eq, 23.25 mmol), DMAP (950 mg, 0.5 eq, 7.75 mmol) and DIPEA (4.0 mL, 1.5 eq, 23.25 mmol) in DCM (20.0 mL) at room temperature, overnight. Crude product was purified by ISCO column chromatography on silica column eluting with DCM and 10% MeOH in DCM (×2) to obtain 4.1 g of the desired product 14-21 based on ¹H NMR.

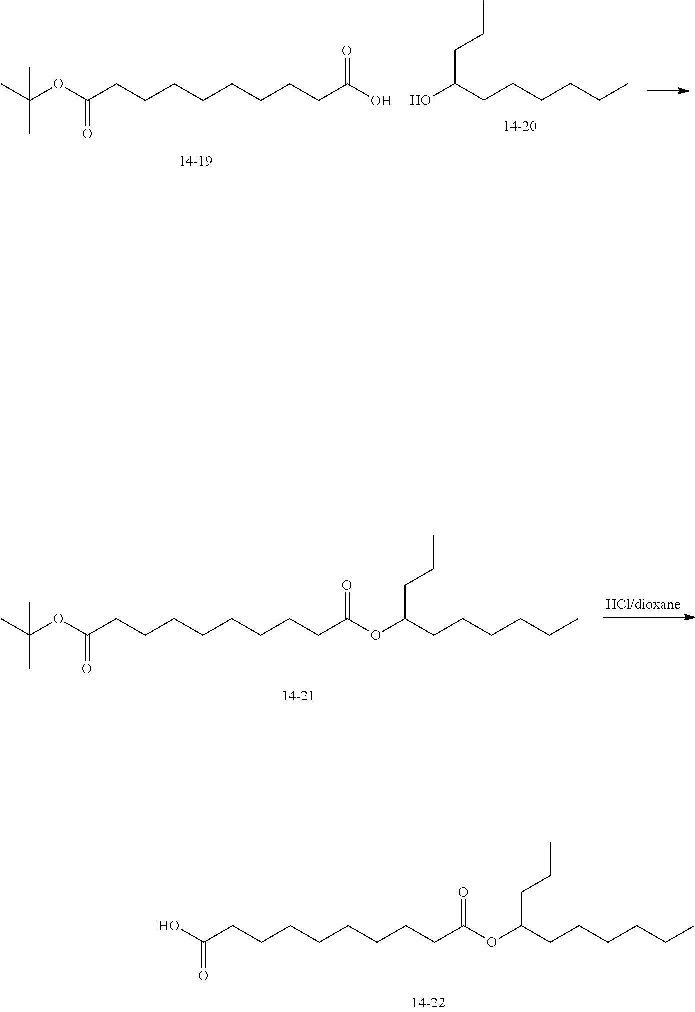 [SMILES: CC(C)(C)OC(=O)CCCCCCCCC(=O)O ; CCCCCCC(O)CCC ; CCCCCCC(CCC)OC(=O)CCCCCCCCC(=O)OC(C)(C)C ; CCCCCCC(CCC)OC(=O)CCCCCCCCC(=O)O]

Intermediate 14-21 (4.1 g, 1.0 eq, 10.3 mmol) was deprotected in 15 mL 4N HCl/Dioxane to yield product 14-22. Crude product was purified by ISCO column chromatography on silica eluting with Hexanes and Ethyl acetate to obtain 2.7 g (77%) of pure 14-22 based on ¹H NMR.

### Synthesis of Tail Group Intermediate 2

Starting material 14-23 ( ) was reacted with 14-24 (3.4 g, 1.5 eq, 26.0 mmol) using EDCI (5.0 g, 1.5 eq, 26.0 mmol), DMAP (292 mg, 0.15 eq, 2.6 mmol) and DIPEA (4.5 mL, 1.5 eq, 26.0 mmol) in DCM (100 mL), at room temperature, overnight to obtain protected intermediate 2′. Crude product was purified by Silica column chromatography eluting with Hexanes:Ethyl Acetate to obtain pure Intermediate 2′ (5.84 g, 84%) characterized by mass spectrometry and ¹H NMR

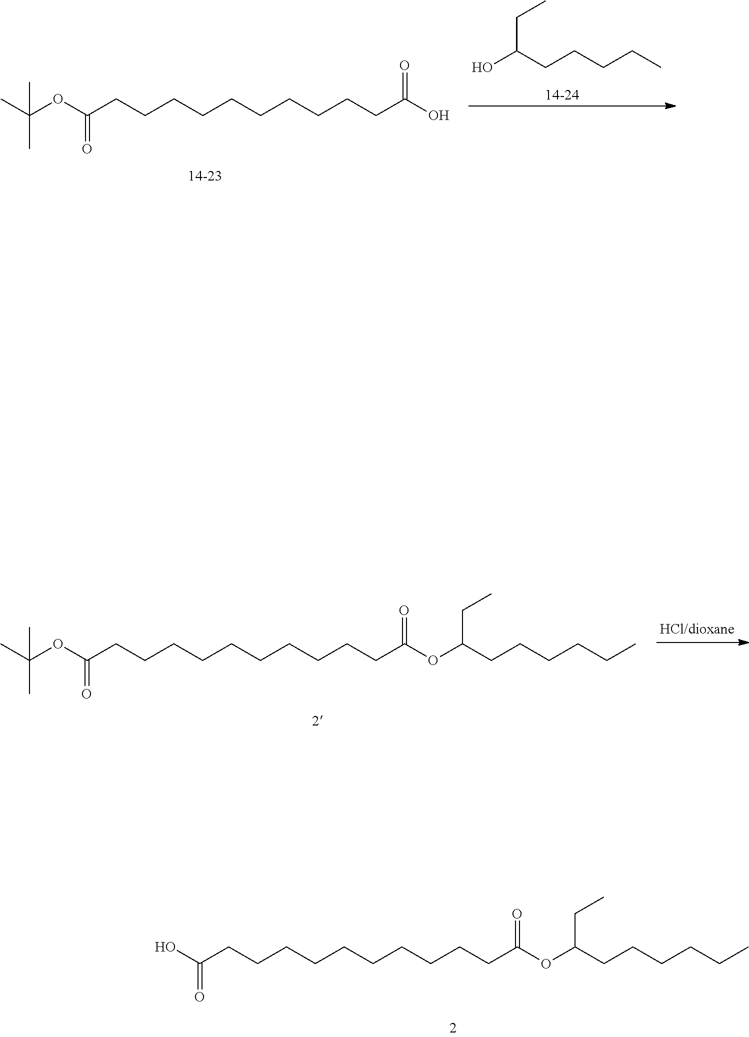

Protected intermediate 2′ (5.84 g, 1.0 eq, 17.0 mmol) was deprotected in 4.OM HCl in Dioxane/DCM (40 mL/10 mL) at room temperature, overnight to obtain acid intermediate 10. Crude product was purified by silica column chromatography with DCM:10% MeOH in DCM+1% NH₄OH to obtain 4.47 g (82%) characterized by ¹H NMR and Mass Spectrometry.

### Synthesis of Lipid 43

Intermediate 9′a was reacted with intermediate 10 (723 mg, 3.0 eq, 2.3 mmol) using EDCI (875 mg, 6.0 eq, 4.56 mmol), DIPEA (794 μL, 6.0 eq, 4.56 mmol) and DMAP (51 mg, 0.6 eq, 0.46 mmol) in DCM (30 mL) at room temperature, overnight.

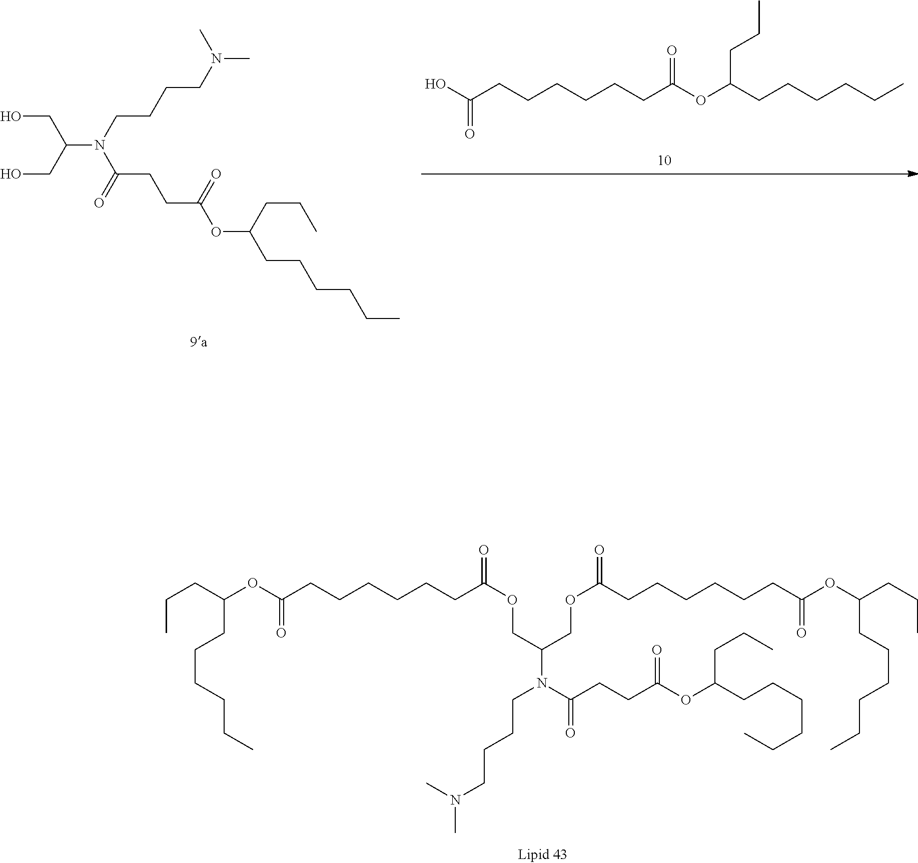 [SMILES: CCCCCCC(CCC)OC(=O)CCC(=O)N(CCCCN(C)C)C(CO)CO ; CCCCCCC(CCC)OC(=O)CCCCCCC(=O)O ; CCCCCCC(CCC)OC(=O)CCCCCCC(=O)OCC(COC(=O)CCCCCCC(=O)OC(CCC)CCCCCC)N(CCCCN(C)C)C(=O)CCC(=O)OC(CCC)CCCCCC]

Crude product was purified on silica column eluting with DCM:10% MeOH in DCM to obtain 600 mg of product with impurities. The compound was repurified on silica column eluting with Hexanes:Ethyl acetate to obtain 320 mg of purified compound based on LC-ELSD Purity >98% and UPLC-CAD purity >90%; characterized by ¹H NMR and Mass Spectrometry.

### Synthesis of Lipid 46

Intermediate 14-22 (1.27 g, 1.0 eq, 3.72 mmol) was converted to the corresponding acid chloride 14-22′, using Oxalyl chloride (1.1 mL, 3.4 eq, 12.6 mmol) and DMF (40 μL), in Toluene (5.0 mL) at room temperature, overnight.

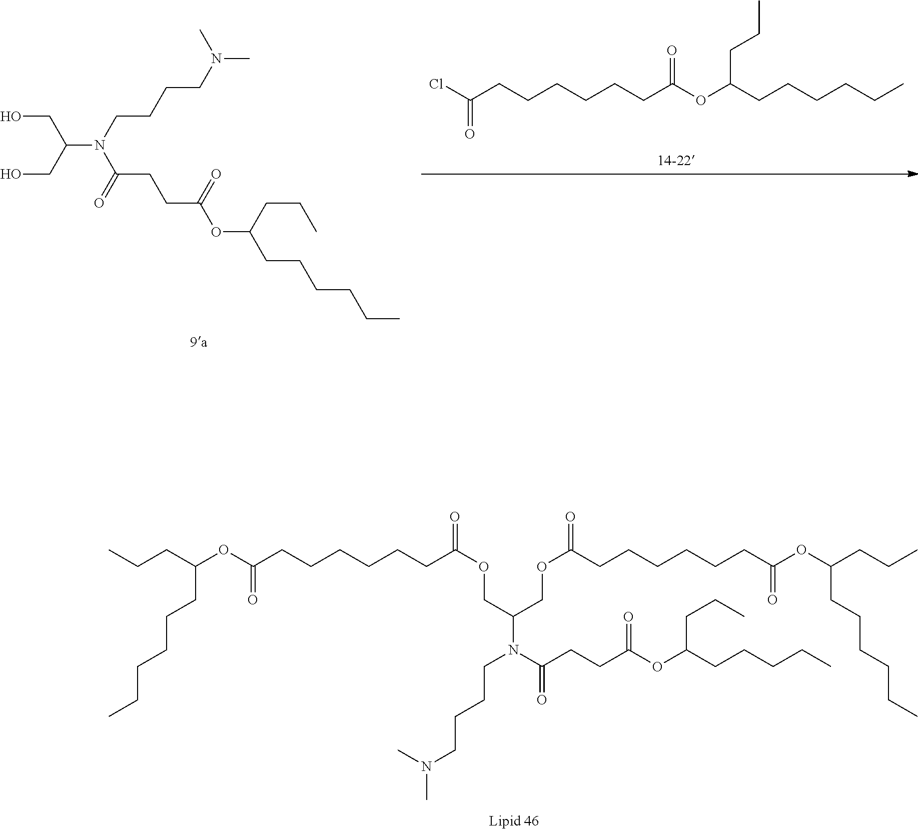 [SMILES: CCCCCCC(CCC)OC(=O)CCC(=O)N(CCCCN(C)C)C(CO)CO ; CCCCCCC(CCC)OC(=O)CCCCCCC(=O)Cl ; CCCCCCC(CCC)OC(=O)CCCCCCC(=O)OCC(COC(=O)CCCCCCC(=O)OC(CCC)CCCCCC)N(CCCCN(C)C)C(=O)CCC(=O)OC(CCC)CCCCC]

Intermediate 9′a (263 mg, 1.0 eq, 0.61 mmol) was reacted with crude 14-22′ (1.32 g, 6.0 eq, 3.66 mmol) using TEA (0.85 mL, 10.0 eq, 6.1 mmol) in Toluene (5.0 mL) at room temperature, overnight. Crude product was purified by ISCO column chromatography on silica column eluting with Hexanes and Ethyl acetate to afford pure Lipid 46 (308 mg, 47%) characterized by UPLC-CAD (>99%), ¹H NMR and Mass Spectrometry.

### Synthesis of Lipid 52

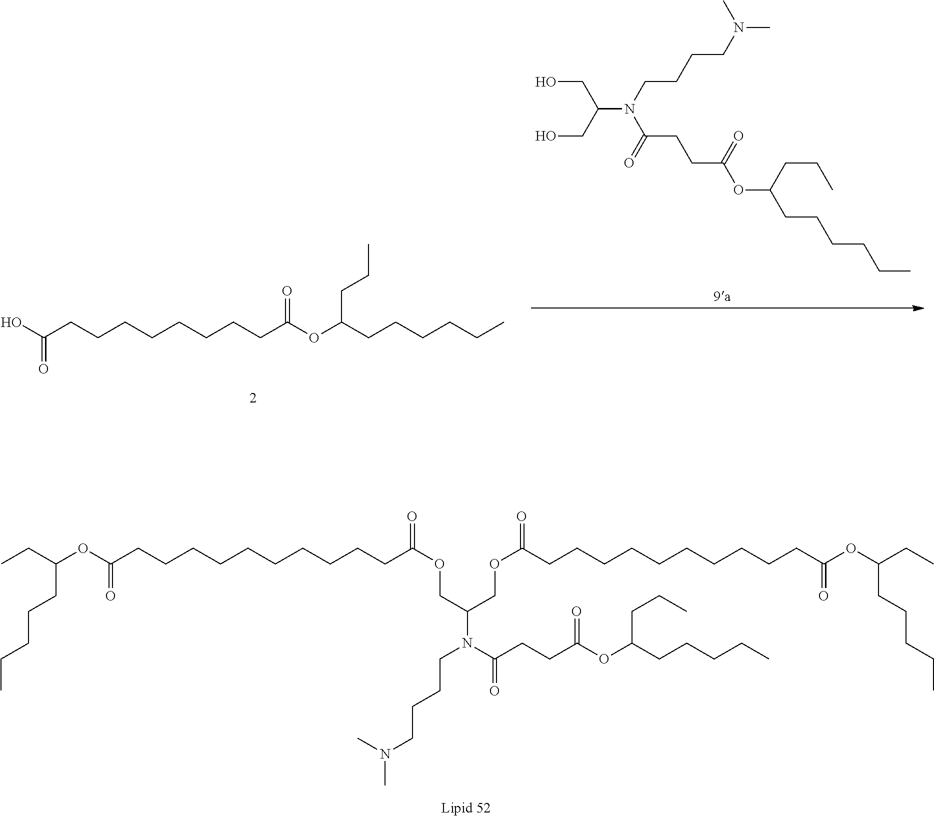 [SMILES: CCCCCCC(CCC)OC(=O)CCCCCCCCC(=O)O ; CCCCCCC(CCC)OC(=O)CCC(=O)N(CCCCN(C)C)C(CO)CO ; CCCCCC(CC)OC(=O)CCCCCCCCCCC(=O)OCC(COC(=O)CCCCCCCCCCC(=O)OC(CC)CCCCC)N(CCCCN(C)C)C(=O)CCC(=O)OC(CCC)CCCCC]

Intermediate 2 (330 mg, 1.0 eq, 0.76 mmol) was reacted with diol intermediate 9′a (786 mg (3.0 eq, 2.3 mmol) using EDCI (875 mg, 6.0 eq, 4.56 mmol), DIPEA (794 μL, 6.0 eq, 4.56 mmol) and DMAP (51 mg, 0.6 eq, 0.46 mmol) in DCM (30 mL) at room temperature, overnight. Crude product was purified on silica column eluting with Hexanes: EtOAc to obtain 690 mg of product with impurities. The compound was repurified on silica column eluting with DCM:10% MeOH in DCM to obtain 600 mg of product with impurities. The compound was repurified on silica column eluting with Hexanes:Ethyl acetate to obtain 480 mg of purified Lipid 52 (LC-ELSD Purity >99%. UPLC purity >90%) and characterized by ¹H NMR and MS.

### Example 2. Preparation of LNPs by Microfluidic Mixing Using Exemplary Lipids

This Example describes the production of mRNA-loaded LNPs using exemplary materials and microfluidic mixing process.

LNPs encapsulating an mRNA payload were prepared by mixing an aqueous mRNA solution and an ethanolic lipid blend solution (containing the ionizable lipid, DSPC, DPG-PEG and Cholesterol at lipid ratios shown in TABLE 2) using an in-line microfluidic mixing process. The mRNA stock solution was diluted in pH 4 acetate buffer (yielding a 400 μg/mL solution of mRNA) in 65 mM pH 4 acetate buffer. The lipid components were dissolved in anhydrous ethanol at the relative ratios set forth in TABLE 2 below.

TABLE 2

| | | Ratio of Lipid | | Theoretical |
|---|---|---|---|---|
| | | to mRNA (nmol | Concentration in | LNP Lipid |
| | | lipid/100 μg | Lipid Solution | Composition |
| Lipid | Source | mRNA) | (mM) | (mol %) |
| Ionizable Lipid | — | 1,500 | 18 | 49.2449 |
| Cholesterol | Dishman Netherlands | 1,200 | 14.4 | 39.3959 |
| DSPC | Avanti Polar Lipids, Alabama, U.S. | 300 | 3.6 | 9.849 |
| DPG-PEG(2000) | NOF America, New York, U.S. | 46 | 0.55 | 1.5102 |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directly mounted into the luer ports of the mixing cartridge. The two solutions were then mixed at a 3:1 v/v ratio of mRNA solution (3.75 mL) to lipid solution (1.25 mL) at a total flow rate of 9 mL/min using the NanoAssemblr Ignite (the ratios, volumes, and flow rates can vary). The resulting suspension was held at room temperature for a minimum of 5 minutes before proceeding to ethanol removal and buffer exchange.

Following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with MBS exchange buffer (25 mM pH 6.5 MES buffer with 150 mM NaCl). The LNP suspension (5 mL) was then loaded into the device and centrifuged at 500 RCF until the volume was reduced by half (2.5 mL). The suspension was then diluted with exchange buffer (2.5 mL of MBS) to bring the suspension back to the original volume. This process of two-fold concentration and two-fold dilution was repeated five additional times for a total of six discontinuous diafiltration steps. The retentate containing the LNPs in MBS was recovered from the centrifugal ultrafiltration device, mixed with sucrose to a final sucrose concentration of 10% w/v, and then filtered using a 0.2 μm PES syringe filter. The LNPs were either used right away, or stored frozen at −80° C. until further use.

### Example 3. Preparation of LNPs by Microfluidic Mixing Using Exemplary Lipids—Characterization of LNPs

This Example describes the characterization of LNPs produced in Example 2.

Samples of the LNPs produced in Example 2 were characterized to determine the average hydrodynamic diameter, zeta potential, and mRNA content (total and dye-accessible mRNA). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer.

RNA content of the nanoparticles is measured using Thermo Fisher Quant-iT RiboGreen RNA Assay Kit. Dye accessible RNA, which includes both un-encapsulated RNA and accessible RNA at the LNP surface, is measured by diluting the nanoparticles to approximately 1 μg/mL mRNA using HEPES buffered saline, and then adding Quant-iT reagent to the mixture. Total RNA content is measured by disrupting a nanoparticle suspension by dilution of the stock LNP batch (typically at ≥40 μg/mL RNA) in 0.5% Triton solution in HEPES buffered saline to obtain a 1 μg/mL RNA solution (final nominal concentration based on formulation input values) and subsequent heating at 60° C. for 30 minutes followed by addition of Quant-It reagent. RNA is quantified by measuring fluorescence at 485/535 nm, and concentration is determined relative to a contemporaneously run RNA standard curve. Exemplary results are set forth in TABLE 3.

TABLE 3

| | | DLS Z- | | Zeta | Zeta | |
|---|---|---|---|---|---|---|
| | | Avg. | | Potential | Potential | Dye- |
| | Ionizable Lipid/ | Diameter | DLS | at pH 5.5 | at pH 7.4 | Accessible |
| LNP Lot Number | mRNA Payload/Dye | (nm) | PDI | (mV) | (mV) | mRNA (%) |
| EXP22020613-NAM | ALC-0315 GFP/DiI C18-5DS | 80.3 | 0.09 | 5.93 | −4.07 | 13.4 |
| EXP22020613-NPM | Lipid 15/GFP/DiI C18-5DS | 83.9 | 0.09 | 16.4 | 0.729 | 8.53 |
| EXP22020613-NUM | Lipid 26/GFP/DiI C18-5DS | 82.2 | 0.05 | 3.65 | −3 | 8.23 |
| EXP22020613-NFM | Lipid 25A/GFP/DiI C18-5DS | 70.7 | 0.04 | 4.81 | −4.49 | 8.14 |
| EXP22020613-NGM | Lipid 27/GFP/DiI C18-5DS | 83.2 | 0.05 | 4.14 | −2.37 | 8.03 |
| EXP22020613-NHM | Lipid 28/GFP/DiI C18-5DS | 108 | 0.13 | 7 | −2.45 | 8.23 |
| EXP22020613-NKM | Lipid 40/GFP/DiI C18-5DS | 94.4 | 0.04 | 5.46 | −2.34 | 8.05 |

### Example 4. Preparation of VHH Conjugates to Enable M2 Macrophage Targeting

An anti-CD206 VHH targeting moiety was conjugated to DSPE-PEG(2k)-maleimide via covalent coupling between the maleimide group and a thiol functionality of a C-terminal cysteine residue on the protein. The protein (3-4 mg/mL), after buffer exchange into pH 7.4 phosphate buffered saline (PBS 137 mM NaCl, 2.7 mM KCl, 10 mM Na₂HPO₄, and 1.8 mM KH₂PO₄) with 5 mM ethylenediaminetetraacetic acid (EDTA), was reduced using 2 mM Tris(2-carboxyethyl)phosphine hydrochloride (TCEP) for 1 hour at room temperature. The reduced protein was isolated using a 7 kDa molecular weight cutoff (MWCO) SEC column to remove TCEP and buffer exchanged into fresh PBS with 5 mM EDTA.

The conjugation reaction was initiated by addition of a 10 mg/mL micellar suspension of DSPE-PEG2k-maleimide and 30 mg/mL DSPE-PEG2k4OCH₃ (1:4 mol ratio is used) in PBS. The conjugation reaction is carried out using 2-4 mg/mL protein and a 1-2 molar excess of maleimide at 37° C. for 2 hours followed by incubation at room temperature for an additional 12-16 hours.

The production of the resulting conjugate was monitored by HPLC and the reaction quenched in 2 mM cysteine. The resulting conjugate (DSPE-PEG-VHH) is isolated using a 100 kDa or 50 kDa MWCO Millipore Regenerated Cellulose membrane filtration using pH 7.0 HEPES buffer saline (25 mM HEPES, 150 mM NaCl) and stored at 4° C. prior to use. After quenching, the final micelle composition consists of a mixture of DSPE-PEG2k-VHH, DSPE-PEG-maleimide (cysteine terminated), and DSPE-PEG2k-OCH₃. The ratio of the three components is approximately DSPE-PEG-Fab:DSPE-PEG-maleimide (cysteine terminated):DSPE-PEG-OCH₃=1:0-1.5:4-10 (by mol)).

### Example 5. Preparation of LNPs Containing M2 Macrophage Targeting Group

This Example describes the incorporation of an M2 Macrophage targeting conjugate into preformed LNPs.

aCD206 VHH Sequence Identification (SEQ ID 170):

QVQLQESGGGLVQAGGSLRLSCAASGFTDDDYDIGWFRQAPGKERE
GVSCISSSDGSTYYADSVKGRFTISSDNAKNTVYLQMNSLKPEDT
AVYYCAADFFRWDSGSYYVRGCRHATYDYWGQGTQVTVSSTSFVP
VFLPAKPTTTPAPRPPTPAPTIASQPLSLRPEASRPAAGGAVHTR
GLDFAGGCHHHHHH

LNPs from Example 2 and 3 and DSPE-PEG2k-anti-CD206 VHH conjugate (SEQ ID X) (prepared using the methods described in Example 4) were combined in a 15 mL conical tube at a ratio of 0.084 g VHH conjugate per 1 g of mRNA and diluted with MBS (25 mM pH 6.5 MES buffer with 150 mM NaCl) and 49 wt % sucrose solution to a final mRNA concentration of 0.2 mg/mL and sucrose concentration of 10% w/v. The tube was placed into a ThermoMixer pre-heated to 37° C. and then mixed at 300 rpm for 4 hours at 37° C. The resulting targeted LNP suspension was subsequently filtered using a 0.2 μm PES syringe filter and then either used immediately or stored frozen at −80° C. Exemplary targeted LNP properties are shown in Table 4.

TABLE 4

| | | | | Parent LNP Post 1X | | Targeted LNP Post 1X | |
|---|---|---|---|---|---|---|---|
| | | | | Freeze-Thaw cycle | | Freeze-Thaw cycle | |
| | Ionizable Lipid/ | Parent LNP | | (−70 C.) | | (−70 C.) | |
| LNP Lot | mRNA Payload/ | DLS Z-Avg. | DLS | DLS Z-Avg. | DLS | DLS Z-Avg. | DLS |
| Number | Dye | Diameter (nm) | PDI | Diameter (nm) | PDI | Diameter (nm) | PDI |
| EXP22020613- | ALC-0315 | 80.3 | 0.09 | 127.0 | 0.10 | 130.4 | 0.12 |
| NAM | GFP/DiI C18-5DS | | | | | | |
| EXP22020613- | Lipid 15/GFP/DiI | 83.9 | 0.09 | 95.8 | 0.07 | 103.4 | 0.11 |
| NPM | C18-5DS | | | | | | |
| EXP22020613- | Lipid 26/GFP/DiI | 82.2 | 0.05 | 89.5 | 0.08 | 92.2 | 0.12 |
| NUM | C18-5DS | | | | | | |
| EXP22020613- | Lipid 25A/GFP/DiI | 70.7 | 0.04 | 87.6 | 0.05 | 89.6 | 0.06 |
| NFM | C18-5DS | | | | | | |
| EXP22020613- | Lipid 27/GFP/DiI | 83.2 | 0.05 | 90.9 | 0.03 | 94.5 | 0.07 |
| NGM | C18-5DS | | | | | | |
| EXP22020613- | Lipid 28/GFP/DiI | 108 | 0.13 | 185.3 | 0.05 | 348.0 | 0.22 |
| NHM | C18-5DS | | | | | | |
| EXP22020613- | Lipid 40/GFP/DiI | 94.4 | 0.04 | 100.4 | 0.05 | 117.1 | 0.13 |
| NKM | C18-5DS | | | | | | |

### Example 6. Method for Freezing (and Thaw) Process for LNP Suspension and LNP Characterization Post Freeze-Thaw

LNP suspension was mixed with a solution of 49 wt/% sucrose solution in water and additional storage buffer (if needed) to achieve a final sample containing LNPs at approximately 45 pig/mL and sucrose at approximately 9.6 wt/%. Aliquots of approximately 0.05 mL in 1.5 mL centrifuge tubes were then prepared from the final LNP sample containing sucrose. The aliquots were then placed in a −80° C. freezer for at least 2 h to freeze the samples. After freezing, an aliquot was thawed by placing it at room temperature for at least 10 min. The aliquot was then mixed by vortexing at 2500 rpm for approximately 5 s. The thawed material was then analyzed for size by DLS as described in Example 3.

### Example 7. Physiochemical Properties of LNPs Based on Lipids 15, 26, 25A, 27, 28, 40 and Comparator Lipid ALC-0315

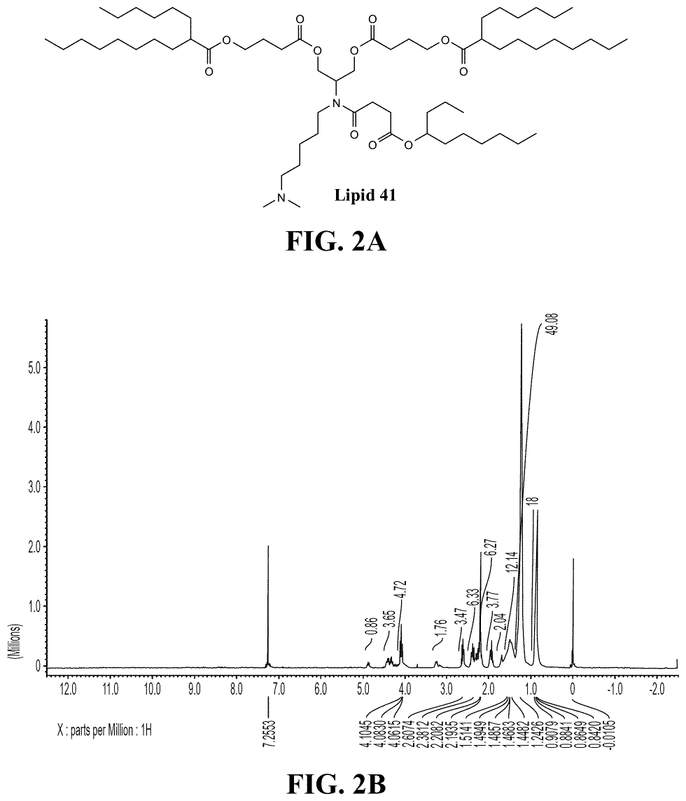

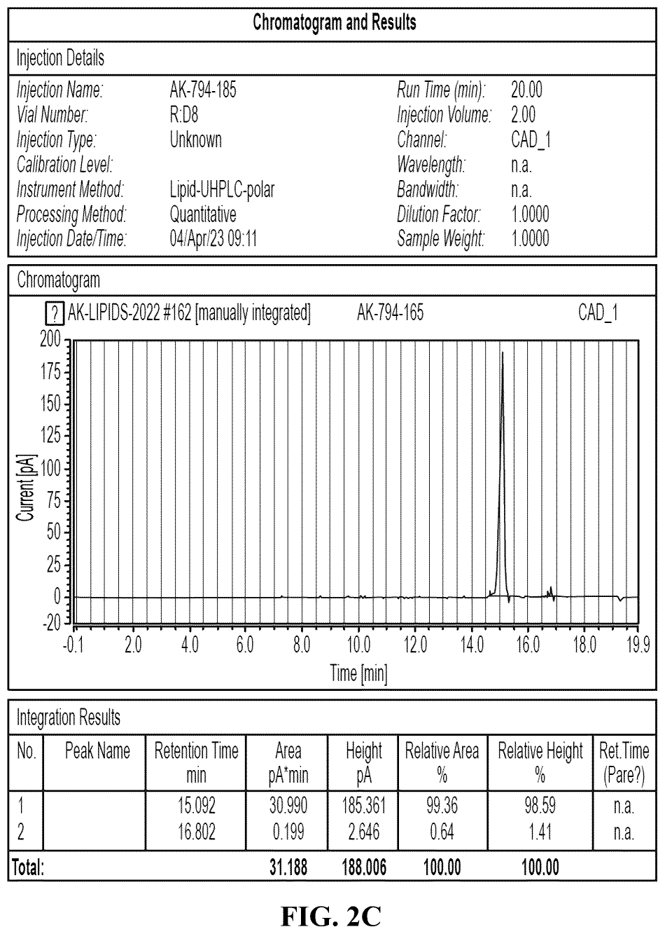

Lipids 15, 26, 25A, 27, 28, 40 and comparator lipid ALC-0315 encapsulating GFP-mRNA (TriLink Biotechnologies Inc.) were prepared and characterized using methods described in Examples 2 to 6 (LNP characterization data is shown in Table 3, Example 3, and Table 4, Example 5 above). As seen in FIG. 2A, the initial mixing step and subsequent buffer exchange into pH 6.5 MBS resulted in LNP sizes ≤100 nm (DLS) with all the lipids tested except lipid 28 that resulted in larger LNP diameters (108 nm). In all cases LNP diameter increased upon 1× Freeze-thaw cycle, however, most lipids (except ALC-0315 and Lipid 28) retained particle diameters below 100 nm. As seen in FIG. 2B, the polydispersity index (DLS) in all cases remained below 1.15 both pre- and post-1× freeze-thaw cycle. Notably, as seen in FIG. 2C, the LNP charge properties of lipids 25A, 26, 27, 28, and 40 exhibited a distinct negative charge at physiological pH (7.4); and a relative week position charge at pH 5.5, unlike Lipid 15. Without wishing to be bound by any particular theory, this characteristic charge may be attributed to the tail group chemistry and could impact LNP biodistribution. Furthermore, comparing the charge properties of Lipid 26 and Lipid 40 derived LNPs, a shift to more positive charge under acidic pH (5.5) conditions is observed for Lipid 40 LNPs. Without wishing to be bound by any particular theory, this shift toward more positive Zeta Potential (DLS) values may be attributed to the additional methylene (—CH₂—) in Lipid 40 head group relative to Lipid 26. The additional methylene (—CH₂—) was designed to impart stronger basic character to the tertiary amine moiety of Lipid 40 and thereby shift the LNP Apparent pKₐ upwards. Such shift in LNP apparent pKₐ may impart a more positive charge under acidic endosomal pH conditions and improve the ability for endosomal escape by enhancing LNP fusion with endosomal membranes. As seen in FIG. 2D, all lipids tested resulted in high GFP mRNA encapsulation efficiency (<15% Dye accessible RNA) and >90% RNA recovery). In summary, all lipids tested resulted in viable mRNA encapsulation, LNP diameters <110 nm and good freeze-thaw stability.

### Example 8. Physiochemical Properties of Targeted LNPs Based on Lipids 15, 26, 25A, 17, 28, 40 and Comparator Lipid ALC-0315 Synthesized by Post-Insertion of αCD206 VHH Lipid Conjugate

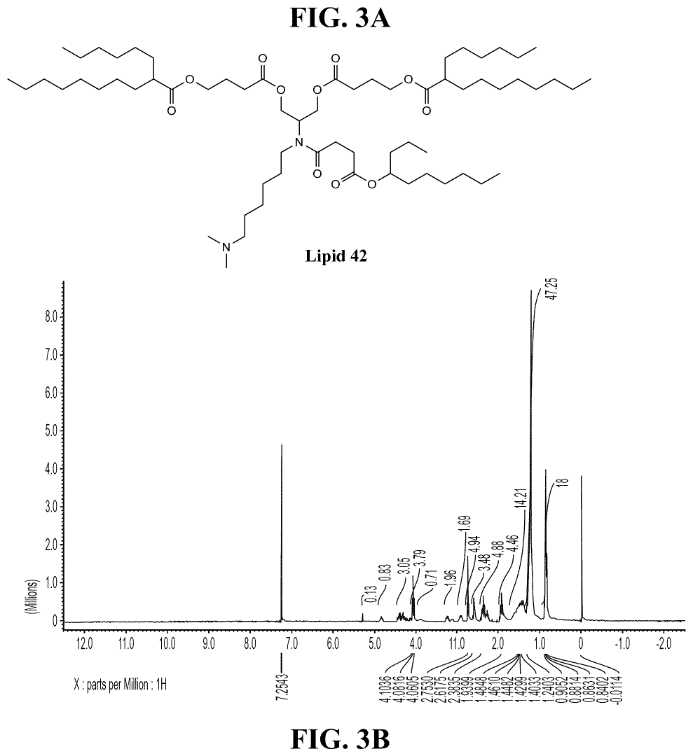

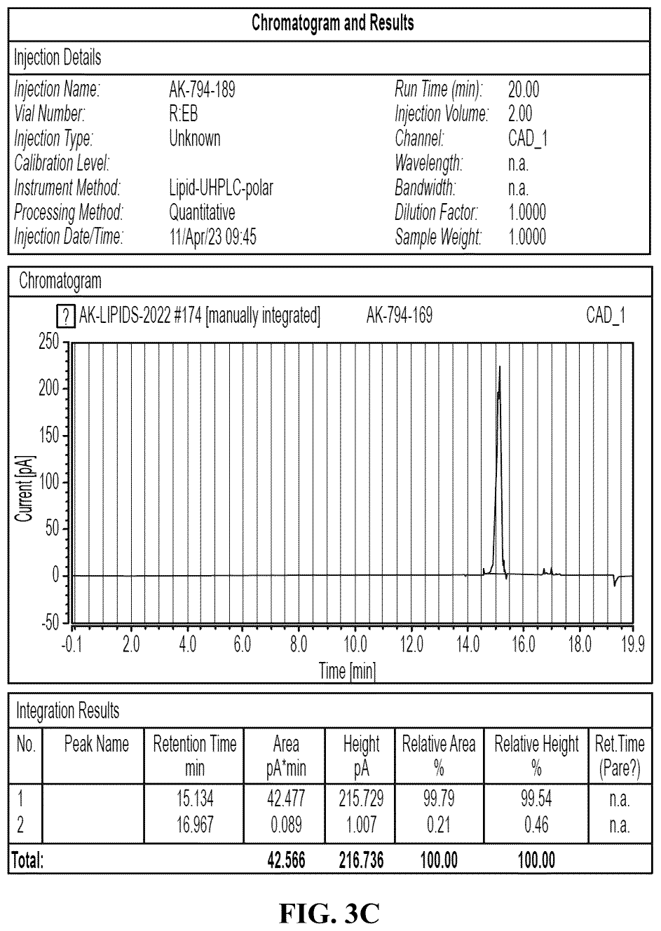

Parent LNPs produced using methods described in Example 2 and αCD206 VHH conjugate produced using methods described in Example 4 were used to produce αCD206 targeted LNPs using methods described in Example 5. This example describes the physiochemical properties of αCD206 targeted LNPs. As seen in FIG. 3A, the hydrodynamic diameter (DLS) of targeted LNPs based on Lipids 15, 25A, 26, 27, 28 and 40 are below 120 nm. Lipid ALC-0315 resulted in slight larger targeted LNPs (˜130 nm) and Lipid 28 resulted in significantly larger targeted LNPs upon insertion of the αCD206 VHH conjugated (>350 nm). As seen in FIG. 3B, the polydispersity index (DLS) of all lipids (except lipid 28) remained below 1.15 suggesting no significant change in the size distribution of targeted LNPs relative the non-targeted parent particles. Furthermore, the targeted LNPs remained stable to 1× Freeze-Thaw cycle based on the LNP diameter and PDI (DLS) measurements pre- and post 1× Freeze-Thaw cycle.

### Example 9. Physiochemical Properties of LNPs Based on Lipids 15, 17A, 18A, 19A, 21A, 20A, 46 and 40

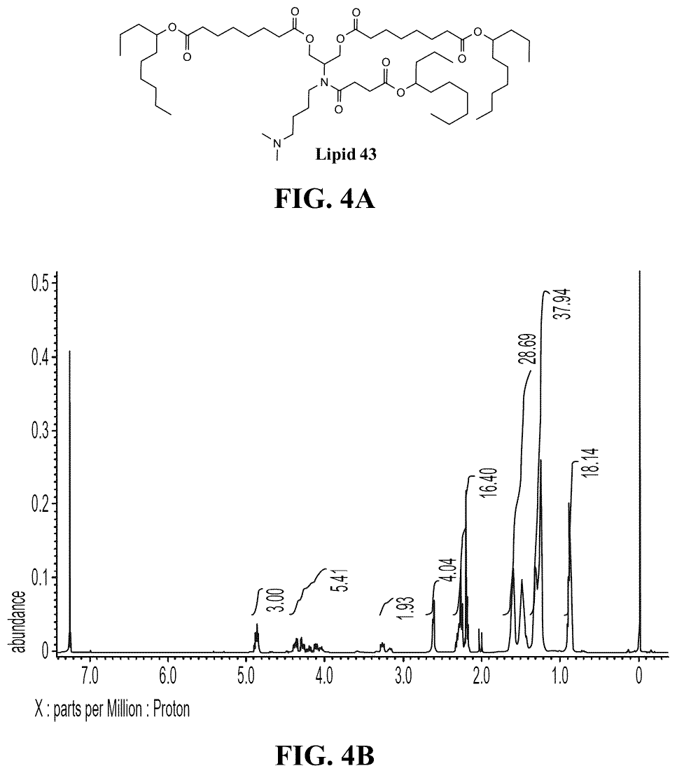

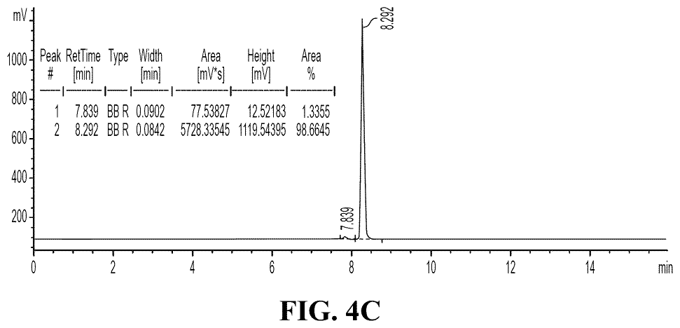

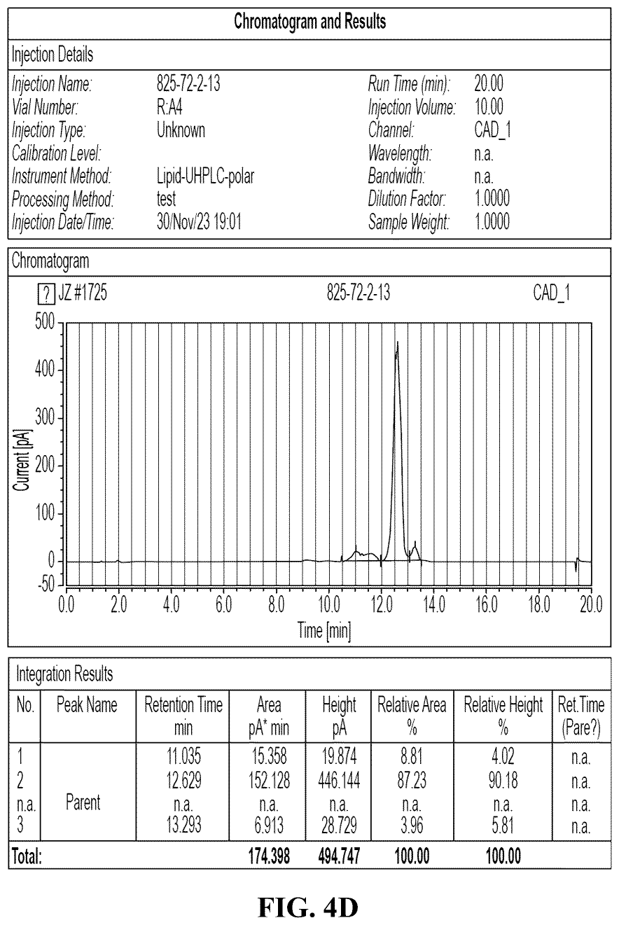

LNPs based on Lipids 15, 17A, 18A, 19A, 21A, 20A, 46 and 40, encapsulating GFP-mRNA (TriLink Biotechnologies Inc.) were prepared and characterized using methods described in Examples 2 to 6. As seen in FIG. 4A, the initial mixing step and subsequent buffer exchange into pH 6.5 MBS resulted in LNP sizes ≤120 nm (DLS) with all the Lipids tested except lipids 17A and 18A that resulted in larger LNP diameters (˜150 nm). In all cases LNP diameter decreased slightly upon 1× Freeze-thaw cycle. As seen in FIG. 4B, the polydispersity index (DLS) in all cases remained below 1.15 both pre- and post-1× freeze-thaw cycle, suggesting no significant change of LNP size distribution under the stress of 1× freeze-thaw cycle. Notably, as seen in FIG. 4C, lipid 46 exhibited a weak negative charge at physiological pH (7.4); and a relative strong positive charge at pH 5.5, similar to the charge properties of Lipid 15. In contrast, Lipid 20A LNPs exhibit a relatively strong negative charge at physiological pH (7.4) and a weak positive charge at pH 5.5. Comparing the charge properties of Lipid 20A and Lipid 46 derived LNPs, a shift to more positive charge under acidic pH (5.5) conditions is observed for Lipid 46 LNPs. Without wishing to be bound by any particular theory, this shift toward more positive Zeta Potential (DLS) values may be attributed to the additional methylene (—CH₂—) in Lipid 46 head group relative to Lipid 20A. The additional methylene (—CH₂—) was designed to impart stronger basic character to the tertiary amine moiety of Lipid 46 and thereby shift the LNP Apparent pKₐ upwards. Such shift in LNP Apparent pKₐ may impart a more positive charge under acidic endosomal pH conditions and improve the ability for endosomal escape by enhancing LNP fusion with endosomal membranes. As seen in FIG. 4D, all lipids resulted in high GFP mRNA encapsulation efficiency (<15% Dye accessible RNA) and >90% RNA recovery). In summary, all lipids tested resulted in viable mRNA encapsulation and freeze-thaw stability. However, as seen in FIG. 4A, Lipid 17A and 18A resulted in relatively large LNP diameter (140-150 nm) LNP diameters relative to the more hydrophobic Lipids in this group, i.e., lipids 19A, 20A, 21A and 46 in this group of lipids.

### Example 10. Method for the Primary Human Monocyte Cell Transfection with GFP/DiI Labelled LNPs

CD14+ Monocytes were isolated from frozen peripheral blood mononuclear cells using a Human Monocyte Isolation Kit from STEMCELL. Monocytes were resuspended after isolation in Macrophage base media (50 mL RPMI+10% HI-FBS with 50 ng/mL of human M-CSF) for M0 Macrophage differentiation. Monocytes were then plated into 6-well plates at 2E6cells/mL. Cells were allowed to rest for 2 days in a 37° C. incubator, 5% CO₂, and then media was changed. On day 5, media was removed from the wells and 2 ml of cold PBS+10 mM EDTA was added per well. The 6 well plates were incubated for 10 minutes on ice to detach the macrophages. The macrophages were then gently scraped from each well using a silicone scraper and the scraped cells were transferred to a tube with Macrophage base media. The cells were counted and resuspended at 200,000 cells/mL in M2 polarization media (50 mL RPMI+10% HI-FBS with 50 ng/mL human M-CSF and 10 ng/mL human IL-4). The cells were then plated in a flat-bottom 96-well plate at 20,000 cells per well. Cells were incubated in a 37° C. incubator, 5% CO₂ for 24 hrs. The next day cells were transfected by gently adding 10 uL of a 11 ug/mL (by mRNA) nanoparticle suspension, resulting in a final mRNA concentration of 1 μg/mL (unless otherwise noted). Cells were gently mixed with a pipette and then incubated for 24 hours in a 37° C. incubator, 5% CO₂. After incubation, the cells were scraped and diluted with FACS buffer (BD 554657) and analyzed using a Novocyte Penteon Flow Cytometer (Agilent). Data were analyzed using FlowJo software from BD biosciences.

### Example 11. GFP Protein Expression in M2 Macrophages Using LNP Transfection of GFP mRNA Using LNPs Based on Lipids 15, 26, 25A, 27, 28, 40 and Comparator Lipid ALC-0315

This example compares the GFP protein expression resulting from LNP's derived from Lipids 15, 26, 25A, 27, 28, 40 and comparator lipid ALC-0315. Nanoparticles bearing GFP encoding mRNA (and a fluorescent dye label (DiI-C18-5DS)) were produced using the microfluidic mixing and buffer exchange processes described in Example 2 to 6. An aCD206 VHH-conjugate was incorporated into the parent LNPs to obtain the final Antibody targeted LNP formulation using the process described in Example 5. Particles thus produced were tested in vitro in M2 macrophages derived from primary human PBMC's (using protocol described in Example 10 above) to assess reported gene expression.

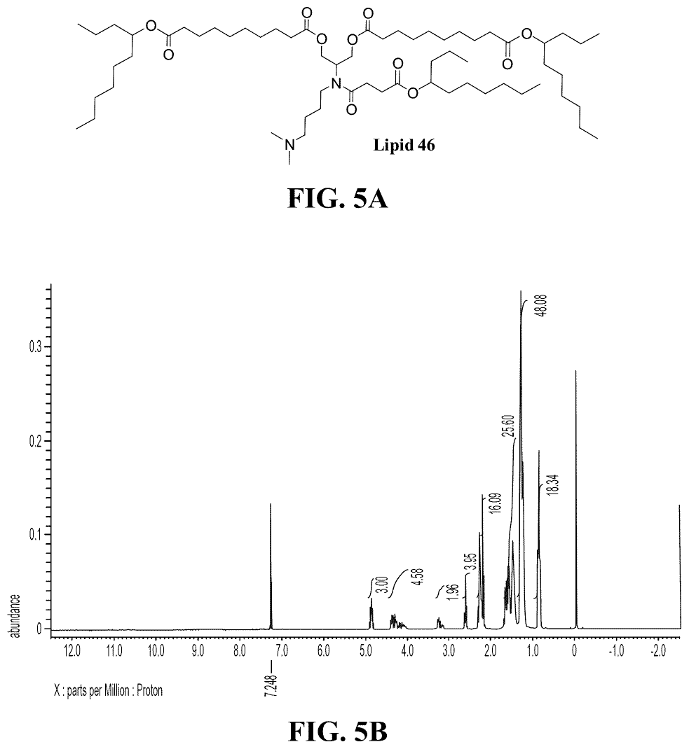

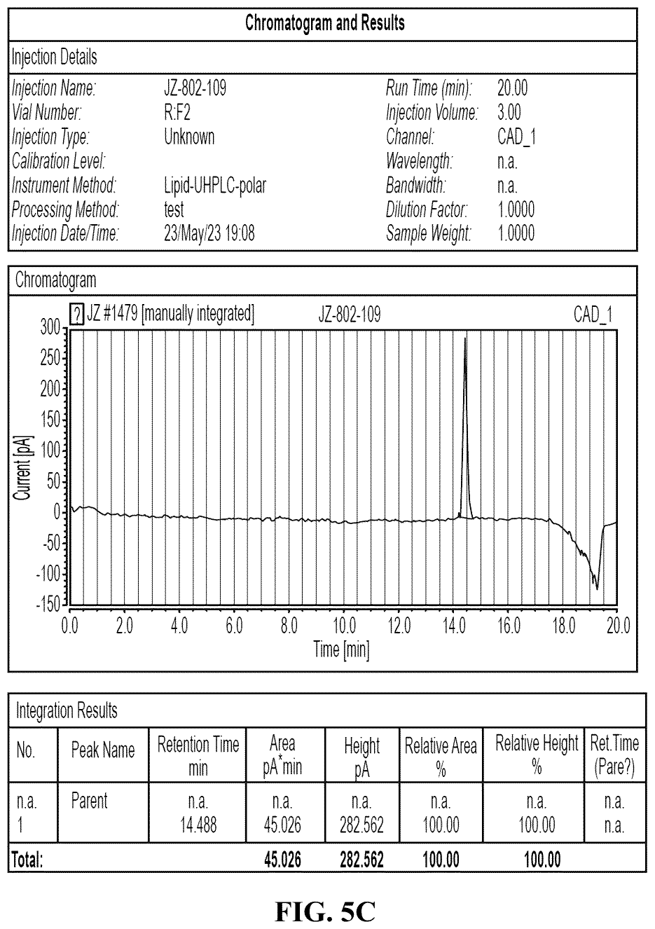

Lipid 25A targeted LNPs resulted in relatively low protein expression as shown by both a lower % GFP+ Macrophages (FIG. 5A) of 50% relative to other lipids that resulted in >80% GFP+ Macrophages. Additionally, comparison of the Mean Fluorescence Intensity (MFI) of the transfected Macrophages (see FIG. 5C) reveals a relatively low level of GFP protein expression in LNPs resulting from Lipids 25A, 26 and 27, possibly suggesting a non-optimal tail group chemistry for either LNP stability in macrophage transfection medium or for fusion of these lipids with endosomal membranes as either of these would limit the cytosolic delivery of the GFP mRNA payload. Notably, GFP protein expression improved significantly with LNPs resulting from Lipids 40 and 28. Comparing of the head group chemistry of Lipid 26 and Lipid 40, the improved performance of Lipid 40 LNPs (relative to Lipid 26 LNPs) is attributed to the additional methylene (—CH₂—) in Lipid 40 head group relative to Lipid 26. The additional methylene (—CH₂—) was designed to impart stronger basic character to Lipid 40 and thereby improve the ability for endosomal escape by enhancing the fusogenicity of Lipid 40 LNPs under acidic endosome pH conditions. Comparing of the head group chemistry of Lipid 27 and Lipid 28, the improved performance of Lipid 28 LNPs (relative to Lipid 26 LNPs) may be attributed to the 3-octanol derived side chain versus the 4-decanol derived side chain (of Lipid 27). However, since there are significant differences between the average diameters (DLS) of Lipid 27 LNPs (˜120 nm) and Lipid 28 LNPs (>350 nm), the observed change GFP expression levels may be the result of other factors (beyond changes to lipid efficiency resulting from improve endosomal escape).

### Example 12. GFP Protein Expression in M2 Macrophages Using LNP Transfection of GFP mRNA Using LNPs Based on Lipids 15, 17A, 18A, 19A, 21A, 20A, 46, and 40

This example compares the GFP protein expression resulting from LNP's derived from Lipids 15, 17A, 18A, 19A, 21A, 20A, 46 and 40. Nanoparticles bearing GFP encoding mRNA (and a fluorescent dye label (DiI-C18-5DS)) were produced using the microfluidic mixing and buffer exchange processes described in Example 2 to 6. An aCD206 VHH-conjugate was incorporated into the parent LNPs to obtain the final Antibody targeted LNP formulation using the process described in Example 5. Particles thus produced were tested in vitro in M2 macrophages derived from primary human PBMC's (from two human donors—Donor 108 and Donor 282—and using protocol described in Example 10 above) to assess reported gene expression. In this experiment, both non-targeted parent LNPs and aCD206 targeted LNPs were tested in vitro in M2 macrophages.

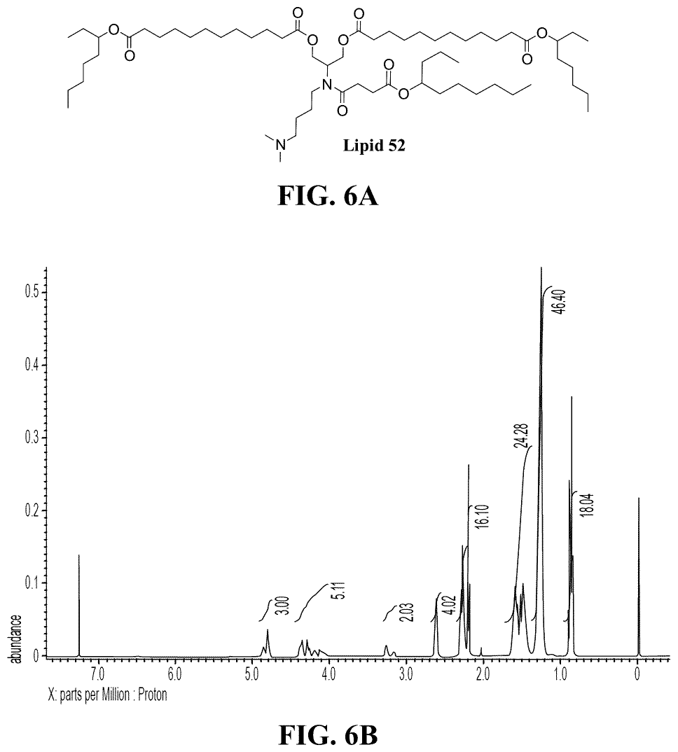

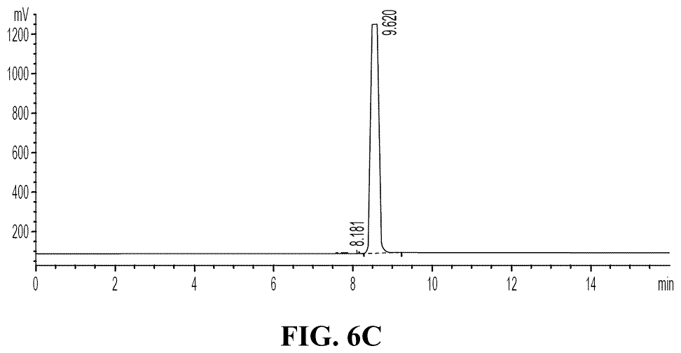

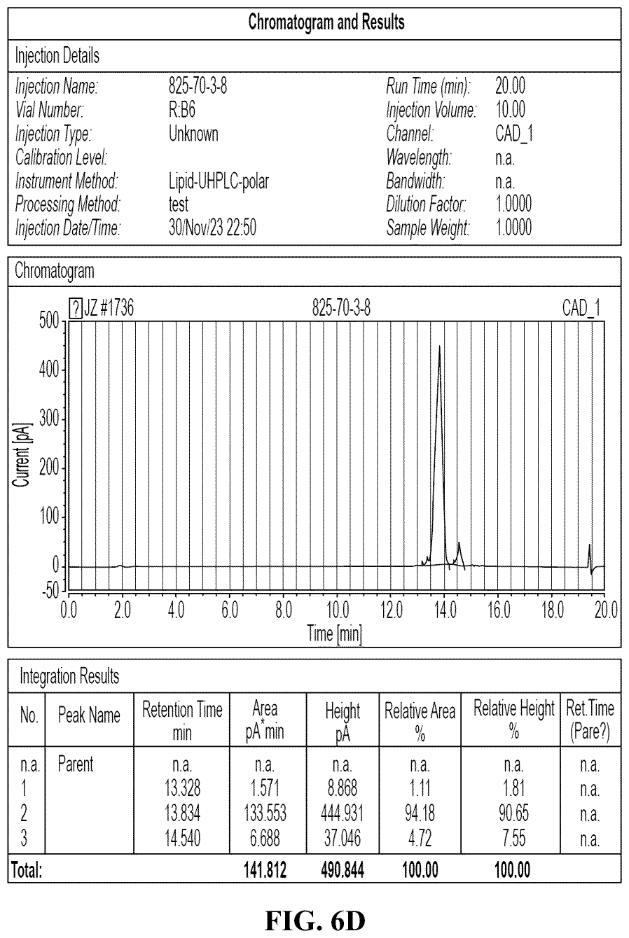

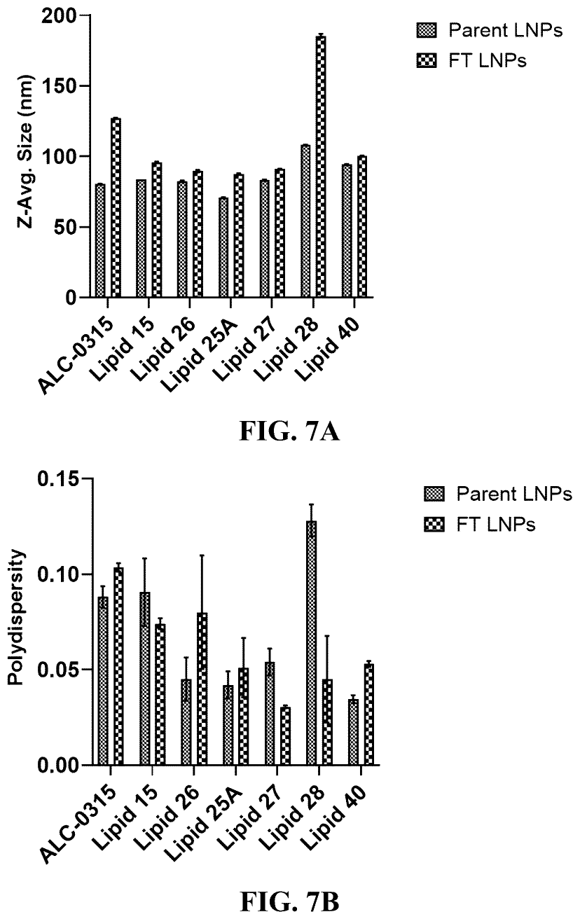

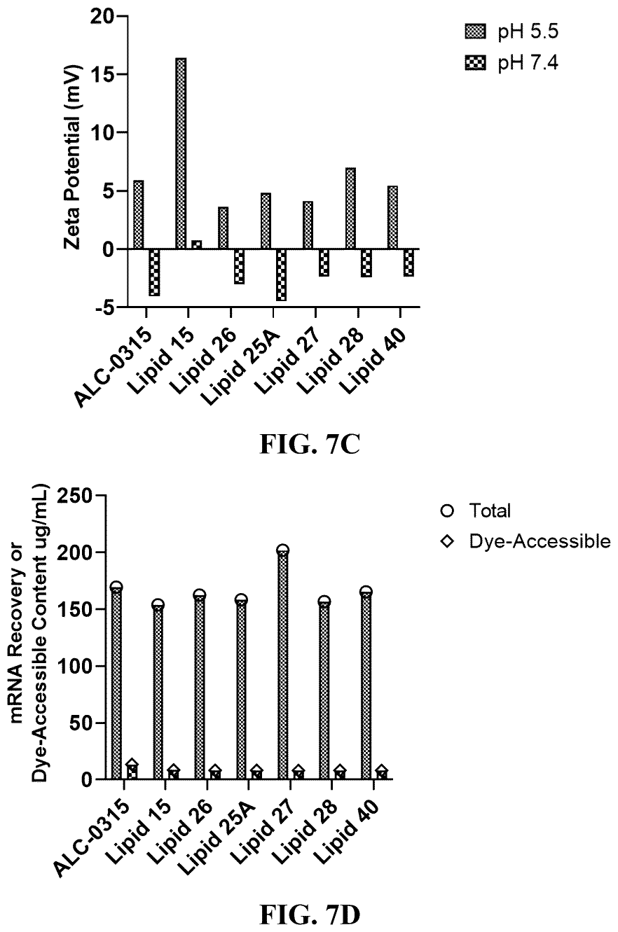

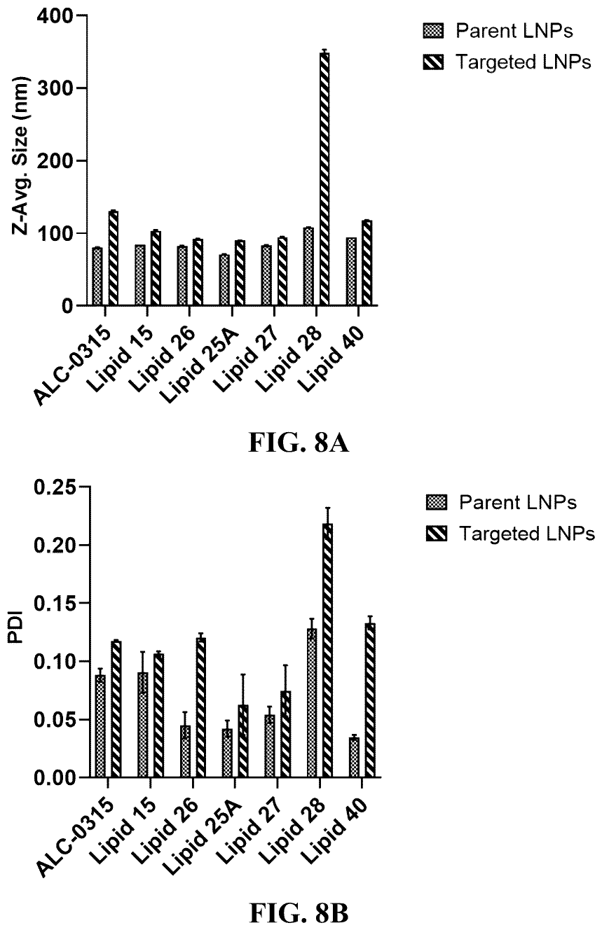

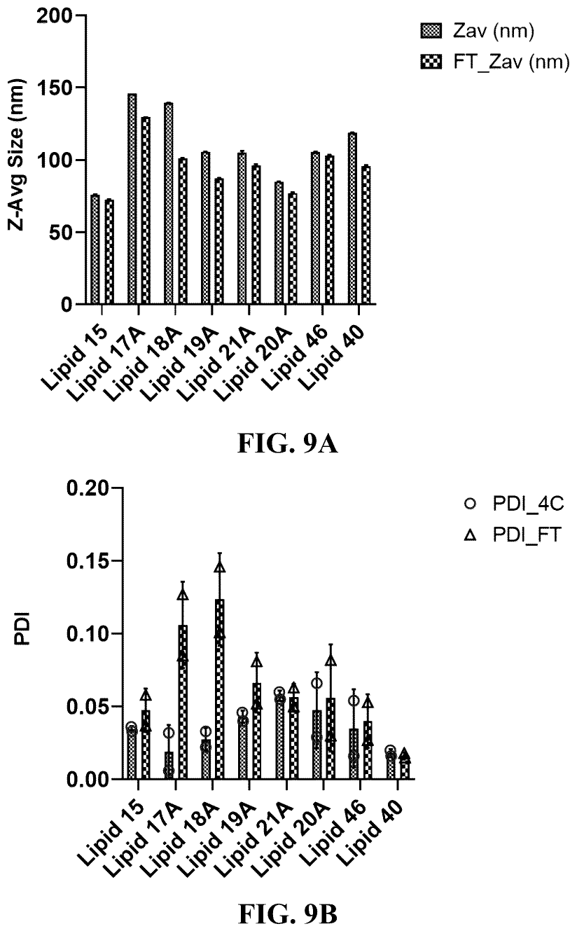

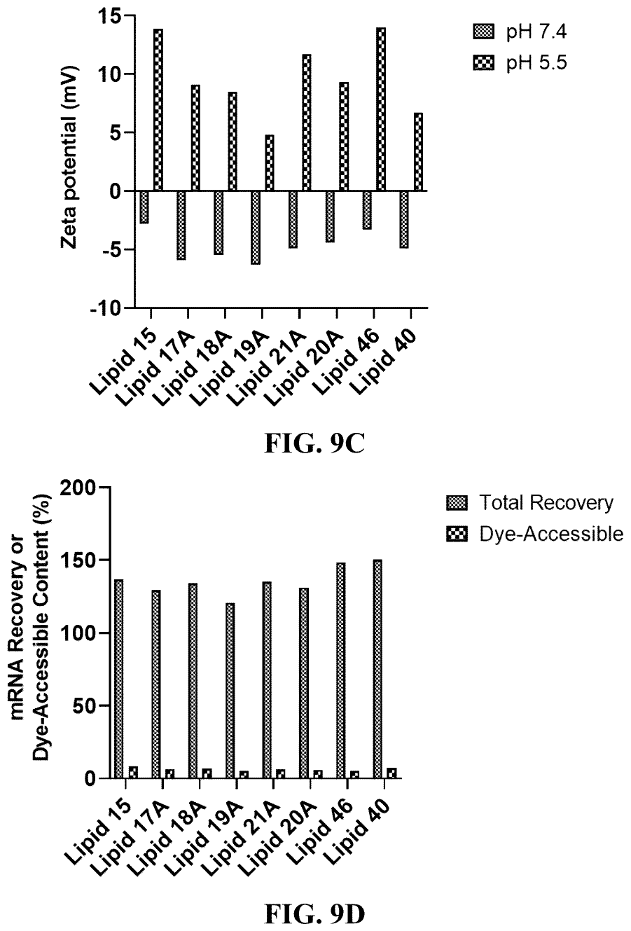

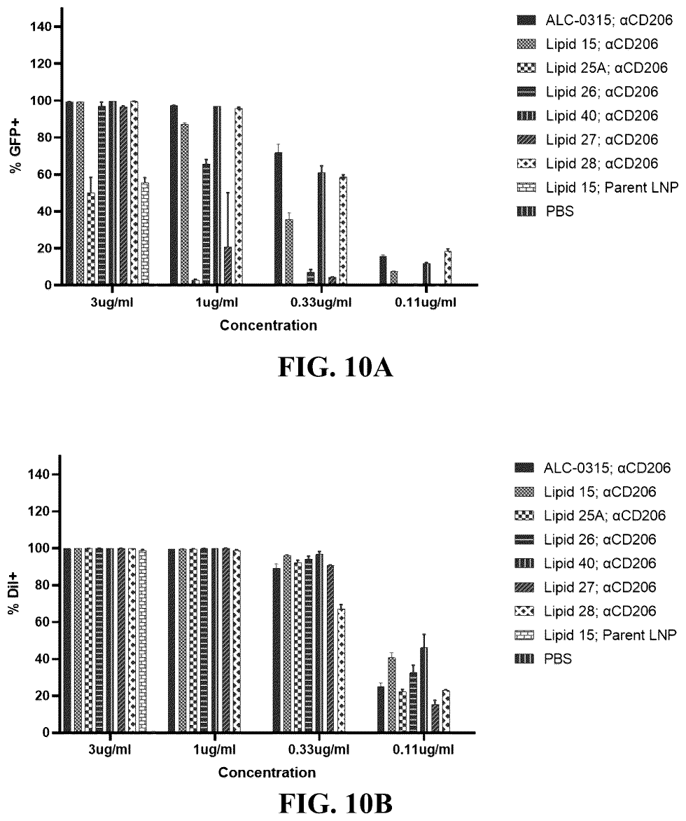

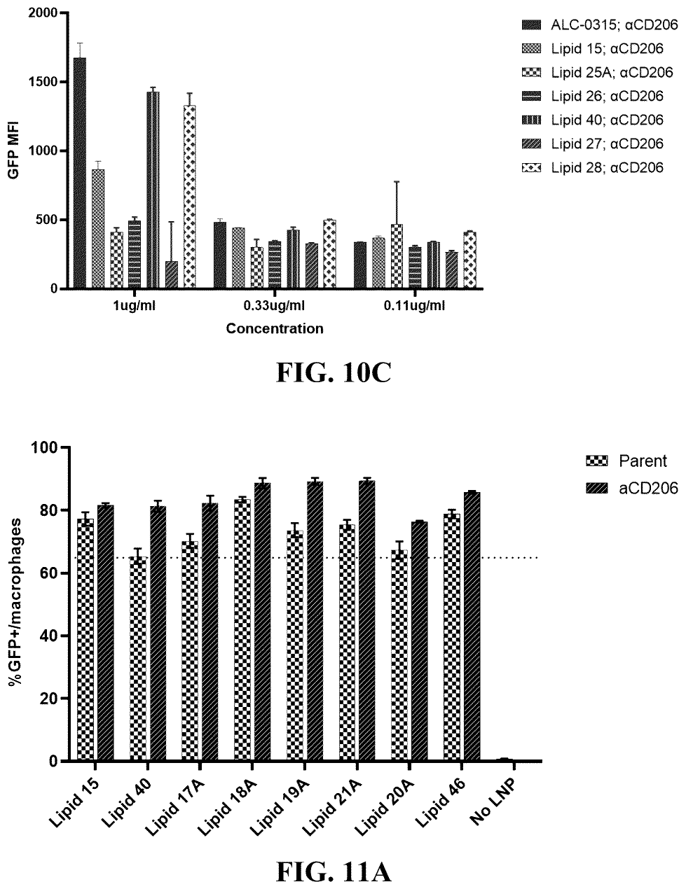

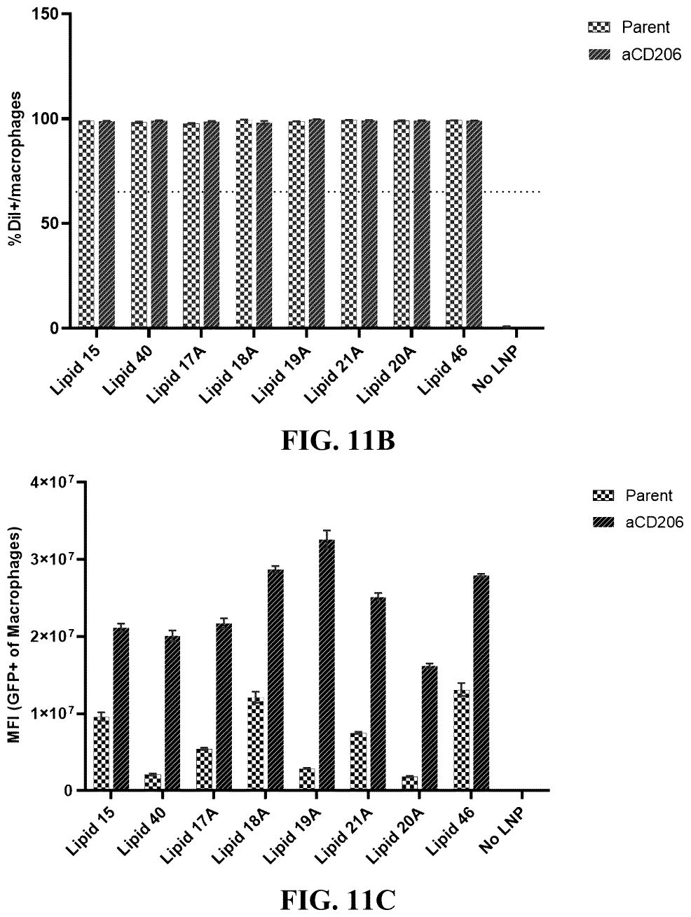

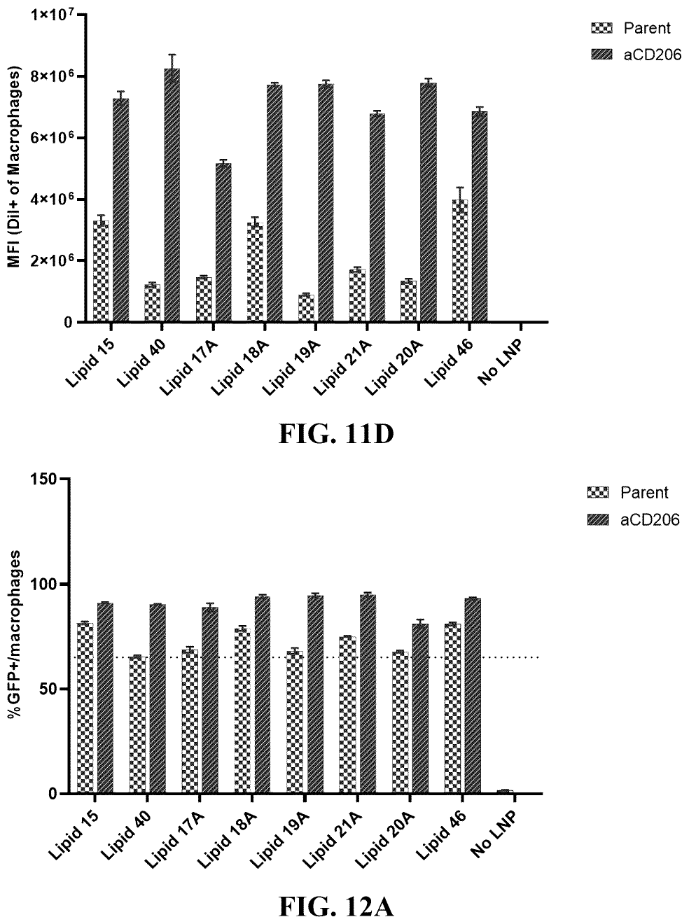

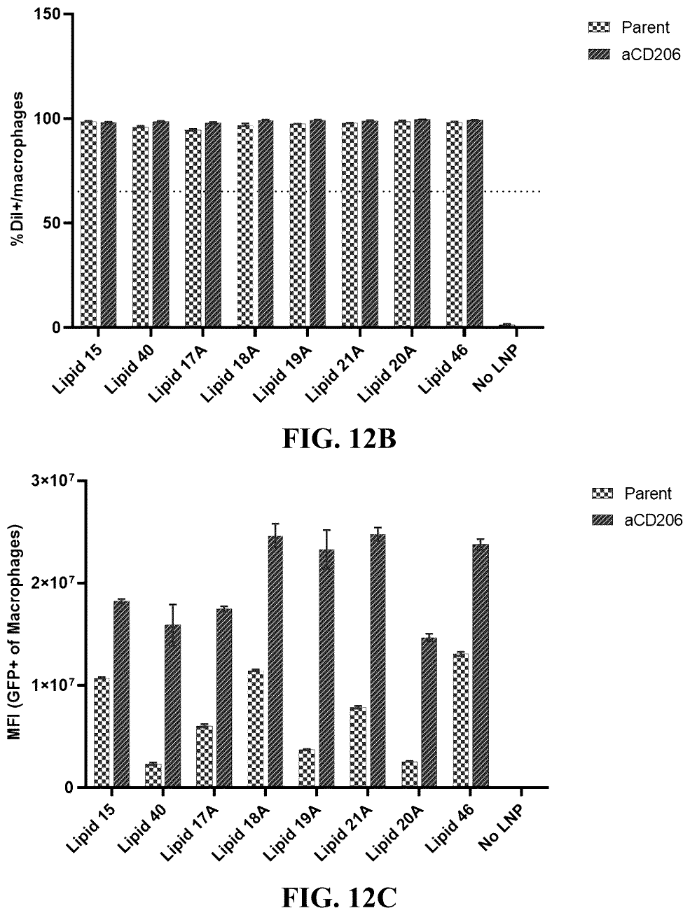

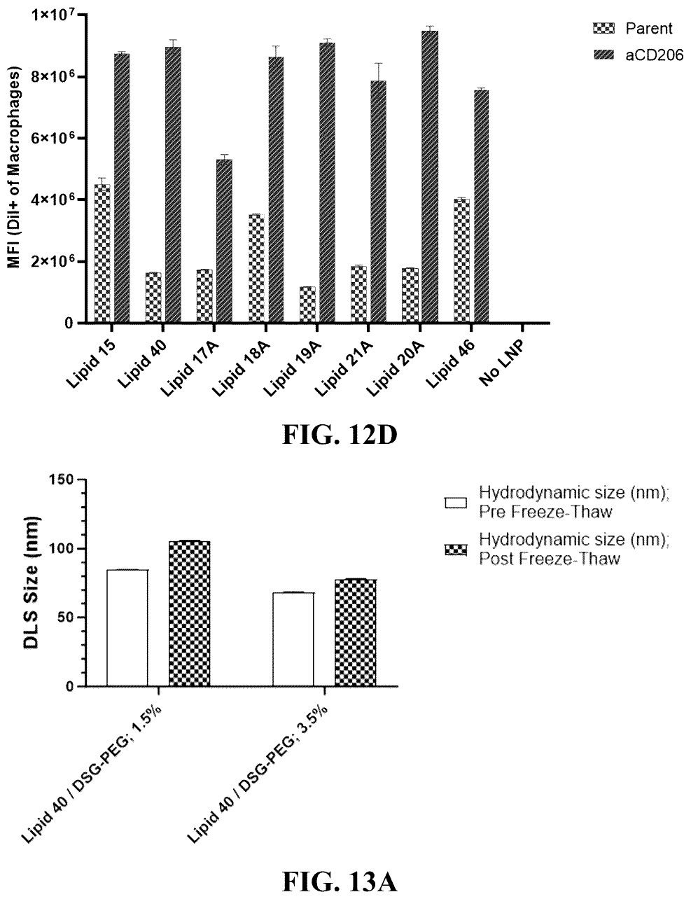

LNPs derived from all of the lipids in this group showed widespread expression in the macrophage population with >60% cells being GFP+ at the 1 μg/mL dose level in both donors 108 and 282 (FIGS. 6A and 7A); however relative to the non-targeted parent LNPs, the targeted LNPs transfected slightly higher percentage of Macrophages suggesting greater access to the cytosolic compartment via the aCD206 targeted endosomal pathway relative to uptake via phagocytosis.

As seen in FIGS. 6C and 7C, significant differences in the Mean Fluorescence Intensity (MFI) of the transfected Macrophages was observed among the parent LNPs derived from this group of lipids. Lipids 40, 19A and 20A resulted in the lowest levels of expression, while Lipids 15, 18A and 26 resulted in the highest levels of GFP protein expression with the parent LNPs. In this group, a 2-to-11-fold difference between protein expression was observed between non-targeted LNPs and aCD206 targeted LNPs (2 fold with Lipids 15 and 46; 11 fold with Lipid 19A) suggesting differences between the propensity for phagocytosis with Lipids 15 and 46 chemistries resulting in greater uptake and cytosolic delivery of non-targeted LNPs.

Comparison of GFP protein expression achieved with targeted LNPs (see FIGS. 6C and 7C) derived from Lipids 17A, 18A, 19A and 20A, suggests the optimal tail group chemistry for efficient cytosolic access and cytosolic release of mRNA is derived from suberic (octanedioic) acid relative to both less hydrophobic (succinic and adipic acids) and more hydrophobic (sebacic acid) tail groups.

Comparing of the head group chemistry of Lipid 20A and Lipid 46, the improved performance of Lipid 46 LNPs (relative to Lipid 26 LNPs) is attributed to the additional methylene (—CH2-) in Lipid 46 head group relative to Lipid 20A. The additional methylene (—CH2-) group was designed to impart stronger basic character to Lipid 46 and thereby improve the ability for endosomal escape by enhancing the fusogenicity of Lipid 46 LNPs under acidic endosome pH conditions. Hence, despite non-optimal tail group chemistry (sebacic acid derived), significant improvement in protein expression could be achieved via the change in head group chemistry and charge properties of Lipid 46 LNPs.

TABLE 5
Lipids for comparison

| | |
|---|---|
| 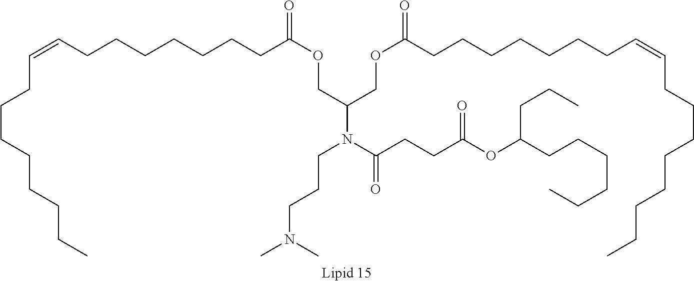 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC] | (Lipid 15) |
| 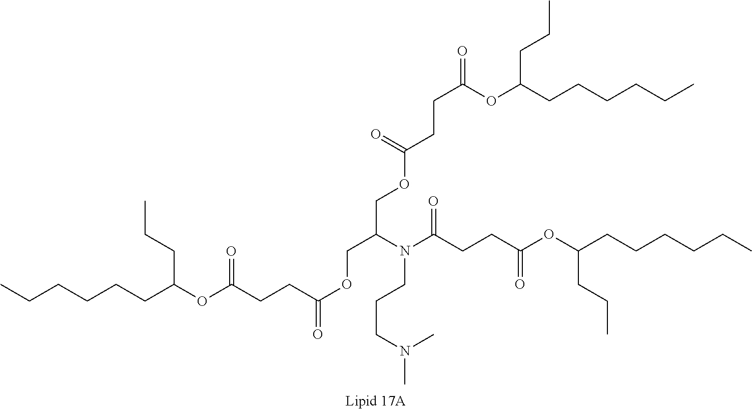 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC] | (Lipid 17A) |
| 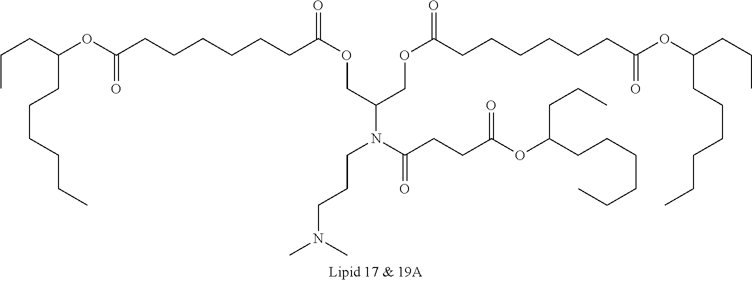 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC] | (Lipids 17 and 19A) |
| 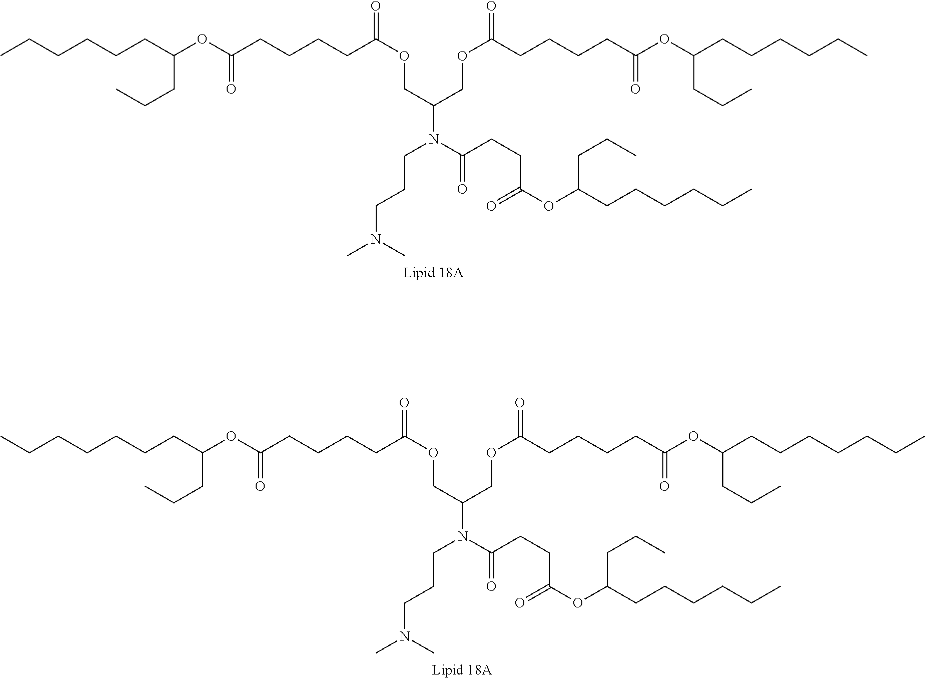 | (Lipid 18A) |
| 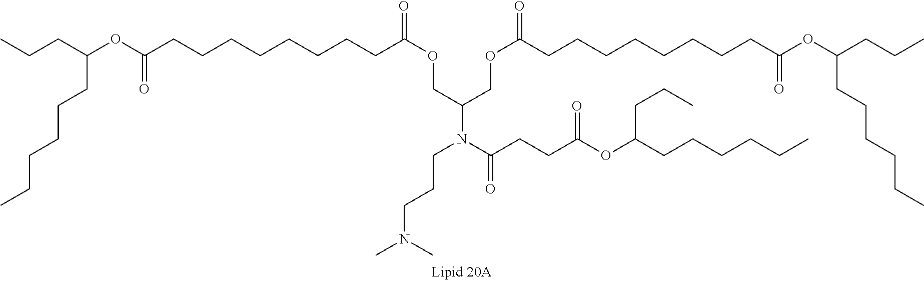 [SMILES: CCCCCCC(CCC)OC(=O)CCCCCCCCC(=O)OCC(COC(=O)CCCCCCCCC(=O)OC(CCC)CCCCCC)N(CCCN(C)C)C(=O)CCC(=O)OC(CCC)CCCCCC] | (Lipid 20A) |
| 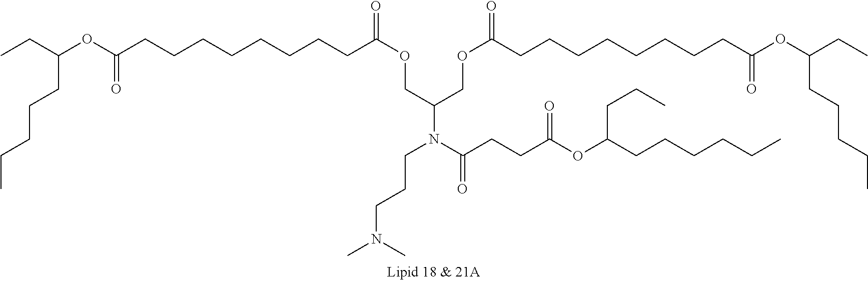 [SMILES: CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC] | (Lipids 18 and 21A) |
| 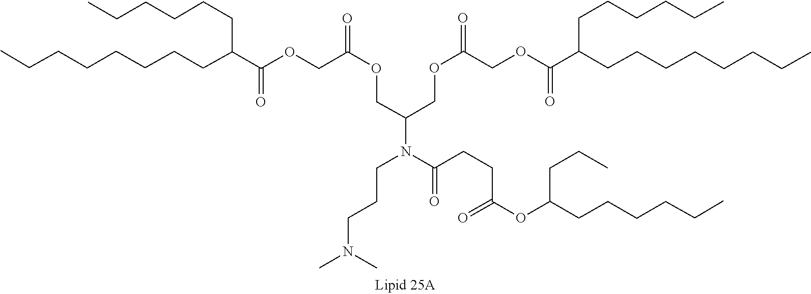 [SMILES: CCCCCCCCC(CCCCCC)C(=O)OCC(=O)OCC(COC(=O)COC(=O)C(CCCCCC)CCCCCCCC)N(CCCN(C)C)C(=O)CCC(=O)OC(CCC)CCCCCC] | (Lipid 25A) |
| 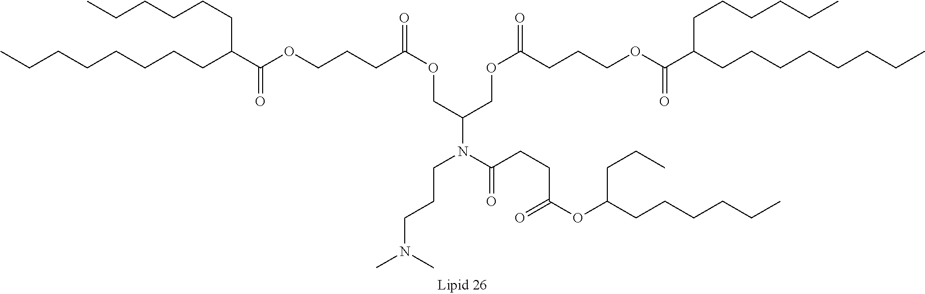 [SMILES: CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC] | (Lipid 26) |
| 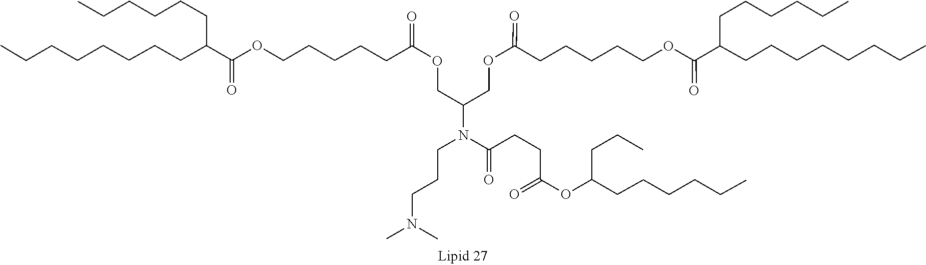 [SMILES: CCCCCCCCC(CCCCCC)C(=O)OCCCCCC(=O)OCC(COC(=O)CCCCCOC(=O)C(CCCCCC)CCCCCCCC)N(CCCN(C)C)C(=O)CCC(=O)OC(CCC)CCCCCC] | (Lipid 27) |
| 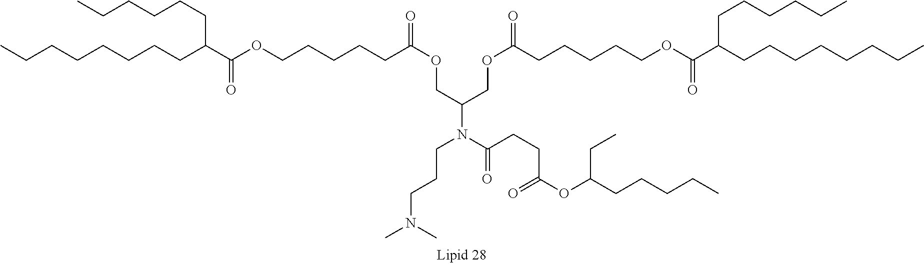 [SMILES: CCCCCCCCC(CCCCCC)C(=O)OCCCCCC(=O)OCC(COC(=O)CCCCCOC(=O)C(CCCCCC)CCCCCCCC)N(CCCN(C)C)C(=O)CCC(=O)OC(CC)CCCCC] | (Lipid 28) |

### Example 13. Method for Formulation of mCherry-LNPs Based on Lipid 40, DPG-PEG or DSG-PEG Lipid

This Example describes the production of mCherry mRNA-loaded LNPs using exemplary materials, a microfluidic mixing process, and centrifugal filtration methods for purification and buffer exchange. The lipid solution composition, compositions of mCherry mRNA solution buffer and final LNP buffers are as described in Example 1 and detailed in Table 3. Exemplary mCherry LNP batch information is provided in Table 6.

TABLE 6
Composition of mCherry-mRNA Lipid 40 LNPs

| | | | | Mole % Lipid compositions |
|---|---|---|---|---|
| | Nominal | | | (Ionizable |
| | Batch | Ionizable | | Lipid:Cholesterol:DSPC:PEG- |
| # | Size (ug) | Lipid | PEG-Lipid* composition; | Lipid:DiI-C18(5)-DS |
| 1 | 600 | Lipid 40 | DSG-PEG | 49.2:39.4:9.8:1.5:0.01 |
| 2 | 600 | Lipid 40 | DSG-PEG (Disteoroylglycerol PEG-2000) | 49.3:37.4:9.8:3.5:0.01 |
| 3 | 600 | Lipid 40 | DSG-PEG (Disteoroylglycerol PEG-2000) | 49.2:37.4:9.8:3.5:0.01 |
| 4 | 600 | Lipid 40 | DPG-PEG (Disteoroylglycerol PEG-2000) | 49.2:37.4:9.8:3.5:0.01 |

*DSG-PEG (Di-steoroylglycerol PEG-2000) or DPG-PEG (Di-palmitoylglycerol PEG-2000)

### Example 14. Physiochemical Properties of mCherry-LNPs Based on Lipid 40 and 1.5 and 3.5 Mol % DSG-PEG Lipid

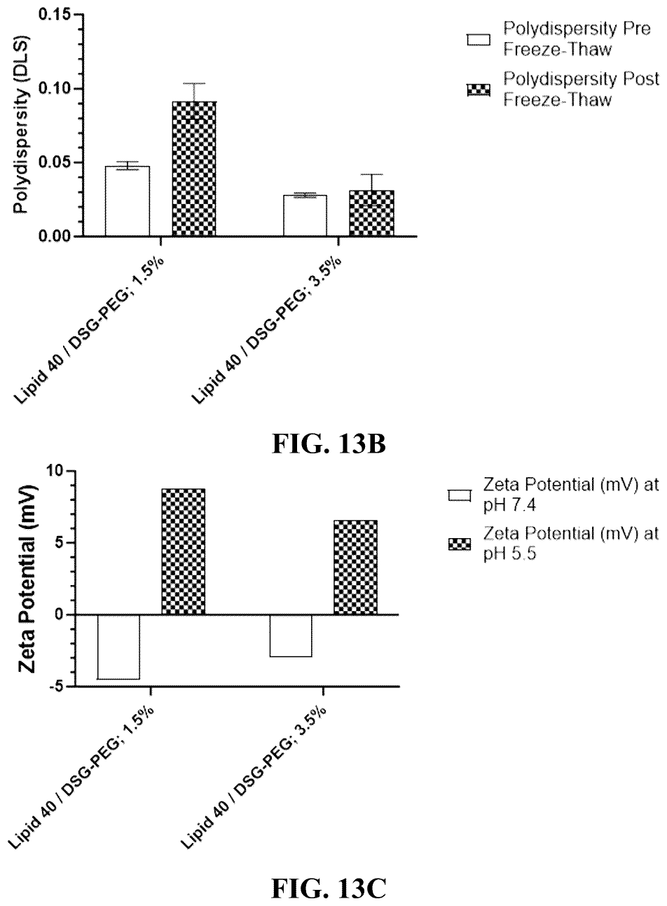

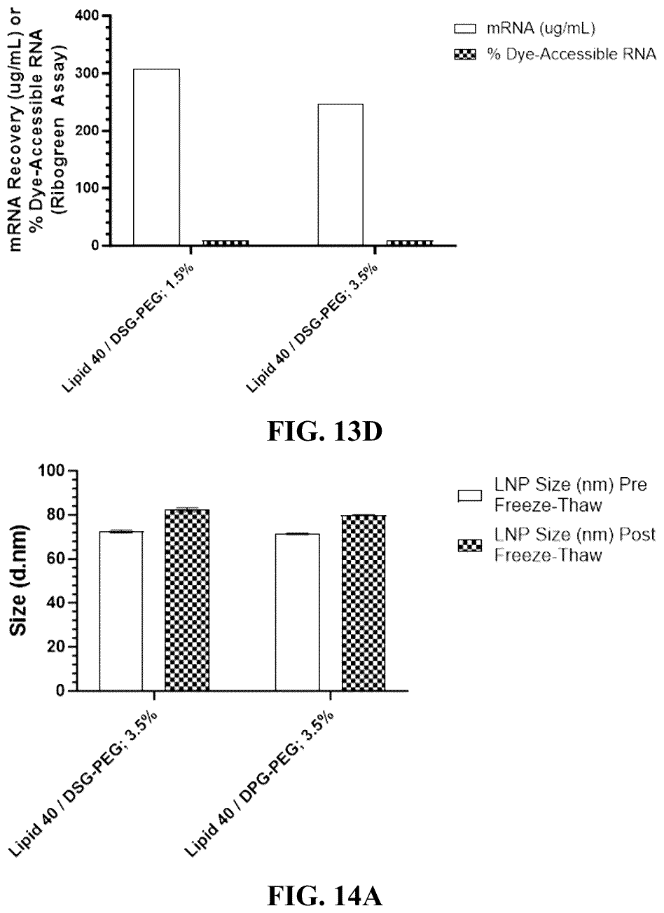

Lipid 40 LNPs encapsulating mCherry-mRNA were prepared and characterized using methods described in Examples 2 to 6. As seen in FIGS. 13A and 13B LNP diameter increased slightly upon 1× Freeze-thaw cycle, and LNP polydispersity index (DLS) remained below 0.10 both pre- and post-1× freeze-thaw cycle. As expected, increasing PEG-lipid density from 1.5 to 3.5 mol % in the LNP composition resulted in drop in LNP size (post 1× Freeze-Thaw) from ˜110 nm to ˜80 nm. As seen in FIG. 13C, the Lipid 40 LNP charge exhibited a distinct negative charge at physiological pH (7.4); and a positive charge at pH 5.5 with both values slightly diminished at the higher 3.5 mole % PEG-lipid density. As seen in FIG. 13D, at both 1.5 and 3.5 mole % PEG-Lipid density, high mCherry-mRNA encapsulation efficiency (<10% Dye accessible RNA) and >90% RNA recovery was observed. In summary, all lipids tested resulted in viable mRNA encapsulation, LNP diameters <110 nm and good freeze-thaw stability.

### Example 15. Physiochemical Properties of mCherry-LNPs Based on Lipid 40 and 3.5 Mol % DPG-PEG and DSG-PEG Lipids

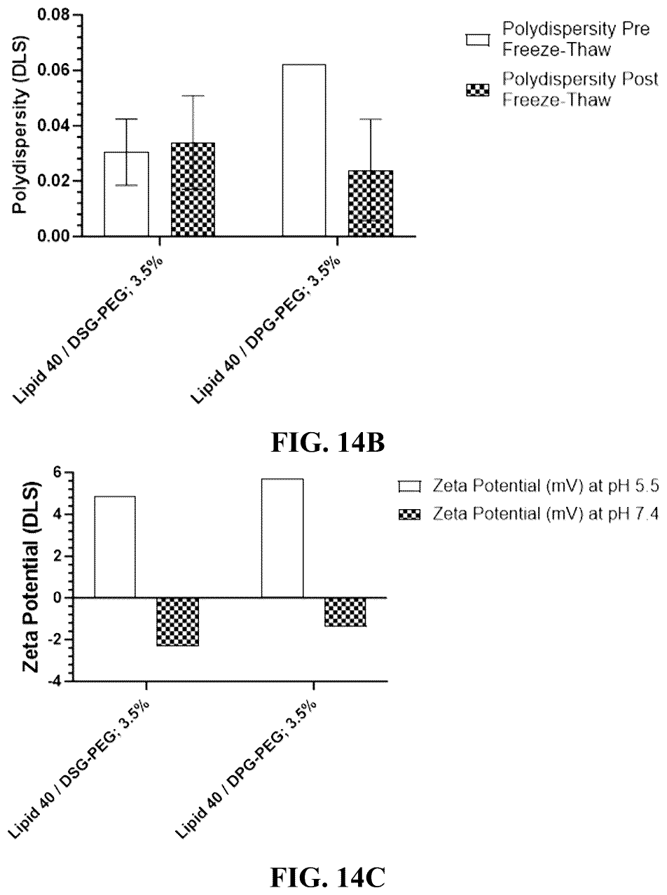

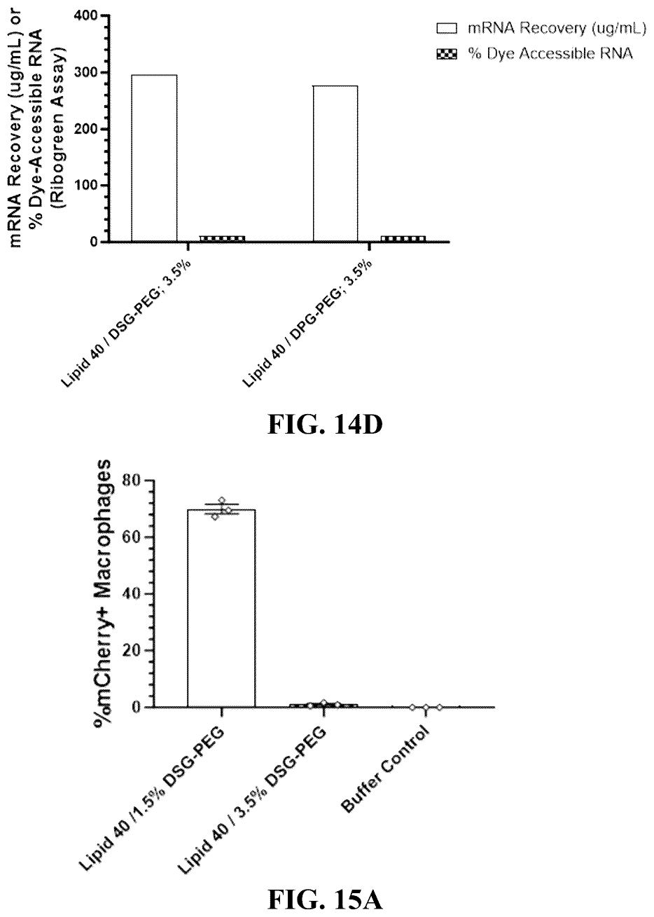

Lipid 40 LNPs encapsulating mCherry-mRNA were prepared and characterized using methods described in Examples 2 and 3. As seen in FIGS. 14A and 14B LNP diameter increased slightly upon 1× Freeze-thaw cycle, and LNP polydispersity index (DLS) remained below 0.10 both pre- and post-1× freeze-thaw cycle. As expected, at 3.5 mol % PEG-lipid in the LNP composition, both lipid anchor chemistries (Di-palmitoyl and Di-stearoyl) resulted in similar LNP sizes (post 1× Freeze-Thaw) of ˜80 nm. As seen in FIG. 14C, the Lipid 40 LNP charge exhibited a slightly negative charge at physiological pH (7.4); and a positive charge at pH 5.5. As seen in FIG. 14D, both PEG-Lipid chemistries (DPG- and DSG-) resulted in high mCherry-mRNA encapsulation efficiency (<10% Dye accessible RNA) and >90% RNA recovery.

### Example 16. Method for Formulation and Storage of Hemagglutinin mRNA-LNPs Based on Ionizable Lipids 40, 46, ALC-0315, SM-102& CL-1191 and 1.5 Mol % DMG-PEG Lipid

An ethanolic mixture of PEG-lipid, ionizable lipid, cholesterol, and DSPC was vigorously mixed with an aqueous solution of mRNA in an acidic buffer, with a fixed nitrogen/phosphate (N/P) ratio (shown in Table 7 below) and under controlled flow rates, to yield a suspension of LNPs. After dialysis/diafiltration and ultrafiltration into a suitable diluent system, the suspension was filtered, diluted to final concentrations, and stored at −80° C. until use. The molar ratios of PEG-lipid:ionizable lipid:cholesterol:DSPC are shown in Table 7 (see Example 17 below).

### Example 17. Methods for Physiochemical Characterization of Hemagglutinin mRNA-LNPs Based on Ionizable Lipids 40, 46, ALC-0315, SM-102 & CL-1191 and 1.5 Mol % DMG-PEG Lipid

The particle size and polydispersity index (PDI) of the LNPs were characterized using a Dynamic Light Scattering (DLS) Zetasizer (Malvern, UK). The particle size was measured as the intensity-weighted mean hydrodynamic diameter. Quant-iT RiboGreen RNA Assay Kit (Invitrogen) was used to assess mRNA encapsulation efficiency following the manufacturer's instruction. The mRNA encapsulation efficiency (EE) was calculated using the following equation: Encapsulation Efficiency (EE) (%)=(Total mRNA (mg/(mL))−Free mRNA (mg/mL))/(Total mRNA ((mg)/mL))×100%.

TABLE 7
Lipid composition and NP ratios used for formulation of HAI-mRNA
LNPs based on Ionizable Lipids 40, 46, SM-102, and ALC-0315.

| Formulation | Cationic | Composition | |
|---|---|---|---|
| Lot # | Lipid (CL) | (DMG-PEG2k:CL:Chol:DSPC) | N/P |
| 1 | Lipid 40 | 1.5:50:38.5:10 | 6 |
| 2 | Lipid 46 | 1.5:50:38.5:10 | 6 |
| 3 | SM-102 | 1.5:50:38.5:10 | 5.67 |
| 4 | ALC-0315 | 1.8:47.5:40.7:10 | 6 |

### Example 18. Physiochemical Properties of Hemagglutinin mRNA-LNPs Based on Ionizable Lipids 40, 46, ALC-0315, SM-102 & CL-1191 and 1.5 Mol % DMG-PEG Lipid

Table 8 lists the size (hydrodynamic radius by DLS), and polydispersity index (DdI) of hemagglutinin mRNA LNPs based on lipids 40, 46, SM-102 and ALC-0315 ionizable lipids using 1.5 mole % (or 1.8 mole % for ALC-0315 LNPs) DMG-PEG lipid and stored in 10% trehalose containing buffer. Lipid 40 and 46 LNPs resulted in LNPs of ˜100 nm size and high encapsulation efficiency (defined as the percent of dye accessible RNA in total encapsulated RNA).

TABLE 8
Formulation parameters, physiochemical properties (DLS Size
and Polydispersity), and mRNA encapsulation in HA-mRNA LNPs
based on Ionizable Lipids 40, 46, ALC-0315, and SM-102

| Formulation | Cationic | | Size | | |
|---|---|---|---|---|---|
| Lot # | Lipid (CL) | Final Buffer | (nm) | PdI | EE (%) |
| 1 | Lipid 40 | 10% Trehalose | 102 | 0.15 | 97 |
| 2 | Lipid 46 | 10% Trehalose | 101 | 0.01 | 98 |
| 3 | SM-102 | 10% Trehalose | 111 | 0.14 | 93 |
| 4 | ALC-0315 | 10% Trehalose | 75 | 0.12 | 97 |

### Example 19. Methods of Measurement of In Vitro mCherry Protein Expression in M2 Macrophages

CD14+ Monocytes were isolated from frozen peripheral blood mononuclear cells using a Human Monocyte Isolation Kit (StemCell #100-0697). Monocytes were resuspended after isolation in macrophage base media (50 mL RPMI+10% HI-FBS with 50 ng/mL of human M-CSF) for M0 macrophage differentiation. Monocytes were then plated into 6-well plates at 2 million cells/mL. Cells were allowed to rest for 2 days in a 37° C. incubator, 5% CO₂, and then media was changed. On day 5, media was removed from the wells and 2 mL of cold PBS+10 mM EDTA was added per well. The 6-well plates were incubated for 10 minutes on ice to detach the macrophages. The macrophages were then gently scraped from each well using a silicone scraper and the scraped cells were transferred to a tube with Macrophage base media. The cells were counted and resuspended at 200,000 cells/mL in M2 polarization media (50 mL RPMI+10% HI-FBS with 50 ng/mL human M-CSF and 10 ng/mL human IL-4). The cells were then plated in a flat-bottom 96-well plate at 20,000 or 40,000 cells per well. Cells were incubated in a 37° C. incubator, 5% CO₂ for 24 hours. The next day cells were transfected by gently adding 10 μL of a 11 μg/mL (by mRNA) nanoparticle suspension, resulting in a final mRNA concentration of 1 μg/mL (unless otherwise noted). Cells were gently mixed with a pipette and then incubated for 24 hours in a 37° C. incubator, 5% CO₂. After incubation, the cells were scraped and diluted with FACS buffer (BD Biosciences #554657) and analyzed using either a FACSymphony (BD Biosciences) running BD FACSDiva software or a NovoCyte Penteon (Agilent Technologies) running NovoExpress software. LNP-cell association was evaluated by DiI (APC) expression, and mRNA payload translation was assessed by either mCherry (PE-CF594) or eGFP (FITC) expression. All data collected were analyzed using FlowJo 10.8.2 software and GraphPad Prism version 10.0.

### Example 20. mCherry Protein Expression in M2 Macrophages Using mCherry/DiI-mRNA LNPs Based on Lipid 40 and 1.5 and 3.5 Mol % DSG-PEG Lipid

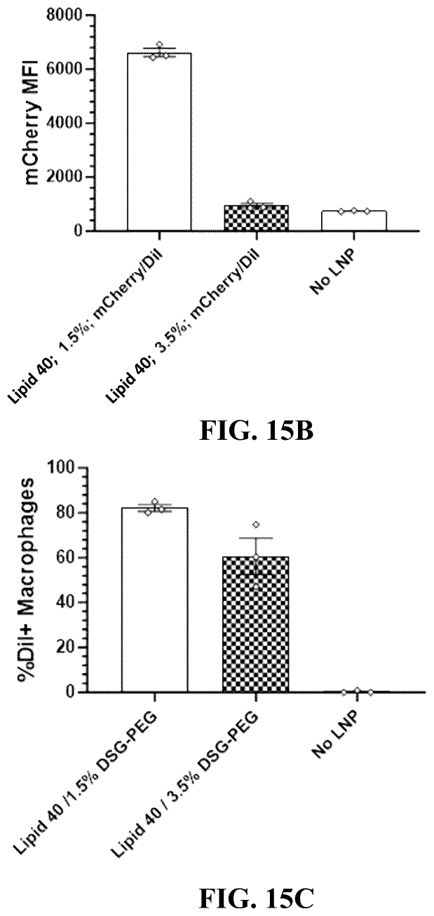

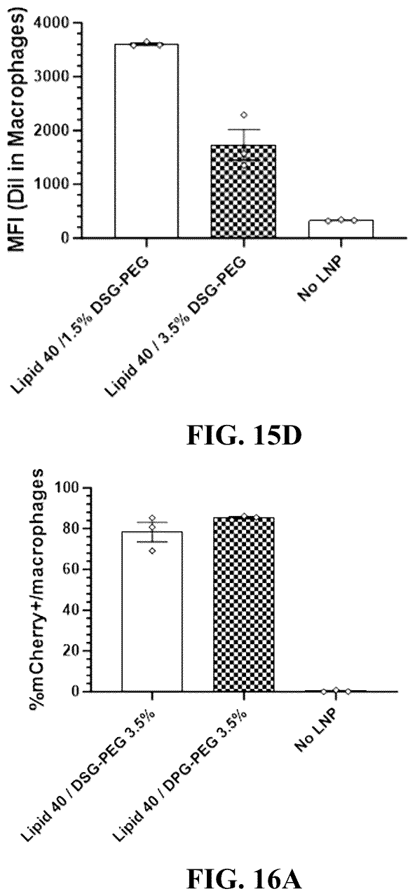

mCherry-mRNA Lipid 40 LNPs were dosed to M2 macrophages (derived from human PBMC Donor #2208430009) at 1 ug/mL (to 40,000 cells/well) and mCherry expression and LNP uptake were determined by FACS analysis at 24 hours using methods described in Example 19 above. mCherry expression was expressed as percent of mCherry+ cells and the mean fluorescence intensity (MFI) of mCherry signal in the entire macrophage cell population. LNP association with macrophages was expressed as the percent of DiI+ cells and MFI of the DiI signal in the entire macrophage cell population. As seen in FIG. 15A and FIG. 15B, a large fraction (˜70%) of macrophages were mCherry positive with the Lipid 40, 1.5 mole % PEG-DSG formulation while low level of mCherry expression (<5% mCherry+, and background-like mCherry MFI values) was observed with the 3.5 mole % DSG-PEG formulation. LNP association at the two PEG-densities, however, was relatively similar (˜60% DiI+ cells with the 3.5 mole % PEG formulation, relative to ˜80% with 1.5 mole % composition) with a roughly 2-fold difference in DiI MFI value (˜1500 a.u. with 3.5% mole % composition versus ˜3500 a.u. with the 1.5 mole % composition). This suggests that high PEG-density LNPs associate with macrophages, however, uptake into macrophages and/or escape from endosomes (or phagosomes) is limited, this limits cytosolic delivery of the mRNA payload and results in low levels of mCherry protein expression. As seen in Example 21 below, uptake into macrophages (and protein expression) is also PBMC donor dependent and this impact of PEG-density on protein expression vary in human PBMC derived M2 macrophages.

### Example 21. mCherry Protein Expression in M2 Macrophages Using mCherry/DiI-mRNA LNPs Based on Lipid 40 and 3.5 Mol % DPG-PEG and 3.5 Mol % DSG-PEG Lipids

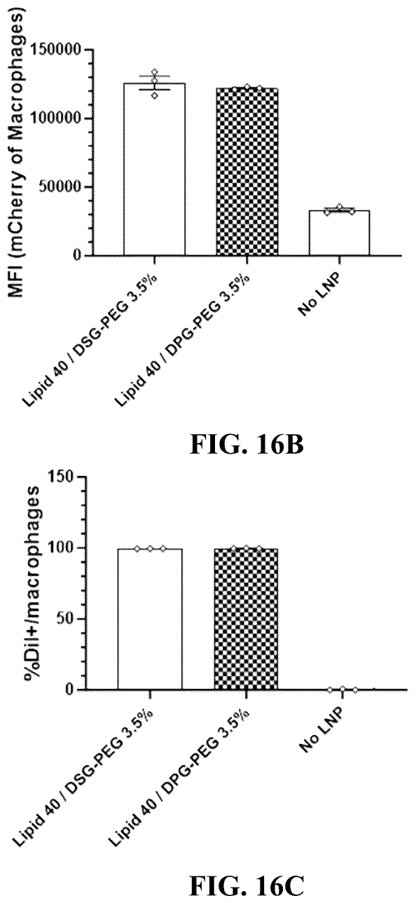

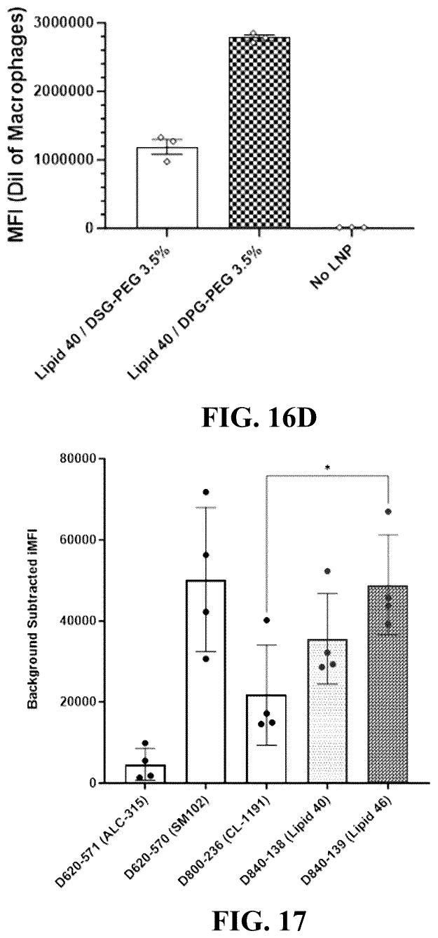

mCherry-mRNA Lipid 40 LNPs were dosed to M2 macrophages (derived from human PBMC Donor #889013852) at 1 ug/mL (to 40,000 cells/well) and mCherry expression and LNP uptake were determined by FACS analysis at 24 hours using methods described in Example 19 above. mCherry expression was expressed as percent of mCherry+ cells and the mean fluorescence intensity (MFI) of mCherry signal in the entire macrophage cell population. While LNP association was expressed as the percent of DiI+ cells and MFI of the DiI signal in the cell population. As seen in FIG. 16A and FIG. 16B, in this PBMC donor (#889013852), Lipid 40 LNPs based on 3.5 mol % DPG-PEG and 3.5 mole % DSG-PEG resulted in strong mCherry expression (>70% mCherry+ macrophages and mCherry MFI ˜4-fold above background levels). Similarly, both lipid anchor compositions resulted in strong LNP association to macrophages. Thus, unlike the result in PBMC donor #2208430009 (of Example 20), here both LNP association (DiI positivity and DiI MFI) and mCherry protein expression are high with the Lipid 40/3.5 mole % DSG-PEG LNP composition. It is also notable that the Di-palmitoyl (DPG) lipid anchor results in 2-fold greater LNP association compared to the Di-stearoyl (DSG) lipid anchor. Examples 28 through 32 report the in vivo protein expression using Lipid 40 LNPs formulated with both DPG and DSG lipid anchors at low (1.5 mole %) and high (3.5 mole %) PEG-lipid density.

### Example 22. Methods of Measurement of In Vivo mCherry Protein Expression in MC-38 Tumor Engrafted C57BL/6 Mice

### Mice Strain

C57BL/6 mice were purchased from The Jackson Laboratory (Strain 000664). 8 week-old naïve female mice were enrolled in the study with an initial body weight ranging from 18.1-22.8 grams.

### Tumor Engraftment, LNP Administration Protocol

The MC38 colorectal tumor cell line was chosen for engraftment due to the abundance of macrophages in MC38 tumors and dependable growth in syngeneic mouse models. MC38 cells were thawed and grown in DMEM+10% FBS until there were sufficient cells for mouse engraftment. Cells were washed and resuspended in a mixture of 50% DPBS and 50% Matrigel, then inoculated subcutaneously at 1 million cells/mouse in the right flank of the mice. Mice were monitored and tumor growth measured via calipers 2-3 days. When tumors reached an average size of 188.6-215.05 mm³, LNPs were administered via intravenous (IV) injection.

Frozen LNPs were thawed 30 minutes prior to dosing and kept on wet ice until administered. LNPs were mixed well immediately prior to dosing to ensure even distribution of particles. At time zero, a single dose was administered to mice in each group (n=4) via IV tail vein injection.

Blood collection was performed to determine lipid pharmacokinetics at 10 minutes, 6 hours and 24 hours post IV injection. At 24 hours post LNP injection, mice were sacrificed via CO₂ inhalation followed by cervical dislocation as a secondary method, and tissues were collected for lipid biodistribution and flow cytometry. Tissue samples destined for lipid biodistribution analysis were snap frozen in liquid nitrogen and stored at −80° C. until further analyses. Tissue samples destined for flow cytometry analysis were kept in RPMI media on wet ice until processing.

TABLE 9
Study design 1

| | # | | | Dosing | Blood collection | Tissue |
|---|---|---|---|---|---|---|
| Group | Mice | LNP | LNP dose | Regimen | timepoints (PK) | collection |
| 1 | 4 | Vehicle | — | Single dose | 10 min; 6 hrs; 24 hrs | 24 hrs |
| 2 | 4 | Lipid 15; 1.5% DSG- | 1.5 mg/kg | Single dose | 10 min; 6 hrs; 24 hrs | 24 hrs |
| | | PEG; mCherry/DiI | | | | |
| 3 | 4 | Lipid 40; 1.5% DSG- | 1.5 mg/kg | Single dose | 10 min; 6 hrs; 24 hrs | 24 hrs |
| | | PEG; mCherry/DiI | | | | |

TABLE 10
Study design 2

| | # | | | Dosing | Blood collection | Tissue |
|---|---|---|---|---|---|---|
| Group | Mice | LNP | LNP dose | Regimen | timepoints (PK) | collection |
| 1 | 4 | Vehicle | — | Single dose | 10 min; 6 hrs; 24 hrs | 24 hrs |
| 2 | 4 | Lipid 40; 3.5% DSG- | 1 mg/kg | Single dose | 10 min; 6 hrs; 24 hrs | 24 hrs |
| | | PEG; mCherry/DiI | | | | |
| 3 | 4 | Lipid 40; 3.5% DPG- | 1 mg/kg | Single dose | 10 min; 6 hrs; 24 hrs | 24 hrs |
| | | PEG; mCherry/DiI | | | | |

### Tissue and Blood Sample Collection and Processing Protocol.

For PK analysis at 10 min and 6 hrs, mice were anesthetized and retro-orbitally bled. 50-100 μL blood was collected in a K₃EDTA mini collection tube (Microvette #20.1341.102). Blood was kept on wet ice until all samples were collected. Plasma from whole blood was separated via centrifugation (1000×g for 10 minutes), and carefully transferred into separate tubes. Plasma was immediately frozen down at −20° C. and kept frozen until future analyses.

Blood and tissue collection. At terminal endpoint 24 hours post dose, mice were sacrificed via CO₂ inhalation, followed by cervical dislocation as a secondary method. 300-500 μL of blood was collected in a K₃EDTA mini collection tube (Microvette #20.1341.102). Blood was kept on wet ice until all samples were collected, and then split in half for flow cytometry and PK analyses. Blood samples destined for PK analysis was separated via centrifugation (1000×g for 10 minutes), and plasma was carefully transferred into separate tubes. Plasma was immediately frozen down at −20° C. and kept frozen until future analyses. Blood destined for flow cytometry was kept on wet ice until processed. Spleen, liver, lungs, leg bones (femur and tibia), and MC38 tumors were harvested and kept in separate RPMI-containing tubes on wet ice until processed.

Blood processing for flow cytometry. Blood was transferred from EDTA-coated tubes into a 2 mL deep-well plate. VersaLyse lysing solution (Beckman Coulter #A09777) was added to blood and incubated for 10 minutes at room temperature. Plates were spun down at 1600 rpm for 4 minutes, then supernatants were carefully aspirated. These steps were repeated twice (three total rounds of VersaLyse), and cell pellets were finally resuspended in FACS buffer (BD Biosciences #554657) and transferred to a 96-well U-bottom plate. Cells were kept on ice until ready for flow staining.

MC38 Tumor processing for flow cytometry. All tumors were kept on ice in RPMI media while awaiting processing. Mouse tumor dissociation kit (Miltenyi #130-096-730) stock reagents were reconstituted and a master mix of tumor dissociation buffer was formulated according to the manufacturer's instructions. 2.5 mL of dissociation buffer was added to gentleMACS c-Tubes (Miltenyi #130-096-334). Individual tumors were roughly chopped with a razor blade and then transferred to the c-Tubes, ensuring all pieces were submerged in the dissociation buffer. The c-Tubes were attached upside-down onto the sleeve of a gentleMACS dissociator and heating sheaths were then fit over the c-Tubes. Protocol 37C_m_TDK_1 was run on the dissociator, according to the manufacturer's instructions. After termination of the program, the dissociated tumor was poured over a 70 μm cell strainer (Corning #352350) into a 50 mL conical tube. Both c-Tube and cell strainer were then rinsed with RPMI media. With a pestle, the remaining tumor lysate was mashed through the cell strainer, followed by an additional rinse with RPMI media. The resulting cell lysate was spun down at 1800 rpm for 4 minutes, and the supernatant was aspirated. For red blood cell lysis, tumor lysates were resuspended in 3 mL of ACK lysis buffer (Gibco #A10492-01) and incubated at room temperature for 5 minutes. 20 mL of RPMI media was added to stop the reaction, and then tubes were spun down and supernatant aspirated. Cells were resuspended in RPMI and counted. 5 million tumor cells were resuspended in FACS staining buffer (BD Biosciences #554657) and transferred to a 96-well U-bottom plate. Cells were kept on ice until ready for flow staining.

Spleen processing for flow cytometry. Spleens were added to a 70 μm cell strainer (Corning #352350) fit onto a 50 mL conical tube and wet with RPMI media. With a pestle, spleens were crushed through the cell strainer and rinsed with cold RPMI media. Cells were spun down at 1800 rpm for 4 minutes, and supernatants were discarded. For red blood cell lysis, the cell pellet was resuspended in 3 mL of ACK lysis buffer (Gibco #A10492-01) and incubated at room temperature for 5 minutes. 20 mL of RPMI media was added to stop the reaction, and then tubes were spun down and supernatant discarded. 2 million cells were resuspended in FACS staining buffer (BD Biosciences #554657) and transferred to a 96-well U-bottom plate. Cells were kept on ice until ready for flow staining.

Liver processing for flow cytometry. All livers were kept on ice in RPMI media while awaiting processing. Mouse liver dissociation kit (Miltenyi #130-105-807) stock reagents were reconstituted and a master mix of liver dissociation buffer was formulated according to the manufacturer's instructions. 5 mL of dissociation buffer was added to gentleMACS c-Tubes (Miltenyi #130-096-334). Individual livers were roughly chopped with a razor blade and then transferred to the c-Tubes, ensuring all pieces were submerged in the dissociation buffer. The c-Tubes were attached upside-down onto the sleeve of a gentleMACS dissociator and heating sheaths were then fit over the c-Tubes. Protocol 37C_m_LIDK_1 was run on the dissociator, according to the manufacturer's instructions. After termination of the program, the dissociated liver lysate was poured over a 70 μm cell strainer (Corning #352350) into a 50 mL conical tube. Both c-Tube and cell strainer were then rinsed with RPMI media. With a pestle, any remaining liver was mashed through the cell strainer, followed by an additional rinse with RPMI media. The resulting cell lysate was spun down at 1800 rpm for 4 minutes, and the supernatant was aspirated. For red blood cell lysis, liver lysates were resuspended in 3 mL of ACK lysis buffer (Gibco #A10492-01) and incubated at room temperature for 5 minutes. 20 mL of RPMI media was added to stop the reaction, and then tubes were spun down and supernatant aspirated. Cells were resuspended in RPMI and counted. 5 million cells were resuspended in FACS staining buffer (BD Biosciences #554657) and transferred to a 96-well U-bottom plate. Cells were kept on ice until ready for flow staining.

Lung processing for flow cytometry. All lungs were kept on ice in RPMI media while awaiting processing. Mouse lung dissociation kit (Miltenyi #130-095-927) stock reagents were reconstituted and a master mix of lung dissociation buffer was formulated according to the manufacturer's instructions. 2.5 mL of dissociation buffer was added to gentleMACS c-Tubes (Miltenyi #130-096-334). Individual lungs were rinsed once in ice cold PBS, roughly chopped with a razor blade and then transferred to the c-Tubes, ensuring all pieces were submerged in the dissociation buffer. The c-Tubes were attached upside-down onto the sleeve of a gentleMACS dissociator and heating sheaths were then fit over the c-Tubes. Protocol 37C_m_LDK_1 was run on the dissociator, according to the manufacturer's instructions. After termination of the program, the dissociated lung lysate was poured over a 70 μm cell strainer (Corning #352350) into a 50 mL conical tube. Both c-Tube and cell strainer were then rinsed with Buffer S from the dissociation kit. With a pestle, any remaining lung was mashed through the cell strainer, followed by an additional rinse with Buffer S. The resulting cell lysate was spun down at 1800 rpm for 4 minutes, and the supernatant was aspirated. For red blood cell lysis, cell pellets were resuspended in 3 mL of ACK lysis buffer (Gibco #A10492-01) and incubated at room temperature for 5 minutes. 20 mL of RPMI media was added to stop the reaction, and then tubes were spun down and supernatant aspirated. The entire cell pellets were resuspended in FACS staining buffer (BD Biosciences #554657) and transferred to a 96-well U-bottom plate. Cells were kept on ice until ready for flow staining.

Bone Marrow processing for flow cytometry. All leg bones were kept on ice while awaiting processing. Eppendorf tube apparatus was prepared by first poking a hole through the bottom of a small 0.6 mL tube with an 18-21 g needle. This was then nestled into a 1.5 mL Eppendorf tube and 100 μL of cold PBS was added to the inner tube. Leg bones were rinsed in ice-cold PBS, and then had one end of each bone cut off with a razor blade. The leg bones were then placed in the nestled inner tube cut side down and the whole Eppendorf apparatus was then centrifuged hard at 14,000 rcf for 30 seconds. The bone marrow collected in the outer Eppendorf tube was resuspended in 1 mL ACK lysis buffer (Gibco #A10492-01) and incubated at room temperature for 5 minutes. The bones remaining in the small inner tube were discarded. After 5 minutes of incubation, 10 mL of RPMI media was added to stop the RBC lysis reaction, and cells were spun down at 1800 rpm for 4 minutes, then the supernatant was discarded. Cells were resuspended in cold FACS buffer (BD Biosciences #554657) and transferred to a 96-well U-bottom plate. Cells were kept on ice until ready for flow staining.

### Immunophenotyping Analysis

Immune cells from blood and organs were prepared for immunophenotyping via flow cytometry by the processes stated above (tissue processing and red blood cell lysis). Cells were washed in PBS then labeled with Fixable Viability Dye eFluor780 (eBiosciences #65-0865-14) for 10 minutes at room temperature. Cells were then Fc-blocked (Biolegend #101320) for 5 minutes at room temperature, followed by surface staining for 30 minutes at room temperature with specific antibodies (clones and dilutions) detailed in Table 11. LNP-cell association was evaluated by DiI expression, and mRNA payload translation was assessed by mCherry expression.

TABLE 11

| | | | Di- | | Catalog |
|---|---|---|---|---|---|
| Antigen | Fluorophore | Clone | lution | Vendor | # |
| DiI | APC | — | N/A | N/A | N/A |
| mCherry | PE-CF594 | — | N/A | N/A | N/A |
| (mRNA) | | | | | |
| CD122 | PE-Cy7 | TM-β1 | 1:100 | Biolegend | 123216 |
| CD3e | BB700 | 145-2C11 | 1:200 | BD Biosciences | 566495 |
| F4/80 | FITC | BM8 | 1:100 | Biolegend | 123108 |
| CD11b | BV786 | M1/70 | 1:100 | Biolegend | 101243 |
| Ly6C | BV711 | HK1.4 | 1:100 | Biolegend | 128037 |
| CD31 | BV605 | 390 | 1:100 | Biolegend | 102427 |
| CD206 | BV421 | C068C2 | 1:100 | Biolegend | 141717 |
| CD45 | BUV805 | 30-F11 | 1:300 | BD Biosciences | 748370 |
| Ly6G | BUV661 | 1A8 | 1:100 | BD Biosciences | 741587 |
| CD19 | BUV395 | 1D3 | 1:300 | BD Biosciences | 565965 |

Compensation for each fluorochrome was performed in the multicolor flow panels using positive and negative compensation beads (Invitrogen #01-2222-42). Fluorescence minus one (FMO) samples and unstained controls were included to determine the level of background fluorescence and to set the gates for the negative cell populations versus the positive cell populations.

All samples were acquired on a FACSymphony (BD Biosciences) running BD FACSDiva software. All data collected were analyzed using FlowJo 10.8.2 software and GraphPad Prism version 10.0.

### Example 23. Methods of Measurement of Lipid 40 Mouse Plasma from mCherry-LNP Dosed MC-38 Tumor Engrafted C57BL/6 Mice

Lipid PK LC-MS method: To understand ionizable lipid (Lipid 40) pharmacokinetics, an LC-MS/MS based bioanalytical method was used. Lipids from mouse plasma samples were extracted into 50/50 Isopropyl alcohol (IPA)/acetonitrile. DOTMA was used as the internal standard to correct for extraction efficiencies. The extracted samples were then chromatographically separated using a phenyl column on Shimadzu UPLC system. A gradient elution using 0.1% formic acid in acetonitrile/IPA (80/20) and 0.1% formic acid in water was used for separation in a 3-minute method. Finally, the separated Lipid 40 was detected at an MRM transition of 1079.8 >737.50 (secondary: 1079.8 >939.3) and DOTMA was detected at a transition of 633.5 >55.1 (secondary 634.5 >69.1) on a Sciex QTRAP 6500 mass spectrometry system. The data was analyzed using Analyst 1.6.2 software.

### Example 24. Methods of Measurement of Hemagglutinin (HA) Protein Expression in Human Dendritic Cells

The method for HA expression splits into two major processes, namely, transfection and staining.

Transfection: Human Dendritic Cells (primary cell line from Stem Cell Technologies—Catalog #200-0370) are thawed the day prior to transfection (Day 0)—in this, DCs are removed from liquid nitrogen storage and placed on dry ice. One at a time, frozen cells are placed in 37° C. water bath until just starting to thaw (˜1 minute). They are then resuspended in warmed cell media, spun at 1500 RPM for 5 minutes at 25° C. post which the supernatant is removed, and the cells are resuspended again in media. This wash is then repeated one more time post which they are incubated overnight at 37° C./5% CO2 in a humidified incubator. The following day (Day 1), formulations are thawed and diluted in relevant diluent to 10× the final concentration before diluting to 1× in cell growth medium. Before transfection, cells are spun at 1500 RPM for 5 minutes at 25° C. post which the supernatant is removed, resuspended and cells are counted with Trypan Blue. Cells are then diluted to 500,000 cells/ml. Final transfection mix and cells are then combined in a 96 well U-bottom plate followed by incubation at 20±0.5 hours at 37° C./5% CO2 in a humidified incubator.

Staining: Following our designated incubation endpoint (Day 2), plate is spun down at 1500 RPM to pellet cells post which the supernatant is discarded. Cells are then washed with cell staining buffer. This step is repeated. Cells are resuspended in an antibody specific for Influenza A HA and then incubated for 1 hour at 4° C. Wash and spin steps from before are repeated. Cells are resuspended in Alexa-647 conjugated secondary antibody and Live-Dead Viability Stain for 1 h at 4° C. Wash and spin steps from before are repeated. Cells can be resuspended in cell staining buffer and run on the flow cytometer immediately or optionally fixed for 10 minutes in 2% PFA in cell staining buffer and run later. Analysis for the images is carried out using FlowJo software by gating for cells á single cells á live cells á HA positive cells from which percent live, percent positive and iMFI is reported as the expression data.

### Example 25. Methods of Measurement of Hemagglutinin (HA) Protein Expression in Human Skeletal Muscle Cells

The method for HA expression splits into two major processes, namely, transfection and immunofluorescence.

Transfection: Human skeletal muscle cells (primary cell line from Lonza Bioscience—CC 2561) are seeded the day prior to transfection (Day 0) post which they are incubated overnight at 37° C./5% C02 in a humidified incubator. The following day (Day 1), formulations are thawed and diluted in relevant diluent to 10× the final concentration before diluting to 1× in cell growth medium. Post preparation of the final dilution mixture of LNP and growth media, old media is aspirated from the plated cells and replaced by the treatment media following with they are allowed to stand for fifteen minutes to avoid edge effects. Cells are then incubated for 20±0.5 hours at 37° C./5% CO2 in a humidified incubator.

Immunofluorescence and imaging: Following our designated incubation endpoint, cells are fixed post PBS wash with cold 4% paraformaldehyde followed by another round of PBS wash. The cells are then subjected to permeabilization using 0.004% Digitonin, followed by PBS washes and then blocked with 10% goat serum for 1 hour at room temperature. Post block, cells are incubated overnight with an antibody specific for Influenza A HA at 4° C. with gentle shaking. The following day (Day 2) cells are washed and incubated with an Alexa-647 conjugated secondary antibody, CellMask Blue, and NucBlue for 1 hour at room temperature. Image acquisition is carried out on Operetta CLS high content imaging microscope using a 20× Water objective. Analysis for the images is carried out using Harmony High Content Imaging and Analysis software that segments cells based on NucBlue/CellMask Blue staining and exports mean fluorescence intensity of the signal per cell which is reported as the expression data.

### Example 26. Hemagglutinin (HA) Protein Expression in Human Dendritic Cells Upon Transfection Using Ha-mRNA LNPs Based on Lipids 40, 46, ALC-0315, SM-102 & CL-1191and 1.5 Mol % (or 1.8 Mol % for ALC-0315 LNPs) DMG-PEG

HA-mRNA LNPs based on Lipid 40, 46, ALC-0315, CL-1191 and SM-102 Ionizable Lipids were dosed to human dendritic cells and HA protein expression levels measured at the 20-hour timepoint using methods described in Example 24 above. As seen in FIG. 17, 1 ug HA-mRNA dosed to 1E6 cells using Lipid 46 LNPs and SM-102 LNPs resulted in high levels of HAI titer values (reflecting high expression of HA protein), while Lipid 40 LNPs resulted in similar levels of HA expression to an internal benchmark lipid CL-1191 LNPs at the same dose level of 1 ug HA-mRNA in 1E6 cells (at the 20-hour timepoint). Thus, Lipid 46 LNPs outperformed other internal lipid compositions tested.

### Example 27. Hemagglutinin (HA) Protein Expression in Human Skeletal Muscle Cells Upon Transfection Using HA-mRNA LNPs Based on Lipids 40, 46 & CL-1191 and 1.5 Mol % DMG-PEG

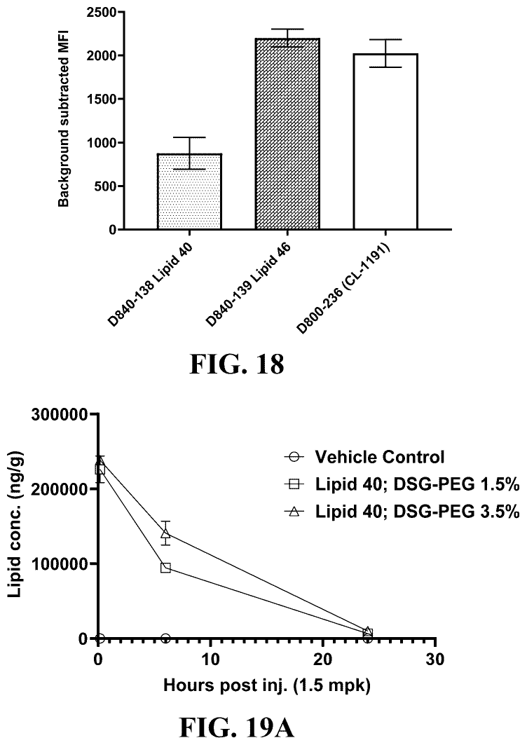

HA-mRNA LNPs based on Lipid 40, 46 and CL-1191 Ionizable Lipids were dosed to human skeletal muscle cells and HA protein expression levels measured at the 20-hour timepoint using methods described in Example 25 above. As seen in FIG. 18, 1 ug HA-mRNA dosed to 1E6 cells using Lipid 46 and CL-1191 LNPs resulted in roughly twice the level of HAI titer values (reflecting high expression of HA protein) relative to Lipid 40 LNPs at the same dose level of 1 ug HA-mRNA in 1E6 cells (at the 20-hour timepoint).

### Example 28. In Vivo Blood and Tissue Distribution of Lipid 40 LNPs in MC-38 Tumor Engrafted C57BL/6 Mice is Dependent on PEG Density

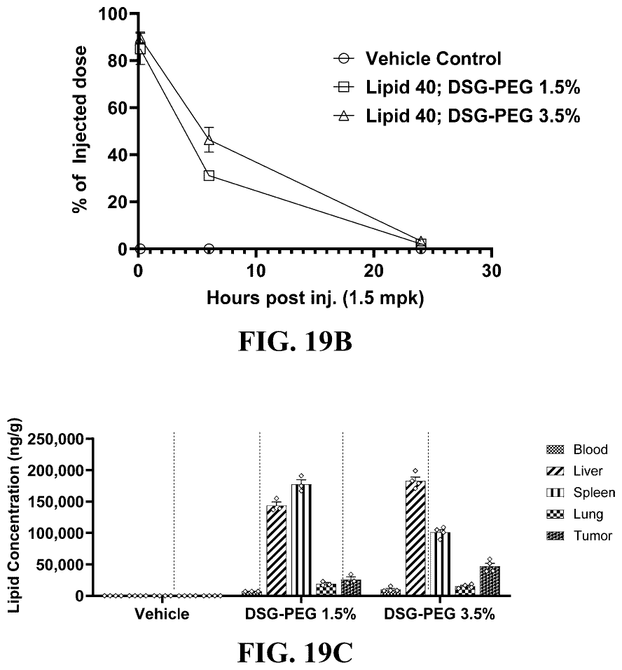

LNPs derived from Lipid 40 ionizable lipid bearing an mCherry mRNA payload formulated using 1.5 or 3.5 mole % DSG-PEG were dosed intravenously (at 1.5 mg/Kg) to C57BL/6 mice engrafted with MC-38 tumor. Ionizable lipid concentration in mouse plasma was monitored by mass spectrometric analysis 10 minutes, 6 and 24 hours post dose. As shown in FIG. 19A and FIG. 19B, a larger fraction (50% of injected dose) of injected dose remained in circulation with the 3.5 mol % DSG-PEG formulation and a lower fraction (30%) remained in circulation with 1.5 mole % DSG-PEG formulation. Additionally, as seen in FIG. 19C, 24 hours post dose, a higher PEG-density of 3.5 mole % results in greater LNP distribution to the liver (relative to the spleen), while the lower PEG-density of 1.5 mole % PEG-lipid resulted in roughly equal amounts of LNP delivery to the liver and spleen. The observed differential distribution may be attributed to the smaller size and/or more neutral charge of the Lipid 40 LNPs at the higher PEG-density of 3.5 mole % PEG-lipid.

### Example 29. In Vivo Blood and Tissue Distribution of Lipid 40 LNPs in MC-38 Tumor Engrafted C57BL/6 Mice is Dependent on Chemistry of Lipid Anchor in PEG-Lipid Conjugate Used in LNP Formulation

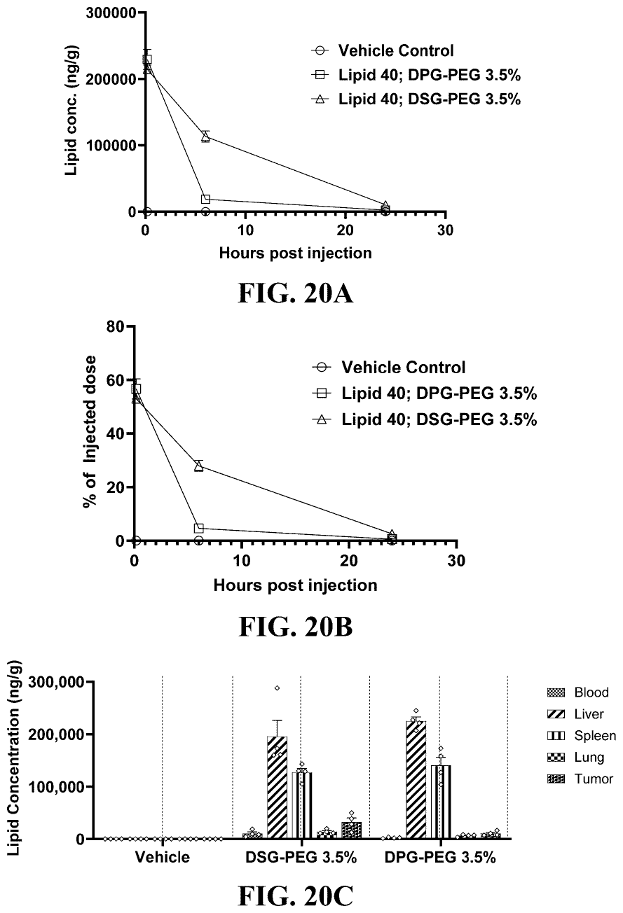

LNPs derived from Lipid 40 ionizable lipid bearing an mCherry mRNA payload formulated using either DSG-PEG (3.5 mole %) or DPG-PEG (3.5 mole %) were dosed intravenously (at 1.0 mg/Kg) to C57BL/6 mice engrafted with MC-38 tumor. Ionizable lipid concentration in mouse plasma was monitored by mass spectrometric analysis at 10 minutes, 6 and 24 hours post dose. As shown in FIG. 20A and FIG. 20B, a larger fraction of injected dose remained in circulation with the distearoyl glycerol PEG conjugate (DSG-PEG). Thus, a higher fraction of LNPs remain in the blood compartment with the DSG-PEG composition (30% of injected dose at the 6-hour time point) versus <10% of injected dose with the more rapidly diffusing DPG-PEG composition. With the more hydrophobic DSG-lipid anchor, diffusion of the PEG-lipid conjugate away from the LNP surface is relatively slow, this provides a relatively stable PEG corona for a longer period of time relative to the less hydrophobic dipalmitoyl glycerol lipid anchor. As expected, the more stable PEG corona enables longer residence time in the blood compartment (possibly due to slower kinetics of association of complement proteins with the LNP surface and therefore delayed detection and clearance by immune cells). Additionally, as seen in FIG. 20C, 24 hours post dose, both DPG and DSG PEG-lipid compositions (at this relative high PEG-density of 3.5 mole %) result in greater LNP distribution to the liver (relative to the spleen). This indicates that irrespective of the rate of PEG-lipid desorption from the LNP surface (and rate of LNP clearance from the blood compartment), smaller LNP size and more neutral LNP surface charge result in favorable LNP uptake by cells in the liver.

### Example 30. In Vivo mCherry Protein Expression Resulting from Lipid 40 LNPs is Dependent on Density of PEG-Lipid Conjugate Used in the LNP Formulation

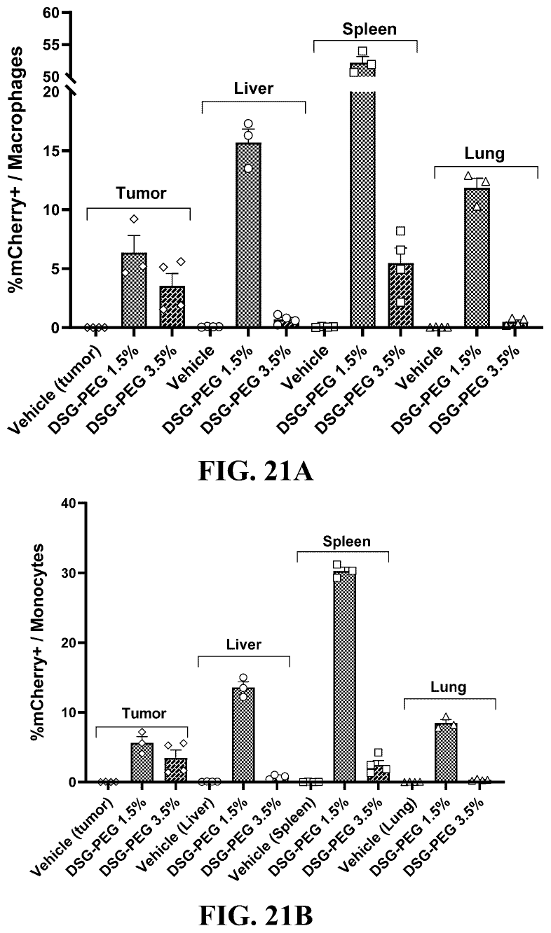

LNPs derived from Lipid 40 ionizable lipid bearing an mCherry mRNA payload formulated using 1.5 or 3.5 mole % DSG-PEG were dosed intravenously (at 1.5 mg/kg) to C57BL/6 mice engrafted with MC-38 tumor and mCherry expression in macrophages and monocytes in liver, spleen, lung, and tumor tissues was determined 24 hours post dose by FACS analysis. As shown in FIG. 21A and FIG. 21B, the 1.5 mol % DSG-PEG LNP formulation resulted in higher mCherry expression in both macrophages (FIG. 21A) and monocytes (FIG. 21B) in all four tissues analyzed (liver, spleen, lung, and tumor) relative to the 3.5 mole % formulation i.e., a higher PEG-density (of 3.5% DSG-PEG) resulted in loss of LNP activity. The 1.5% DSG-PEG formulation resulted in ˜50% mCherry+ macrophages in the spleen, compared to <5% mCherry+ macrophages with the 3.5% DSG-PEG formulation. This loss of LNP activity at 3.5 mole % PEG-lipid composition is potentially due to a combination of inefficient LNP uptake and/or poor cytosolic delivery of the mRNA payload. Overall, a significant fraction of both macrophages and monocytes in spleen, liver and lung tissues were successfully transfected with the 1.5 mole % DSG-PEG composition.

### Example 31. Cell-Type Specificity of Lipid 40/1.5% DSG-PEG mCherry LNPs Protein Expression in Macrophages and Monocytes

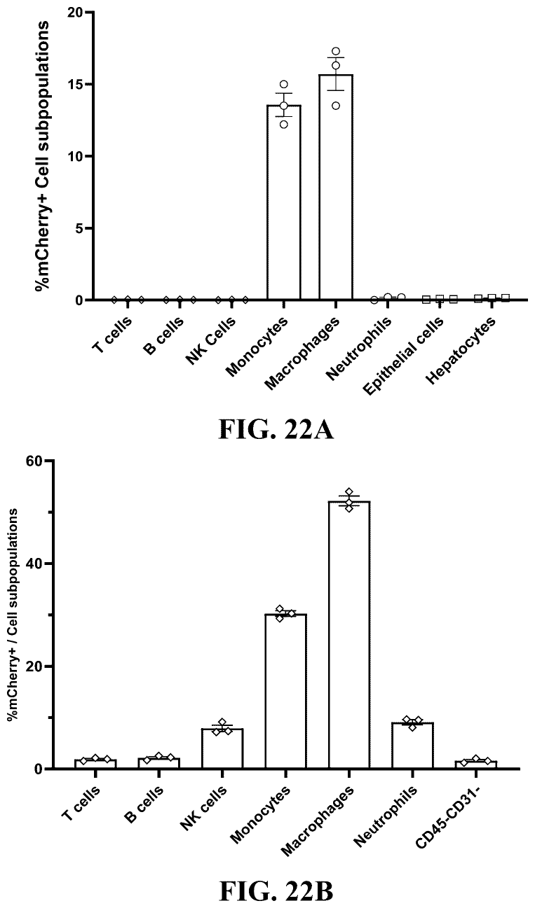

LNPs derived from Lipid 40 ionizable lipid bearing an mCherry mRNA payload formulated using 1.5 mole % DSG-PEG were dosed intravenously (1.5 mg/kg) to C57BL/6 mice engrafted with MC-38 tumor and mCherry expression was determined 24 hours post dose in various relevant cell types in liver and spleen tissues by FACS analysis. As shown in FIG. 22A, the 1.5 mol % DSG-PEG LNP formulation resulted in mCherry expression in macrophages and monocytes (FIG. 22A) in the liver, however, no significant mCherry expression was observed in other immune cells analyzed (T-cells, NK-cells, B-cells, and neutrophils). Similarly, no significant mCherry expression was in the non-immune cells in the liver, i.e., endothelial cells and hepatocytes. As shown in FIG. 22B, the 1.5 mol % DSG-PEG LNP formulation also resulted in mCherry expression in macrophages and monocytes in the spleen, while no significant mCherry expression was observed in other immune cells (T-cells, NK-cells, B-cells, or neutrophils) or non-immune cells (CD45−/CD31−) in the spleen.

### Example 32. In Vivo Mcherry Protein Expression Resulting from Lipid 40 LNPs is Dependent on Chemistry of the Lipid-Anchor in the PEG-Lipid Conjugate

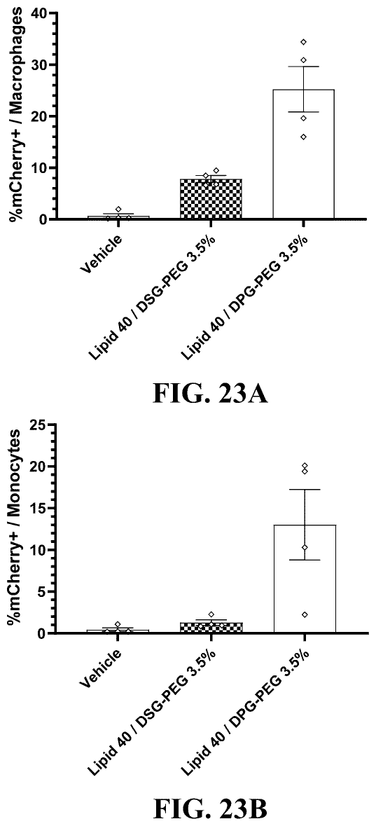

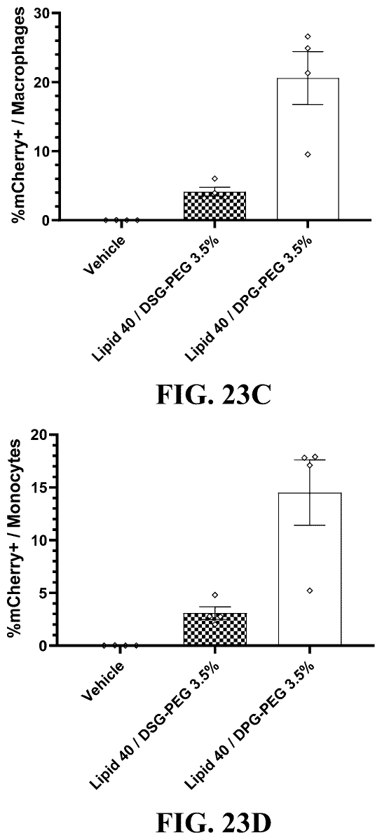

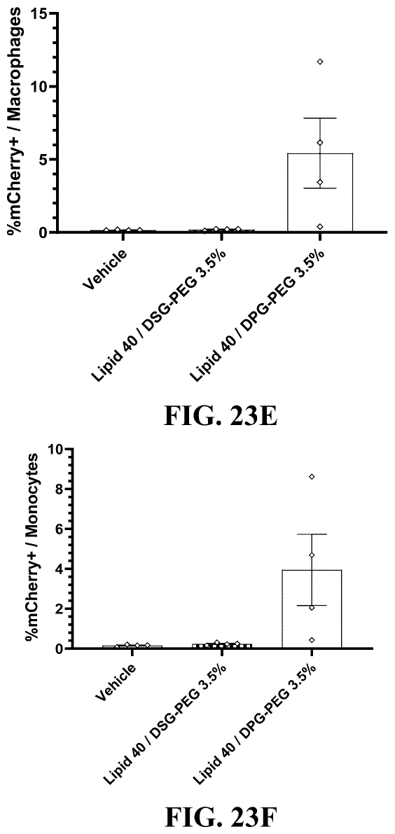

LNPs derived from Lipid 40 ionizable lipid bearing an mCherry mRNA payload formulated using 3.5 mole % DSG-PEG or 3.5 mole % DPG-PEG were dosed intravenously (at 1.5 mg/kg) to C57BL/6 mice engrafted with MC-38 tumor and mCherry expression in macrophages and monocytes in liver, spleen, lung, and tumor tissues was determined 24 hours post dose by FACS analysis. The 3.5 mole % DPG-PEG formulation resulted in good levels of mCherry expression in both macrophages and monocytes relative to the 3.5 mole % DSG-PEG formulation. This trend was observed in the liver (FIG. 23A and FIG. 23B), spleen (FIG. 23C and FIG. 23D), and lung tissues (FIG. 23E and FIG. 23F). Upon i.v. dosing, more efficient diffusion of the less hydrophobic di-palmitoyl lipid anchor of DPG-PEG away from the LNP surface likely results in lower effective PEG-density of LNPs prior to uptake by cell in distal tissues. Thus, consistent with the observation reported in Example 30, this lower effective PEG-density results in more efficient cellular uptake and escape from the endosomes (phagosomes) and thus higher cytosolic availability of the mRNA payload. Thus, DPG-PEG or more rapidly diffusing dimyristoyl lipid anchor of DMG-PEG lipid may enable efficient in vivo protein expression with Lipid 40 LNPs.

### INCORPORATION BY REFERENCE

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, scientific articles, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A compound of Formula (I): 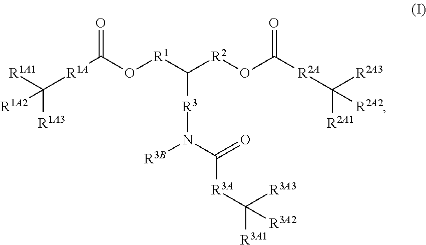
or a salt thereof, wherein:
R¹ and R² are each C₁₋₃ alkylene;
R³ is C₁₋₃ alkylene or a bond;
R^{1A} and R^{2A} are each a bond or C₁₋₁₀ alkylene;
R^{3A} is a bond or C₁₋₃ alkylene;
R^{1A1}, R^{2A1}, R^{3A1}, and R^{3A2} are each H;
R^{1A2} and R^{2A2} are each H, —(CH₂)₀₋₅C(O)OR^{a1}, or —(CH₂)₀₋₅OC(O)R^{a2};
R^{1A3} and R^{2A3} are each H, —(CH₂)₀₋₅C(O)OR^{a1}, or —(CH₂)₀₋₅OC(O)R^{a2};
R^{3A3} is —C(O)OR^{a1};
R^{a1} and R^{a2} are each independently C₁₋₂₀ alkyl;
R^{3B} is 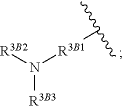
R^{3B1} is C₄₋₆ alkylene; and
R^{3B2} and R^{3B3} are each C₁₋₃ alkyl.

2. The compound of claim 1, or a salt thereof, wherein R¹ and R² are each methylene.

3. The compound of claim 1 or 2, or a salt thereof, wherein R^{1A} and R^{2A} are each a bond, —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, or —(CH₂)₁₀—.

4. The compound of any one of claims 1 to 3, or a salt thereof, wherein R^{3A} is a bond, —CH₂—, or —(CH₂)₂—.

5. The compound of any one of claims 1 to 4, or a salt thereof, wherein R^{1A2} and R^{2A2} are each —OC(O)(C₁₋₁₅ alkyl), —C(O)O(C₁₋₁₅ alkyl), —OC(O)CH(C₁₋₁₀ alkyl)(C₁₋₁₀ alkyl), —C(O)OCH(C₁₋₁₀ alkyl)(C₁₋₁₀ alkyl), —(CH₂)C(O)O(C₁₋₁₀ alkyl), or —(CH₂)OC(O)(C₁₋₁₀ alkyl).

6. The compound of claim 5, or a salt thereof, wherein R^{1A2} and R^{2A2} are each

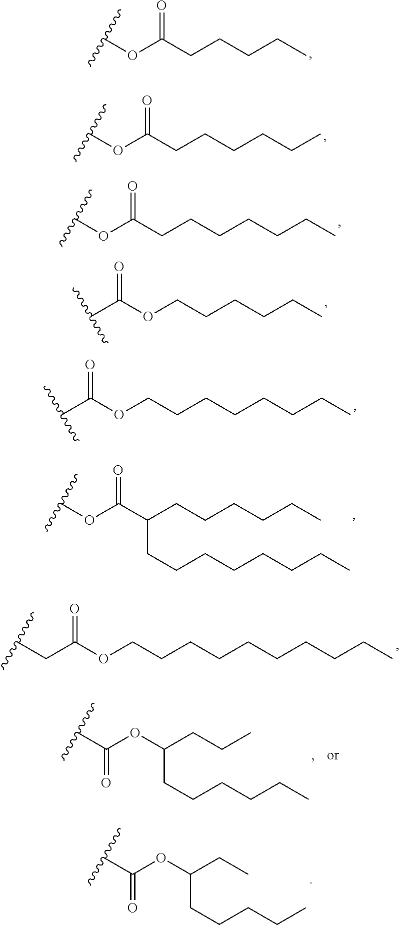

7. The compound of any one of claims 1 to 6, or a salt thereof, wherein R^{1A3} and R^{2A3} are each H, —OC(O)(C₁₋₁₅ alkyl), or —C(O)O(C₁₋₁₅ alkyl).

8. The compound of claim 7, or a salt thereof, wherein R^{1A} and R^{2A3} are each H,

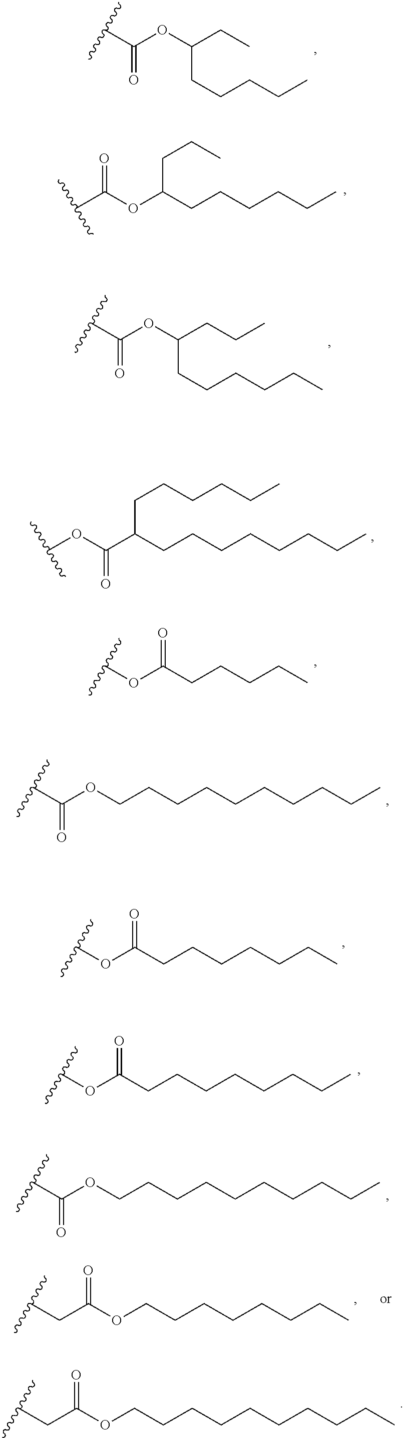

9. The compound of any one of claims 1 to 8, or a salt thereof, wherein R^{3A3} is —C(O)OCH(C₁₋₅ alkyl)(C₁₋₁₀ alkyl).

10. The compound of claim 9, or a salt thereof, wherein R^{3A3} is

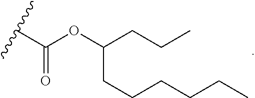 [SMILES: *C(=O)OC(CCC)CCCCCC]

11. The compound of any one of claims 1 to 10, or a salt thereof, wherein R^{3B1} is —(CH₂)₄—, —(CH₂)₅—, or —(CH₂)₆—.

12. The compound of any one of claims 1 to 11, or a salt thereof, wherein R^{3B2} and R^{3B3} are each methyl.

13. The compound of claim 1, or a salt thereof, wherein the compound is selected from Table 1.

14. The compound of claim 1, or a salt thereof, wherein the compound is

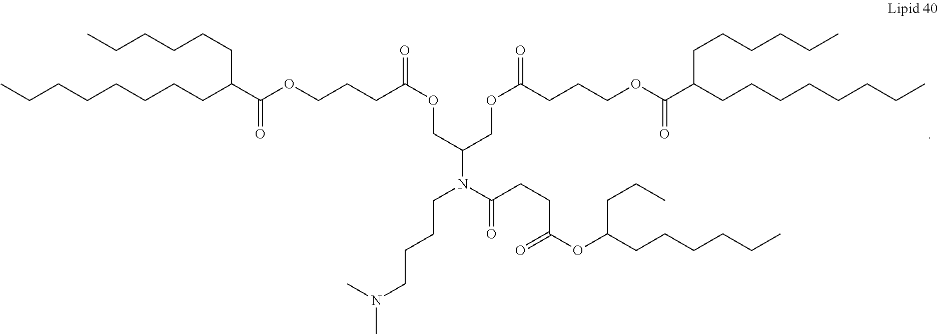 [SMILES: CCCCCCCCC(CCCCCC)C(=O)OCCCC(=O)OCC(COC(=O)CCCOC(=O)C(CCCCCC)CCCCCCCC)N(CCCCN(C)C)C(=O)CCC(=O)OC(CCC)CCCCCC]

15. The compound of claim 1, or a salt thereof, wherein the compound is

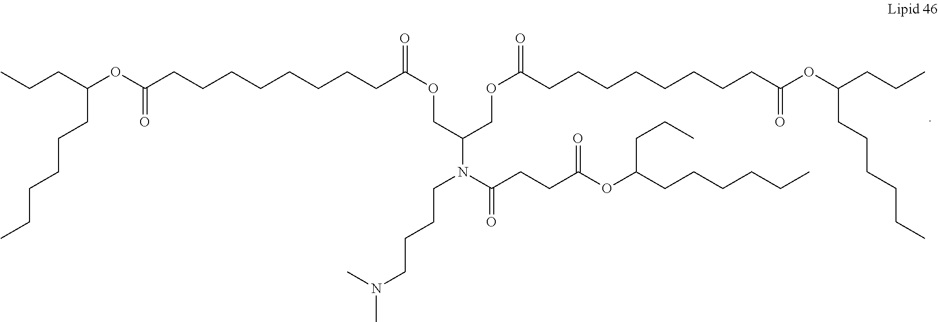 [SMILES: CCCCCCC(CCC)OC(=O)CCCCCCCCC(=O)OCC(COC(=O)CCCCCCCCC(=O)OC(CCC)CCCCCC)N(CCCCN(C)C)C(=O)CCC(=O)OC(CCC)CCCCCC]

16. A lipid nanoparticle (LNP) comprising a lipid blend for targeted delivery of a nucleic acid into an immune cell, the lipid blend comprising:
(a) a lipid-immune cell targeting group conjugate comprising the compound of Formula (II): [Lipid]-[optional linker]-[immune cell targeting group],
(b) an ionizable cationic lipid comprising the compound of any one of claims 1-15, or a salt thereof, and
(c) a nucleic acid, wherein the nucleic acid is encapsulated in the LNP.

17. The LNP of claim 16, wherein the immune cell targeting group comprises an antibody that binds a macrophage antigen, a monocyte antigen, and/or a dendritic antigen.

18. The LNP of claim 17, wherein the macrophage comprises an M1 macrophage, an M2 macrophage, or both.

19. The LNP of claim 17 or 18, wherein the macrophage comprises an M2a macrophage, an M2b macrophage, an M2c macrophage, or any combination thereof.

20. The LNP of claim 17, wherein the macrophage antigen comprises CDIIB, CD68, CD80, CD86, TRL-2, TRL-4, iNOS, MHC-IL, CD163, CD206, CD209, FIZZ1, or Ym1/2, or any combination thereof.

21. The LNP of claim 20, wherein the macrophage antigen comprises CD206.

22. The LNP of any one of claims 16 to 21, wherein the immune cell targeting group is covalently coupled to a lipid in the lipid blend via a polyethylene glycol (PEG) containing linker.

23. The LNP of any one of claims 16 to 22, wherein the lipid blend further comprises one or more of a structural lipid, a neutral phospholipid, and a free PEG-lipid.

24. The LNP of any one of claims 16 to 23, wherein the LNP has a mean diameter in the range of about 50 to about 200 nm.

25. The LNP of any one of claims 16 to 24, wherein the LNP has a polydispersity index in a range from 0.01 to 0.1.

26. The LNP of any one of claims 16 to 25, wherein the LNP has a zeta potential of from about +0 mV to about +10 mV at pH 5.5, or from about −5 mV to about 0 mV at pH 7.4.

27. The LNP of any one of claims 16 to 26, wherein the nucleic acid is DNA or RNA.

28. The LNP of claim 27, wherein the RNA is an mRNA.

29. The LNP of claim 28, wherein the mRNA encodes a receptor, a growth factor, a hormone, a cytokine, an antibody, an antigen, an enzyme, or a vaccine.

30. The LNP of claim 28, wherein the mRNA encodes a polypeptide capable of regulating immune response in the immune cell.

31. The LNP of claim 28, wherein the mRNA encodes a polypeptide capable of reprogramming the immune cell.

32. The LNP of claim 31, wherein the mRNA encodes polypeptide capable of reprogramming an M2 macrophage to an M1 macrophage.

33. The LNP of any one of claims 16 to 32, wherein the immune cell targeting group comprises an antibody, and the antibody is a Fab or an immunoglobulin single variable domain.

34. The LNP of any one of claims 16 to 32, wherein the immune cell targeting group comprises a Fab, F(ab′)2, Fab′-SH, Fv, or scFv fragment.

35. The LNP of any one of claims 16 to 34, wherein the LNP is for delivering a nucleic acid into an immune cell, and wherein the LNP binds a first macrophage antigen, and also binds a second macrophage antigen.

36. The LNP of claim 35, wherein the LNP comprises two conjugates, wherein the first conjugate comprises an antibody that binds the first macrophage antigen, and the second conjugate comprises an antibody that binds the second macrophage antigen.

37. The LNP of claim 35, wherein the LNP comprises one conjugate, and the conjugate comprises a bispecific antibody that binds both the first macrophage antigen and the second macrophage antigen.

38. The LNP of claim 37, wherein the bispecific antibody is an immunoglobulin single variable domain or Fab-ScFv.

39. The LNP of any one of claims 16 to 34, wherein the LNP binds to a first antigen on the surface of the first type of immune cell, and also binds to a second antigen on the surface of the second type of immune cell.

40. The LNP of claim 39, wherein the first type of immune cell is a first macrophage, and the second type of immune cell is a second macrophage, a T-cell, or an NK cell.

41. The LNP of claim 39 or 40, wherein the LNP comprises two conjugates, and the first conjugate comprises a first antibody that binds to the first antigen of the first type of immune cell, and the second conjugate comprises a second antibody that binds to the second antigen of the second type of immune cell.

42. The LNP of claim 39 or 40, wherein the LNP comprises one conjugate, and the conjugate comprises a bispecific antibody, and the bispecific antibody binds to both the first antigen on the first type of immune cell, and the second antigen on the second type of immune cells.

43. The LNP of any one of claims 16 to 34, wherein the bispecific antibody is an immunoglobulin single variable domain or a Fab-ScFv.

44. The LNP of any one of claims 16 to 34, wherein the LNP is for delivering a nucleic acid into an immune cell, and wherein the immune cell targeting group comprises a single antibody that binds to CD206.

45. The LNP of any one of claims 16 to 34, wherein the LNP is for delivering a nucleic acid into both a macrophage and a T-cell or both a macrophage and an NK cell, wherein the immune cell targeting group binds to both (i) CD206 and (ii) one of CD3, CD7, CD8, and CD56.

46. A method of targeting the delivery of a nucleic acid to an immune cell of a subject, comprising contacting the immune cell with the LNP of any one of claims 16 to 45, wherein the LNP comprises the nucleic acid.

47. A method of expressing a polypeptide of interest in a targeted immune cell of a subject, comprising contacting the immune cell with the LNP of any one of claims 16 to 45, wherein the LNP comprises a nucleic acid encoding the polypeptide.

48. A method of modulating cellular function of a target immune cell of a subject, comprising administering to the subject the LNP of any one of claims 16 to 45, wherein the LNP comprises a nucleic acid modulates the cellular function of the immune cell.

49. A method of treating, ameliorating, or preventing a symptom of a disorder or disease in a subject in need thereof, comprising administering to the subject an LNP for delivering a nucleic acid into an immune cell of the subject, wherein the LNP is any one of claims 16 to 45, wherein the LNP comprises the nucleic acid.

50. The method of claim 49, wherein the disorder is an immune disorder, an inflammatory disorder, or cancer.

51. The method of claim 49, wherein the nucleic acid encodes an antigen for use in a therapeutic or prophylactic vaccine for treating or preventing cancer.


SEQUENCE LISTING (170 sequences)

SEQ ID NO: 1 - protein, 234 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 2 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADSS

SEQ ID NO: 3 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAESS

SEQ ID NO: 4 - protein, 229 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 5 - protein, 235 aa, synthetic construct
MDMRVPAQLLGLLLLWLPGAKCDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 6 - protein, 230 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 7 - protein, 213 aa, synthetic construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 8 - protein, 227 aa, synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 9 - protein, 214 aa, synthetic construct
DIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 10 - protein, 231 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 11 - protein, 219 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 12 - protein, 227 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 13 - protein, 214 aa, synthetic construct
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 14 - protein, 226 aa, synthetic construct
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 15 - protein, 214 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 226 aa, synthetic construct
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 17 - protein, 214 aa, synthetic construct
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 18 - protein, 227 aa, synthetic construct
QVQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 19 - protein, 219 aa, synthetic construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 20 - protein, 229 aa, synthetic construct
QVQLQQPGTELVRPGSSVKLSCKASGYTFTSYWVNWVKQRPDQGLEWIGRIDPYDSETHYNQKFTDKAISTIDTSSNTAYMQLSTLTSDASAVYYCSRSPRDSSTNLADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 21 - protein, 214 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 22 - protein, 227 aa, synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGAGAYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 23 - protein, 214 aa, synthetic construct
DIVMTQSPSSLSASVGDRVTITCRTSRSISAALAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 24-25 - skipped (2 entries)

SEQ ID NO: 26 - protein, 248 aa, synthetic construct
QVQLQQSGPGLVKPSQTLSLTCAISGDSVSSNSAAWNWIRQSPSNWIRQSPSGLEWLGRTYYRSKWYNDYAVSVKSRITINPDTSKNQFSLQLNSVTPEDTAVYYCARENIAAWTWAFDIWGQGTMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 27 - protein, 214 aa, synthetic construct
EIVMTQSPGTLSLSPGERATLSCRASQSVSSSYLAWYQQKPGLAPRLLIYDTSLRATDIPDRFSGSGSGTAFTLTISRLEPEDFAVYYCQQYGSSPTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 28 - protein, 238 aa, synthetic construct
EVQLVQSGAEVKKPGSSVKVSCKASGGTFTGYYMHWVRQAPGQGLEWMGWINPNSGGTNYAQKFQGRVTMTRDTSISTAYMELSRLRSDDTAVYYCARDLSSGYSGYFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 29 - protein, 219 aa, synthetic construct
DVVMTQSPLSLPVTPGEPASISCRSSQSLLHSNGYNYLNWYLQKPGQSPQLLIYLGSNRASGVPDRFSGSGSGTDFTLKISRVEGEDVGDYYCMQALQSPFTFGQGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 30 - protein, 238 aa, synthetic construct; identical to SEQ ID NO: 28

SEQ ID NO: 31 - protein, 219 aa, synthetic construct
DVVMTQSPLSLPVTPGEPASISCRSSQSLLHSNGYNFLDWYLQKPGQSPQLLIYLGSNRASGVPDRFSGSGSGTDFTLKISRVEADDVGVYYCMQSLQTPWTFGHGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 32 - protein, 233 aa, synthetic construct
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 33 - protein, 219 aa, synthetic construct
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 34 - protein, 232 aa, synthetic construct
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGEIMDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 35 - protein, 214 aa, synthetic construct
DILLTQSPAILSVSPGERVSFSCRASQRIGTSIHWYQQRTTGSPRLLIKYASESISGIPSRFSGSGSGTDFTLSINSVESEDVADYYCQQSHGWPFTFGGGTKLEIERTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 36 - protein, 233 aa, synthetic construct
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 37 - protein, 214 aa, synthetic construct
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 38 - protein, 234 aa, synthetic construct; identical to SEQ ID NO: 1

SEQ ID NO: 39 - protein, 215 aa, synthetic construct; identical to SEQ ID NO: 3

SEQ ID NO: 40 - protein, 234 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 41 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 42 - protein, 226 aa, synthetic construct
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 43 - protein, 214 aa, synthetic construct
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 227 aa, synthetic construct
QVQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 45 - protein, 219 aa, synthetic construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 240 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 47 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 48 - protein, 234 aa, synthetic construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDNYSLDYWGQGTTLTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 49 - protein, 213 aa, synthetic construct
QIVLTQSPSSLSASLGEKVTMTCRASSSVSYMNWYQQKPGTSPKRWIYDTSKVASGVPDRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 50 - protein, 234 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 51 - protein, 216 aa, synthetic construct
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLGQPKANPTVTLFPPSSEELQANKATLVCLISDFYPGAVTVAWKADGSPVKAGVETTKPSKQSNNKYAACSYLSLTPEQWKSHRSYSCQVTHEGSTVEKTVAPTESS

SEQ ID NO: 52 - protein, 237 aa, synthetic construct
EVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 53 - protein, 214 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 54 - protein, 234 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 55 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 56 - protein, 230 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 57 - protein, 213 aa, synthetic construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 58 - protein, 226 aa, synthetic construct
EVQLVESGGGLVQPGASLKLSCVASGFTFSDYWMSWVRQTPGKPMEWIGHIKYDGSYTNYAPSLKNRFTISRDNAKTTLYLQMSNVRSEDSATYYCAREAPGAASYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 59 - protein, 219 aa, synthetic construct
DVVLTQTPVAQPVTLGDQASISCRSSQSLVHSNGNTYLEWFLQKPGQSPQLLIYKVSNRFSGVPDRFIGSGSGSDFTLKISRVEPEDWGVYYCFQGTHDPYTFGAGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 60 - protein, 229 aa, synthetic construct
EVQLQQSGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 61 - protein, 214 aa, synthetic construct
DIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 62 - protein, 230 aa, synthetic construct
QVQLQQPGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 63 - protein, 219 aa, synthetic construct
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 64 - protein, 236 aa, synthetic construct
EVQLVESGGGLVQPGGSLRLSCAASGYSFTGHWMNWVRQAPGKGLEWVGMIHPSDSETRYNQKFKDRFTISVDKSKNTLYLQMNSLRAEDTAVYYCARGIYFYGTTYFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 65 - protein, 214 aa, synthetic construct
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 66 - protein, 235 aa, synthetic construct
EVQLVESGGDLVQPGRSLRLSCAASGFTFSDYYMSWVRQAPGKGLEWVAAIDNDGGSISYPDTVKGRFTISRDNAKNSLYLQMNSLRVEDTALYYCARQGRLRRDYFDYWGQGTLVTVSTASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 67 - protein, 218 aa, synthetic construct
DIQMTQSPSSLSASVGDRVTITCRASESVDSYGNSFMHWYQQKPGKAPKLLIYRASNLESGVPSRFSGSGSGTDFTFTISSLQPEDIATYYCQQSNEDPLTFGQGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 68 - protein, 239 aa, synthetic construct
EVQLVESGGGLVQPGGSLRLSCATSGYTFTEYTMHWMRQAPGKGLEWVAGINPKNGGTSHNQRFMDRFTISVDKSTSTAYMQMNSLRAEDTAVYYCARWRGLNYGFDVRYFDVWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 69 - protein, 214 aa, synthetic construct
DIQMTQSPSSLSASVGDRVTITCRASQDINNYLNWYQQKPGKAPKLLIYYTSTLHSGVPSRFSGSGSGTDYTLTISSLQPEDFATYYCQQGNTLPPTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 70 - protein, 237 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 71 - protein, 481 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGESGGGGSGGGGSGGGGSQVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSGGGGSGGGGSGGGGSGGGGSDIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 72 - protein, 231 aa, synthetic construct; identical to SEQ ID NO: 10

SEQ ID NO: 73 - protein, 219 aa, synthetic construct; identical to SEQ ID NO: 11

SEQ ID NO: 74 - protein, 235 aa, synthetic construct
EVQLVESGGGLVQPGGSLRLSCAASGFTFSNYAMSWVRQAPGKRLEWVSAISDHSTNTYYPDSVKGRFTISRDNAKNTLYLQMNSLRAEDTAVYYCARKYGGDYDPFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 75 - protein, 214 aa, synthetic construct
DIQMTQSPSSLSASVGDRVTITCQASQDINNYIAWYQHKPGKGPKLLIHYTSTLQPGIPSRFSGSGSGRDYTLTISSLQPEDFATYYCLQYDNLLFTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 76 - protein, 136 aa, synthetic construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTLVTVSSGGCGGHHHHHH

SEQ ID NO: 77 - protein, 132 aa, synthetic construct
EVQLVESGGGLVQAGGSLRLSCAASGSTFSDYGVGWFRQAPGKGREFVADIDWNGEHTSYADSVKGRFATSRDNAKNTAYLQMNSLKPEDTAVYYCAADALPYTVRKYNYWGQGTQVTVSSGGCGGHHHHHH

SEQ ID NO: 78 - protein, 138 aa, synthetic construct
EVQLVESGGGPVQAGGSLRLSCAASGRTYRGYSMGWFRQAPGKEREFVAAIVWSGGNTYYEDSVKGRFTISRDNAKNIMYLQMTSLKPEDSATYYCAAKIRPYIFKIAGQYDYWGQGTLVTVSSAGGGSGGHHHHHHC

SEQ ID NO: 79 - protein, 133 aa, synthetic construct
EVQLVESGGGLVQPGGSLRLSCAASGDIYKSFDMGWYRQAPGKQRDLVAVIGSRGNNRGRTNYADSVKGRFTISRDGTGNTVYLLMNKLRPEDTAIYYCNTAPLVAGRPWGRGTLVTVSSGGGSGGHHHHHHC

SEQ ID NO: 80 - protein, 138 aa, synthetic construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGCHHHHHHHH

SEQ ID NO: 81 - protein, 128 aa, synthetic construct
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGCGGHHHHHH

SEQ ID NO: 82 - protein, 132 aa, synthetic construct
EVQLVESGGGLVQPGGSLRLSCAASGSIFRLHTMEWYRRTPETQREWVATITSGGTTNYPDSVKGRFTISRDDTKKTVYLQMNSLKPEDTAVYYCHAVATEDAGFPPSNYWGQGTLVTVSSGGCGGHHHHHH

SEQ ID NO: 83 - protein, 135 aa, synthetic construct
EVQLVESGGGPVQAGGSLRLSCAASGRTYRGYSMGWFRQAPGREREFVAAIVWSDGNTYYEDSVKGRFTISRDNAKNTMYLQMTSLKPEDSATYYCAAKIRPYIFKIAGQYDYWGQGTLVTVSSGGCGGHHHHHH

SEQ ID NO: 84 - protein, 235 aa, synthetic construct
EVKLVESGGGLVQPGRSLRLSCAASGFNFYAYWMGWVRQAPGKGLEWIGEIKKDGTTINYTPSLKDRFTISRDNAQNTLYLQMTKLGSEDTALYYCAREERDGYFDYWGQGVMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 85 - protein, 215 aa, synthetic construct
QFVLTQPNSVSTNLGSTVKLSCKRSTGNIGSNYVNWYQQHEGRSPTTMIYRDDKRPDGVPDRFSGSIDRSSNSALLTINNVQTEDEADYFCQSYSSGIVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 86 - protein, 229 aa, synthetic construct
EVQLQQSGPELVKPGASVKMSCKASGYTFTSYVIQWVKQKPGQGLEWIGSINPYNDYTKYNEKFKGKATLTSDKSSITAYMEFSLTSEDSALYCARWGDGNYWGRGTLTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 87 - protein, 211 aa, synthetic construct
DIEMTQSPAIMSASLGERVTMTCTASSSVSSSYFHWYQKPGSSPKLCIYSTSNLASGVPPRFSGSGSTSYSLTISMEAEDAATYFCHQYHRSPTFGGGTKLETKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 88 - protein, 238 aa, synthetic construct
QVQLKESGPGLVAPSQSLSITCTVSGFSLTGYGVNWVRQPPGKGLEWLGMIWGDGSTDYNSALKSRLSITKDNSKSQVFLKMNSLQTDDTARYYCARDGYSNFHYYVMDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 89 - protein, 219 aa, synthetic construct
DIVLTQSPASLAVSLGQRATISCRASESVEYYVTSLMQWYQQKPGQPPKLLISAASNVESGVPARFSGSGSGTDFSLNIHPVEEDDIAMYFCQQSRKVPWTFGGGTKLEIKRRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 90 - protein, 237 aa, synthetic construct
QVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 91 - protein, 218 aa, synthetic construct
DIVLTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 92 - protein, 234 aa, synthetic construct
QVQLQESGPGLVKPSETLSLTCAVSGFSLTSYGVHWIRQPGKGLEWLGVIWPGTNFNSALMSRLTISEDTSKNQVSLKLSSVTAADTAVYCARDRAYGNYLYAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 93 - protein, 210 aa, synthetic construct
DIQMTQSPSLSASVGDRVTITCRASESVEYVTSLMQWYQKPGKAPKLLIYAASNVDSGVPSRFSGSGTDFTLTISLQPEDIATYCQSRKVPFTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 94 - protein, 233 aa, synthetic construct
QVQLVQSGAEVKKPGASVKVSCKASGYTFTDYKIHWVRQAPGQGLEWIGYIYPYSGSSDYNQKFKSRATLTVDNSISTAYMELSRLRSDDTAVYYCARGGDAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 95 - protein, 213 aa, synthetic construct
DIQMTQSPSSLSASVGDRVTITCGASENIYGALNWYQRKPGKAPKLLIYGATNLADGVPSRFSGSGSGRDYTLTISSLQPEDFATYFCQNILGTWTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 96 - protein, 234 aa, synthetic construct
EVQLVESGGGLVQPGGSLRLSCAASGFTFNIYYMSWVRQAPGKGLELVAAINPDGGNTYYPDTVKGRFTISRDNAKNSLYLQMNSLRAEDTAVYYCARYGGPGFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 97 - protein, 215 aa, synthetic construct
ENVLTQSPATLSLSPGERATLSCSASSSVSYMHWYQQKPGQAPRLWIYDTSKLASGIPARFSGSGSRNDYTLTISSLEPEDFAVYYCFPGSGFPFMYTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 98 - protein, 262 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSGGGGSGGGGSGGGGSGGGGSDIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKGGGSGGCGGHHHHHH

SEQ ID NO: 99 - protein, 243 aa, synthetic construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 100 - protein, 8 aa, synthetic construct
GSTFSDYG

SEQ ID NO: 101 - protein, 8 aa, synthetic construct
IDWNGEHT

SEQ ID NO: 102 - protein, 14 aa, synthetic construct
AADALPYTVRKYNY

SEQ ID NO: 103 - protein, 4 aa, synthetic construct
KERE

SEQ ID NO: 104 - protein, 4 aa, synthetic construct
KQRE

SEQ ID NO: 105 - protein, 4 aa, synthetic construct
GLEW

SEQ ID NO: 106 - protein, 5 aa, synthetic construct
KEREL

SEQ ID NO: 107 - protein, 5 aa, synthetic construct
KEREF

SEQ ID NO: 108 - protein, 5 aa, synthetic construct
KQREL

SEQ ID NO: 109 - protein, 5 aa, synthetic construct
KQREF

SEQ ID NO: 110 - protein, 5 aa, synthetic construct
KEREG

SEQ ID NO: 111 - protein, 5 aa, synthetic construct
KQREW

SEQ ID NO: 112 - protein, 5 aa, synthetic construct
KQREG

SEQ ID NO: 113 - protein, 4 aa, synthetic construct
TERE

SEQ ID NO: 114 - protein, 5 aa, synthetic construct
TEREL

SEQ ID NO: 115 - protein, 4 aa, synthetic construct
TQRE

SEQ ID NO: 116 - protein, 5 aa, synthetic construct
TQREL

SEQ ID NO: 117 - protein, 4 aa, synthetic construct
KECE

SEQ ID NO: 118 - protein, 5 aa, synthetic construct
KECEL

SEQ ID NO: 119 - protein, 5 aa, synthetic construct
KECER

SEQ ID NO: 120 - protein, 4 aa, synthetic construct
KQCE

SEQ ID NO: 121 - protein, 5 aa, synthetic construct
KQCEL

SEQ ID NO: 122 - protein, 4 aa, synthetic construct
RERE

SEQ ID NO: 123 - protein, 5 aa, synthetic construct
REREG

SEQ ID NO: 124 - protein, 4 aa, synthetic construct
RQRE

SEQ ID NO: 125 - protein, 5 aa, synthetic construct
RQREL

SEQ ID NO: 126 - protein, 5 aa, synthetic construct
RQREF

SEQ ID NO: 127 - protein, 5 aa, synthetic construct
RQREW

SEQ ID NO: 128 - protein, 4 aa, synthetic construct
QERE

SEQ ID NO: 129 - protein, 5 aa, synthetic construct
QEREG

SEQ ID NO: 130 - protein, 4 aa, synthetic construct
QQRE

SEQ ID NO: 131 - protein, 5 aa, synthetic construct
QQREW

SEQ ID NO: 132 - protein, 5 aa, synthetic construct
QQREL

SEQ ID NO: 133 - protein, 5 aa, synthetic construct
QQREF

SEQ ID NO: 134 - protein, 4 aa, synthetic construct
KGRE

SEQ ID NO: 135 - protein, 5 aa, synthetic construct
KGREG

SEQ ID NO: 136 - protein, 4 aa, synthetic construct
KDRE

SEQ ID NO: 137 - protein, 5 aa, synthetic construct
KDREV

SEQ ID NO: 138 - protein, 5 aa, synthetic construct
DECKL

SEQ ID NO: 139 - protein, 5 aa, synthetic construct
NVCEL

SEQ ID NO: 140 - protein, 4 aa, synthetic construct
GVEW

SEQ ID NO: 141 - protein, 4 aa, synthetic construct
EPEW

SEQ ID NO: 142 - protein, 4 aa, synthetic construct
GLER

SEQ ID NO: 143 - protein, 4 aa, synthetic construct
DQEW

SEQ ID NO: 144 - protein, 4 aa, synthetic construct
DLEW

SEQ ID NO: 145 - protein, 4 aa, synthetic construct
GIEW

SEQ ID NO: 146 - protein, 4 aa, synthetic construct
ELEW

SEQ ID NO: 147 - protein, 4 aa, synthetic construct
GPEW

SEQ ID NO: 148 - protein, 4 aa, synthetic construct
EWLP

SEQ ID NO: 149 - protein, 4 aa, synthetic construct
GPER

SEQ ID NO: 150-151 - skipped (2 entries)

SEQ ID NO: 152 - protein, 5 aa, synthetic construct
  VARIANT 1..5: note = May be present in repeats of 1, 2, 3, 4, 5, 6, 7 or more
GGGGS

SEQ ID NO: 153 - skipped

SEQ ID NO: 154 - protein, 5 aa, synthetic construct
GGGGS

SEQ ID NO: 155 - protein, 7 aa, synthetic construct
SGGSGGS

SEQ ID NO: 156 - protein, 8 aa, synthetic construct
GGGGSGGS

SEQ ID NO: 157 - protein, 9 aa, synthetic construct
GGGGSGGGS

SEQ ID NO: 158 - protein, 10 aa, synthetic construct
GGGGSGGGGS

SEQ ID NO: 159 - protein, 15 aa, synthetic construct
GGGGSGGGGSGGGGS

SEQ ID NO: 160 - protein, 18 aa, synthetic construct
GGGGSGGGGSGGGGSGGS

SEQ ID NO: 161 - protein, 20 aa, synthetic construct
GGGGSGGGGSGGGGSGGGGS

SEQ ID NO: 162 - protein, 25 aa, synthetic construct
GGGGSGGGGSGGGGSGGGGSGGGGS

SEQ ID NO: 163 - protein, 30 aa, synthetic construct
GGGGSGGGGSGGGGSGGGGSGGGGSGGGGS

SEQ ID NO: 164 - protein, 35 aa, synthetic construct
GGGGSGGGGSGGGGSGGGGSGGGGSGGGGSGGGGS

SEQ ID NO: 165 - protein, 40 aa, synthetic construct
GGGGSGGGGSGGGGSGGGGSGGGGSGGGGSGGGGSGGGGS

SEQ ID NO: 166 - protein, 15 aa, synthetic construct
EPKSCDKTHTCPPCP

SEQ ID NO: 167 - protein, 24 aa, synthetic construct
GGGGSGGGSEPKSCDKTHTCPPCP

SEQ ID NO: 168 - protein, 12 aa, synthetic construct
EPKTPKPQPAAA

SEQ ID NO: 169 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 170 - protein, 195 aa, synthetic construct
QVQLQESGGGLVQAGGSLRLSCAASGFTDDDYDIGWFRQAPGKEREGVSCISSSDGSTYYADSVKGRFTISSDNAKNTVYLQMNSLKPEDTAVYYCAADFFRWDSGSYYVRGCRHATYDYWGQGTQVTVSSTSFVPVFLPAKPTTTPAPRPPTPAPTIASQPLSLRPEASRPAAGGAVHTRGLDFAGGCHHHHHH